(19) United States
(12) Patent Application Publication
(10) Pub. No.: US 2024/0237528 A1
(43) Pub. Date: Jul. 11, 2024

(54) LIGHT EMITTING ELEMENT AND AMINE COMPOUND FOR THE SAME

(71) Applicant: Samsung Display Co., LTD., Yongin-si (KR)

(72) Inventors: Ichiro IMADA, Yokohama (JP); Taku IMAIZUMI, Yokohama (JP); Ichinori TAKADA, Yokohama (JP)

(21) Appl. No.: 18/472,020

(22) Filed: Sep. 21, 2023

(30) Foreign Application Priority Data
Dec. 19, 2022 (KR) 10-2022-0178244

(51) Int. Cl. H10K 85/60; H10K 50/15; H10K 50/18

(52) U.S. Cl. CPC H10K 85/6576; H10K 50/156; H10K 50/181; H10K 85/615; H10K 85/633; H10K 85/636; H10K 85/6574

(57) ABSTRACT

Provided is a light emitting element including a first electrode, a second electrode on the first electrode, and at least one functional layer between the first electrode and the second electrode. At least one functional layer may include an amine compound represented by Formula 1 below. A light emitting element including the amine compound of an embodiment may have reduced driving voltage and increased lifespan.

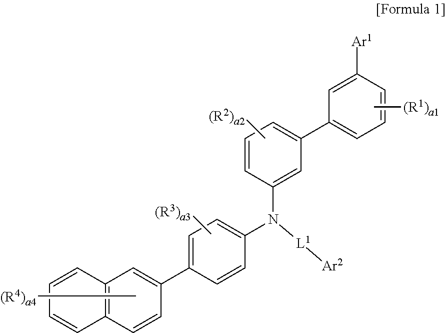

### CROSS-REFERENCE TO RELATED APPLICATIONS

This application claims priority to and the benefit of Korean Patent Application No. 10-2022-0178244, filed on Dec. 19, 2022, the entire contents of which are hereby incorporated by reference.

### BACKGROUND

### 1. Field

Embodiments of the present disclosure herein relate to a light emitting element and an amine compound used therein.

### 2. Description of the Related Art

As image display devices, organic electroluminescence display devices and the like have been actively developed. The organic electroluminescence display devices and the like are display devices including so-called self-luminescent light emitting elements in which holes and electrons injected from a first electrode and a second electrode recombine in an emission layer, and thus a luminescent material in the emission layer emits light to provide a display.

For application of light emitting elements to display devices, there is a demand for greater lifespan, and development of materials, for light emitting elements, capable of stably attaining such characteristics is being substantially continuously conducted.

### SUMMARY

Embodiments of the present disclosure provide a light emitting element having reduced driving voltage and increased lifespan.

Embodiments of the present disclosure also provide an amine compound as a material for a light emitting element, which reduces driving voltage and increases lifespan.

An embodiment of the present disclosure provides a light emitting element including a first electrode, a second electrode on the first electrode, and at least one functional layer between the first electrode and the second electrode and containing an amine compound represented by Formula 1 below.

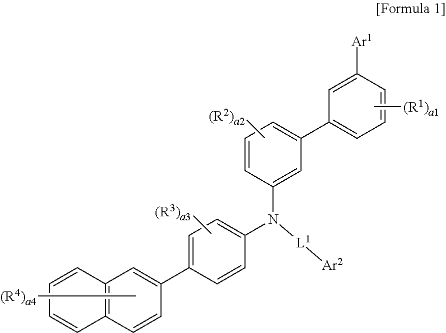

In Formula 1 above, Ar¹ is a substituted or unsubstituted aryl group having 6 to 15 ring-forming carbon atoms, or a substituted or unsubstituted first heteroaryl group having 5 to 30 ring-forming carbon atoms, the first heteroaryl group includes only one hetero atom, and the one hetero atom is an oxygen atom or a sulfur atom, Ar² is a substituted or unsubstituted aryl group having 6 to 30 ring-forming carbon atoms, a substituted or unsubstituted dibenzofuran group, or a substituted or unsubstituted dibenzothiophene group, a1 to a3 are each independently an integer of 0 to 4, a4 is an integer of 0 to 7, R¹, R³, and R⁴ are each independently a hydrogen atom, a deuterium atom, a substituted or unsubstituted alkyl group having 1 to 10 carbon atoms, or a substituted or unsubstituted aryl group having 6 to 30 ring-forming carbon atoms, R² is a hydrogen atom, a deuterium atom, or a substituted or unsubstituted alkyl group having 1 to 10 carbon atoms, L¹ is a direct linkage or a substituted or unsubstituted arylene group having 6 to 30 ring-forming carbon atoms, Ar¹ does not include two or more carbon atoms containing sp3 hybridized orbitals as a ring-forming atom, when a1 to a4 are all 0, Ar² is not a 1-dibenzothiophene group, when a1 to a4 are all 0, L¹-Ar² is not an unsubstituted biphenyl group, and the amine compound does not include a fluorenyl group in which an alkyl group is substituted on the 9th carbon atom of the fluorenyl group.

In an embodiment, in Formula 1 above, Ar¹ and Ar² may each independently be a substituted or unsubstituted phenyl group, a substituted or unsubstituted naphthyl group, a substituted or unsubstituted phenanthryl group, a substituted or unsubstituted dibenzofuran group, or a substituted or unsubstituted dibenzothiophene group.

In an embodiment, in Formula 1, Ar¹ may be represented by any one selected from among AR1-1 to AR1-12 below.

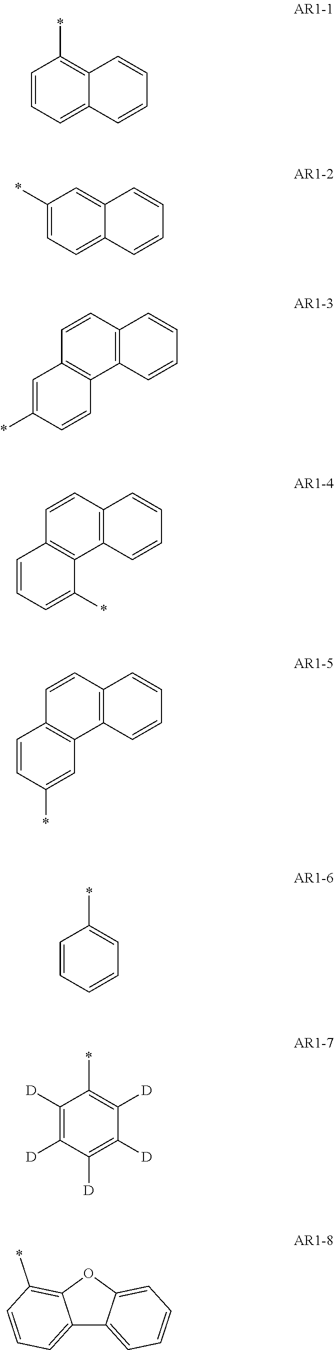

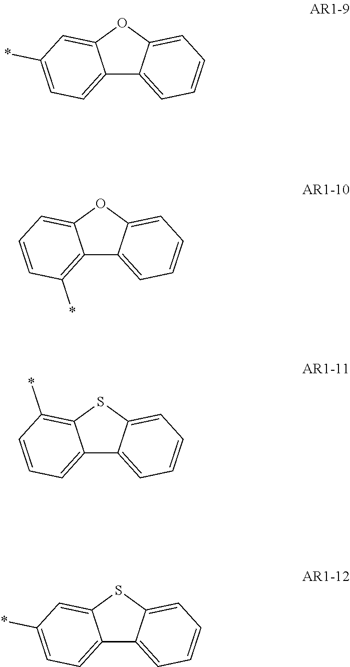 [SMILES: *c1ccc2c(c1)oc1ccccc12 ; *c1cccc2oc3ccccc3c12 ; *c1cccc2c1sc1ccccc12 ; *c1ccc2c(c1)sc1ccccc12]

In AR1-7 above, D is a deuterium atom.

In an embodiment, in Formula 1 above, Ar² may be represented by any one selected from among AR2-1 to AR2-24 below.

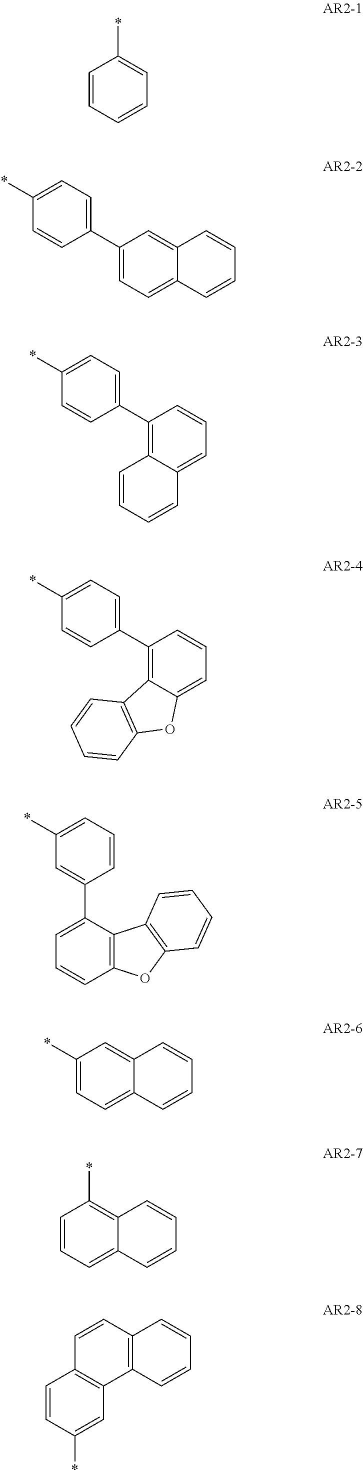

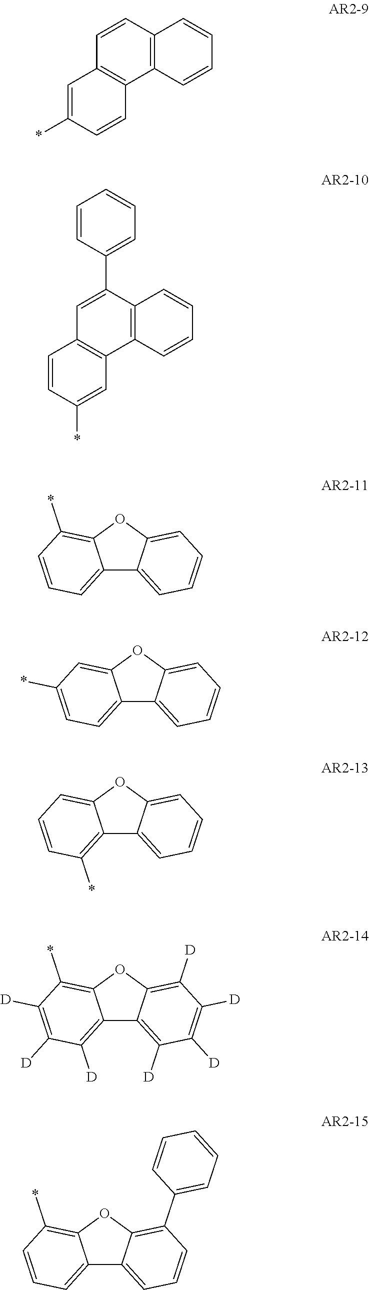

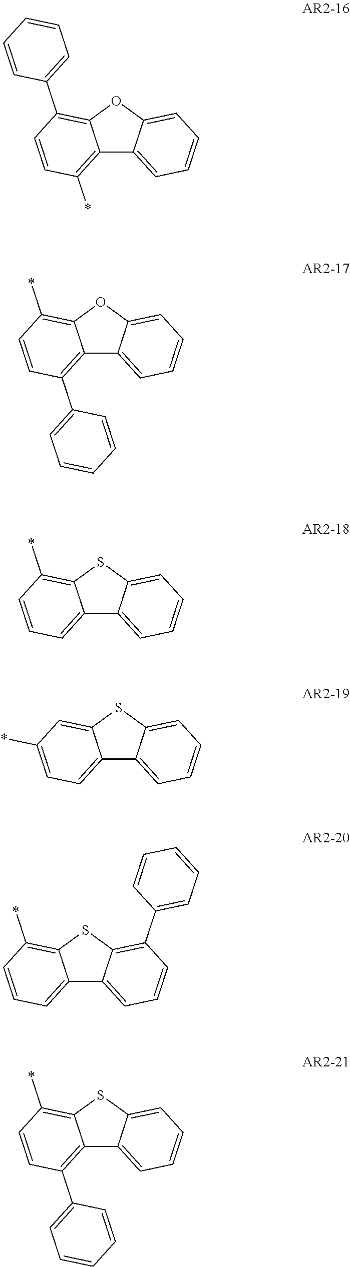 [SMILES: *c1ccc(-c2ccccc2)c2oc3ccccc3c12 ; *c1ccc(-c2ccccc2)c2c1oc1ccccc12 ; *c1cccc2c1sc1ccccc12 ; *c1ccc2c(c1)sc1ccccc12 ; *c1cccc2c1sc1c(-c3ccccc3)cccc12 ; *c1ccc(-c2ccccc2)c2c1sc1ccccc12]

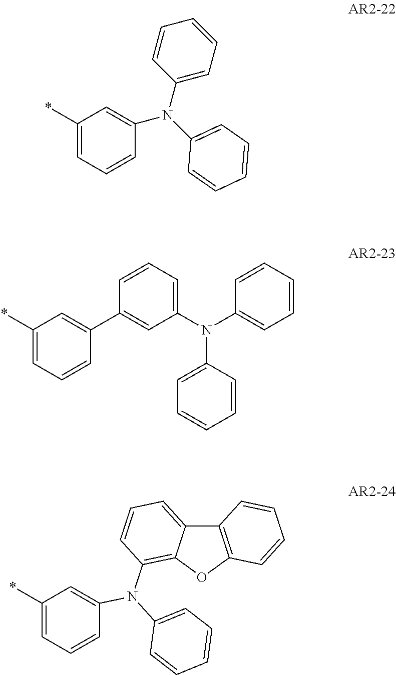 [SMILES: *c1cccc(N(c2ccccc2)c2ccccc2)c1 ; *c1cccc(-c2cccc(N(c3ccccc3)c3ccccc3)c2)c1 ; *c1cccc(N(c2ccccc2)c2cccc3c2oc2ccccc23)c1]

In AR2-14 above, D is a deuterium atom.

In Formula 1 above, L¹ may be a direct linkage, a substituted or unsubstituted phenylene group, or a substituted or unsubstituted divalent phenanthryl group.

In an embodiment, the amine compound represented by Formula 1 above may be represented by any one selected from among Formulas 1-1 to 1-4 below.

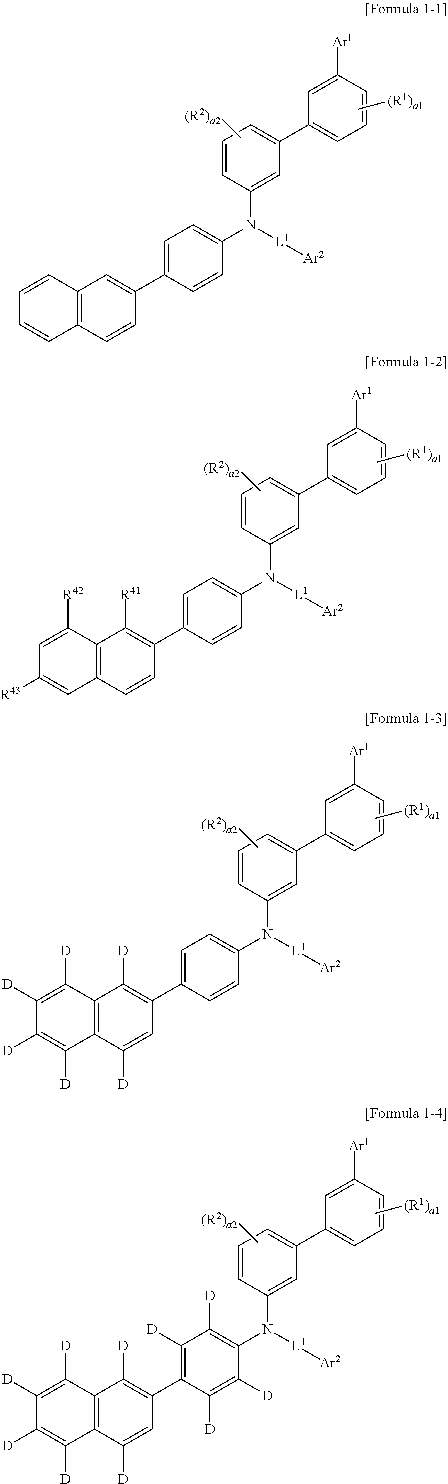

In Formula 1-2 above, at least one selected from R⁴¹ to R⁴³ is an unsubstituted phenyl group, and the others are hydrogen atoms, in Formulas 1-3 and 1-4 above, D is a deuterium atom, and in Formulas 1-1 and 1-4 above, Ar¹, Ar², a1, a2, R¹, R², and L¹ are the same as defined with respect to Formula 1-2 above.

In an embodiment, the amine compound represented by Formula 1-2 above may be represented by any one selected from among Formulas 1-2 Å to 1-2D below.

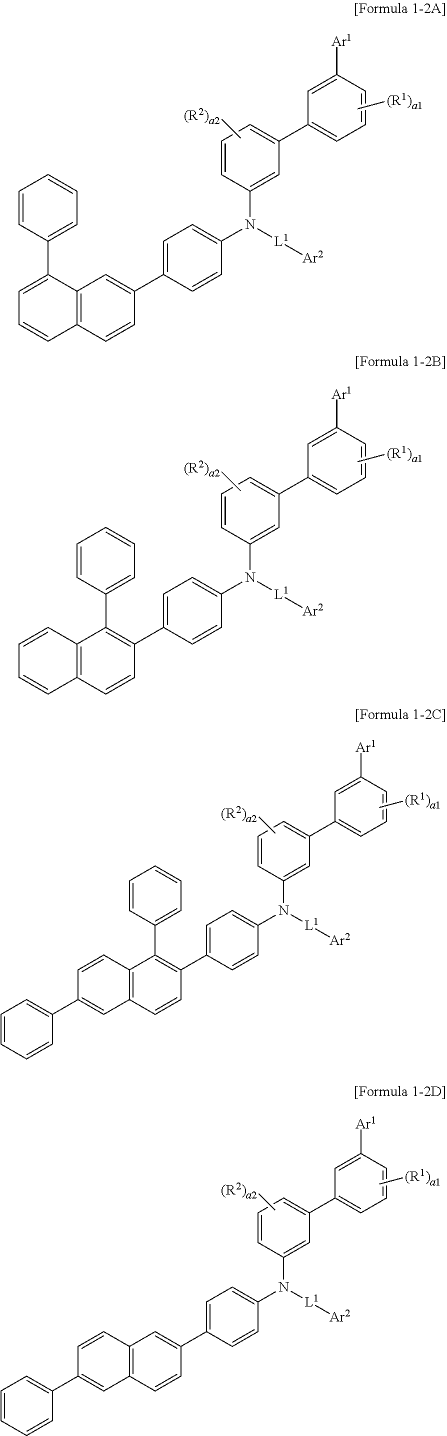

In Formulas 1-2 Å to 1-2D above, Ar1, Ar², a1, a2, R¹, R², and L¹ are the same as defined with respect to Formula 1-2 above.

In an embodiment, the amine compound may be a monoamine compound or a diamine compound.

In an embodiment, the at least one functional layer may include an emission layer, a hole transport region between the first electrode and the emission layer, and an electron transport region between the emission layer, and the hole transport region may include the amine compound.

In an embodiment, the hole transport region may include a hole injection layer on the first electrode, a hole transport layer on the hole injection layer, and an electron blocking layer on the hole transport layer, and at least one selected from the hole injection layer, the hole transport layer, and the electron blocking layer may include the amine compound.

In an embodiment, the hole transport region may include a first hole transport layer and a second hole transport layer, which are sequentially stacked between the first electrode and the emission layer, the first hole transport layer and the second hole transport layer may include different hole transport materials, and the second hole transport layer may include the amine compound.

In an embodiment of the present disclosure, provided is an amine compound represented by Formula 1 above.

### BRIEF DESCRIPTION OF THE DRAWINGS

The accompanying drawings are included to provide a further understanding of the subject matter of the present disclosure, and are incorporated in and constitute a part of this specification. The drawings illustrate embodiments of the present disclosure and, together with the description, serve to explain principles of the present disclosure. In the drawings:

FIG. 1 is a plan view showing a display device according to an embodiment;

FIG. 2 is a cross-sectional view showing a portion corresponding to line I-I′ of FIG. 1;

FIG. 3 is a cross-sectional view schematically showing a light emitting element according to an embodiment;

FIG. 4 is a cross-sectional view schematically showing a light emitting element according to an embodiment;

FIG. 5 is a cross-sectional view schematically showing a light emitting element according to an embodiment;

FIG. 6 is a cross-sectional view schematically showing a light emitting element according to an embodiment;

FIG. 7 is a cross-sectional view schematically showing a light emitting element according to an embodiment;

FIG. 8 is a cross-sectional view showing a display device according to an embodiment;

FIG. 9 is a cross-sectional view showing a display device according to an embodiment;

FIG. 10 is a cross-sectional view showing a display device according to an embodiment;

FIG. 11 is a cross-sectional view showing a display device according to an embodiment; and

FIG. 12 is a view showing a vehicle in which a display device according to an embodiment is provided.

### DETAILED DESCRIPTION

The subject matter of the present disclosure may be modified in many alternate forms, and thus, example embodiments will be illustrated in the drawings and described in the detailed description. It should be understood, however, that it is not intended to limit the present disclosure to the particular forms disclosed, but rather, is intended to cover all modifications, equivalents, and alternatives falling within the spirit and scope of the present disclosure.

As used herein, when an element (or a region, a layer, a portion, and the like) is referred to as being “on,” “connected to,” or “coupled to” another element, it indicates that the element may be directly on/connected to/coupled to the other element, or that a third element may be therebetween.

Like reference numerals refer to like elements. In addition, in the drawings, the thickness, the ratio, and the dimensions of elements may be exaggerated for an effective description of technical contents. The term “and/or,” includes all combinations of one or more of which associated configurations may be defined.

It will be understood that, although the terms first, second, etc. may be used herein to describe various elements, these elements should not be limited by these terms. These terms are only used to distinguish one element from another element. For example, a first element may be referred to as a second element, and similarly, a second element may be referred to as a first element without departing from the spirit or scope of the present disclosure. The singular forms are intended to include the plural forms as well, unless the context clearly indicates otherwise.

In addition, terms such as “below,” “lower,” “above,” “upper,” and the like are used to describe the relationship of the configurations shown in the drawings. The terms are used as a relative concept and are described with reference to the direction indicated in the drawings.

It should be understood that the terms “comprise”, or “have” are intended to specify the presence of stated features, integers, steps, operations, elements, components, or combinations thereof in the disclosure, but do not preclude the presence or addition of one or more other features, integers, steps, operations, elements, components, or combinations thereof.

Unless otherwise defined, all terms (including technical and scientific terms) used herein have the same meaning as commonly understood by one of ordinary skill in the art to which the present disclosure pertains. It is also to be understood that terms defined in commonly used dictionaries should be interpreted as having meanings consistent with the meanings in the context of the related art, and are expressly defined herein unless they are interpreted in an ideal or overly formal sense. Also, terms, such as those defined in commonly used dictionaries, should be interpreted as having a meaning that is consistent with their meaning in the context of the relevant art and should not be interpreted in an idealized or overly formal sense unless expressly so defined herein.

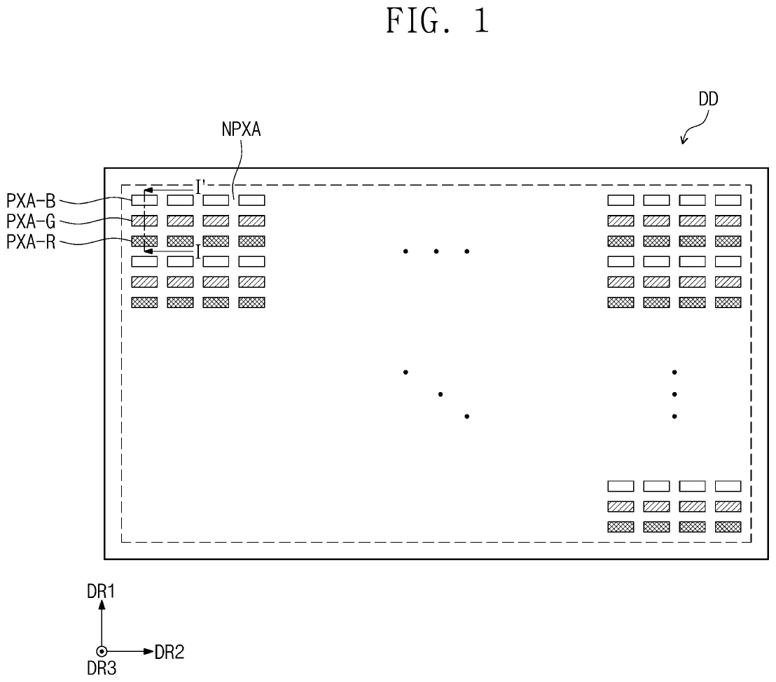

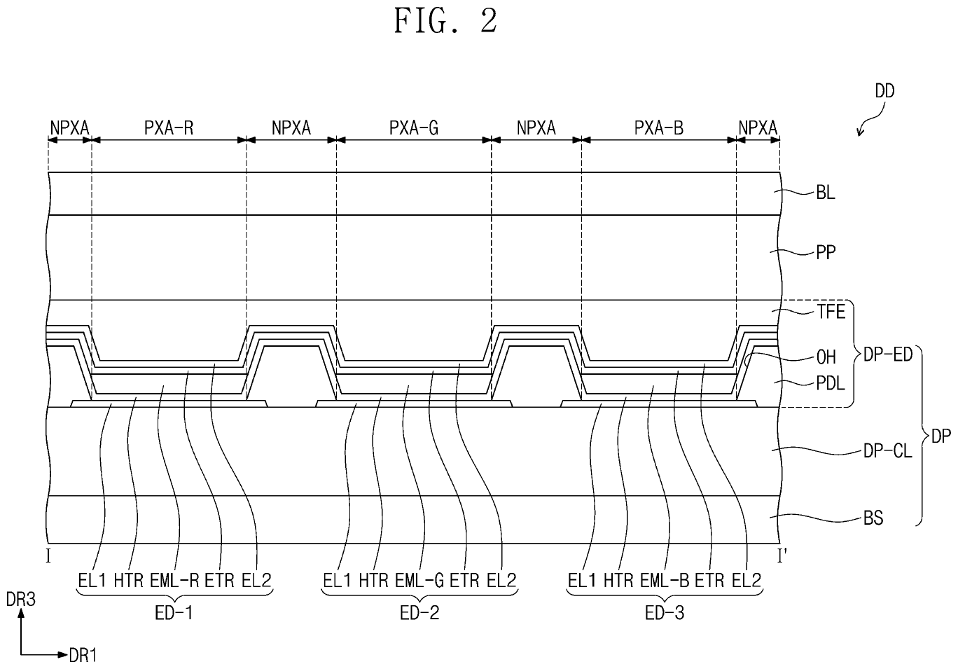

Hereinafter, embodiments of the present disclosure will be described with reference to the accompanying drawings. FIG. 1 is a plan view showing an embodiment of a display device DD. FIG. 2 is a cross-sectional view of a display device DD of an embodiment. FIG. 2 is a cross-sectional view showing a portion corresponding to line I-I′ of FIG. 1.

A display device DD may include a display panel DP and an optical layer PP on the display panel DP. The display panel DP may include light emitting elements ED-1, ED-2, and ED-3. The display device DD may include a plurality of light emitting elements ED-1, ED-2, and ED-3. The optical layer PP may be on the display panel DP to control reflected light in the display panel DP due to external light. The optical layer PP may include, for example, a polarizing layer and/or a color filter layer. In some embodiments, unlike what is shown in the drawings, the optical layer PP may be omitted in the display device DD of an embodiment.

A base substrate BL may be on the optical layer PP. The base substrate BL may be a member providing a base surface on which the optical layer PP is provided. The base substrate BL may be a glass substrate, a metal substrate, a plastic substrate, and/or the like. However, the embodiment of the present disclosure is not limited thereto, and the base substrate BL may be an inorganic layer, an organic layer, or a composite material layer. In some embodiments, unlike what is shown, the base substrate BL may be omitted.

The display device DD according to an embodiment may further include a filling layer. The filling layer may be between a display element layer DP-ED and the base substrate BL. The filling layer may be an organic material layer. The filling layer may include at least one selected from among an acrylic resin, a silicone-based resin, and an epoxy-based resin.

The display panel DP may include a base layer BS, a circuit layer DP-CL provided on the base layer BS, and a display element layer DP-ED. The display element layer DP-ED may include pixel defining films PDL, a plurality of light emitting elements ED-1, ED-2, and ED-3 between the pixel defining films PDL, and an encapsulation layer TFE on the plurality of light emitting elements ED-1, ED-2, and ED-3.

The base layer BS may be a member providing a base surface in which the display element layer DP-ED is provided. The base layer BS may be a glass substrate, a metal substrate, a plastic substrate, and/or the like. However, the embodiment of the present disclosure is not limited thereto, and the base layer BS may be an inorganic layer, an organic layer, or a composite material layer.

In an embodiment, the circuit layer DP-CL may be on the base layer BS, and the circuit layer DP-CL may include a plurality of transistors. The transistors may each include a control electrode, an input electrode, and an output electrode. For example, the circuit layer DP-CL may include a switching transistor and a driving transistor for driving the plurality of light emitting elements ED-1, ED-2 and ED-3 of the display element layer DP-ED.

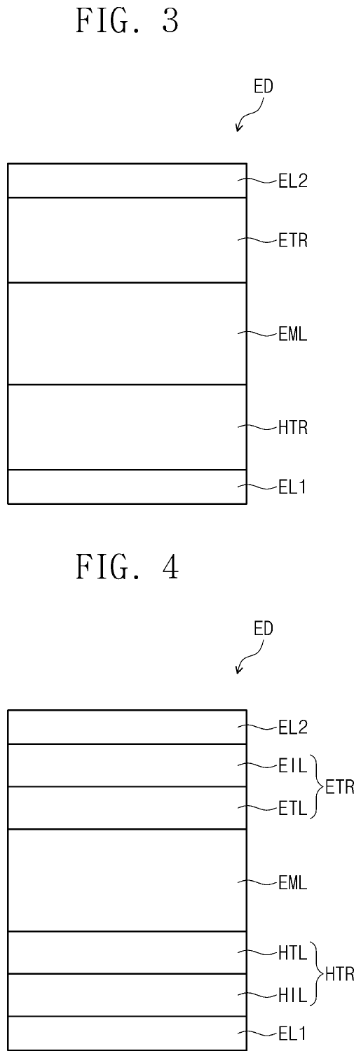

The light emitting elements ED-1, ED-2, and ED-3 may each have a structure of a light emitting element ED according to an embodiment of FIGS. 3-7, which will be described later. The light emitting elements ED-1, ED-2, and ED-3 may each include a first electrode EL1, a hole transport region HTR, emission layers EML-R, EML-G, and EML-B, an electron transport region ETR, and a second electrode EL2.

FIG. 2 shows an embodiment in which the emission layers EML-R, EML-G, and EML-B of the light emitting elements ED-1, ED-2, and ED-3 are in openings OH defined in the pixel defining films PDL, and the hole transport region HTR, the electron transport region ETR, and the second electrode EL2 are provided as a common layer throughout the light emitting elements ED-1, ED-2, and ED-3. However, the embodiment of the present disclosure is not limited thereto, and unlike what is shown in FIG. 2, in an embodiment, the hole transport region HTR and the electron transport region ETR may be provided to be patterned inside the openings OH defined in the pixel defining films PDL. For example, in an embodiment, the hole transport region HTR, the emission layers EML-R, EML-G, and EML-B, and the electron transport region ETR, and the like of the light emitting elements ED-1, ED-2, and ED-3 may be patterned and provided through an inkjet printing method.

The encapsulation layer TFE may cover the light emitting elements ED-1, ED-2 and ED-3. The encapsulation layer TFE may seal the display element layer DP-ED. The encapsulation layer TFE may be a thin film encapsulation layer. The encapsulation layer TFE may be a single layer or a laminated layer of a plurality of layers. The encapsulation layer may include at least one insulating layer. The encapsulation layer TFE according to an embodiment may include at least one inorganic film (hereinafter, an encapsulation inorganic film). In addition, the encapsulation layer TFE according to an embodiment may include at least one organic film (hereinafter, an encapsulation organic film) and at least one encapsulation inorganic film.

The encapsulation inorganic film may protect the display element layer DP-ED from moisture/oxygen, and the encapsulation organic film may protect the display element layer DP-ED from foreign substances such as dust particles. The encapsulation inorganic film may include silicon nitride, silicon oxy nitride, silicon oxide, titanium oxide, aluminum oxide, and/or the like, but is not particularly limited thereto. The encapsulation organic film may include an acrylic compound, an epoxy-based compound, and/or the like. The encapsulation organic film may include a photopolymerizable organic material, and is not particularly limited.

The encapsulation layer TFE may be on the second electrode EL2, and may fill the openings OH.

Referring to FIGS. 1-2, the display device DD may include non-light emitting regions NPXA and light emitting regions PXA-R, PXA-G, and PXA-B. The light emitting regions PXA-R, PXA-G, and PXA-B may each be a region that emits light generated from each of the light emitting elements ED-1, ED-2, and ED-3. The light emitting regions PXA-R, PXA-G, and PXA-B may be spaced apart from each other when viewed on a plane.

The light emitting regions PXA-R, PXA-G, and PXA-B may each be a region separated by the pixel defining films PDL. The non-light emitting regions NPXA may be regions between neighboring light emitting regions PXA-R, PXA-G, and PXA-B, and may correspond to the pixel defining film PDL. In some embodiments, as described herein, the light emitting regions PXA-R, PXA-G, and PXA-B may each correspond to a pixel. The pixel defining film PDL may separate the light emitting elements ED-1, ED-2 and ED-3. The emission layers EML-R, EML-G, and EML-B of the light emitting elements ED-1, ED-2 and ED-3 may be provided and separated in openings OH defined by the pixel defining films PDL.

The light emitting regions PXA-R, PXA-G, and PXA-B may be divided into a plurality of groups according to the color of light generated from the light emitting elements ED-1, ED-2, and ED-3. In the display device DD of an embodiment shown in FIGS. 1-2, three light emitting regions PXA-R, PXA-G, and PXA-B which emit red light, green light, and blue light, respectively, are shown as an example. For example, the display device DD of an embodiment may include a red light emitting region PXA-R, a green light emitting region PXA-G, and a blue light emitting region PXA-B, which are distinct from one another.

In the display device DD according to an embodiment, the plurality of light emitting elements ED-1, ED-2, and ED-3 may emit light having different wavelength ranges. For example, in an embodiment, the display device DD may include a first light emitting element ED-1 that emits red light, a second light emitting element ED-2 that emits green light, and a third light emitting element ED-3 that emits blue light. In some embodiments, the red light emitting region PXA-R, the green light emitting region PXA-G, and the blue light emitting region PXA-B of the display device DD may correspond to the first light emitting element ED-1, the second light emitting element ED-2, and the third light emitting element ED-3, respectively.

However, the embodiment of the present disclosure is not limited thereto, and the first to third light emitting elements ED-1, ED-2 and ED-3 may emit light in the same wavelength range or emit light in at least one different wavelength range. For example, the first to third light emitting elements ED-1, ED-2, and ED-3 all may emit blue light.

The light emitting regions PXA-R, PXA-G, and PXA-B in the display device DD according to an embodiment may be arranged in the form of a stripe. Referring to FIG. 1, a plurality of red light emitting regions PXA-R, a plurality of green light emitting regions PXA-G, and a plurality of blue light emitting regions PXA-B may each be arranged along a second directional axis DR2. In some embodiments, the red light emitting region PXA-R, the green light emitting region PXA-G, and the blue light emitting region PXA-B may be alternately arranged in turn along a first directional axis DR1.

FIGS. 1-2 show that the light emitting regions PXA-R, PXA-G, and PXA-B are all similar in size, but the embodiment of the present disclosure is not limited thereto, and the light emitting regions PXA-R, PXA-G and PXA-B may be different in size from each other according to wavelength range of emitted light. In some embodiments, the areas of the light emitting regions PXA-R, PXA-G, and PXA-B may refer to areas when viewed on a plane defined by the first directional axis DR1 and the second directional axis DR2.

In some embodiments, the arrangement of the light emitting regions PXA-R, PXA-G, and PXA-B is not limited to what is shown in FIG. 1, and the order in which the red light emitting region PXA-R, the green light emitting region PXA-G, and the blue light emitting region PXA-B are arranged may be suitably varied according to display quality characteristics desired or required for the display device DD. For example, the light emitting regions PXA-R, PXA-G, and PXA-B may be arranged in the form of a PENTILE® arrangement structure (e.g., an RGBG matrix, RGBG structure, or RGBG matrix structure) or a DIAMOND PIXEL™ structure. PENTILE® is a duly registered trademark of Samsung Display Co., Ltd. and DIAMOND PIXEL™ is a trademark of Samsung Display Co., Ltd.

In addition, areas of each of the light emitting regions PXA-R, PXA-G, and PXA-B may be different in size from one another. For example, in an embodiment, the green light emitting region PXA-G may be smaller than the blue light emitting region PXA-B in size, but the embodiment of the present disclosure is not limited thereto.

Hereinafter, FIGS. 3-7 are cross-sectional views schematically showing a light emitting element according to an embodiment. The light emitting element ED according to an embodiment may include a first electrode EL1, a hole transport region HTR, an emission layer EML, an electron transport region ETR, and a second electrode EL2.

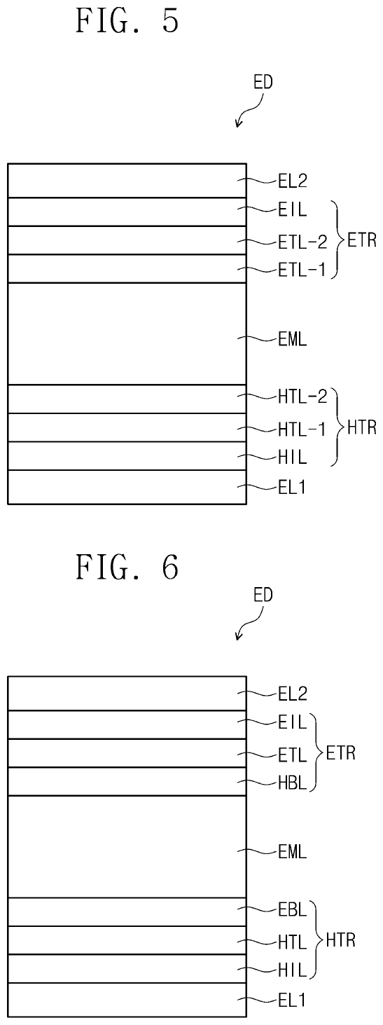

FIG. 4 shows, compared with FIG. 3, a cross-sectional view of a light emitting element ED of an embodiment in which the hole transport region HTR includes a hole injection layer HIL and a hole transport layer HTL, and the electron transport region ETR includes an electron injection layer EIL and an electron transport layer ETL. FIG. 5 shows, compared with FIG. 3, a cross-sectional view of a light emitting element ED of an embodiment in which the hole transport region HTR includes a hole injection layer HIL, a first hole transport layer HTL-1, and a second hole transport layer HTL-2, and the electron transport region ETR includes an electron injection layer EIL, a second electron transport layer ETL-2, and a first electron transport layer ETL-1. In some embodiments, unlike what is shown, any one selected from the first hole transport layer HTL-1 and the second hole transport layer HTL-2 may be omitted. Any one selected from the first electron transport layer ETL-1 and the second electron transport layer ETL-2 may be omitted.

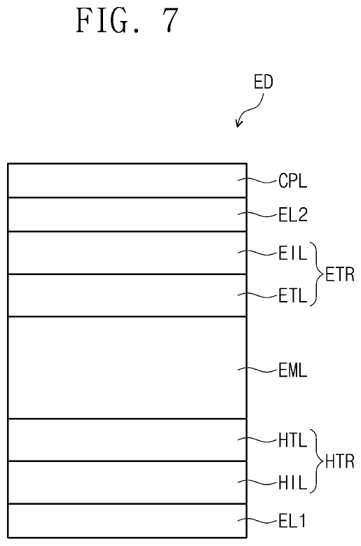

FIG. 6 shows, compared with FIG. 3, a cross-sectional view of a light emitting element ED according to an embodiment in which the hole transport region HTR includes a hole injection layer HIL, a hole transport layer HTL, and an electron blocking layer EBL, and the electron transport region ETR includes an electron injection layer EIL, an electron transport layer ETL, and a hole blocking layer HBL. FIG. 7 shows, compared with FIG. 4, a cross-sectional view of a light emitting element ED according to an embodiment, in which a capping layer CPL on the second electrode EL2 is provided.

The light emitting element ED of an embodiment may include the amine compound of an embodiment in at least one functional layer between a first electrode and a second electrode. At least one functional layer may include a hole transport region HTR, an emission layer EML, and an electron transport region ETR. For example, the hole transport region HTR may include an amine compound of an embodiment. At least one selected from the hole injection layer HIL, the hole transport layer HTL, and the electron blocking layer EBL of the hole transport region HTR may include the amine compound of an embodiment. In some embodiments, of the first and second hole transport layers HTL-1 and HTL-2 of the hole transport region HTR, the second hole transport layer HTL-2 adjacent to the emission layer EML may include the amine compound of an embodiment. The first hole transport layer HTL-1 and the second hole transport layer HTL-2 of the hole transport region HTR may include different hole transport materials.

The amine compound of an embodiment may be a monoamine compound or a diamine compound. The amine compound of an embodiment may include one or two amine groups. The amine compound of an embodiment may include first to third ring groups bonded to an amine group, and the first ring group may be a meta-meta biphenyl group. The second ring group is a phenyl group substituted with a naphthyl group, and the phenyl group may be directly bonded to nitrogen atoms of an amine group. The third ring group may be an aryl group, a dibenzofuran group, or a dibenzothiophene group. Any one selected from the first to third ring groups may include an amine group as a substituent. The light emitting element ED including the amine compound of an embodiment may have reduced driving voltage and increased lifespan.

As used herein, the term “substituted or unsubstituted” may indicate that one is substituted or unsubstituted with at least one substituent selected from the group consisting of a deuterium atom, a halogen atom, a cyano group, a nitro group, an amine group, a silyl group, oxy group, thio group, sulfinyl group, sulfonyl group, carbonyl group, a boron group, a phosphine oxide group, a phosphine sulfide group, an alkyl group, an alkenyl group, an alkynyl group, a hydrocarbon ring group, an aryl group, and a heterocyclic group. In addition, each of the substituents presented as an example above may be substituted or unsubstituted. For example, a biphenyl group may be interpreted as an aryl group or as a phenyl group substituted with a phenyl group.

As used herein, the term “bonded to an adjacent group to form a ring” may indicate that one is bonded to an adjacent group to form a substituted or unsubstituted hydrocarbon ring, or a substituted or unsubstituted heterocycle. The hydrocarbon ring includes an aliphatic hydrocarbon ring and an aromatic hydrocarbon ring. The heterocycle includes an aliphatic heterocycle and an aromatic heterocycle. The hydrocarbon ring and the heterocycle may be monocyclic or polycyclic. In addition, the rings formed by being bonded to each other may be connected to another ring to form a spiro structure.

As used herein, the term “adjacent group” may refer to a substituent substituted for an atom which is directly connected to an atom substituted with a corresponding substituent, another substituent substituted for an atom which is substituted with a corresponding substituent, or a substituent sterically positioned at the nearest position to a corresponding substituent. For example, two methyl groups in 1,2-dimethylbenzene may be interpreted as mutually “adjacent groups” and two ethyl groups in 1,1-diethylcyclopentane may be interpreted as mutually “adjacent groups”. In addition, two methyl groups in 4,5-dimethylphenanthrene may be interpreted as mutually “adjacent groups”.

As used herein, examples of a halogen atom may include a fluorine atom, a chlorine atom, a bromine atom, and an iodine atom.

As used herein, an alkyl group may be a linear, branched or cyclic type (or kind). The number of carbon atoms in the alkyl group is 1 to 60, 1 to 50, 1 to 30, 1 to 20, 1 to 10 or 1 to 6. Examples of the alkyl group may include a methyl group, an ethyl group, an n-propyl group, an isopropyl group, an n-butyl group, a s-butyl group, a t-butyl group, an i-butyl group, a 2-ethylbutyl group, a 3,3-a dimethylbutyl group, an n-pentyl group, an i-pentyl group, a neopentyl group, a t-pentyl group, a cyclopentyl group, a 1-methylpentyl group, a 3-methylpentyl group, a 2-ethylpentyl group, a 4-methyl-2-pentyl group, an n-hexyl group, a 1-methylhexyl group, a 2-ethylhexyl group, a 2-butylhexyl group, a cyclohexyl group, a 4-methylcyclohexyl group, a 4-t-butylcyclohexyl group, an n-heptyl group, a 1-methylheptyl group, a 2,2-dimethylheptyl group, a 2-ethylheptyl group, a 2-butylheptyl group, an n-octyl group, a t-octyl group, a 2-ethyloctyl group, a 2-butyloctyl group, a 2-hexyloctyl group, a 3,7-dimethyloctyl group, a cyclooctyl group, an n-nonyl group, an n-decyl group, an adamantyl group, a 2-ethyldecyl group, a 2-butyldecyl group, a 2-hexyldecyl group, a 2-octyldecyl group, an n-undecyl group, an n-dodecyl group, a 2-ethyldodecyl group, a 2-butyldodecyl group, a 2-hexyldocecyl group, a 2-octyldodecyl group, an n-tridecyl group, an n-tetradecyl group, an n-pentadecyl group, an n-hexadecyl group, a 2-ethylhexadecyl group, a 2-butylhexadecyl group, a 2-hexylhexadecyl group, a 2-octylhexadecyl group, an n-heptadecyl group, an n-octadecyl group, an n-nonadecyl group, an n-eicosyl group, a 2-ethyleicosyl group, a 2-butyleicosyl group, a 2-hexyleicosyl group, a 2-octyleicosyl group, an n-henicosyl group, an n-docosyl group, an n-tricosyl group, an n-tetracosyl group, an n-pentacosyl group, an n-hexacosyl group, an n-heptacosyl group, an n-octacosyl group, an n-nonacosyl group, an n-triacontyl group, and the like, but are not limited thereto.

As used herein, an alkenyl group refers to a hydrocarbon group including at least one carbon double bond at a main chain (e.g., in the middle) and/or end (e.g., terminal end) of an alkyl group having 2 or more carbon atoms. The alkenyl group may be linear or branched. The number of carbon atoms is not particularly limited, but may be 2 to 60, 2 to 30, 2 to 20 or 2 to 10. Examples of the alkenyl group include a vinyl group, a 1-butenyl group, a 1-pentenyl group, a 1,3-butadienyl aryl group, a styrenyl group, a styryl vinyl group, and the like, but are not limited thereto.

As used herein, an alkynyl group refers to a hydrocarbon group including at least one carbon triple bond at a main chain (e.g., in the middle) or end (e.g., terminal end) of an alkyl group having 2 or more carbon atoms. The alkynyl group may be linear or branched. The number of carbon atoms is not particularly limited, but may be 2 to 60, 2 to 30, 2 to 20 or 2 to 10. Examples of the alkynyl group may include an ethynyl group, a propynyl group, and the like, but are not limited thereto.

As used herein, a hydrocarbon ring group refers to any suitable functional group and/or substituent derived from an aliphatic hydrocarbon ring. The hydrocarbon ring group may be a saturated hydrocarbon ring group having 5 to 20 ring-forming carbon atoms.

As used herein, an aryl group refers to any suitable functional group and/or substituent derived from an aromatic hydrocarbon ring. The aryl group may be a monocyclic aryl group or a polycyclic aryl group. The number of ring-forming carbon atoms in the aryl group may be 6 to 60, 6 to 30, 6 to 20, or 6 to 15. Examples of the aryl group may include a phenyl group, a naphthyl group, a fluorenyl group, an anthracenyl group, a phenanthryl group, a biphenyl group, a terphenyl group, a quaterphenyl group, a quinquephenyl group, a sexiphenyl group, a triphenylenyl group, a pyrenyl group, a benzofluoranthene group, a chrysenyl group, and the like, but are not limited thereto.

As used herein, a fluorenyl group may be substituted, and two substituents may be bonded to each other to form a spiro structure. An example that the fluorenyl group is substituted is as follows. However, the embodiment of the present disclosure is not limited thereto.

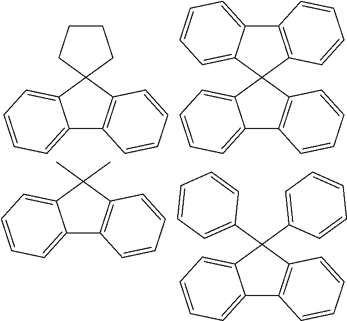

As used herein, a heterocyclic group refers to any suitable functional groups and/or substituents derived from a ring containing at least one selected from B, O, N, P, Si, Se, Te, and S as a hetero atom. The heterocyclic group includes an aliphatic heterocyclic group and an aromatic heterocyclic group. The aromatic heterocyclic group may be a heteroaryl group. The aliphatic heterocyclic group and the aromatic heterocyclic group may be monocyclic or polycyclic.

As used herein, the heterocyclic group may contain at least one selected from B, O, N, P, Si, Se, Te, and S as a hetero atom. When the heterocyclic group contains two or more hetero atoms, the two or more hetero atoms may be the same as or different from each other. The heterocyclic group may be a monocyclic heterocyclic group or a polycyclic heterocyclic group, and include a heteroaryl group. The number of ring-forming carbon atoms in the heterocyclic group may be 2 to 60, 2 to 30, 2 to 20, or 2 to 10.

As used herein, the aliphatic heterocyclic group may contain at least one selected from B, O, N, P, Si, Se, Te, and S as a hetero atom. The number of ring-forming carbon atoms in the aliphatic heterocyclic group may be 2 to 60, 2 to 30, 2 to 20, or 2 to 10. Examples of the aliphatic heterocyclic group include an oxirane group, a thiirane group, a pyrrolidine group, a piperidine group, a tetrahydrofuran group, a tetrahydrothiophene group, a thiane group, a tetrahydropyran group, a 1,4-dioxane group, etc., but are not limited to thereto.

As used herein, the heteroaryl group may contain at least one selected from B, O, N, P, Si, Se, Te, and S as a hetero atom. When the heteroaryl group contains two or more hetero atoms, the two or more hetero atoms may be the same as or different from each other. The heteroaryl group may be a monocyclic heteroaryl group or a polycyclic heteroaryl group. The number of ring-forming carbon atoms in the heteroaryl group may be 2 to 60, 2 to 30, 2 to 20, or 2 to 10. Examples of the heteroaryl group may include a thiophene group, a furan group, a pyrrole group, an imidazole group, a pyridine group, a bipyridine group, a pyrimidine group, a triazine group, a triazole group, an acridyl group, a pyridazine group, a pyrazinyl group, a quinoline group, a quinazoline group, a quinoxaline group, a phenoxazine group, a phthalazine group, a pyrido pyrimidine group, a pyrido pyrazine group, a pyrazino pyrazine group, an isoquinoline group, an indole group, a carbazole group, an N-arylcarbazole group, an N-heteroarylcarbazole group, an N-alkylcarbazole group, a benzoxazole group, a benzoimidazole group, a benzothiazole group, a benzocarbazole group, a benzothiophene group, a dibenzothiophene group, a thienothiophene group, a benzofuran group, a phenanthroline group, a thiazole group, an isoxazole group, an oxazole group, an oxadiazole group, a thiadiazole group, a phenothiazine group, a dibenzosilole group, a dibenzofuran group, and the like, but are not limited thereto.

As used herein, the above description of the aryl group may be applied to an arylene group, except that the arylene group is a divalent group. The above description of the heteroaryl group may be applied to a heteroarylene group, except that the heteroarylene group is a divalent group.

As used herein, a silyl group may refer to one that a silicon atom is bonded to an alkyl group or aryl group as defined above. The silyl group includes an alkyl silyl group and an aryl silyl group. Examples of the silyl group include a trimethylsilyl group, a triethylsilyl group, a t-butyldimethylsilyl group, an ethyldimethylsilyl group, a propyldimethylsilyl group, a triphenylsilyl group, a diphenylsilyl group, a phenylsilyl group, and the like, but are not limited thereto.

As used herein, the number of carbon atoms in a carbonyl group is not particularly limited, but may be 1 to 40, 1 to 30, or 1 to 20. For example, the carbonyl group may have the following structure, but is not limited thereto.

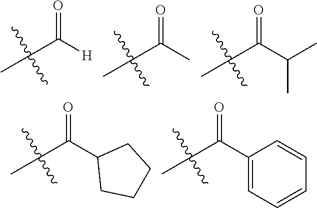

As used herein, the number of carbon atoms in a sulfinyl group and a sulfonyl group is not particularly limited, but may be 1 to 30. The sulfinyl group may include an alkyl sulfinyl group and an aryl sulfinyl group. The sulfonyl group may include an alkyl sulfonyl group and an aryl sulfonyl group.

As used herein, a thio group may include an alkyl thio group and an aryl thio group. The thio group may indicate the one that a sulfur atom is bonded to an alkyl group or an aryl group as defined above. Examples of the thio group may include a methylthio group, an ethylthio group, a propylthio group, a pentylthio group, a hexylthio group, an octylthio group, a dodecylthio group, a cyclopentylthio group, a cyclohexylthio group, a phenylthio group, a naphthylthio group, and the like, but are not limited to thereto.

As used herein, an oxy group may indicate the one that an oxygen atom is bonded to an alkyl group and/or aryl group as defined above. The oxy group may include an alkoxy group and/or an aryl oxy group. The alkoxy group may be linear, branched or cyclic. The number of carbon atoms in the alkoxy group is not particularly limited, but may be, for example, 1 to 20, or 1 to 10. Examples of the oxy group include methoxy, ethoxy, n-propoxy, isopropoxy, butoxy, pentyloxy, hexyloxy, octyloxy, nonyloxy, decyloxy, benzyloxy, and the like, but are not limited thereto.

As used herein, a boron group may refer to one that a boron atom is bonded to an alkyl group or aryl group as defined above. The boron group includes an alkyl boron group and/or an aryl boron group. Examples of the boron group include a dimethylboron group, a diethylboron group, a t-butylmethylboron group, a diphenylboron group, a phenylboron group, etc., but are not limited thereto.

As used herein, the number of carbon atoms in an amine group is not particularly limited, but may be 1 to 30. The amine group may include an alkyl amine group and an aryl amine group. Examples of the amine group include a methylamine group, a dimethylamine group, a phenylamine group, a diphenylamine group, a naphthylamine group, a 9-methyl-anthracenylamine group, a phenyl-dibenzofuranyl-amine group, and the like, but are not limited thereto.

As used herein, the above-described examples of the alkyl group also apply to the alkyl group in an alkylthio group, an alkyl sulfinyl group, an alkyl sulfonyl group, an alkoxy group, an alkyl amine group, an alkyl boron group, an alkyl silyl group, and an alkyl amine group.

As used herein, the above-described examples of the aryl group also apply to the aryl group in an aryloxy group, an arylthio group, an aryl sulfinyl group, an aryl sulfonyl group, an aryl amine group, an aryl boron group, and an aryl silyl group.

As used herein, a direct linkage may refer to a single bond. As used herein, [symbol] and “—*” refer to positions to be connected.

The light emitting element ED according to an embodiment may include an amine compound of an embodiment. The amine compound according to an embodiment may be represented by Formula 1 below.

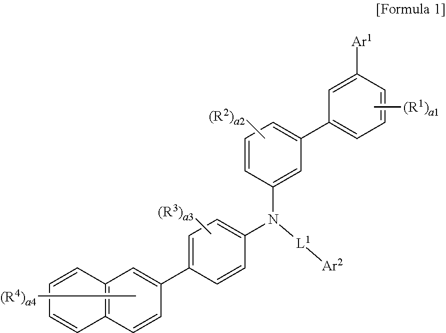

In Formula 1, Ar¹ may be a substituted or unsubstituted aryl group having 6 to 15 ring-forming carbon atoms, or a substituted or unsubstituted first heteroaryl group having 5 to 30 ring-forming carbon atoms. The first heteroaryl group may include only one hetero atom, and the one hetero atom may be an oxygen atom or a sulfur atom.

When the aryl group having more than 15 ring-forming carbon atoms is Ar¹, planarity is high to cause stacking of materials, thereby reducing the movement of charges and increasing driving voltage. When a heteroaryl group including two or more heteroatoms is Ar¹, the charge balance of molecules is not maintained. Ar¹ does not include two or more carbon atoms containing sp3 hybridized orbitals as a ring-forming atom. Ar¹ is a ring group directly or indirectly bonded to an amine group. When the ring group directly or indirectly bonded to an amine group includes two or more carbon atoms containing sp3 hybridized orbitals as ring-forming atoms, the stability of materials is reduced.

Ar² may be a substituted or unsubstituted aryl group having 6 to 30 ring-forming carbon atoms, a substituted or unsubstituted dibenzofuran group, or a substituted or unsubstituted dibenzothiophene group. Ar¹ and Ar² may each independently be a substituted or unsubstituted phenyl group, a substituted or unsubstituted naphthyl group, a substituted or unsubstituted phenanthryl group, a substituted or unsubstituted dibenzofuran group, or a substituted or unsubstituted dibenzothiophene group. For example, at least one selected from Ar¹ and Ar² may be a substituted or unsubstituted phenanthryl group, a substituted or unsubstituted dibenzofuran group, or a substituted or unsubstituted dibenzothiophene group.

For example, L¹ may be a direct linkage, and Ar² may be a phenyl group substituted with a diphenylamine group. L¹ may be a direct linkage, Ar² may be a phenyl group substituted with a first phenyl group, and the first phenyl group may be a phenyl group substituted with a diphenylamine group. L¹ may be a direct linkage, and Ar² may be a biphenyl group substituted with a diphenylamine group. L¹ may be an unsubstituted phenylene group, and Ar² may be a phenyl group substituted with a diphenylamine group. L¹ may be a direct linkage, and Ar² may be a phenyl group substituted with a phenyl-dibenzofuranyl-amine group. However, this is presented as an example, and the embodiment of the present disclosure is not limited thereto.

In an embodiment, Ar¹ may be represented by any one selected from AR1-1 to AR1-12 below. AR1-1 and AR1-2 indicate unsubstituted naphthyl groups, and AR1-3 to AR1-5 indicate unsubstituted phenanthryl groups. AR1-6 and AR1-7 indicate substituted or unsubstituted phenyl groups. AR1-8 to AR1-10 indicate unsubstituted dibenzofuran groups. AR1-11 and AR1-12 indicate unsubstituted dibenzothiophene groups. In AR1-7, D is a deuterium atom.

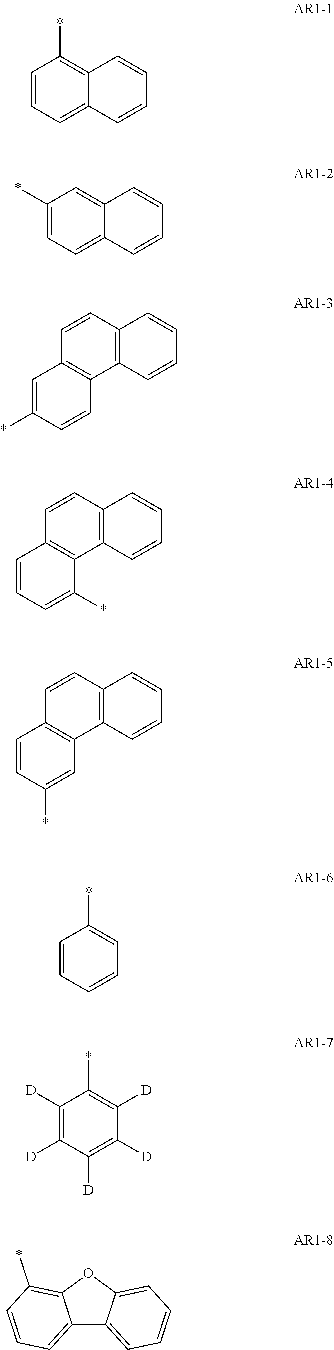

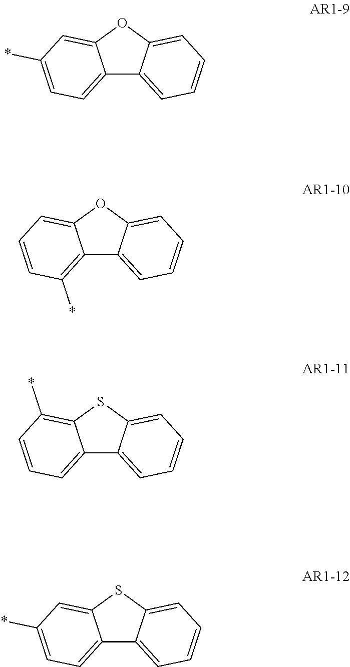 [SMILES: *c1ccc2c(c1)oc1ccccc12 ; *c1cccc2oc3ccccc3c12 ; *c1cccc2c1sc1ccccc12 ; *c1ccc2c(c1)sc1ccccc12]

Ar² may be represented by any one selected from AR2-1 to AR2-24 below. AR2-1 to AR2-5 and AR2-22 to AR2-24 indicate substituted or unsubstituted phenyl groups. AR2-6 and AR2-7 indicate unsubstituted naphthyl groups. AR2-8 to AR2-10 indicate substituted or unsubstituted phenanthryl groups. AR2-11 to AR2-17 indicate substituted or unsubstituted dibenzofuran groups. AR2-18 to AR2-21 indicate substituted or unsubstituted dibenzothiophene groups. In AR2-14, D is a deuterium atom.

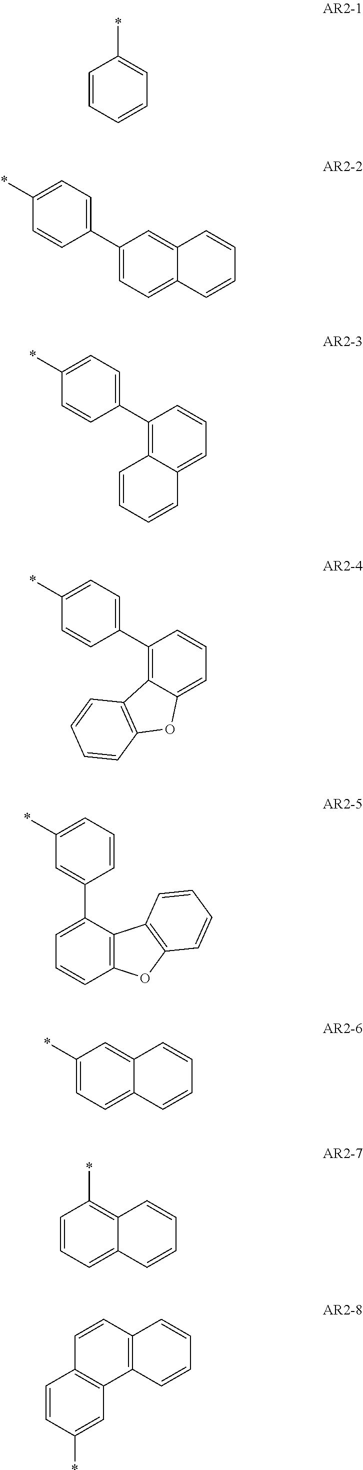

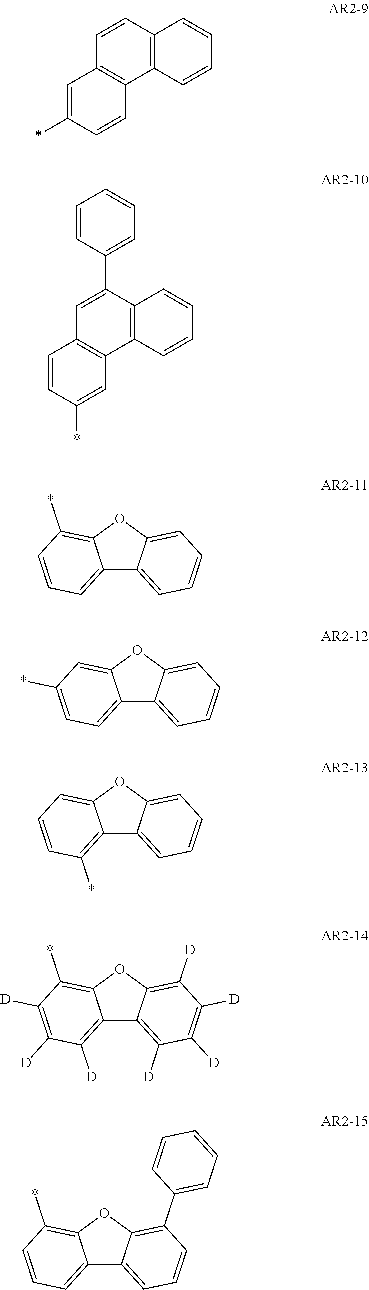

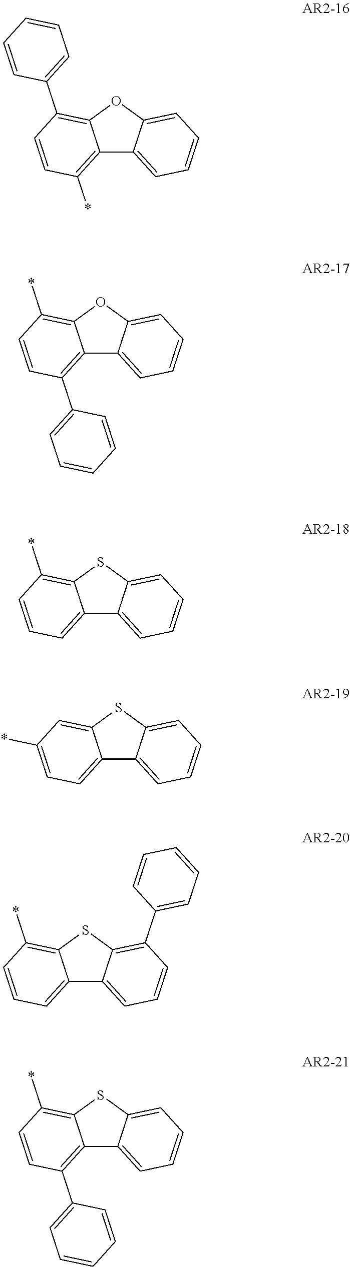 [SMILES: *c1ccc(-c2ccccc2)c2oc3ccccc3c12 ; *c1ccc(-c2ccccc2)c2c1oc1ccccc12 ; *c1cccc2c1sc1ccccc12 ; *c1ccc2c(c1)sc1ccccc12 ; *c1cccc2c1sc1c(-c3ccccc3)cccc12 ; *c1ccc(-c2ccccc2)c2c1sc1ccccc12]

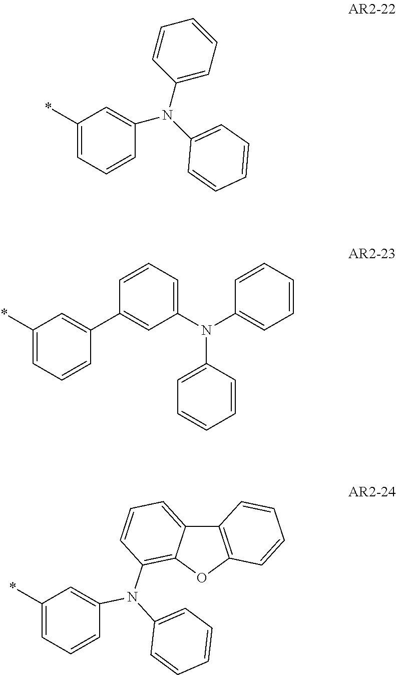 [SMILES: *c1cccc(N(c2ccccc2)c2ccccc2)c1 ; *c1cccc(-c2cccc(N(c3ccccc3)c3ccccc3)c2)c1 ; *c1cccc(N(c2ccccc2)c2cccc3c2oc2ccccc23)c1]

In Formula 1, a1 to a3 may each indecently be an integer of 0 to 4. A₄ may each be an integer of 0 to 7. When a1 is an integer of 2 or greater, a plurality of R¹'s may all be the same or at least one may be different from the others. When a2 is an integer of 2 or greater, a plurality of R²'s may all be the same or at least one may be different from the others. When a3 is an integer of 2 or greater, a plurality of R³'s may all be the same or at least one may be different from the others. When a4 is an integer of 2 or greater, a plurality of R⁴'s may all be the same or at least one may be different from the others.

R¹, R³, and R⁴ may each independently be a hydrogen atom, a deuterium atom, a substituted or unsubstituted alkyl group having 1 to 10 carbon atoms, or a substituted or unsubstituted aryl group having 6 to 30 ring-forming carbon atoms. R² may be a hydrogen atom, a deuterium atom, or a substituted or unsubstituted alkyl group having 1 to 10 carbon atoms.

For example, R¹ may be a hydrogen atom, a deuterium atom, or an unsubstituted phenyl group. R³ may be a hydrogen atom or a deuterium atom. R⁴ may be a hydrogen atom, a deuterium atom, or an unsubstituted phenyl group.

L¹ may be a direct linkage or a substituted or unsubstituted arylene group having 6 to 30 ring-forming carbon atoms. For example, L¹ may be a direct linkage, a substituted or unsubstituted phenylene group, or a substituted or unsubstituted divalent phenanthryl group. L¹ may be an unsubstituted phenylene group or an unsubstituted divalent phenanthryl group, and Ar² may be represented by AR2-1, AR2-6, AR2-7, or AR2-13 described above.

In Formula 1, when a1 to a4 are all 0, Ar² is not a 1-dibenzothiophene group. The 1-dibenzothiophene group is easily affected by electric charges as sulfur atoms, which are ring-forming atoms, have a relatively large atomic radius. In addition, as nitrogen atoms of an amine group is affected by the three-dimensional structure of the 1-dibenzothiophene group, the stability of materials is reduced.

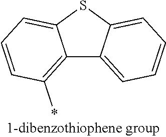 [SMILES: *c1cccc2sc3ccccc3c12]

In addition, in Formula 1, when a1 to a4 are all 0, L¹-Ar² is not an unsubstituted biphenyl group. In some embodiments, in Formula 1, a case where a1 to a4 are all 0, L¹ is a direct linkage, and Ar² is an unsubstituted biphenyl group is excluded. In Formula 1, a case where a1 to a4 are all 0, L¹ is an unsubstituted phenylene group, and Ar² is an unsubstituted phenyl group is excluded. In Formula 1, when a1 to a4 are all 0, L¹-Ar² does not include an unsubstituted biphenyl group represented by LA-1 to LA-3 below.

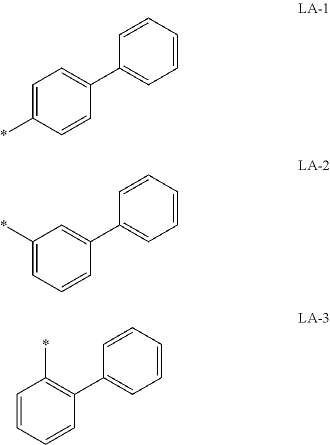 [SMILES: *c1ccc(-c2ccccc2)cc1 ; *c1cccc(-c2ccccc2)c1 ; *c1ccccc1-c1ccccc1]

LA-1 indicates a para-biphenyl group, and the para-biphenyl group has high planarity. LA-2 indicates a meta-biphenyl group, and the meta-biphenyl group has high symmetry. Accordingly, an amine compound to which the para-biphenyl group or the meta-biphenyl group are bonded is stacked to increase the driving voltage of a light emitting element. LA-3 indicates an ortho-biphenyl group, and when the ortho-biphenyl group is bonded to an amine group, distortion take places around nitrogen atoms of the amine group to reduce the stability of materials.

The amine compound of an embodiment does not include a fluorenyl group in which an alkyl group is substituted on the 9^{th} carbon atom. In Formula 1, Ar¹, Ar², L¹, and R¹ to R⁴ do not include a fluorenyl group in which an alkyl group is substituted on the 9^{th} carbon atom (C9). Formula F-1 below indicates a fluorene moiety, C9 indicates the 9^{th} carbon atom of fluorene, and R_{b1} and R_{b2} indicate substituents bonded to the 9^{th} carbon atom. The amine compound of an embodiment does not include a fluorenyl group in which R_{b1} and R_{b2} are alkyl groups in Formula F-1. In Formula F-1, a fluorenyl group in which R_{b1} and R_{b2} are alkyl groups has low chemical stability, and accordingly, a compound containing the fluorenyl group is not suitable as a hole transport material.

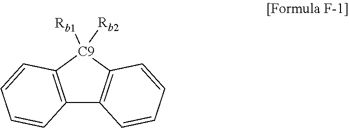

In an embodiment, the amine compound represented by Formula 1 may be represented by any one selected from among Formulas 1-1 to 1-4 below. Formulas 1-1 to 1-4 more specifically indicate or provide R³ and R⁴ in Formula 1.

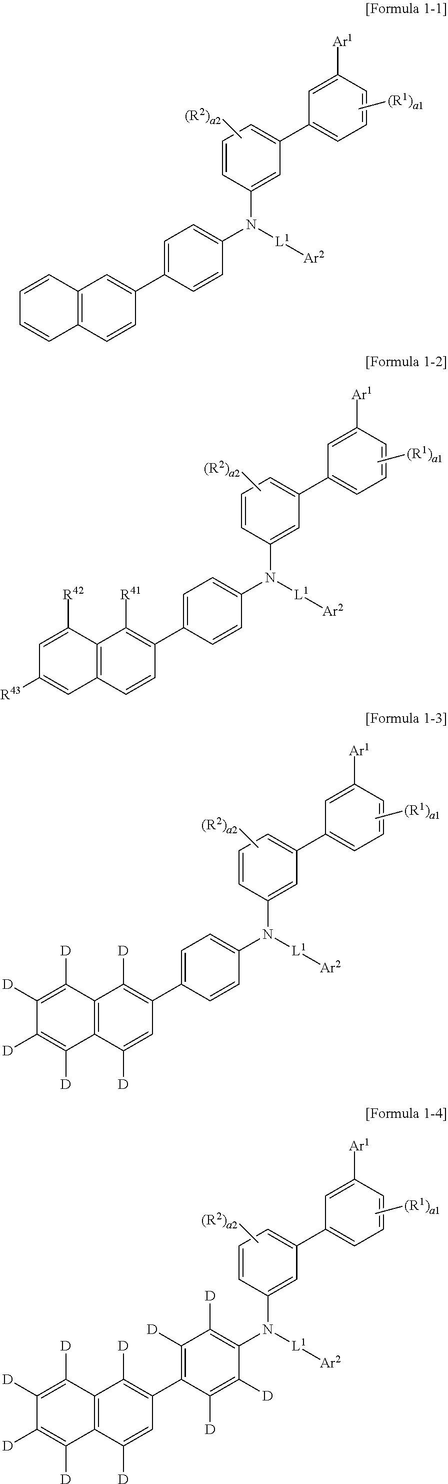

In Formulas 1-1 to 1-4, the same descriptions with respect to Formula 1 may be applied to Ar¹, Ar², a1, a2, R¹, R², and L¹. In Formula 1-2, at least one selected from R⁴¹ to R⁴³ may be an unsubstituted phenyl group, and the others may be hydrogen atoms. In Formulas 1-3 and 1-4, D is a deuterium atom.

Formulas 1-1 to 1-3 may indicate cases in which a3 is 0 in Formula 1. In some embodiments, Formulas 1-1 to 1-3 may indicate cases in which a3 is 4 and four R³'s are hydrogen atoms in Formula 1.

Formula 1-1 may indicate a case in which a4 is 0 or a4 is 7, and seven R⁴'s are hydrogen atoms in Formula 1. Formula 1-2 may indicate a case in which a4 is 1 or greater and at least one R⁴'s is an unsubstituted phenyl group in Formula 1. Formulas 1-3 1-4s may indicate cases in which a4 is 6 and six R⁴'s are deuterium atoms in Formula 1.

In an embodiment, the amine compound represented by Formula 1-2 may be represented by any one selected from among Formulas 1-2 Å to 1-2D below. Formulas 1-2 Å to 1-2D more specifically indicate or provide R⁴¹ to R⁴³ in Formula 1-2.

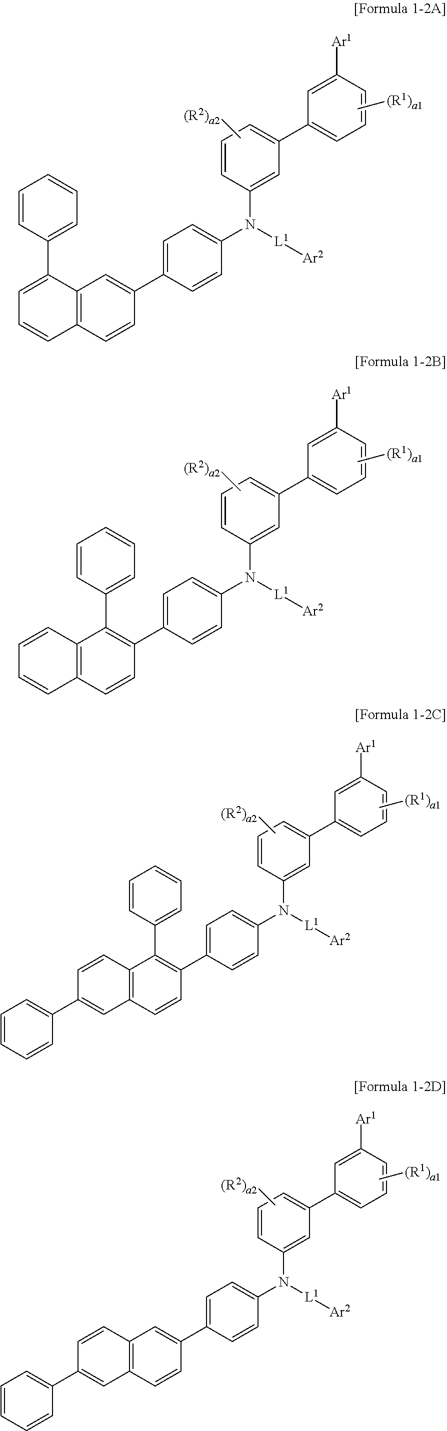

In Formulas 1-2 Å to 1-2D, the same descriptions with respect to Formula 1 may be applied to Ar¹, Ar², a1, a2, R¹, R², and L¹. Formula 1-2A indicates a case in which R⁴² is an unsubstituted phenyl group, and R⁴¹ and R⁴³ are hydrogen atoms in Formula 1-2. Formula 1-2B indicates a case in which R⁴¹ is an unsubstituted phenyl group, and R⁴² and R⁴³ are hydrogen atoms in Formula 1-2. Formula 1-2C indicates a case in which R⁴¹ and R⁴³ are unsubstituted phenyl groups, and R⁴² is a hydrogen atom in Formula 1-2. Formula 1-2B indicates a case in which R⁴³ is an unsubstituted phenyl group, and R⁴¹ and R⁴² are hydrogen atoms in Formula 1-2.

The amine compound of an embodiment may include a core represented by Formula Z1 below. In Formulas Z1, the same descriptions with respect to Formula 1 may be applied to Ar¹, Ar², and L¹.

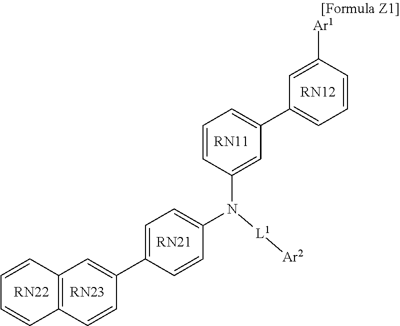

In Formula Z1, RN11, RN12, and RN21 to RN23 are indicated to refer to a ring group. RN 11 and RN12 correspond to the first ring group described above and are meta-meta-biphenyl groups. RN21 corresponds to the second ring group described above and is a phenyl group substituted with a naphthyl group composed of RN22 and RN23. The third cyclic group described above corresponds to Ar². As described in Formula 1, Ar² may be an aryl group, a dibenzofuran group, or a dibenzothiophene group.

The light emitting element ED including the amine compound of an embodiment may have reduced driving voltage and increased lifespan. The amine compound of an embodiment introduces a naphthyl group into a second ring group to maintain the characteristics of the amine group and also improve charge tolerance, thereby contributing to the long lifespan of a light emitting element ED. In addition, the amine compound of an embodiment includes a third ring group that is an aryl group, a dibenzofuran group, or a dibenzothiophene group, and a first ring group that is a meta-meta biphenyl group, and may thus prevent or reduce stacking between materials. Accordingly, it is considered that the amine compound of an embodiment may easily move charges through hopping to reduce the driving voltage of the light emitting element ED.

In the amine compound of an embodiment, as a phenyl group directly bonded to the amine group in a biphenyl group is bonded to a meta position, excessive expansion of the HOMO (highest occupied molecular orbital) energy level at the nitrogen atoms of the amine group may be prevented or reduced. Accordingly, the amine compound of an embodiment may exhibit a HOMO energy level suitable for a hole transport material.

An amine compound of an embodiment may be represented by any one selected from among compounds of Compound Group 1 below. A light emitting element ED of an embodiment may include at least one selected from the compounds from Compound Group 1 below. In Compound Group 1 below, D is a deuterium atom.

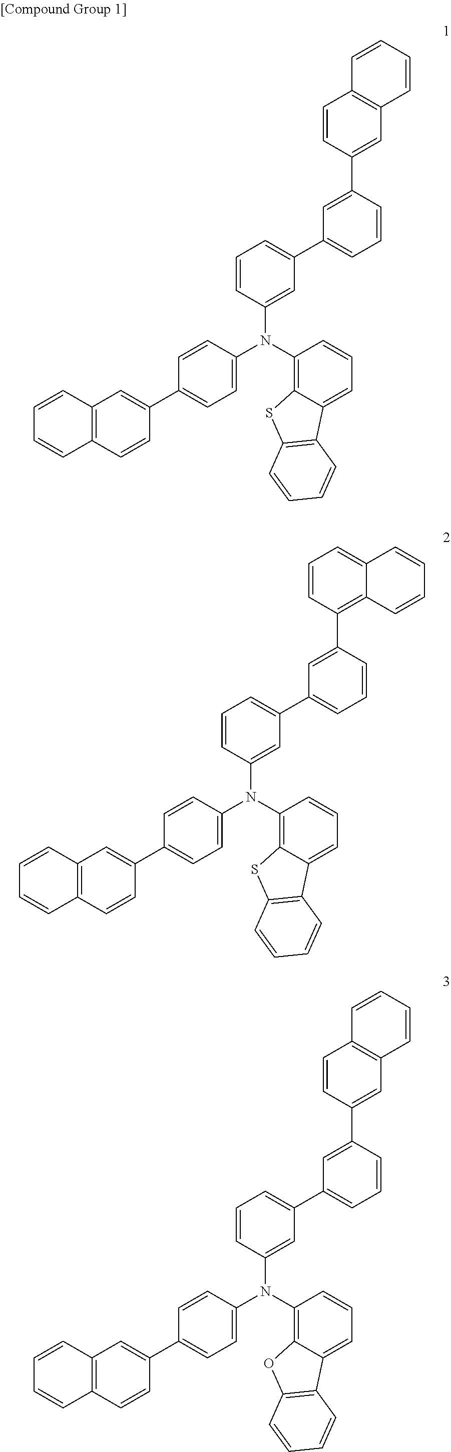 [SMILES: c1cc(-c2cccc(N(c3ccc(-c4ccc5ccccc5c4)cc3)c3cccc4c3sc3ccccc34)c2)cc(-c2ccc3ccccc3c2)c1 ; c1cc(-c2cccc(N(c3ccc(-c4ccc5ccccc5c4)cc3)c3cccc4c3sc3ccccc34)c2)cc(-c2cccc3ccccc23)c1 ; c1cc(-c2cccc(N(c3ccc(-c4ccc5ccccc5c4)cc3)c3cccc4c3oc3ccccc34)c2)cc(-c2ccc3ccccc3c2)c1]

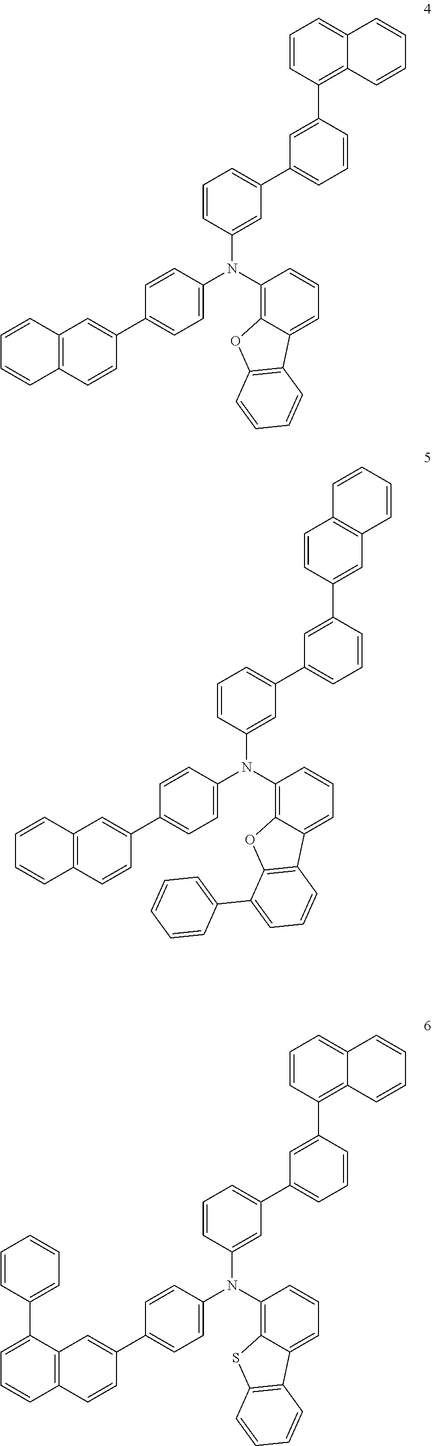 [SMILES: c1cc(-c2cccc(N(c3ccc(-c4ccc5ccccc5c4)cc3)c3cccc4c3oc3ccccc34)c2)cc(-c2cccc3ccccc23)c1 ; c1ccc(-c2cccc3c2oc2c(N(c4ccc(-c5ccc6ccccc6c5)cc4)c4cccc(-c5cccc(-c6ccc7ccccc7c6)c5)c4)cccc23)cc1 ; c1ccc(-c2cccc3ccc(-c4ccc(N(c5cccc(-c6cccc(-c7cccc8ccccc78)c6)c5)c5cccc6c5sc5ccccc56)cc4)cc23)cc1]

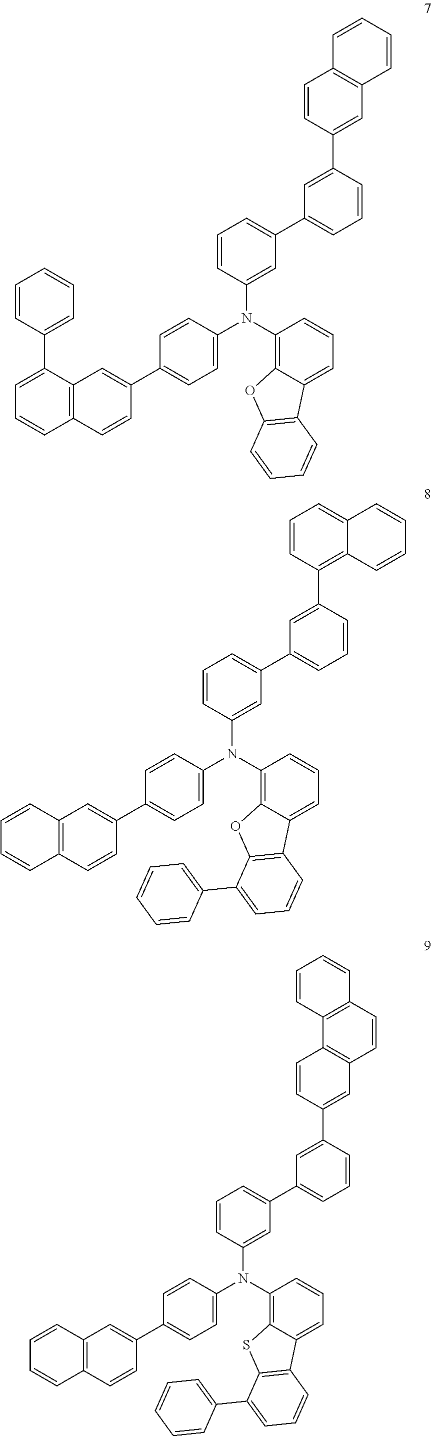

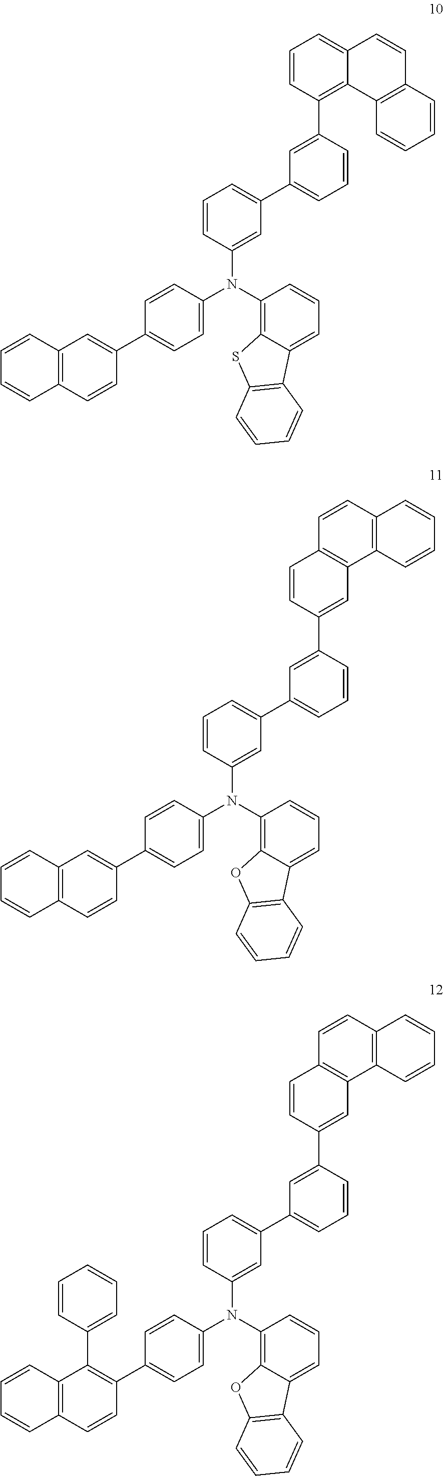 [SMILES: c1cc(-c2cccc(N(c3ccc(-c4ccc5ccccc5c4)cc3)c3cccc4c3sc3ccccc34)c2)cc(-c2cccc3ccc4ccccc4c23)c1 ; c1cc(-c2cccc(N(c3ccc(-c4ccc5ccccc5c4)cc3)c3cccc4c3oc3ccccc34)c2)cc(-c2ccc3ccc4ccccc4c3c2)c1 ; c1ccc(-c2c(-c3ccc(N(c4cccc(-c5cccc(-c6ccc7ccc8ccccc8c7c6)c5)c4)c4cccc5c4oc4ccccc45)cc3)ccc3ccccc23)cc1]

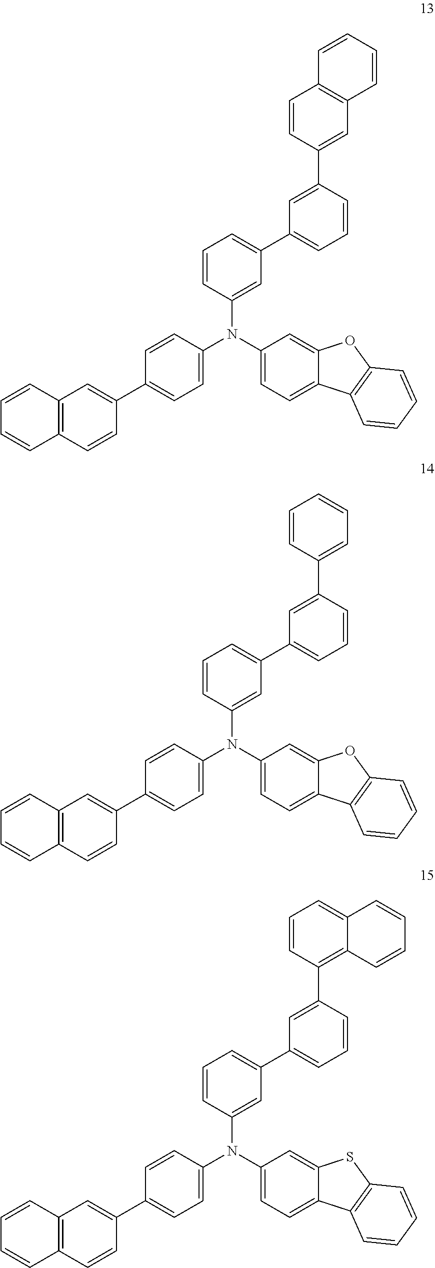 [SMILES: c1cc(-c2cccc(N(c3ccc(-c4ccc5ccccc5c4)cc3)c3ccc4c(c3)oc3ccccc34)c2)cc(-c2ccc3ccccc3c2)c1 ; c1ccc(-c2cccc(-c3cccc(N(c4ccc(-c5ccc6ccccc6c5)cc4)c4ccc5c(c4)oc4ccccc45)c3)c2)cc1 ; c1cc(-c2cccc(N(c3ccc(-c4ccc5ccccc5c4)cc3)c3ccc4c(c3)sc3ccccc34)c2)cc(-c2cccc3ccccc23)c1]

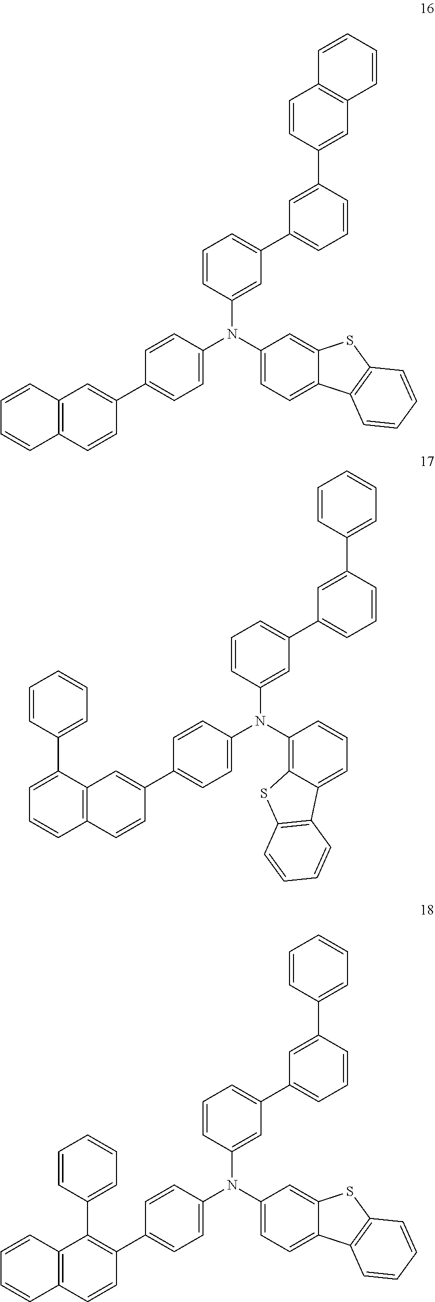 [SMILES: c1cc(-c2cccc(N(c3ccc(-c4ccc5ccccc5c4)cc3)c3ccc4c(c3)sc3ccccc34)c2)cc(-c2ccc3ccccc3c2)c1 ; c1ccc(-c2cccc(-c3cccc(N(c4ccc(-c5ccc6cccc(-c7ccccc7)c6c5)cc4)c4cccc5c4sc4ccccc45)c3)c2)cc1 ; c1ccc(-c2cccc(-c3cccc(N(c4ccc(-c5ccc6ccccc6c5-c5ccccc5)cc4)c4ccc5c(c4)sc4ccccc45)c3)c2)cc1]

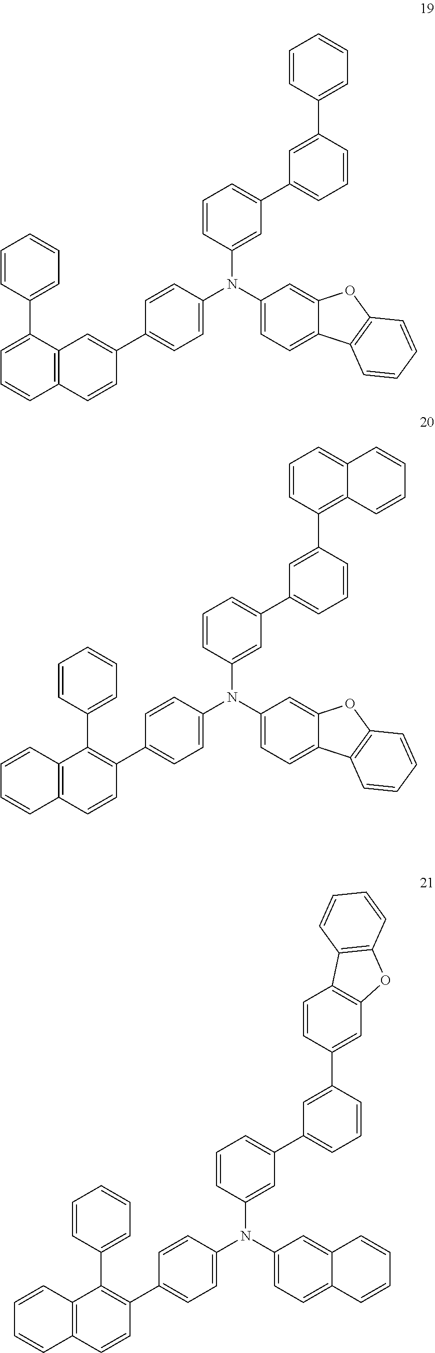 [SMILES: c1ccc(-c2cccc(-c3cccc(N(c4ccc(-c5ccc6cccc(-c7ccccc7)c6c5)cc4)c4ccc5c(c4)oc4ccccc45)c3)c2)cc1 ; c1ccc(-c2c(-c3ccc(N(c4cccc(-c5cccc(-c6cccc7ccccc67)c5)c4)c4ccc5c(c4)oc4ccccc45)cc3)ccc3ccccc23)cc1 ; c1ccc(-c2c(-c3ccc(N(c4cccc(-c5cccc(-c6ccc7c(c6)oc6ccccc67)c5)c4)c4ccc5ccccc5c4)cc3)ccc3ccccc23)cc1]

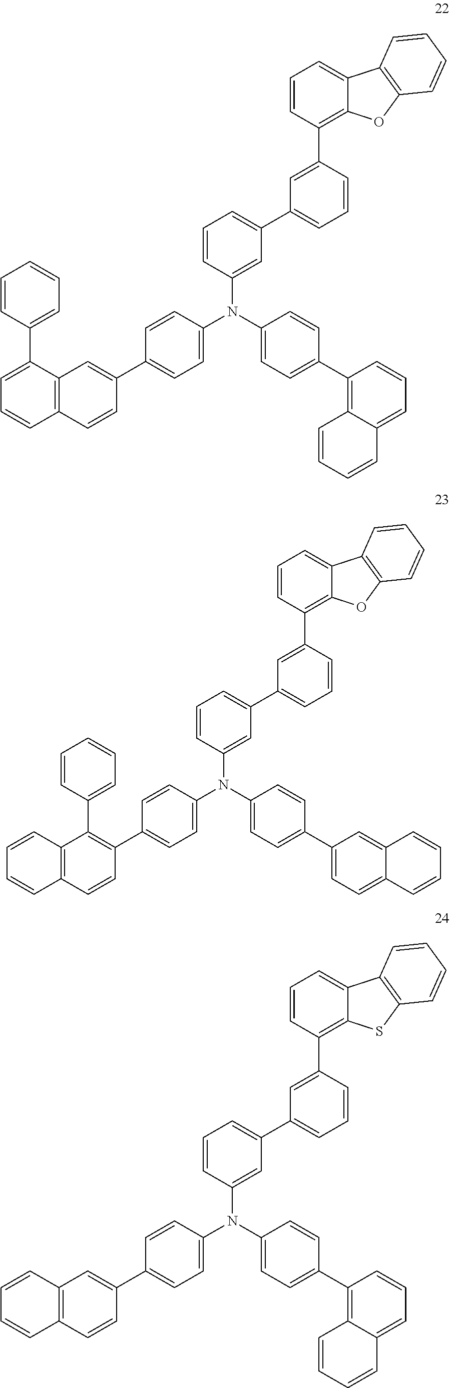 [SMILES: c1ccc(-c2cccc3ccc(-c4ccc(N(c5ccc(-c6cccc7ccccc67)cc5)c5cccc(-c6cccc(-c7cccc8c7oc7ccccc78)c6)c5)cc4)cc23)cc1 ; c1ccc(-c2c(-c3ccc(N(c4ccc(-c5ccc6ccccc6c5)cc4)c4cccc(-c5cccc(-c6cccc7c6oc6ccccc67)c5)c4)cc3)ccc3ccccc23)cc1 ; c1cc(-c2cccc(N(c3ccc(-c4ccc5ccccc5c4)cc3)c3ccc(-c4cccc5ccccc45)cc3)c2)cc(-c2cccc3c2sc2ccccc23)c1]

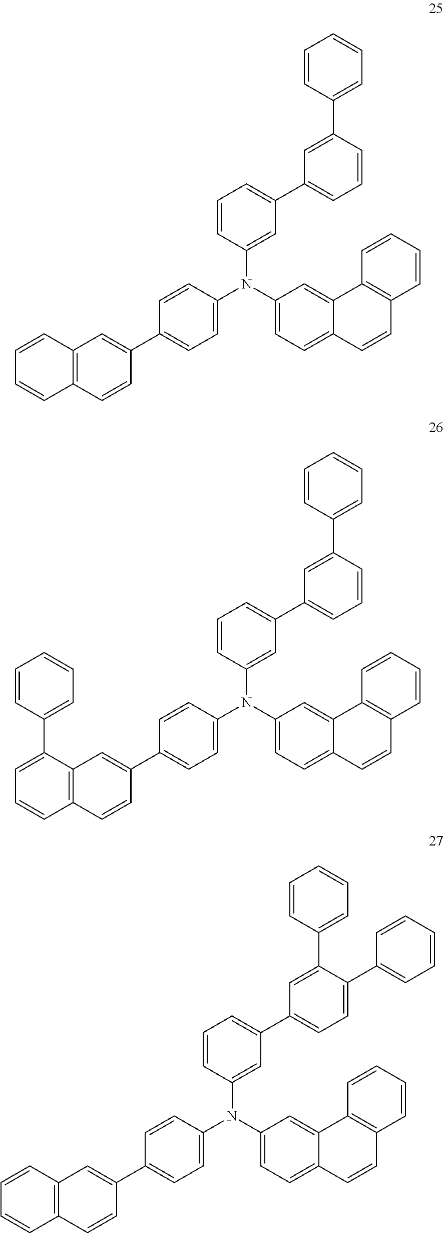 [SMILES: c1ccc(-c2cccc(-c3cccc(N(c4ccc(-c5ccc6ccccc6c5)cc4)c4ccc5ccc6ccccc6c5c4)c3)c2)cc1 ; c1ccc(-c2cccc(-c3cccc(N(c4ccc(-c5ccc6cccc(-c7ccccc7)c6c5)cc4)c4ccc5ccc6ccccc6c5c4)c3)c2)cc1 ; c1ccc(-c2ccc(-c3cccc(N(c4ccc(-c5ccc6ccccc6c5)cc4)c4ccc5ccc6ccccc6c5c4)c3)cc2-c2ccccc2)cc1]

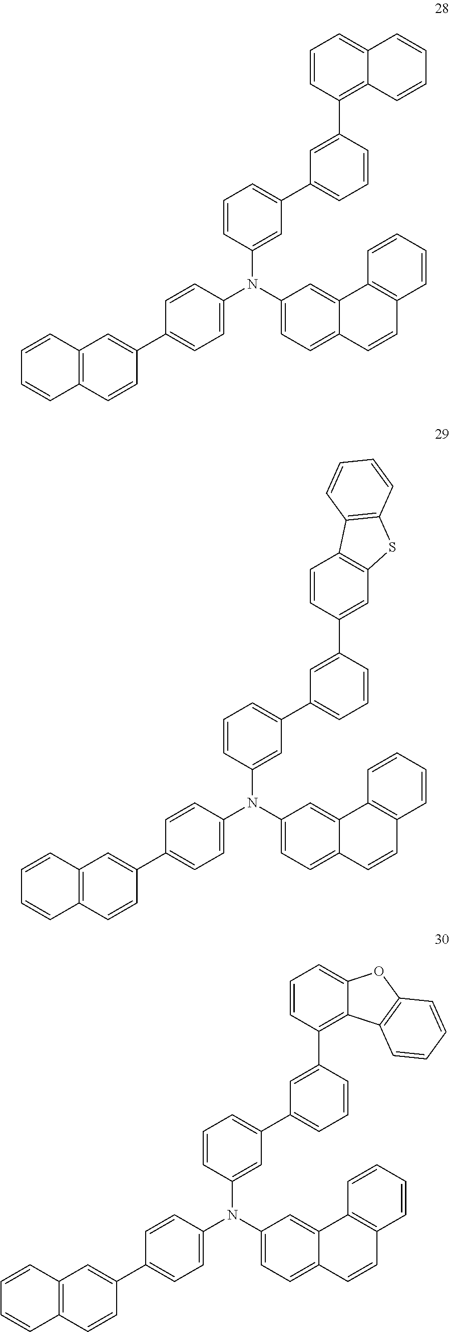 [SMILES: c1cc(-c2cccc(N(c3ccc(-c4ccc5ccccc5c4)cc3)c3ccc4ccc5ccccc5c4c3)c2)cc(-c2cccc3ccccc23)c1 ; c1cc(-c2cccc(N(c3ccc(-c4ccc5ccccc5c4)cc3)c3ccc4ccc5ccccc5c4c3)c2)cc(-c2ccc3c(c2)sc2ccccc23)c1 ; c1cc(-c2cccc(N(c3ccc(-c4ccc5ccccc5c4)cc3)c3ccc4ccc5ccccc5c4c3)c2)cc(-c2cccc3oc4ccccc4c23)c1]

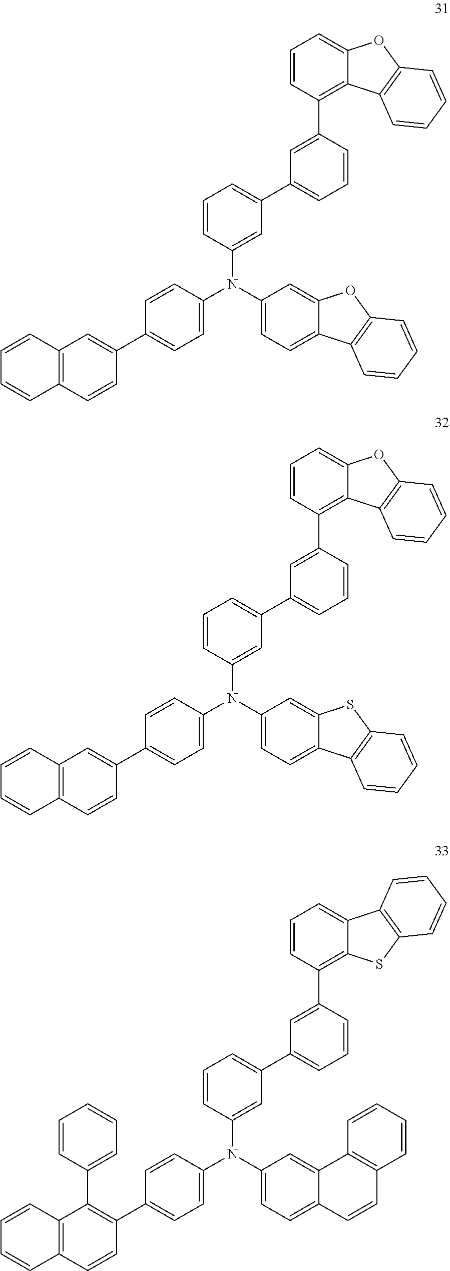 [SMILES: c1cc(-c2cccc(N(c3ccc(-c4ccc5ccccc5c4)cc3)c3ccc4c(c3)oc3ccccc34)c2)cc(-c2cccc3oc4ccccc4c23)c1 ; c1cc(-c2cccc(N(c3ccc(-c4ccc5ccccc5c4)cc3)c3ccc4c(c3)sc3ccccc34)c2)cc(-c2cccc3oc4ccccc4c23)c1 ; c1ccc(-c2c(-c3ccc(N(c4cccc(-c5cccc(-c6cccc7c6sc6ccccc67)c5)c4)c4ccc5ccc6ccccc6c5c4)cc3)ccc3ccccc23)cc1]

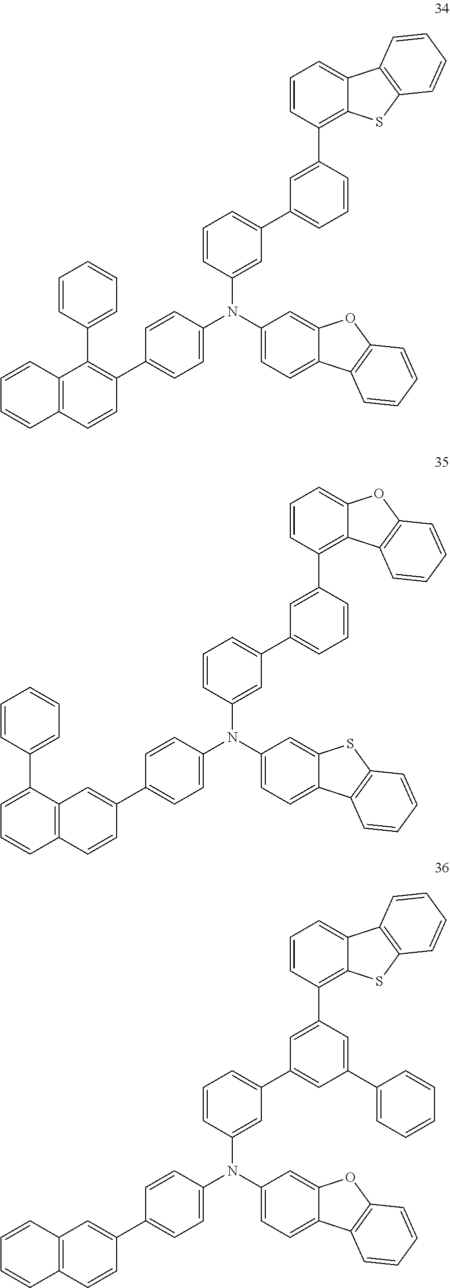 [SMILES: c1ccc(-c2c(-c3ccc(N(c4cccc(-c5cccc(-c6cccc7c6sc6ccccc67)c5)c4)c4ccc5c(c4)oc4ccccc45)cc3)ccc3ccccc23)cc1 ; c1ccc(-c2cccc3ccc(-c4ccc(N(c5cccc(-c6cccc(-c7cccc8oc9ccccc9c78)c6)c5)c5ccc6c(c5)sc5ccccc56)cc4)cc23)cc1 ; c1ccc(-c2cc(-c3cccc(N(c4ccc(-c5ccc6ccccc6c5)cc4)c4ccc5c(c4)oc4ccccc45)c3)cc(-c3cccc4c3sc3ccccc34)c2)cc1]

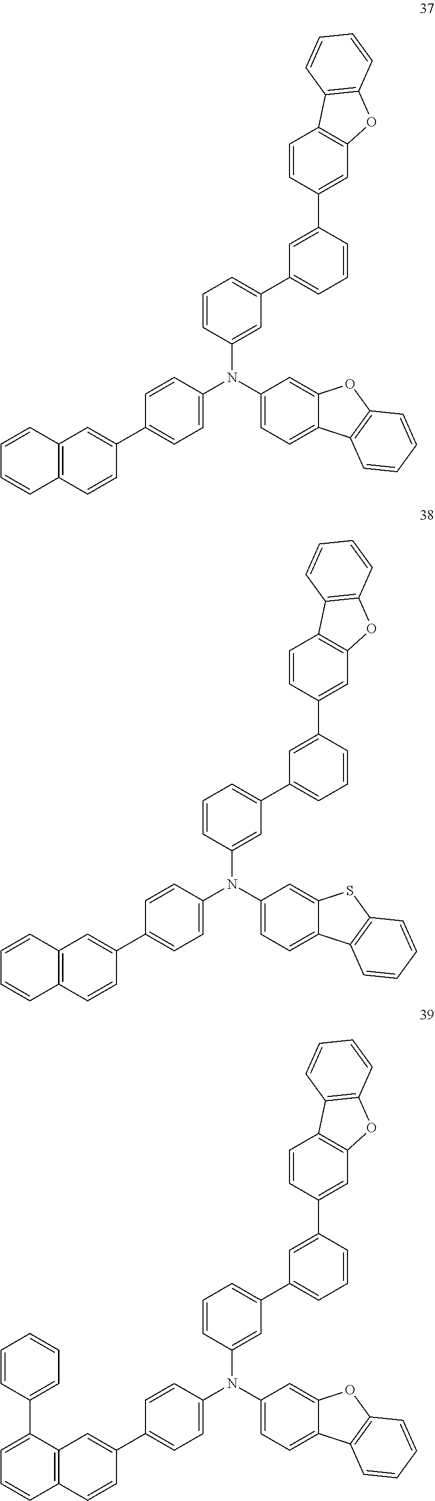 [SMILES: c1cc(-c2cccc(N(c3ccc(-c4ccc5ccccc5c4)cc3)c3ccc4c(c3)oc3ccccc34)c2)cc(-c2ccc3c(c2)oc2ccccc23)c1 ; c1cc(-c2cccc(N(c3ccc(-c4ccc5ccccc5c4)cc3)c3ccc4c(c3)sc3ccccc34)c2)cc(-c2ccc3c(c2)oc2ccccc23)c1 ; c1ccc(-c2cccc3ccc(-c4ccc(N(c5cccc(-c6cccc(-c7ccc8c(c7)oc7ccccc78)c6)c5)c5ccc6c(c5)oc5ccccc56)cc4)cc23)cc1]

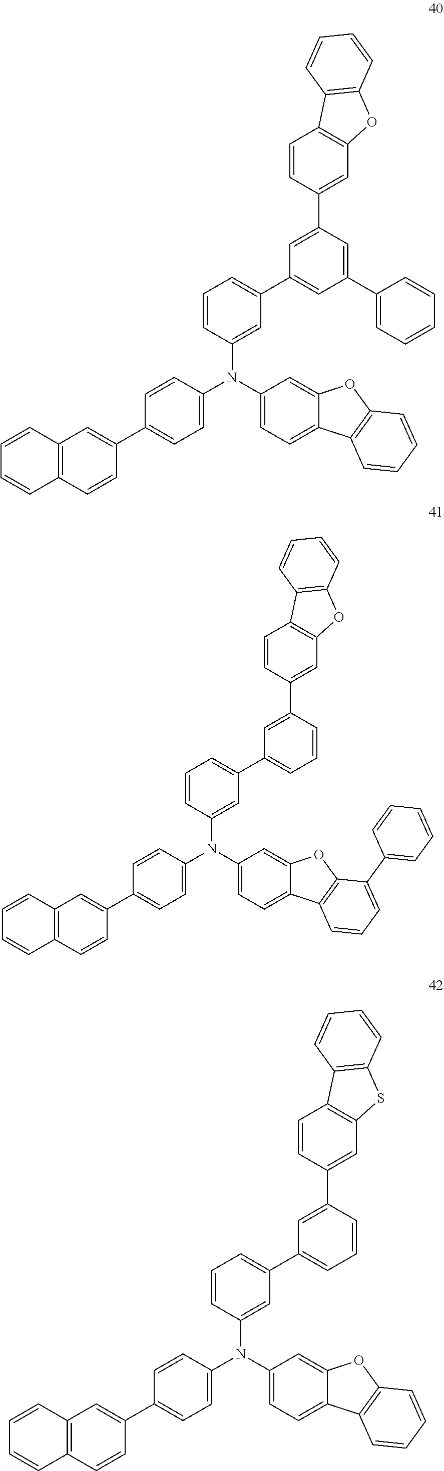 [SMILES: c1ccc(-c2cc(-c3cccc(N(c4ccc(-c5ccc6ccccc6c5)cc4)c4ccc5c(c4)oc4ccccc45)c3)cc(-c3ccc4c(c3)oc3ccccc34)c2)cc1 ; c1ccc(-c2cccc3c2oc2cc(N(c4ccc(-c5ccc6ccccc6c5)cc4)c4cccc(-c5cccc(-c6ccc7c(c6)oc6ccccc67)c5)c4)ccc23)cc1 ; c1cc(-c2cccc(N(c3ccc(-c4ccc5ccccc5c4)cc3)c3ccc4c(c3)oc3ccccc34)c2)cc(-c2ccc3c(c2)sc2ccccc23)c1]

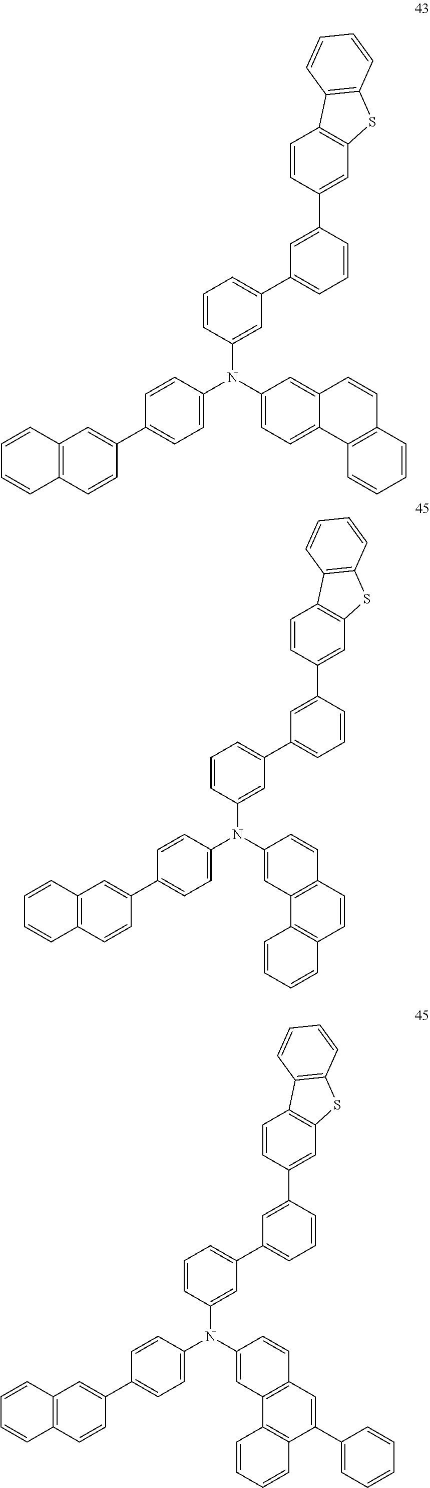

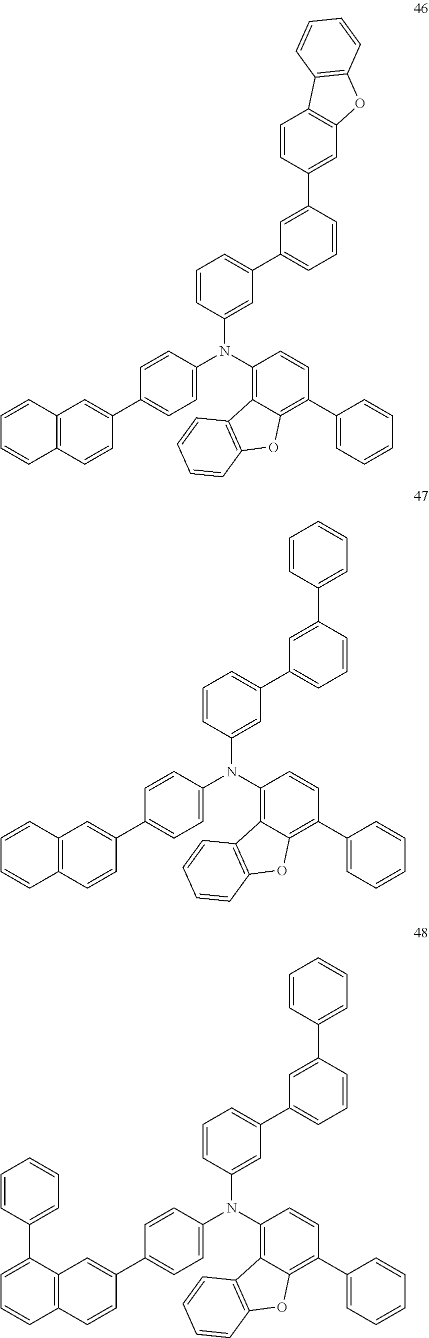 [SMILES: c1ccc(-c2ccc(N(c3ccc(-c4ccc5ccccc5c4)cc3)c3cccc(-c4cccc(-c5ccc6c(c5)oc5ccccc56)c4)c3)c3c2oc2ccccc23)cc1 ; c1ccc(-c2cccc(-c3cccc(N(c4ccc(-c5ccc6ccccc6c5)cc4)c4ccc(-c5ccccc5)c5oc6ccccc6c45)c3)c2)cc1 ; c1ccc(-c2cccc(-c3cccc(N(c4ccc(-c5ccc6cccc(-c7ccccc7)c6c5)cc4)c4ccc(-c5ccccc5)c5oc6ccccc6c45)c3)c2)cc1]

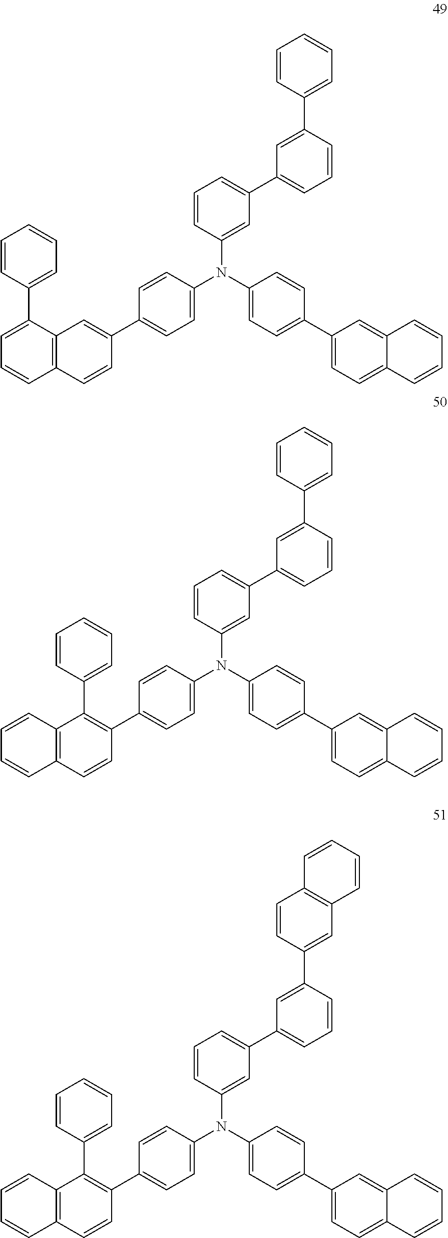 [SMILES: c1ccc(-c2cccc(-c3cccc(N(c4ccc(-c5ccc6ccccc6c5)cc4)c4ccc(-c5ccc6cccc(-c7ccccc7)c6c5)cc4)c3)c2)cc1 ; c1ccc(-c2cccc(-c3cccc(N(c4ccc(-c5ccc6ccccc6c5)cc4)c4ccc(-c5ccc6ccccc6c5-c5ccccc5)cc4)c3)c2)cc1 ; c1ccc(-c2c(-c3ccc(N(c4ccc(-c5ccc6ccccc6c5)cc4)c4cccc(-c5cccc(-c6ccc7ccccc7c6)c5)c4)cc3)ccc3ccccc23)cc1]

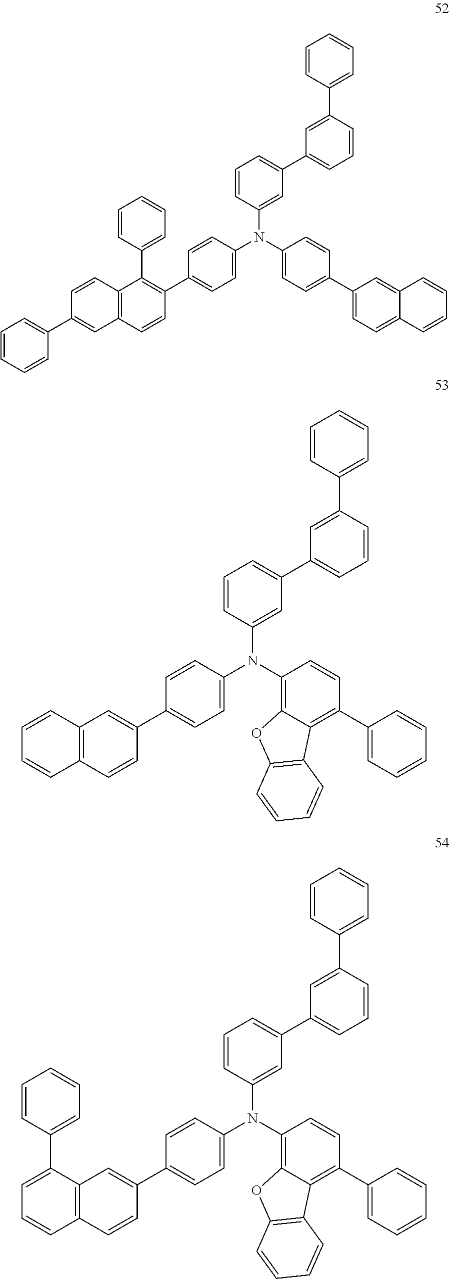 [SMILES: c1ccc(-c2cccc(-c3cccc(N(c4ccc(-c5ccc6ccccc6c5)cc4)c4ccc(-c5ccc6cc(-c7ccccc7)ccc6c5-c5ccccc5)cc4)c3)c2)cc1 ; c1ccc(-c2cccc(-c3cccc(N(c4ccc(-c5ccc6ccccc6c5)cc4)c4ccc(-c5ccccc5)c5c4oc4ccccc45)c3)c2)cc1 ; c1ccc(-c2cccc(-c3cccc(N(c4ccc(-c5ccc6cccc(-c7ccccc7)c6c5)cc4)c4ccc(-c5ccccc5)c5c4oc4ccccc45)c3)c2)cc1]

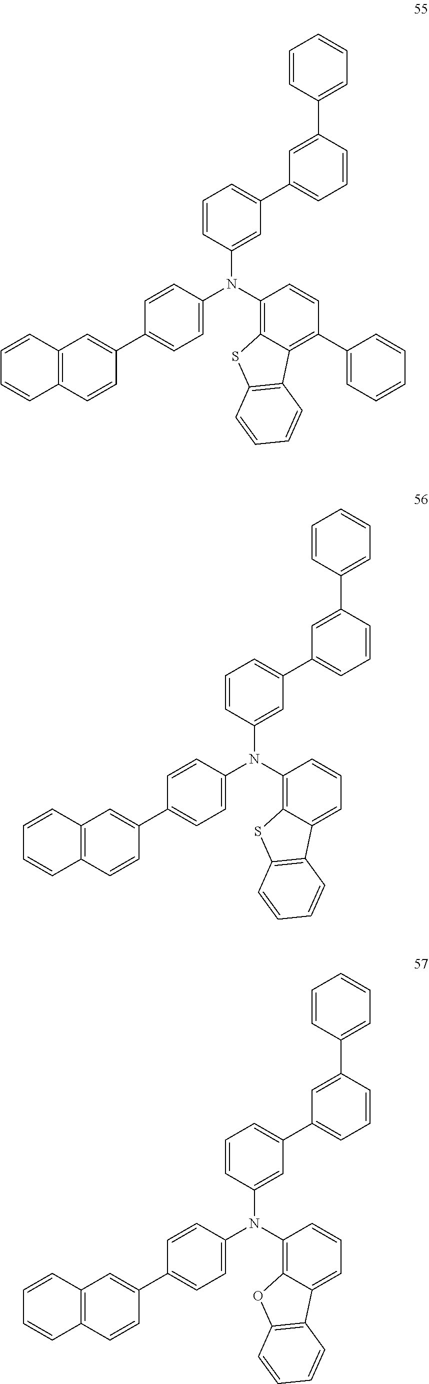 [SMILES: c1ccc(-c2cccc(-c3cccc(N(c4ccc(-c5ccc6ccccc6c5)cc4)c4ccc(-c5ccccc5)c5c4sc4ccccc45)c3)c2)cc1 ; c1ccc(-c2cccc(-c3cccc(N(c4ccc(-c5ccc6ccccc6c5)cc4)c4cccc5c4sc4ccccc45)c3)c2)cc1 ; c1ccc(-c2cccc(-c3cccc(N(c4ccc(-c5ccc6ccccc6c5)cc4)c4cccc5c4oc4ccccc45)c3)c2)cc1]

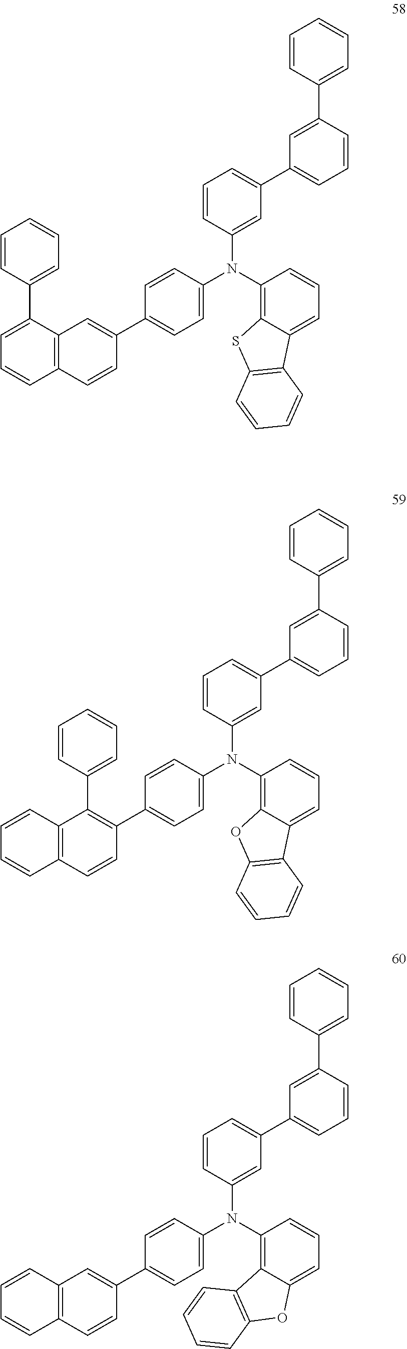 [SMILES: c1ccc(-c2cccc(-c3cccc(N(c4ccc(-c5ccc6cccc(-c7ccccc7)c6c5)cc4)c4cccc5c4sc4ccccc45)c3)c2)cc1 ; c1ccc(-c2cccc(-c3cccc(N(c4ccc(-c5ccc6ccccc6c5-c5ccccc5)cc4)c4cccc5c4oc4ccccc45)c3)c2)cc1 ; c1ccc(-c2cccc(-c3cccc(N(c4ccc(-c5ccc6ccccc6c5)cc4)c4cccc5oc6ccccc6c45)c3)c2)cc1]

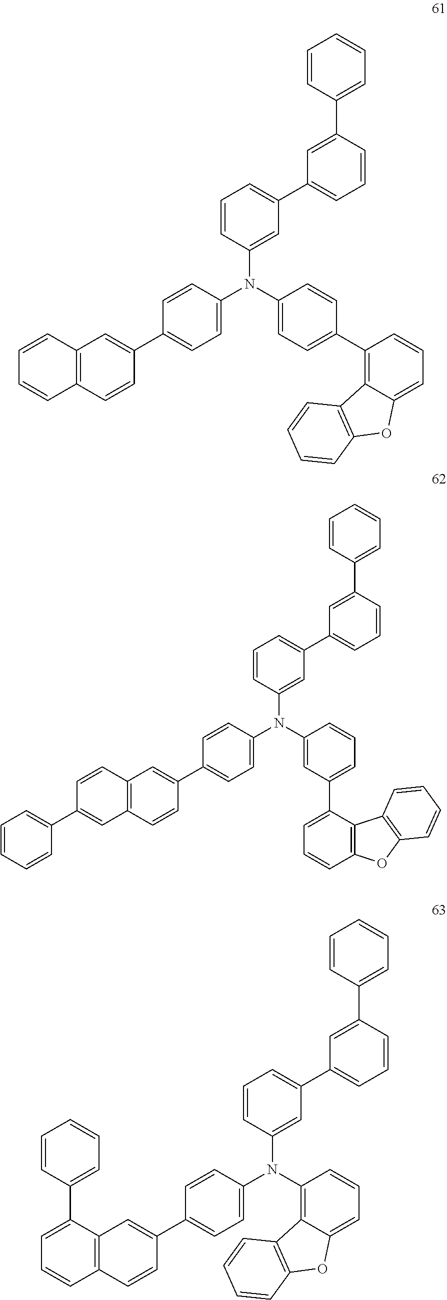

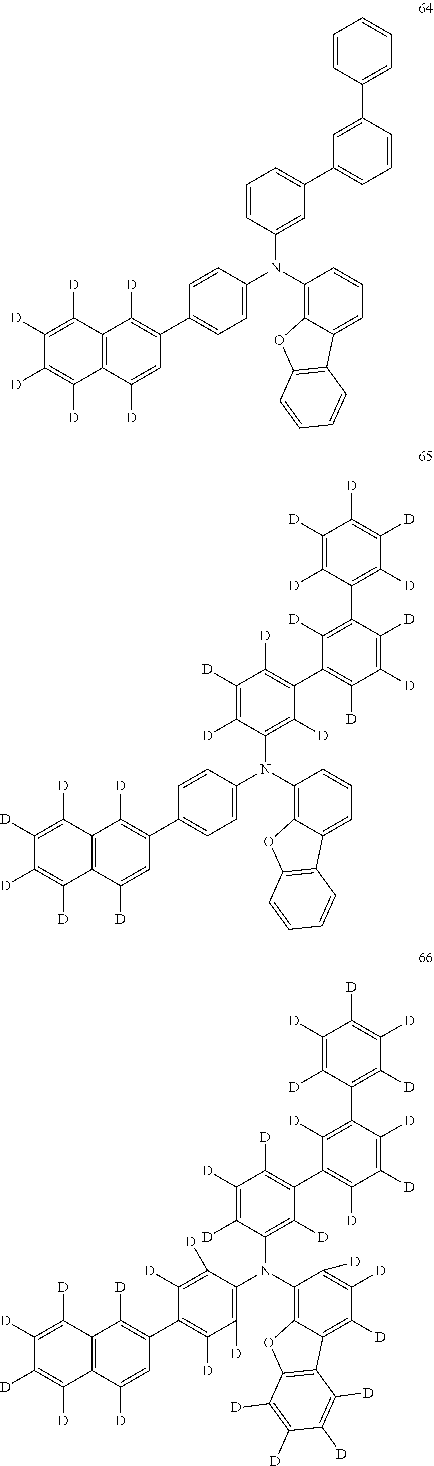

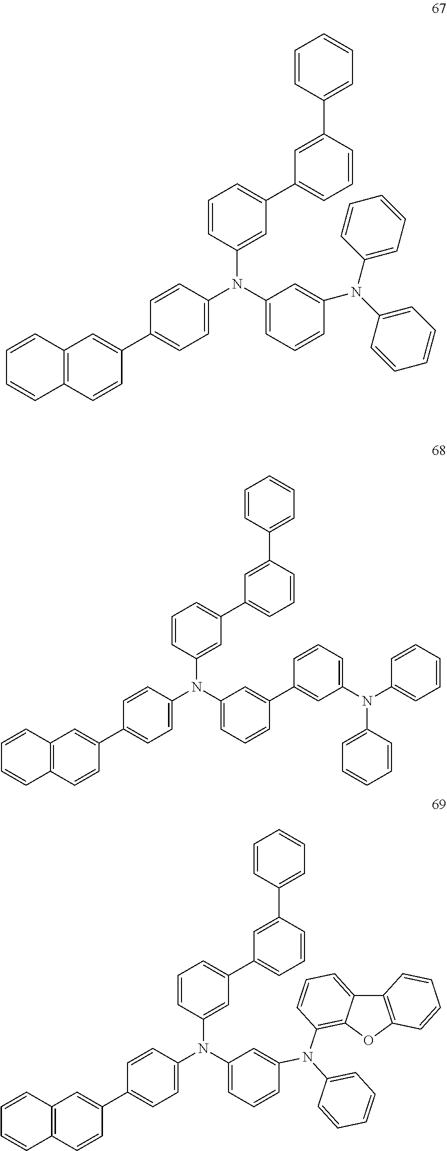 [SMILES: c1ccc(-c2cccc(-c3cccc(N(c4ccc(-c5ccc6ccccc6c5)cc4)c4cccc(N(c5ccccc5)c5ccccc5)c4)c3)c2)cc1 ; c1ccc(-c2cccc(-c3cccc(N(c4ccc(-c5ccc6ccccc6c5)cc4)c4cccc(-c5cccc(N(c6ccccc6)c6ccccc6)c5)c4)c3)c2)cc1 ; c1ccc(-c2cccc(-c3cccc(N(c4ccc(-c5ccc6ccccc6c5)cc4)c4cccc(N(c5ccccc5)c5cccc6c5oc5ccccc56)c4)c3)c2)cc1]

The hole transport region HTR may include at least one selected from among a hole injection layer HIL, a hole transport layer HTL, a buffer layer, a light emitting auxiliary layer, and an electron blocking layer EBL. Also, the hole transport region HTR may include two hole transport layers HTL-1 and HTL-2. Hereinafter, descriptions of the hole transport layer HTL may also be applied to the first hole transport layer HTL-1 and the second hole transport layer HTL-2.

The hole transport region HTR may have a single layer formed of a single material, a single layer formed of a plurality of different materials, or a multilayer structure having a plurality of layers formed of a plurality of different materials. For example, the hole transport region HTR may have a single-layer structure formed of the hole injection layer HIL or the hole transport layer HTL, or a single-layer structure formed of a hole injection material or a hole transport material.

For example, the hole transport region HTR may have a single-layer structure formed of a plurality of different materials, or a structure in which a hole injection layer HIL/hole transport layer HTL, a hole injection layer HIL/hole transport layer HTL/buffer layer, a hole injection layer HIL/buffer layer, a hole transport layer HTL/buffer layer, or a hole injection layer HIL/hole transport layer HTL/electron blocking layer EBL are stacked in order from the first electrode EL1. However, this is presented as an example, and the embodiment of the present disclosure is not limited thereto.

The hole transport region HTR may be formed using various suitable methods such as a vacuum deposition method, a spin coating method, a cast method, a Langmuir-Blodgett (LB) method, an inkjet printing method, a laser printing method, and/or a laser induced thermal imaging (LITI) method.

The hole transport region HTR may include a compound represented by Formula H-1 below.

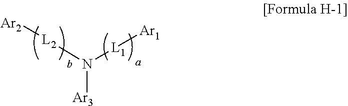

In Formula H-1 above, L₁ and L₂ may each independently be a direct linkage, a substituted or unsubstituted arylene group having 6 to 30 ring-forming carbon atoms, or a substituted or unsubstituted heteroarylene group having 2 to 30 ring-forming carbon atoms. In embodiments of the disclosure, a and b may each independently be an integer of 0 to 10. In some embodiments, when a or b is an integer of 2 or greater, a plurality of L₁'s and L₂'s may each independently be a substituted or unsubstituted arylene group having 6 to 30 ring-forming carbon atoms, or a substituted or unsubstituted heteroarylene group having 2 to 30 ring-forming carbon atoms.

In Formula H-1, Ar₁ and Ar₂ may each independently be a substituted or unsubstituted aryl group having 6 to 30 ring-forming carbon atoms, or a substituted or unsubstituted heteroaryl group having 2 to 30 ring-forming carbon atoms. In addition, in Formula H-1, Ar₃ may be a substituted or unsubstituted aryl group having 6 to 30 ring-forming carbon atoms.

A compound represented by Formula H-1 above may be a monoamine compound. In some embodiments, the compound represented by Formula H-1 may be a diamine compound in which at least one selected from Ar₁ to Ar₃ includes an amine group as a substituent. In addition, the compound represented by Formula H-1 may be a carbazole-based compound including a substituted or unsubstituted carbazole group in at least one selected from Ar₁ and Ar₂ or a substituted or unsubstituted fluorene-based group in at least one selected from Ar₁ and Ar₂.

The compound represented by Formula H-1 may be represented by any one selected from among compounds from Compound Group H below. However, the compounds listed in Compound Group H below are presented as an example, and the compound represented by Formula H-1 is not limited to the those listed in Compound Group H below.

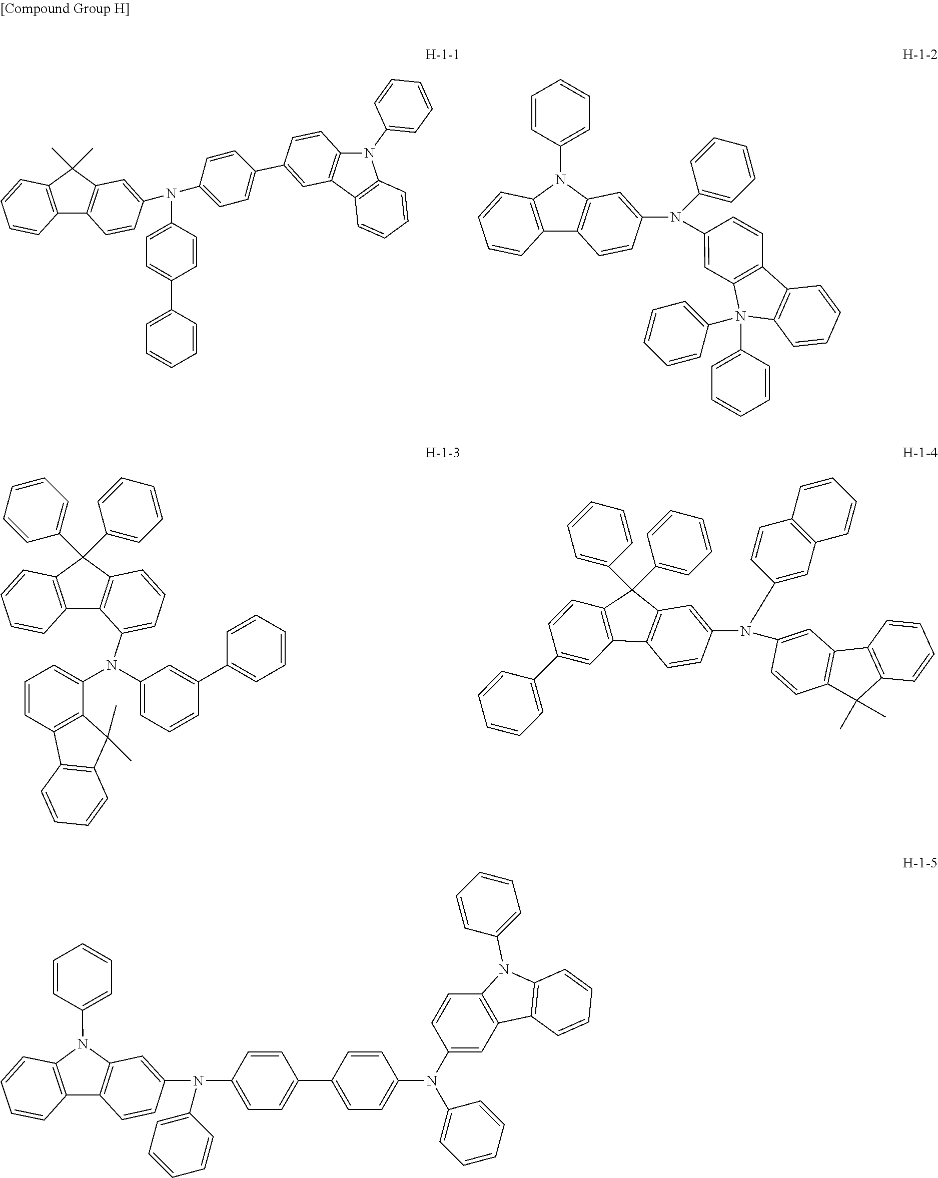

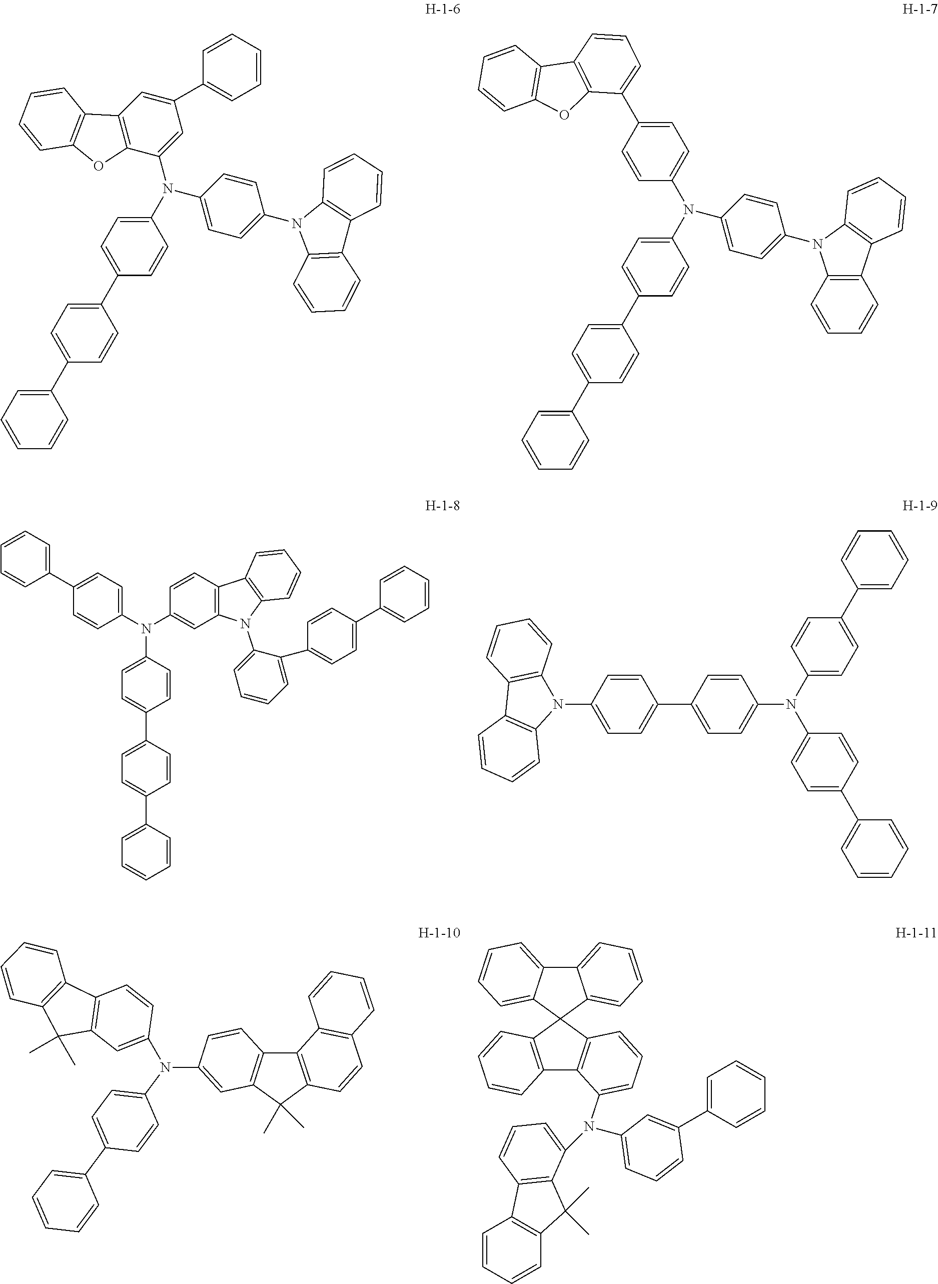

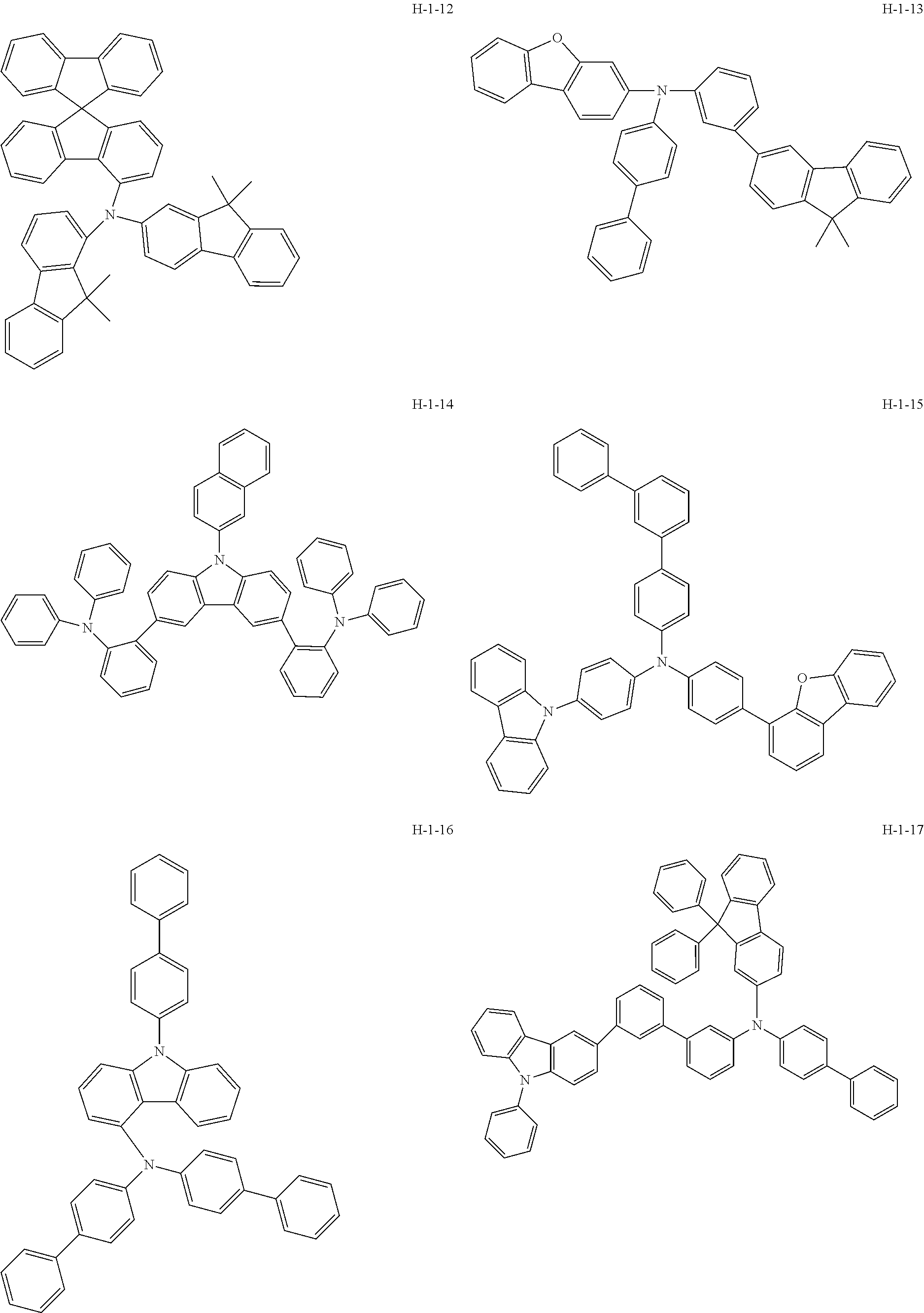

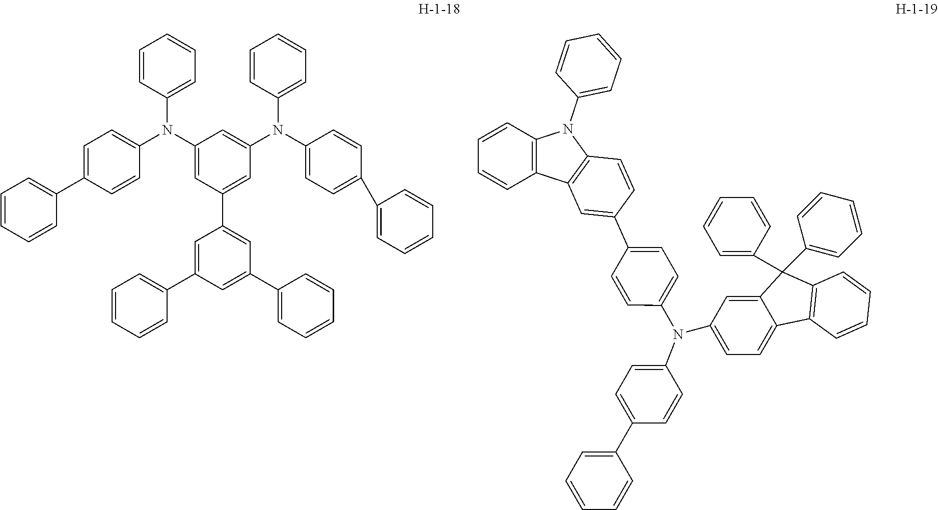 [SMILES: c1ccc(-c2ccc(N(c3ccccc3)c3cc(-c4cc(-c5ccccc5)cc(-c5ccccc5)c4)cc(N(c4ccccc4)c4ccc(-c5ccccc5)cc4)c3)cc2)cc1 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1]

The hole transport region HTR may include a phthalocyanine compound such as copper phthalocyanine, N¹,N¹-([1,1′-biphenyl]-4,4′-diyl)bis(N¹-phenyl-N⁴,N⁴-di-m-tolylbenzene-1,4-diamine) (DNTPD), 4,4′,4″-[tris(3-methylphenyl)phenylamino]triphenylamine (m-MTDATA), 4,4′4″-tris(N,N-diphenylamino)triphenylamine (TDATA), 4,4′,4″-tris[N-(2-naphthyl)-N-phenylamino]-triphenylamine (2-TNATA), poly(3,4-ethylenedioxythiophene)/poly(4-styrenesulfonate) (PEDOT/PSS), polyaniline/dodecylbenzenesulfonic acid (PANI/DBSA), polyaniline/camphor sulfonicacid (PANI/CSA), polyaniline/poly(4-styrenesulfonate) (PANI/PSS), N,N′-di(naphthalene-1-yl)-N,N′-diphenyl-benzidine (NPB), triphenylamine-containing polyetherketone (TPAPEK), 4-isopropyl-4′-methyldiphenyliodonium tetrakis(pentafluorophenyl)borate, dipyrazino[2,3-f: 2′,3′-h] quinoxaline-2,3,6,7,10,11-hexacarbonitrile (HATCN), and the like.

In addition, the hole transport region HTR may include carbazole-based derivatives such as N-phenyl carbazole and/or polyvinyl carbazole, fluorene-based derivatives, N,N′-bis(3-methylphenyl)-N,N′-diphenyl-[1,1′-biphenyl]-4,4′-diamine (TPD), triphenylamine-based derivatives such as 4,4′,4″-tris(N-carbazolyl)triphenylamine (TCTA), 4,4′-cyclohexylidene bis[N,N-bis(4-methylphenyl]benzenamine] (TAPC), 4,4′-bis[N,N′-(3-tolyl)amino]-3,3′-dimethylbiphenyl (HMTPD), 9-(4-tert-butylphenyl)-3,6-bis(triphenylsilyl)-9H-carbazole (CzSi), 9-phenyl-9H-3,9′-bicarbazole (CCP), 1,3-bis(N-carbazol-9-yl)benzene (mCP), 1,3-bis(1,8-dimethyl-9H-carbazol-9-yl)benzene (DCP), and/or the like.

The hole transport region HTR may include an amine compound of an embodiment. In addition, the hole transport region HTR may include the compounds of the hole transport region described above in at least one selected from among the hole injection layer HIL, the hole transport layer HTL, and the electron blocking layer EBL.

The hole transport region HTR may have, for example, a thickness of about 50 Å to about 15000 Å. The hole transport region HTR may have a thickness of about 100 Å to about 10000 Å, for example, about 100 Å to about 5000 Å. When the hole transport region HTR includes the hole injection layer HIL, the hole injection layer HIL may have a thickness of, for example, about 30 Å to about 1000 Å. When the hole transport region HTR includes the hole transport layer HTL, the hole transport layer HTL may have a thickness of about 30 Å to about 1000 Å. When the hole transport region HTR includes the electron blocking layer EBL, the electron blocking layer EBL may have a thickness of, for example, about 10 Å to about 1000 Å. When the thicknesses of the hole transport region HTR, the hole injection layer HIL, the hole transport layer HTL, and the electron blocking layer EBL satisfy the above-described ranges, suitable or satisfactory hole transport properties may be obtained without a substantial increase in driving voltage.

The hole transport region HTR may further include, in addition to the above-described materials, a charge generation material to increase conductivity (e.g., electrical conductivity). The charge generation material may be uniformly or non-uniformly dispersed in the hole transport region HTR. The charge generation material may be, for example, a p-dopant. The p-dopant may include at least one selected from halogenated metal compounds, quinone derivatives, metal oxides, and cyano group-containing compounds, but is not limited thereto. For example, the p-dopant may include halogenated metal compounds such as CuI and/or RbI, quinone derivatives such as tetracyanoquinodimethane (TCNQ) and/or 2,3,5,6-tetrafluoro-7,7′,8,8′-tetracyanoquinodimethane (F4-TCNQ), metal oxides such as tungsten oxides and/or molybdenum oxides, cyano group-containing compounds such as dipyrazino[2,3-f:2′,3′-h] quinoxaline-2,3,6,7,10,11-hexacarbonitrile (HATCN) and/or 4-[[2,3-bis[cyano-(4-cyano-2,3,5,6-tetrafluorophenyl)methylidene]cyclopropylidene]-cyanomethyl]-2,3,5,6-tetrafluorobenzonitrile (NDP9), and/or the like, but is not limited thereto.

As described above, the hole transport region HTR may further include at least one selected from a buffer layer and an electron blocking layer EBL in addition to the hole injection layer HIL and the hole transport layer HTL. The buffer layer may compensate a resonance distance according to wavelengths of light emitted from an emission layer EML, and may thus increase light emitting efficiency. Materials which may be included in the hole transport region HTR may be used as materials included in the buffer layer. The electron blocking layer EBL is a layer that serves to prevent or reduce injection of electrons from the electron transport region ETR to the hole transport region HTR.

Referring back to FIGS. 3-7, the first electrode EL1 may have conductivity. The first electrode EL1 may be formed of a metal material, a metal alloy or a conductive compound (e.g., an electrically conductive compound). The first electrode EL1 may be an anode or a cathode. However, the embodiment of the present disclosure is not limited thereto. In addition, the first electrode EL1 may be a pixel electrode. The first electrode EL1 may be a transmissive electrode, a transflective electrode, or a reflective electrode. The first electrode may include at least one selected from Ag, Mg, Cu, Al, Pt, Pd, Au, Ni, Nd, Ir, Cr, Li, Ca, LiF, Mo, Ti, W, In, Sn, and Zn, two or more compounds selected therefrom, two or more mixtures selected therefrom, and/or an oxide thereof.

When the first electrode EL1 is the transmissive electrode, the first electrode EL1 may include a transparent metal oxide such as indium tin oxide (ITO), indium zinc oxide (IZO), zinc oxide (ZnO), and/or indium tin zinc oxide (ITZO). When the first electrode EL1 is the transflective electrode or the reflective electrode, the first electrode EL1 may include Ag, Mg, Cu, Al, Pt, Pd, Au, Ni, Nd, Ir, Cr, Li, Ca, LiF/Ca (a stack structure of LiF and Ca), LiF/Al (a stack structure of LiF and Al), Mo, Ti, W, a compound thereof, or a mixture thereof (e.g., a mixture of Ag and Mg). In some embodiments, the first electrode EL1 may have a multilayer structure including a reflective film or a transflective film formed of the above-described materials, and a transparent conductive film formed of indium tin oxide (ITO), indium zinc oxide (IZO), zinc oxide (ZnO), indium tin zinc oxide (ITZO), and/or the like. For example, the first electrode EL1 may have a three-layer structure of ITO/Ag/ITO, but is not limited thereto. In addition, the first electrode EL1 may include the above-described metal materials, a combination of two or more metal materials selected from the above-described metal materials, and/or oxides of the above-described metal materials, and the embodiment of the present disclosure is not limited thereto. The first electrode EL1 may have a thickness of about 700 Å to about 10000 Å. For example, the first electrode EL1 may have a thickness of 1000 Å to about 3000 Å.

The emission layer EML may be provided on the hole transport region HTR. The emission layer EML may have, for example, a thickness of about 100 Å to about 1000 Å or about 100 Å to about 300 Å. The emission layer EML may have a single layer formed of a single material, a single layer formed of a plurality of different materials, or a multilayer structure having a plurality of layers formed of a plurality of different materials.

In the light emitting element ED according to an embodiment, the emission layer EML may include an anthracene derivative, a pyrene derivative, a fluoranthene derivative, a chrysene derivative, a dihydrobenzanthracene derivative, and/or a triphenylene derivative. In some embodiments, the emission layer EML may include an anthracene derivative and/or a pyrene derivative.

In the light emitting element ED according to an embodiment, the emission layer EML may include a host and a dopant. For example, the emission layer EML may include a single host and a single dopant. In some embodiments, the emission layer EML may include two or more hosts, sensitizers, and/or dopants. For example, the emission layer EML may include a hole transporting host and an electron transporting host. The emission layer EML may include a phosphorescent sensitizer or a thermally activated delayed fluorescence (TADF) sensitizer as a sensitizer.

In the emission layer EML, the hole transporting host and the electron transporting host may form an exciplex. In this case, the triplet energy of the exciplex formed by the hole transporting host and the electron transporting host corresponds to a difference between Lowest Unoccupied Molecular Orbital (LUMO) energy level of the electron transporting host and Highest Occupied Molecular Orbital (HOMO) energy level of the hole transporting host. For example, the triplet energy level (T1) of the exciplex formed by the hole transporting host and the electron transporting host may have an absolute value of about 2.4 eV to about 3.0 eV. In addition, the triplet energy of the exciplex may have a value smaller than the energy gap of each host material. The exciplex may have a triplet energy of 3.0 eV or less, which is an energy gap between the hole transporting host and the electron transporting host. However, this is presented as an example, and the embodiment of the present disclosure is not limited thereto.

When the emission layer EML includes the hole transporting host, the electron transporting host, a sensitizer, and a dopant, the hole transporting host and the electron transporting host may form an exciplex, and energy may be transferred from the exciplex to the sensitizer and from the sensitizer to the dopant, to thereby emit light. However, this is presented as an example, and materials included in the emission layer EML are not limited thereto. In addition, the hole transporting host and the electron transporting host may not form an exciplex. When the hole transporting host and the electron transporting host do not form an exciplex, energy may be transferred from the hole transporting host and the electron transporting host to the sensitizer and from the sensitizer to the dopant, to thereby emit light.

The emission layer EML may include a compound represented by Formula E-1 below. The compound represented by Formula E-1 below may be used as a fluorescent host material.

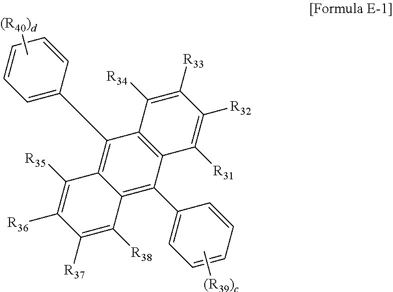

In Formula E-1, R₃₁ to R₄₀ may each independently be a hydrogen atom, a deuterium atom, a halogen atom, a substituted or unsubstituted silyl group, a substituted or unsubstituted thio group, a substituted or unsubstituted oxy group, a substituted or unsubstituted alkyl group having 1 to 10 carbon atoms, a substituted or unsubstituted alkenyl group having 2 to 10 carbon atoms, a substituted or unsubstituted aryl group having 6 to 30 ring-forming carbon atoms, a substituted or unsubstituted heteroaryl group having 2 to 30 ring-forming carbon atoms, or bonded to an adjacent group to form a ring. In some embodiments, R₃₁ to R₄₀ may be bonded to an adjacent group to form a saturated hydrocarbon ring, an unsaturated hydrocarbon ring, a saturated heterocycle, or an unsaturated heterocycle.

In Formula E-1, c and d may each independently be an integer of 0 to 5. When c is an integer of 2 or greater, a plurality of R₃₉'s may all be the same or at least one may be different from the others. When d is an integer of 2 or greater, a plurality of R₄₀'s may all be the same or at least one may be different from the others. Formula E-1 may be represented by any one selected from among compounds E1 to E19 below.

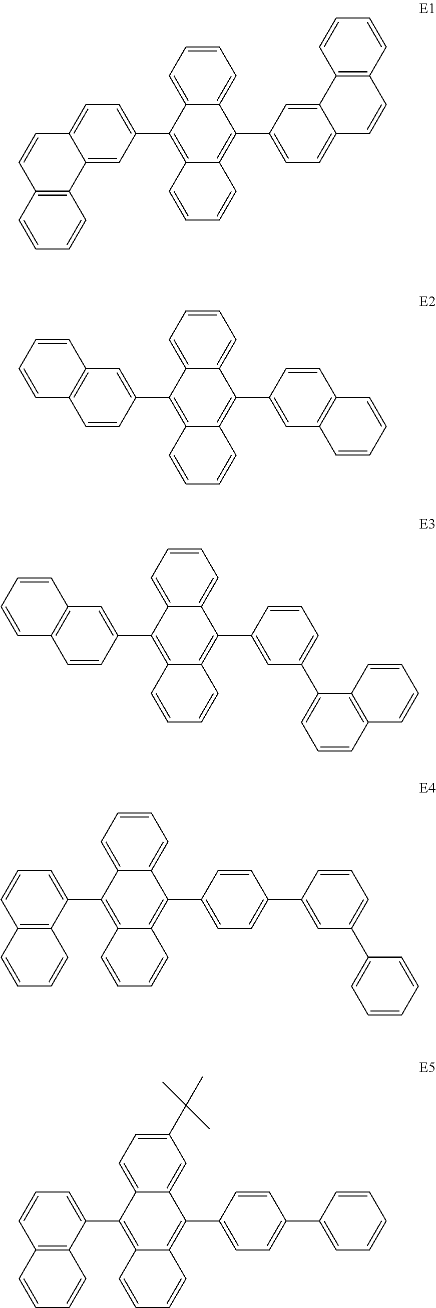

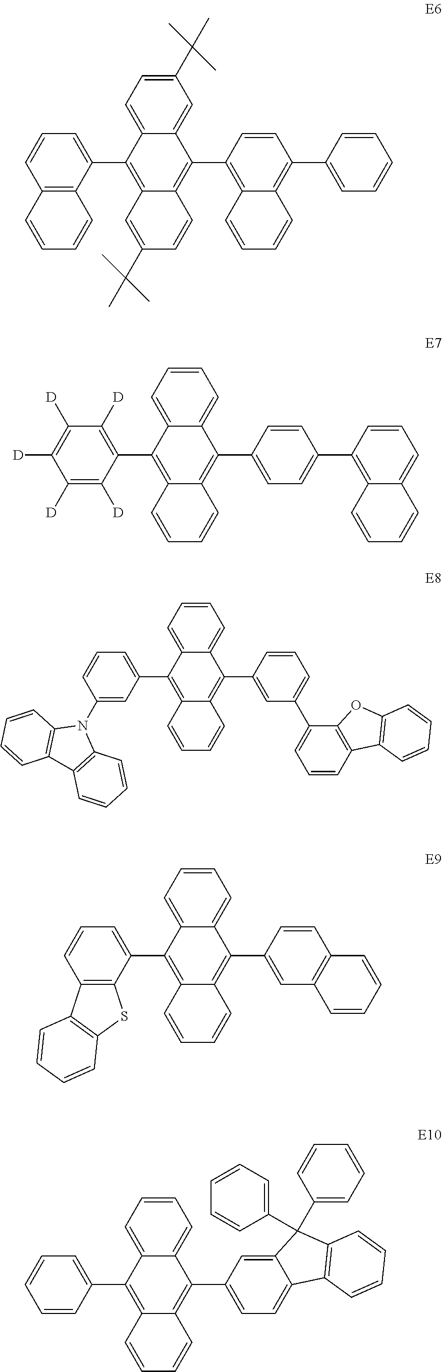

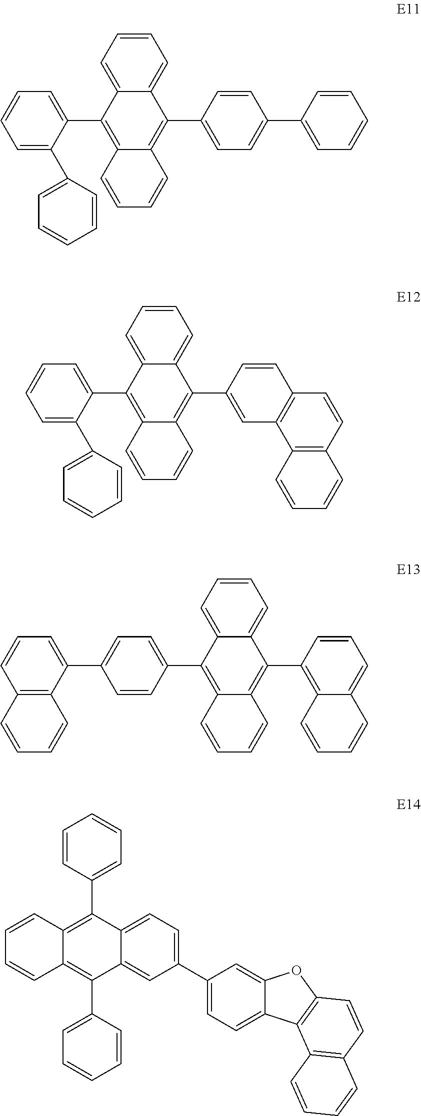

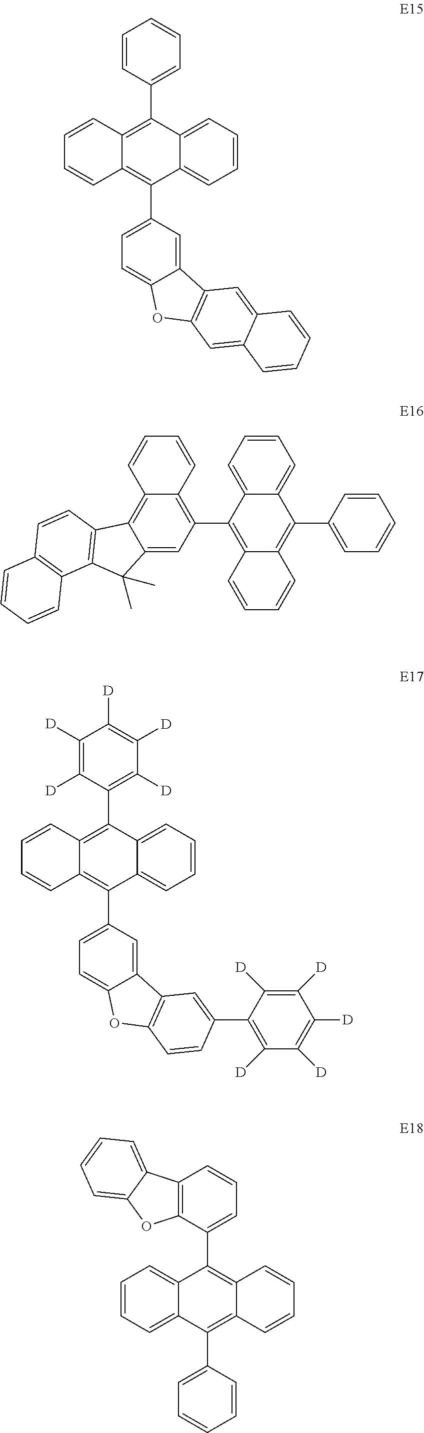

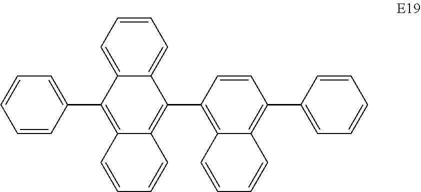 [SMILES: c1ccc(-c2ccc(-c3c4ccccc4c(-c4ccccc4)c4ccccc34)c3ccccc23)cc1]

In an embodiment, the emission layer EML may include at least one selected from a first compound represented by Formula HT-1 below, a second compound represented by Formula ET-1 below, and a third compound represented by Formula M-b below. The first compound may be used as a hole transporting host material of the emission layer EML.

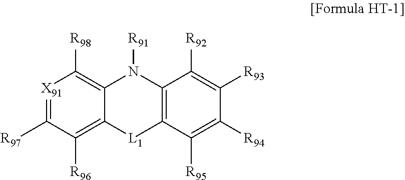

In Formula HT-1, L1 may be a direct linkage, CR₉₉R₁₀₀, or SiR₁₀₁R₁₀₂. In Formula HT-1, X₉₁ may be N or CR₁₀₃. When L1 is a direct linkage and X₉₁ is CR₁₀₃, the second compound represented by Formula HT-1 may include a carbazole group.

R₉₁ to R₁₀₃ may each independently be a hydrogen atom, a deuterium atom, a cyano group, a substituted or unsubstituted silyl group, a substituted or unsubstituted oxy group, a substituted or unsubstituted alkyl group having 1 to 60 carbon atoms, a substituted or unsubstituted aryl group having 6 to 60 ring-forming carbon atoms, or a substituted or unsubstituted heteroaryl group having 2 to 60 ring-forming carbon atoms, or bonded to an adjacent group to form a ring, and For example, R₉₁ may be a substituted phenyl group, an unsubstituted dibenzofuran group, or a substituted fluorenyl group. Any one selected from among R₉₂ to R₉₈ may be a substituted or unsubstituted carbazole group. R₉₄ and R₉₅ may be bonded to each other to form a ring. However, this is presented as an example, and the embodiment of the present disclosure is not limited thereto.

The first compound may be represented by any one selected from among compounds of Compound Group HT below. In Compound Group HT below, D is a deuterium atom, and Ph is a phenyl group.

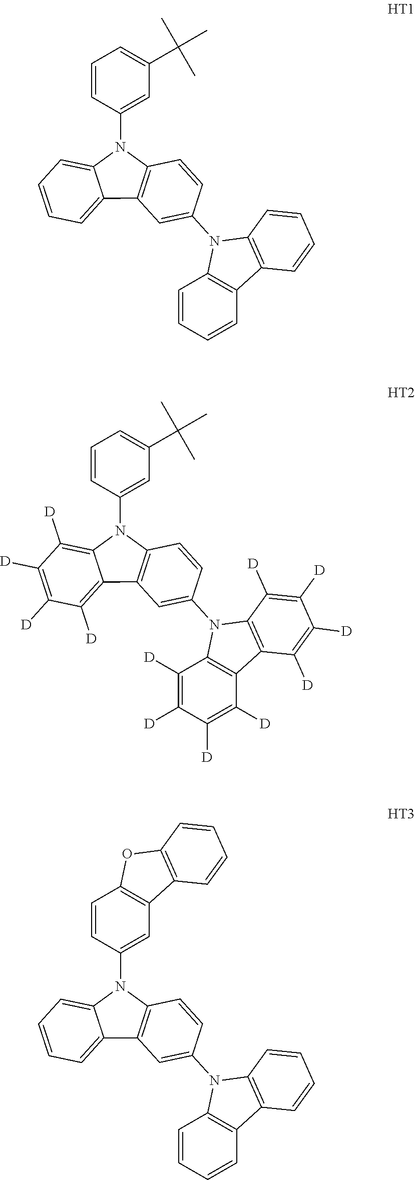 [SMILES: CC(C)(C)c1cccc(-n2c3ccccc3c3cc(-n4c5ccccc5c5ccccc54)ccc32)c1 ; [2H]c1c([2H])c([2H])c2c(c1[2H])c1cc(-n3c4c([2H])c([2H])c([2H])c([2H])c4c4c([2H])c([2H])c([2H])c([2H])c43)ccc1n2-c1cccc(C(C)(C)C)c1 ; c1ccc2c(c1)oc1ccc(-n3c4ccccc4c4cc(-n5c6ccccc6c6ccccc65)ccc43)cc12]

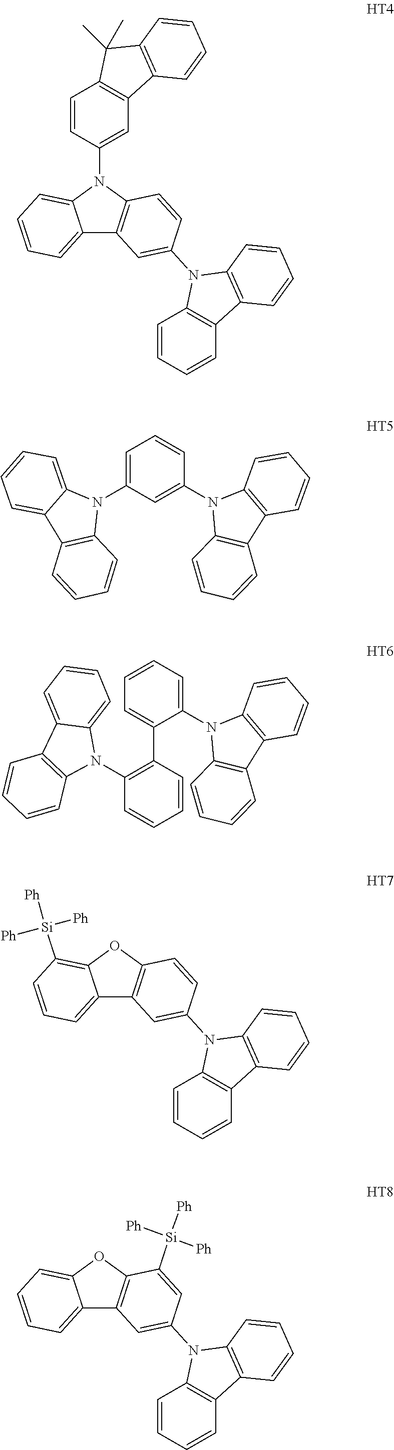 [SMILES: CC1(C)c2ccccc2-c2cc(-n3c4ccccc4c4cc(-n5c6ccccc6c6ccccc65)ccc43)ccc21 ; c1cc(-n2c3ccccc3c3ccccc32)cc(-n2c3ccccc3c3ccccc32)c1 ; c1ccc(-n2c3ccccc3c3ccccc32)c(-c2ccccc2-n2c3ccccc3c3ccccc32)c1 ; c1ccc([Si](c2ccccc2)(c2ccccc2)c2cccc3c2oc2ccc(-n4c5ccccc5c5ccccc54)cc23)cc1 ; c1ccc([Si](c2ccccc2)(c2ccccc2)c2cc(-n3c4ccccc4c4ccccc43)cc3c2oc2ccccc23)cc1]

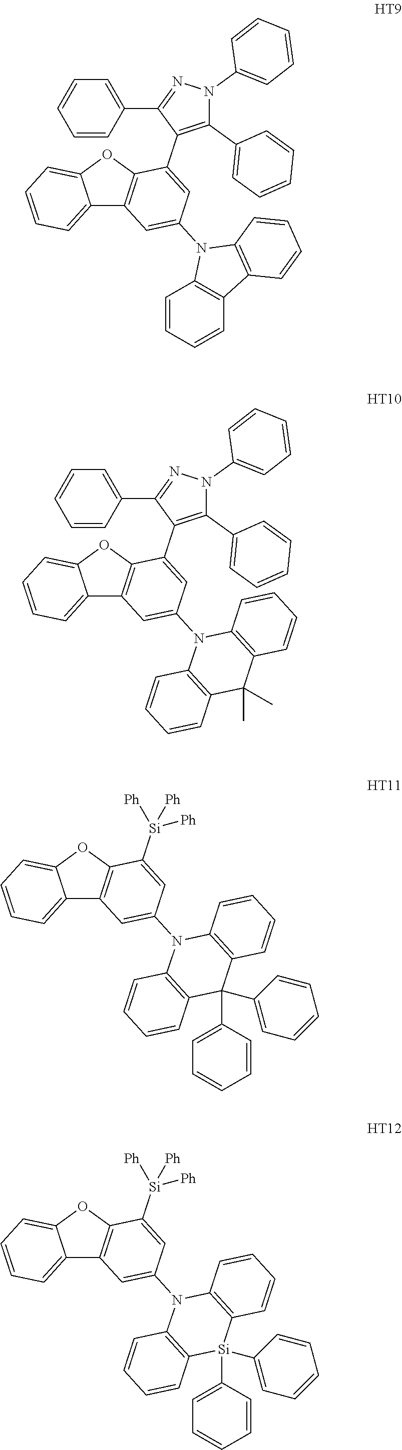 [SMILES: c1ccc(-c2nn(-c3ccccc3)c(-c3ccccc3)c2-c2cc(-n3c4ccccc4c4ccccc43)cc3c2oc2ccccc23)cc1 ; CC1(C)c2ccccc2N(c2cc(-c3c(-c4ccccc4)nn(-c4ccccc4)c3-c3ccccc3)c3oc4ccccc4c3c2)c2ccccc21 ; c1ccc(C2(c3ccccc3)c3ccccc3N(c3cc([Si](c4ccccc4)(c4ccccc4)c4ccccc4)c4oc5ccccc5c4c3)c3ccccc32)cc1 ; c1ccc([Si]2(c3ccccc3)c3ccccc3N(c3cc([Si](c4ccccc4)(c4ccccc4)c4ccccc4)c4oc5ccccc5c4c3)c3ccccc32)cc1]

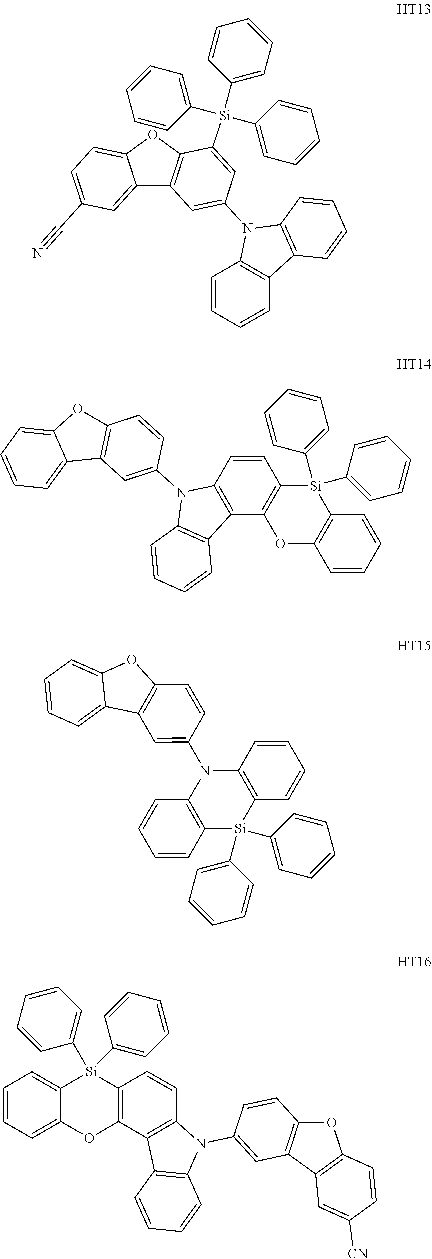 [SMILES: N#Cc1ccc2oc3c([Si](c4ccccc4)(c4ccccc4)c4ccccc4)cc(-n4c5ccccc5c5ccccc54)cc3c2c1 ; c1ccc([Si]2(c3ccccc3)c3ccccc3Oc3c2ccc2c3c3ccccc3n2-c2ccc3oc4ccccc4c3c2)cc1 ; c1ccc([Si]2(c3ccccc3)c3ccccc3N(c3ccc4oc5ccccc5c4c3)c3ccccc32)cc1 ; N#Cc1ccc2oc3ccc(-n4c5ccccc5c5c6c(ccc54)[Si](c4ccccc4)(c4ccccc4)c4ccccc4O6)cc3c2c1]

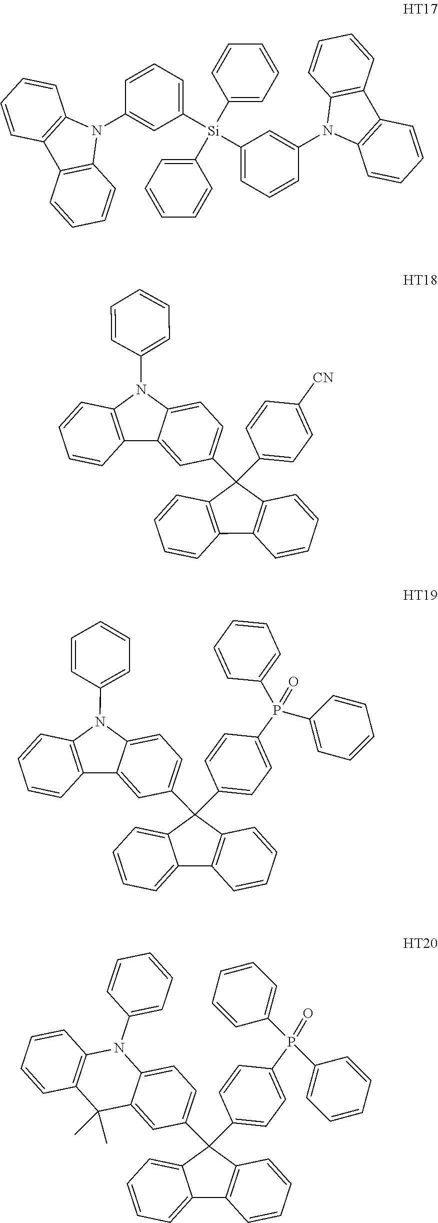 [SMILES: c1ccc([Si](c2ccccc2)(c2cccc(-n3c4ccccc4c4ccccc43)c2)c2cccc(-n3c4ccccc4c4ccccc43)c2)cc1 ; N#Cc1ccc(C2(c3ccc4c(c3)c3ccccc3n4-c3ccccc3)c3ccccc3-c3ccccc32)cc1 ; O=P(c1ccccc1)(c1ccccc1)c1ccc(C2(c3ccc4c(c3)c3ccccc3n4-c3ccccc3)c3ccccc3-c3ccccc32)cc1 ; CC1(C)c2ccccc2N(c2ccccc2)c2ccc(C3(c4ccc(P(=O)(c5ccccc5)c5ccccc5)cc4)c4ccccc4-c4ccccc43)cc21]

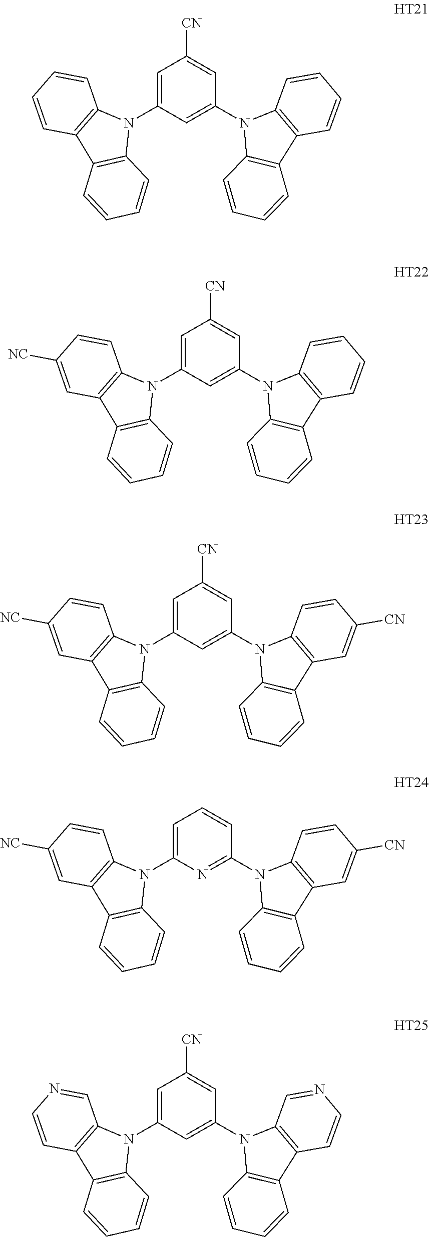 [SMILES: N#Cc1cc(-n2c3ccccc3c3ccccc32)cc(-n2c3ccccc3c3ccccc32)c1 ; N#Cc1cc(-n2c3ccccc3c3ccccc32)cc(-n2c3ccccc3c3cc(C#N)ccc32)c1 ; N#Cc1cc(-n2c3ccccc3c3cc(C#N)ccc32)cc(-n2c3ccccc3c3cc(C#N)ccc32)c1 ; N#Cc1ccc2c(c1)c1ccccc1n2-c1cccc(-n2c3ccccc3c3cc(C#N)ccc32)n1 ; N#Cc1cc(-n2c3ccccc3c3ccncc32)cc(-n2c3ccccc3c3ccncc32)c1]

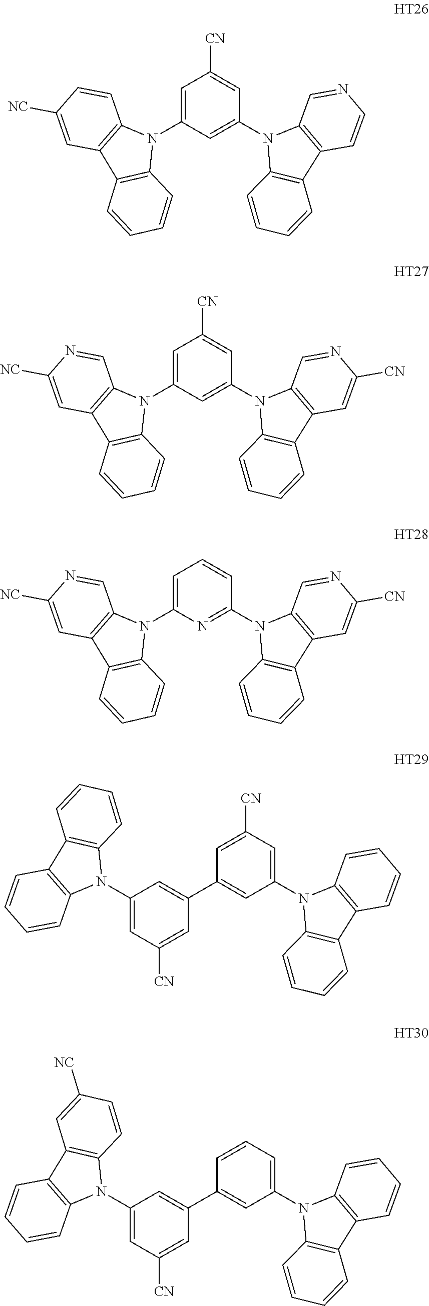 [SMILES: N#Cc1cc(-n2c3ccccc3c3cc(C#N)ccc32)cc(-n2c3ccccc3c3ccncc32)c1 ; N#Cc1cc(-n2c3ccccc3c3cc(C#N)ncc32)cc(-n2c3ccccc3c3cc(C#N)ncc32)c1 ; N#Cc1cc2c3ccccc3n(-c3cccc(-n4c5ccccc5c5cc(C#N)ncc54)n3)c2cn1 ; N#Cc1cc(-c2cc(C#N)cc(-n3c4ccccc4c4ccccc43)c2)cc(-n2c3ccccc3c3ccccc32)c1 ; N#Cc1cc(-c2cccc(-n3c4ccccc4c4ccccc43)c2)cc(-n2c3ccccc3c3cc(C#N)ccc32)c1]

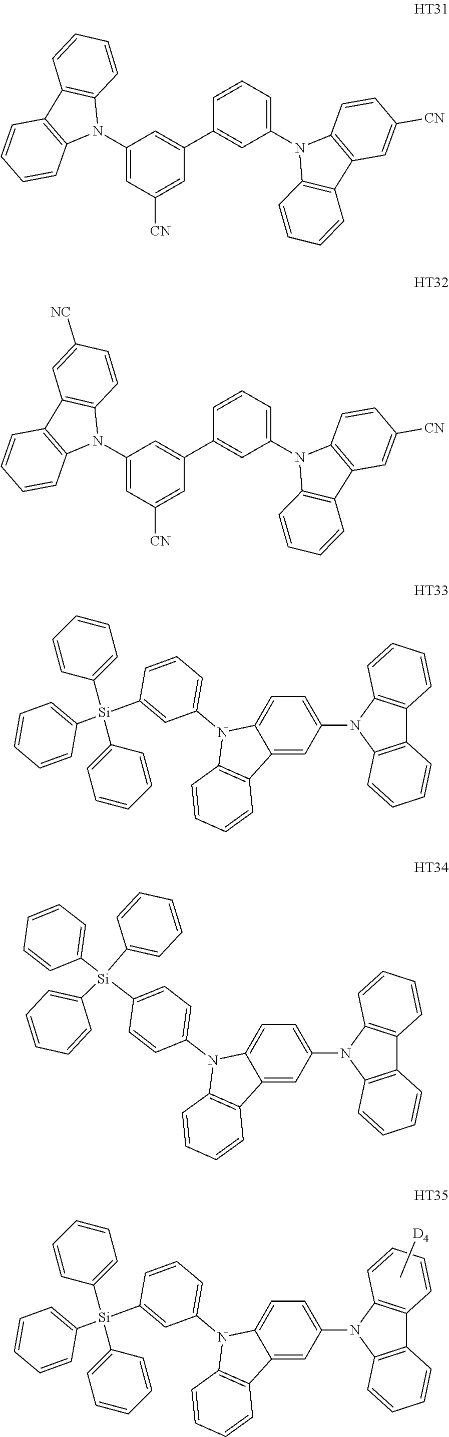

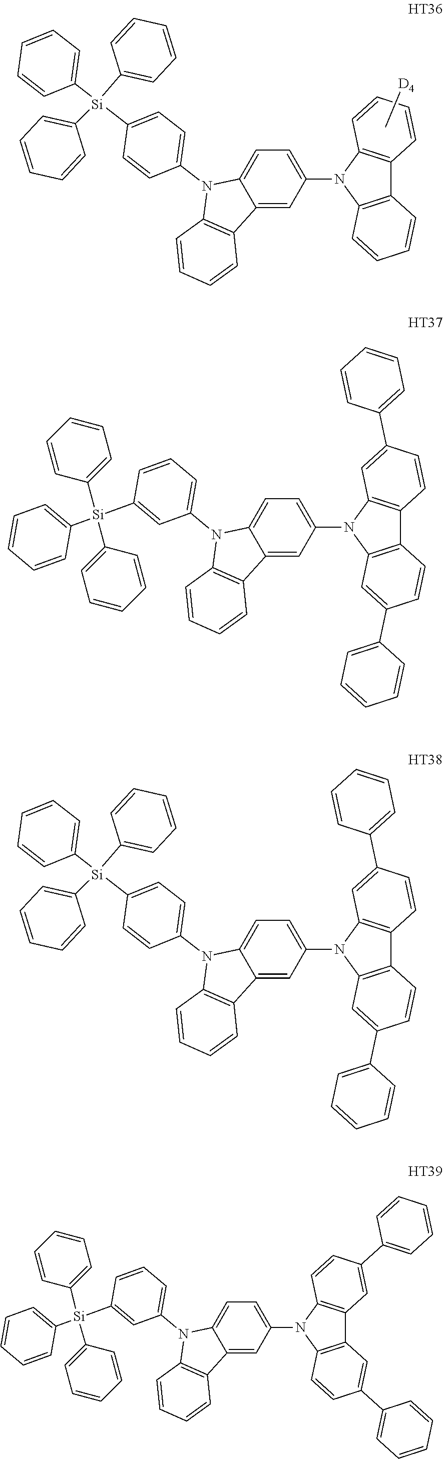

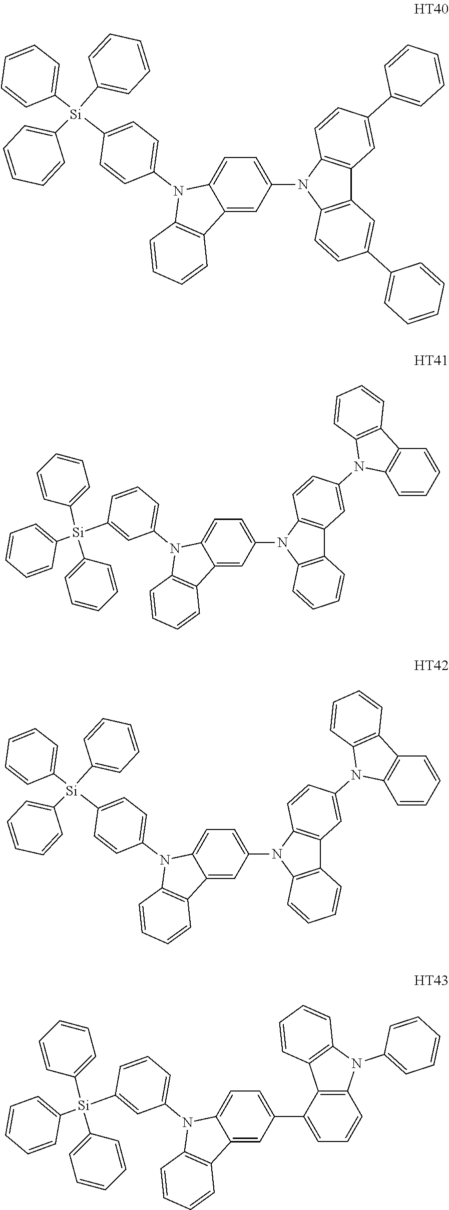 [SMILES: c1ccc(-c2ccc3c(c2)c2cc(-c4ccccc4)ccc2n3-c2ccc3c(c2)c2ccccc2n3-c2ccc([Si](c3ccccc3)(c3ccccc3)c3ccccc3)cc2)cc1 ; c1ccc([Si](c2ccccc2)(c2ccccc2)c2cccc(-n3c4ccccc4c4cc(-n5c6ccccc6c6cc(-n7c8ccccc8c8ccccc87)ccc65)ccc43)c2)cc1 ; c1ccc([Si](c2ccccc2)(c2ccccc2)c2ccc(-n3c4ccccc4c4cc(-n5c6ccccc6c6cc(-n7c8ccccc8c8ccccc87)ccc65)ccc43)cc2)cc1 ; c1ccc(-n2c3ccccc3c3c(-c4ccc5c(c4)c4ccccc4n5-c4cccc([Si](c5ccccc5)(c5ccccc5)c5ccccc5)c4)cccc32)cc1]

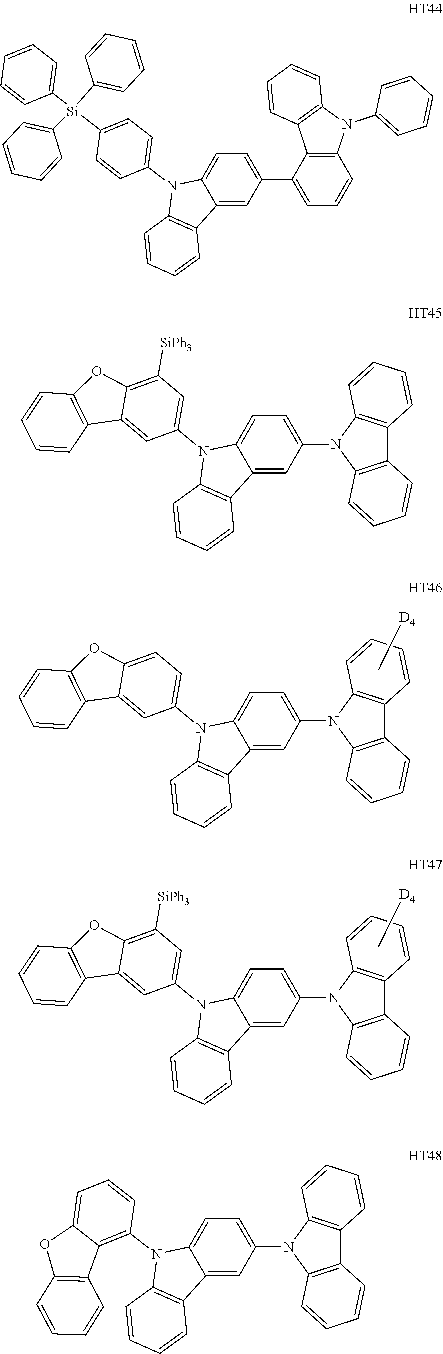

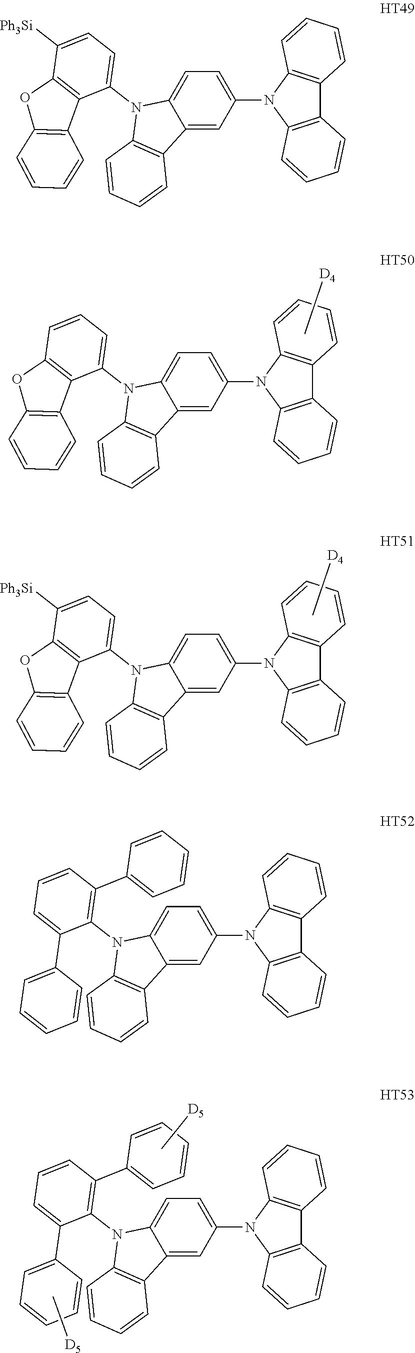

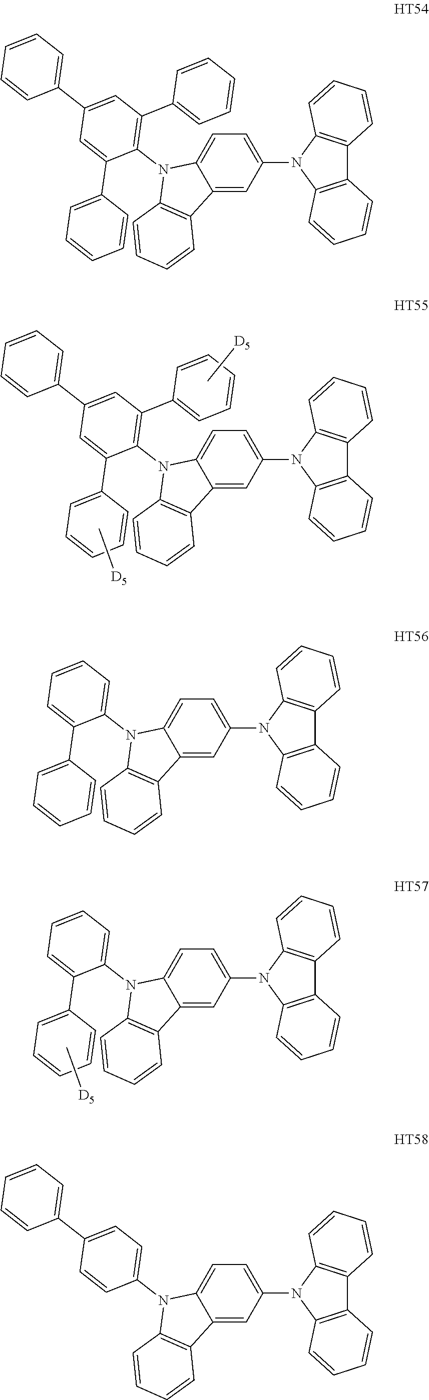

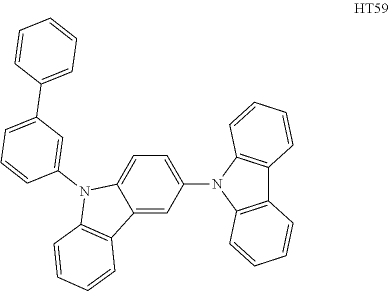 [SMILES: c1ccc(-c2cccc(-n3c4ccccc4c4cc(-n5c6ccccc6c6ccccc65)ccc43)c2)cc1]

In an embodiment, the second compound may be represented by Formula ET-1 below. For example, the emission layer EML may include the second compound as an electron transporting host material.

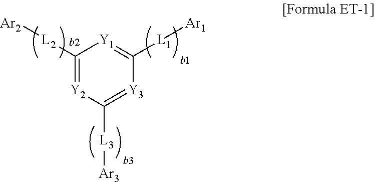

In Formula ET-1, at least one selected from Y₁ to Y₃ may be N and the others may each independently be CRₐ. Rₐ may be a hydrogen atom, a deuterium atom, a substituted or unsubstituted alkyl group having 1 to 20 carbon atoms, a substituted or unsubstituted aryl group having 6 to 60 ring-forming carbon atoms, or a substituted or unsubstituted heteroaryl group having 2 to 60 ring-forming carbon atoms.

When any one selected from Y₁ to Y₃ is N, a second compound represented by Formula ET-1 may include a pyridine group. When any two selected from Y₁ to Y₃ are N, a second compound represented by Formula ET-1 may include a pyrimidine group. When all of Y₁ to Y₃ are N, a second compound represented by Formula ET-1 may include a triazine group.

b1 to b3 may each independently be an integer of 0 to 10. L₁ to L₃ may each independently be a direct linkage, a substituted or unsubstituted arylene group having 6 to 30 ring-forming carbon atoms, or a substituted or unsubstituted heteroarylene group having 2 to 30 ring-forming carbon atoms. When b1 to b3 are an integer of 2 or greater, L₁ to L₃ may be each independently a substituted or unsubstituted arylene group having 6 to 30 ring-forming carbon atoms, or a substituted or unsubstituted heteroarylene group having 2 to 30 ring-forming carbon atoms.

Ar₁ to Ar₃ may each independently be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. For example, Ar₁ to Ar₃ may a substituted or unsubstituted phenyl group or a substituted or unsubstituted carbazole group. However, this is presented as an example, and the embodiment of the present disclosure is not limited thereto.

The second compound may be represented by any one selected from among compounds of Compound Group ET below. The light emitting element ED according to an embodiment may include any one selected from among compounds of Compound Group ET below. In Compound Group ET below, D is a deuterium atom.

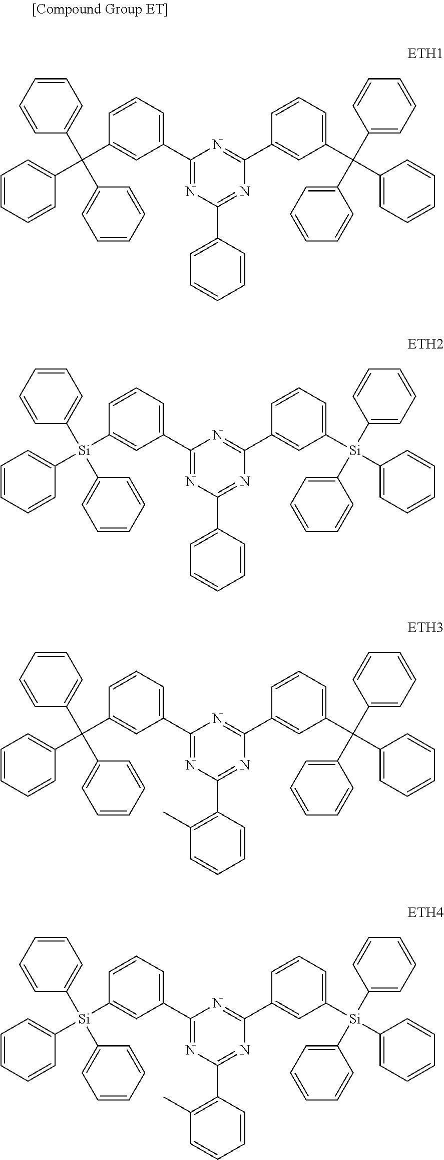 [SMILES: c1ccc(-c2nc(-c3cccc(C(c4ccccc4)(c4ccccc4)c4ccccc4)c3)nc(-c3cccc(C(c4ccccc4)(c4ccccc4)c4ccccc4)c3)n2)cc1 ; c1ccc(-c2nc(-c3cccc([Si](c4ccccc4)(c4ccccc4)c4ccccc4)c3)nc(-c3cccc([Si](c4ccccc4)(c4ccccc4)c4ccccc4)c3)n2)cc1 ; Cc1ccccc1-c1nc(-c2cccc(C(c3ccccc3)(c3ccccc3)c3ccccc3)c2)nc(-c2cccc(C(c3ccccc3)(c3ccccc3)c3ccccc3)c2)n1 ; Cc1ccccc1-c1nc(-c2cccc([Si](c3ccccc3)(c3ccccc3)c3ccccc3)c2)nc(-c2cccc([Si](c3ccccc3)(c3ccccc3)c3ccccc3)c2)n1]

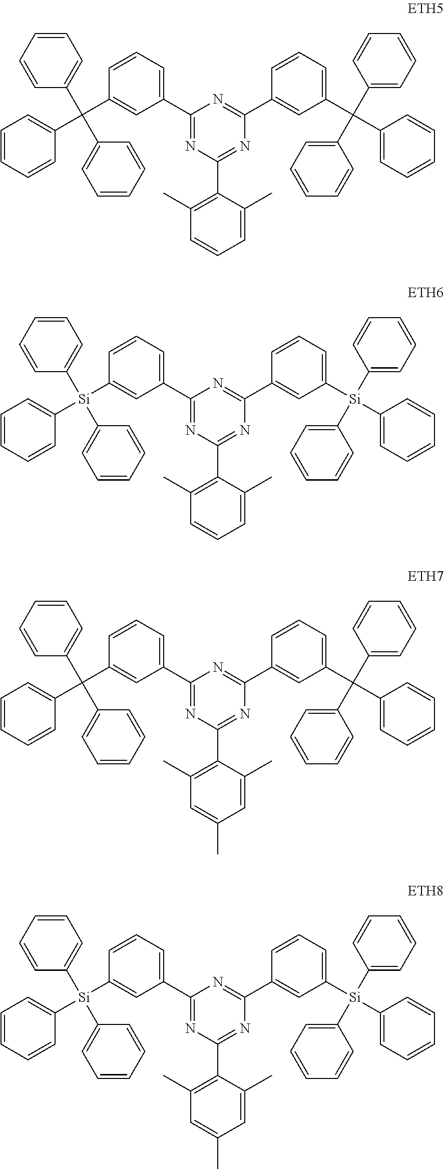 [SMILES: Cc1cccc(C)c1-c1nc(-c2cccc(C(c3ccccc3)(c3ccccc3)c3ccccc3)c2)nc(-c2cccc(C(c3ccccc3)(c3ccccc3)c3ccccc3)c2)n1 ; Cc1cccc(C)c1-c1nc(-c2cccc([Si](c3ccccc3)(c3ccccc3)c3ccccc3)c2)nc(-c2cccc([Si](c3ccccc3)(c3ccccc3)c3ccccc3)c2)n1 ; Cc1cc(C)c(-c2nc(-c3cccc(C(c4ccccc4)(c4ccccc4)c4ccccc4)c3)nc(-c3cccc(C(c4ccccc4)(c4ccccc4)c4ccccc4)c3)n2)c(C)c1 ; Cc1cc(C)c(-c2nc(-c3cccc([Si](c4ccccc4)(c4ccccc4)c4ccccc4)c3)nc(-c3cccc([Si](c4ccccc4)(c4ccccc4)c4ccccc4)c3)n2)c(C)c1]

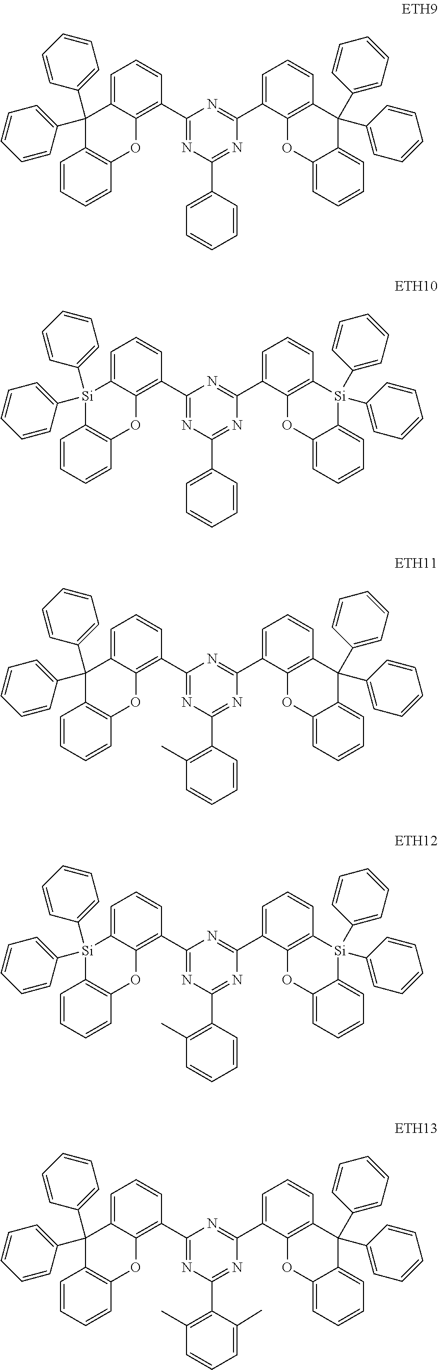

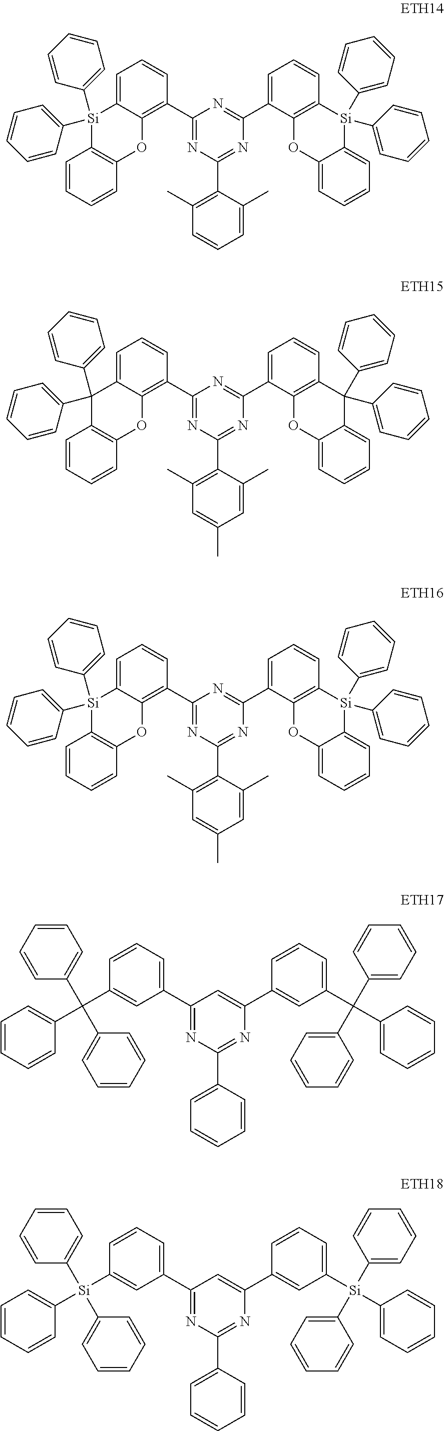

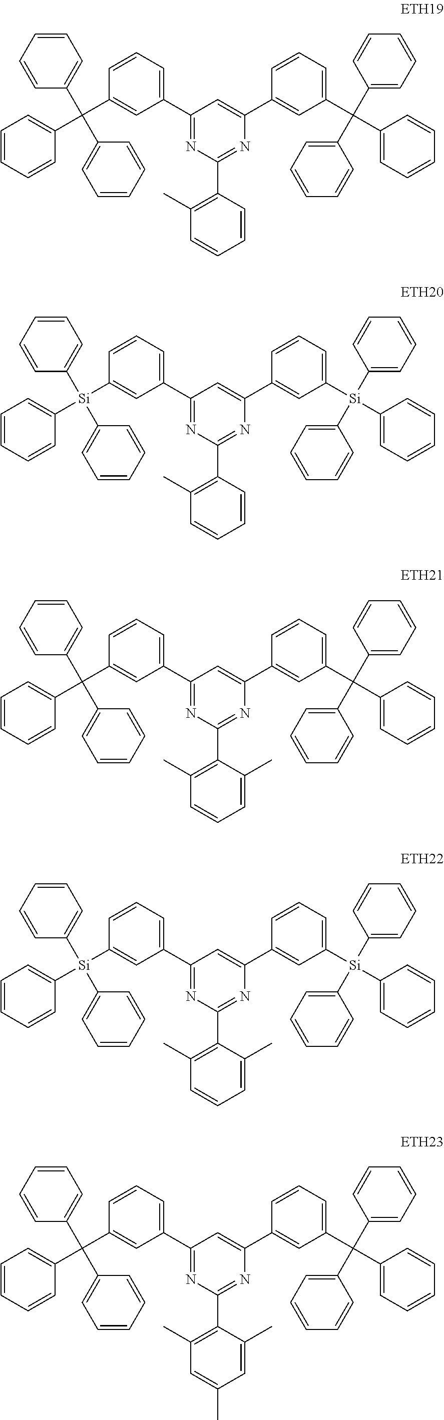

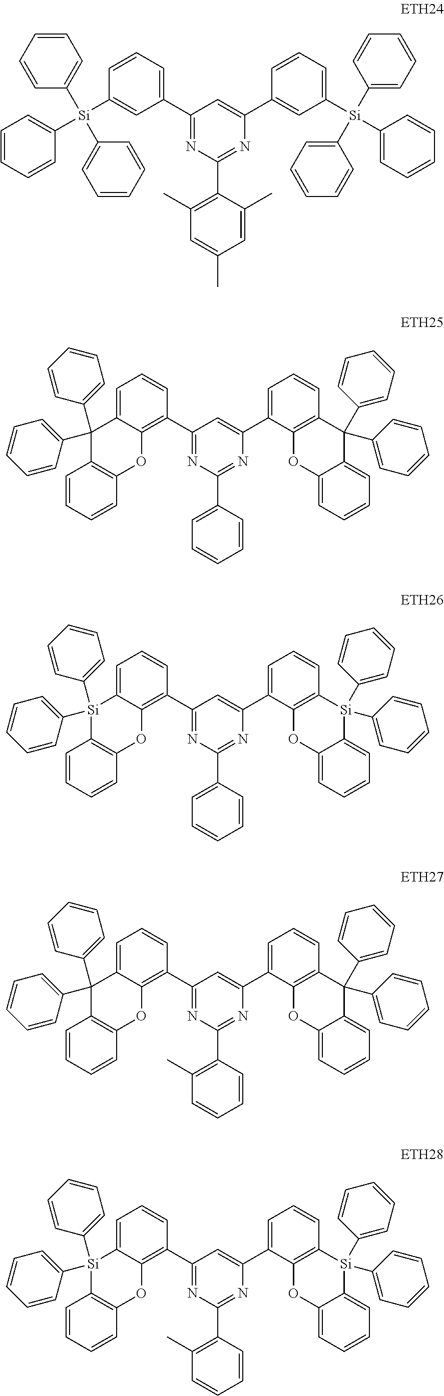

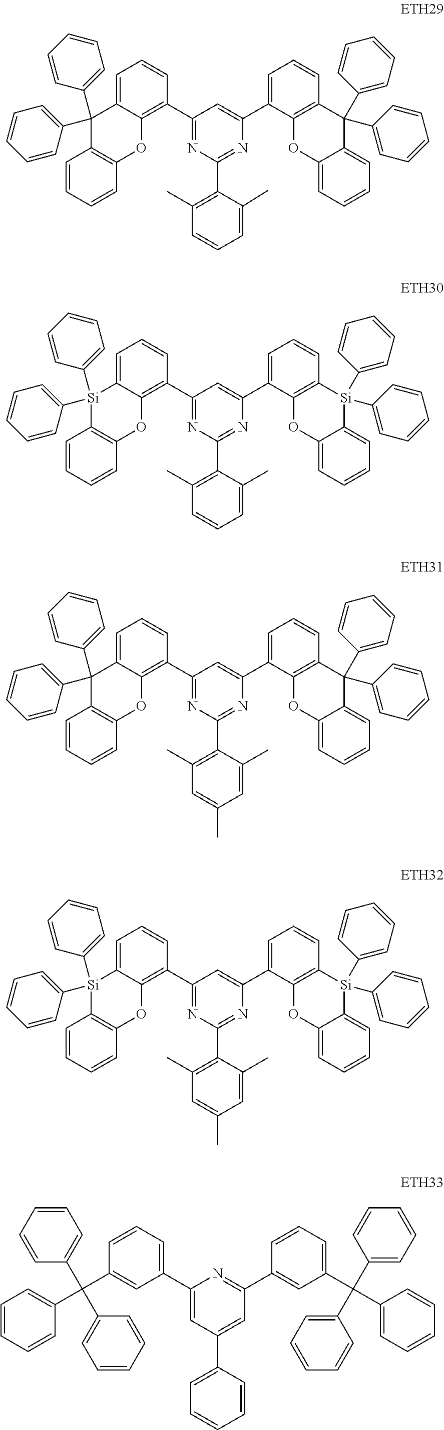

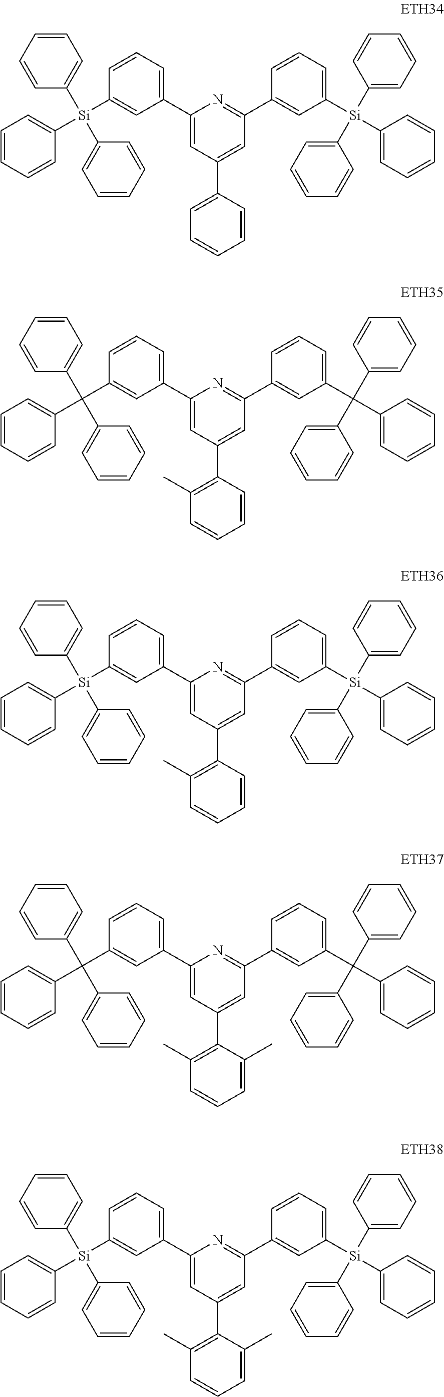

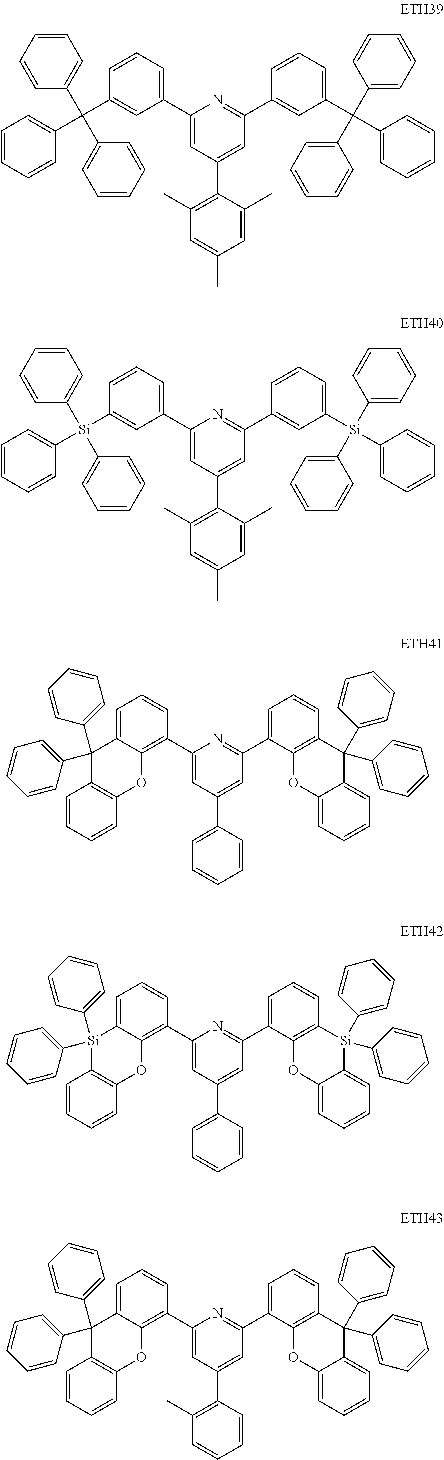

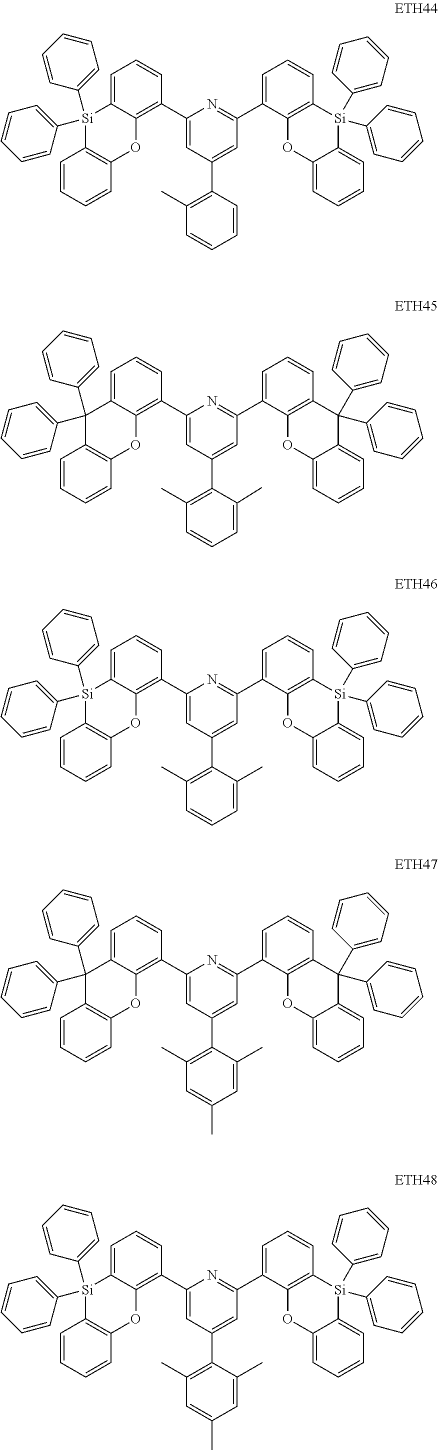

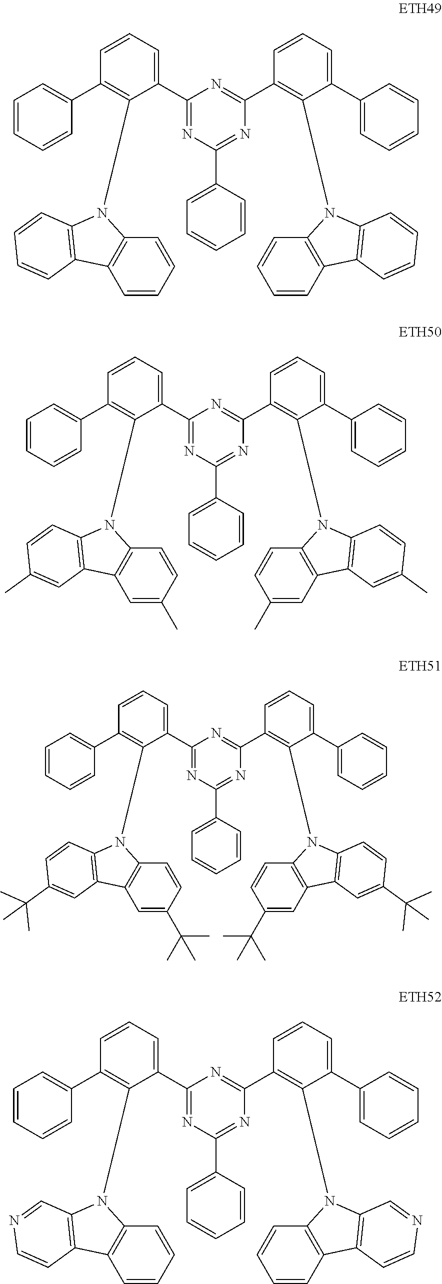

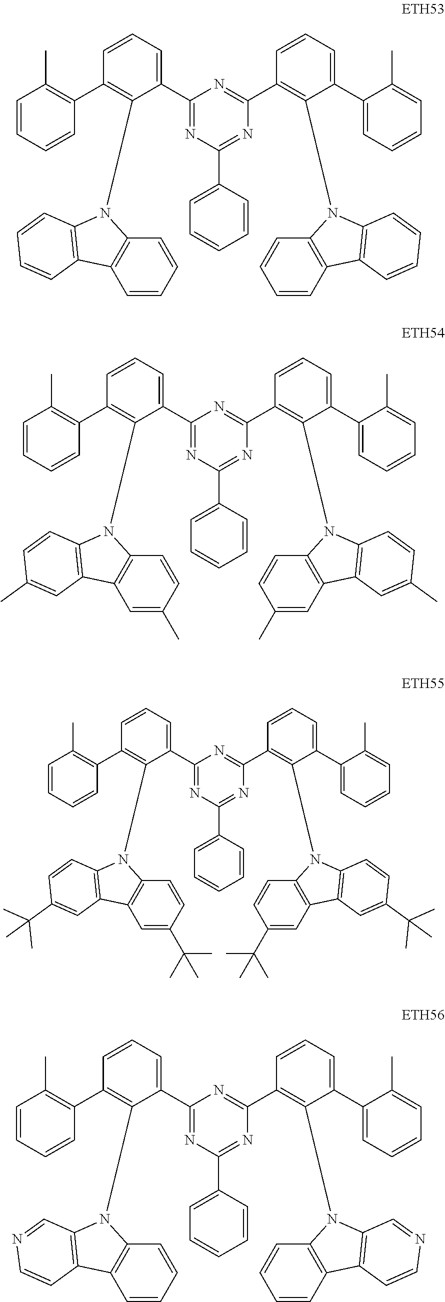

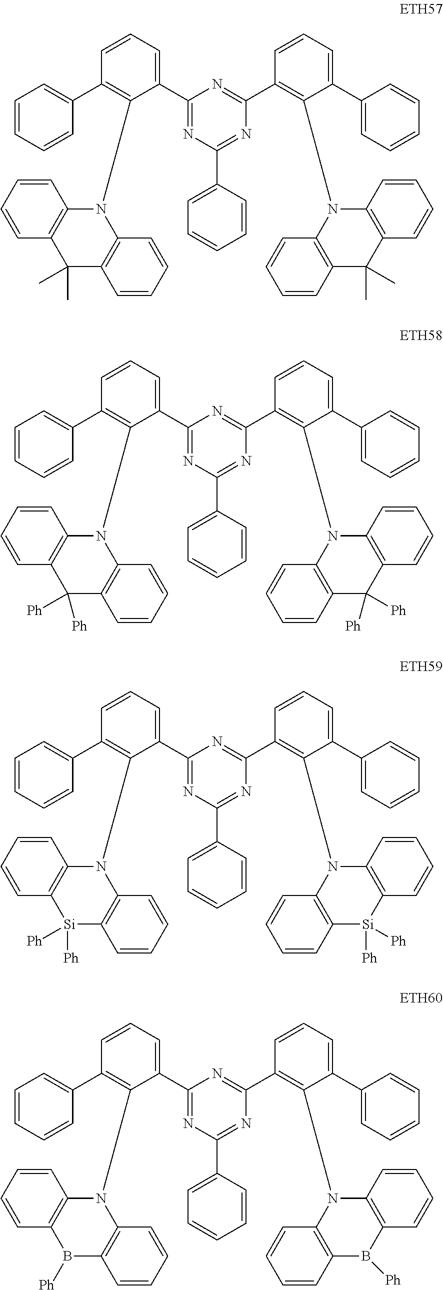

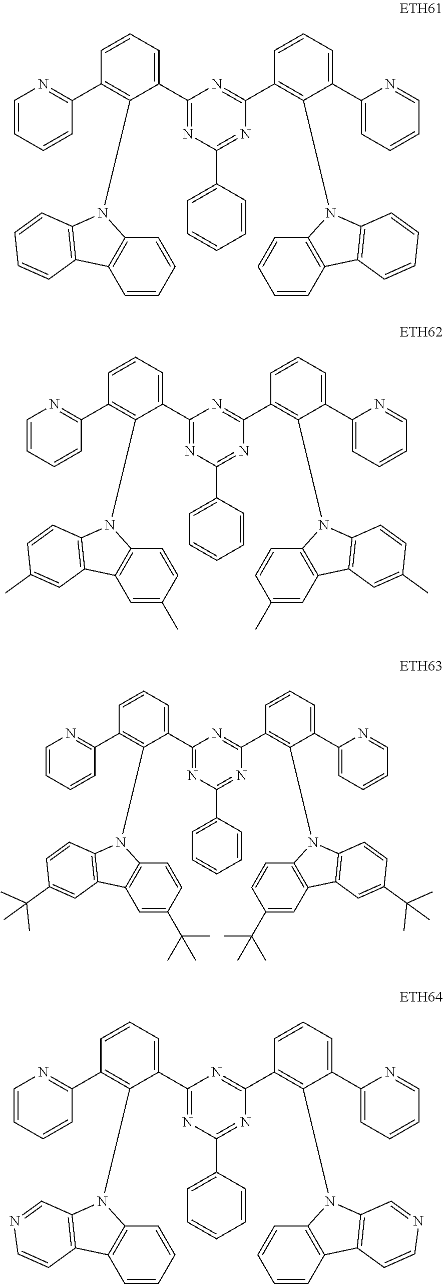

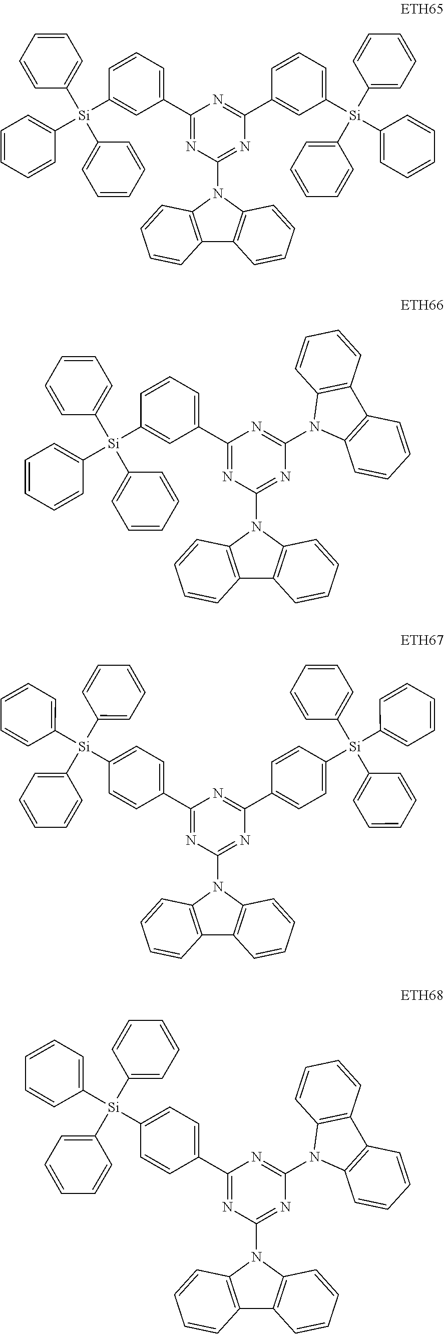 [SMILES: c1ccc([Si](c2ccccc2)(c2ccccc2)c2cccc(-c3nc(-c4cccc([Si](c5ccccc5)(c5ccccc5)c5ccccc5)c4)nc(-n4c5ccccc5c5ccccc54)n3)c2)cc1 ; c1ccc([Si](c2ccccc2)(c2ccccc2)c2cccc(-c3nc(-n4c5ccccc5c5ccccc54)nc(-n4c5ccccc5c5ccccc54)n3)c2)cc1 ; c1ccc([Si](c2ccccc2)(c2ccccc2)c2ccc(-c3nc(-c4ccc([Si](c5ccccc5)(c5ccccc5)c5ccccc5)cc4)nc(-n4c5ccccc5c5ccccc54)n3)cc2)cc1 ; c1ccc([Si](c2ccccc2)(c2ccccc2)c2ccc(-c3nc(-n4c5ccccc5c5ccccc54)nc(-n4c5ccccc5c5ccccc54)n3)cc2)cc1]

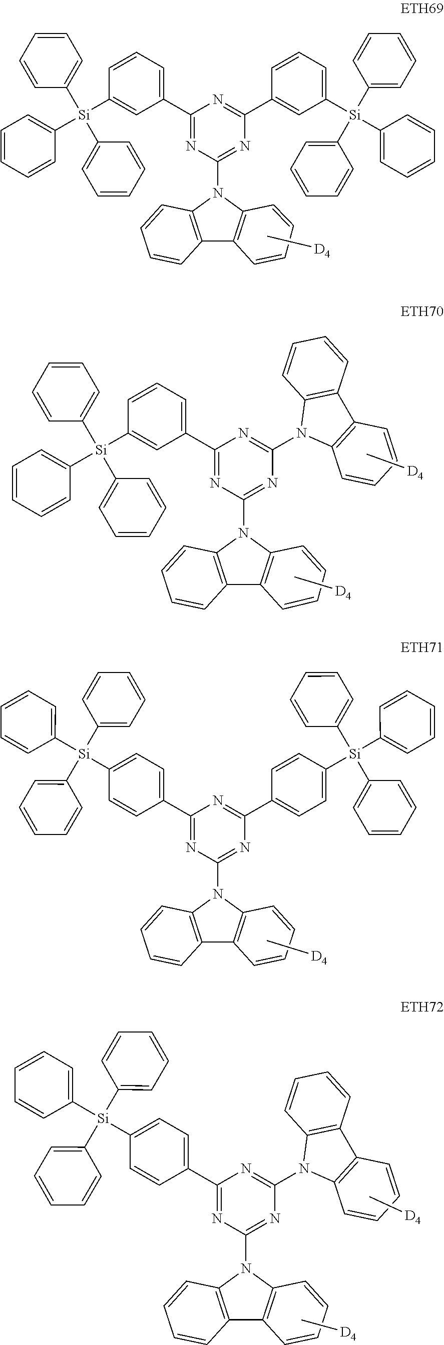

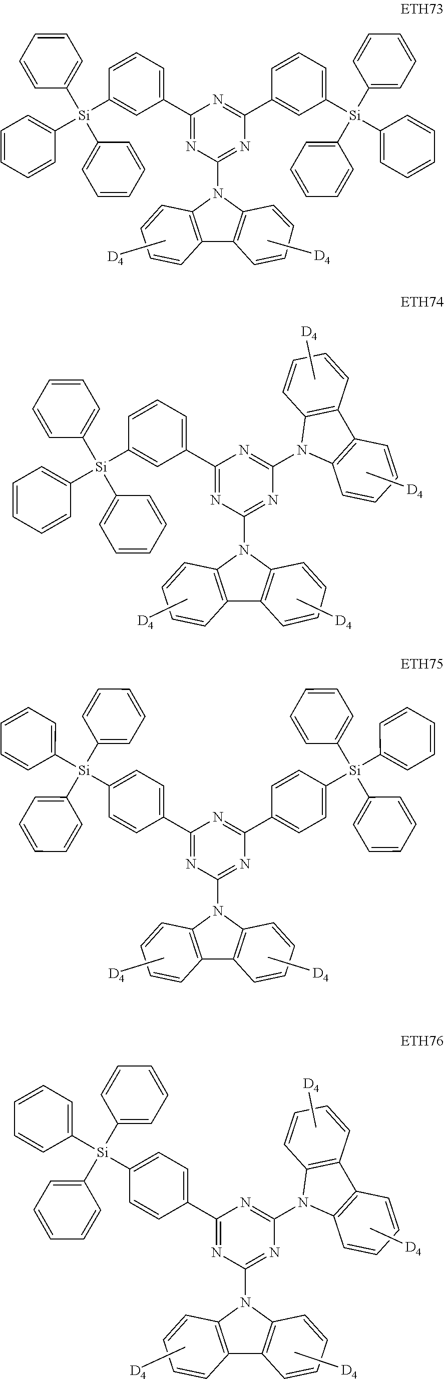

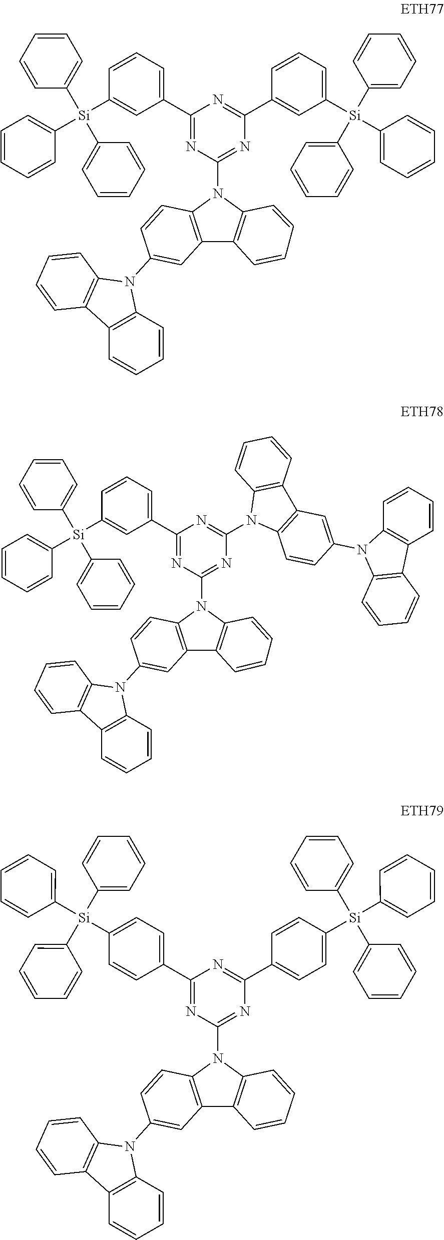 [SMILES: c1ccc([Si](c2ccccc2)(c2ccccc2)c2cccc(-c3nc(-c4cccc([Si](c5ccccc5)(c5ccccc5)c5ccccc5)c4)nc(-n4c5ccccc5c5cc(-n6c7ccccc7c7ccccc76)ccc54)n3)c2)cc1 ; c1ccc([Si](c2ccccc2)(c2ccccc2)c2cccc(-c3nc(-n4c5ccccc5c5cc(-n6c7ccccc7c7ccccc76)ccc54)nc(-n4c5ccccc5c5cc(-n6c7ccccc7c7ccccc76)ccc54)n3)c2)cc1 ; c1ccc([Si](c2ccccc2)(c2ccccc2)c2ccc(-c3nc(-c4ccc([Si](c5ccccc5)(c5ccccc5)c5ccccc5)cc4)nc(-n4c5ccccc5c5cc(-n6c7ccccc7c7ccccc76)ccc54)n3)cc2)cc1]

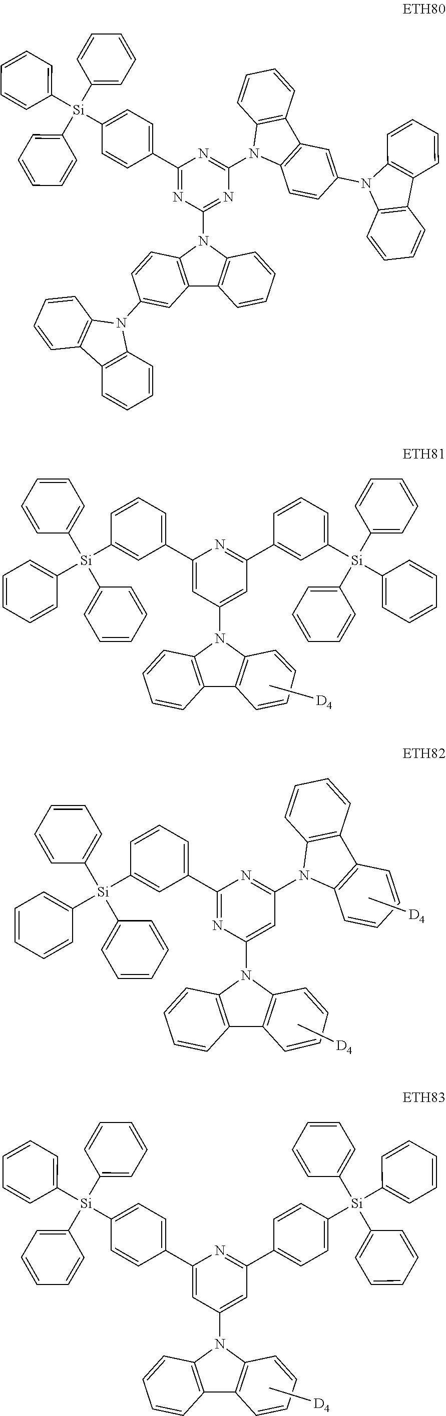

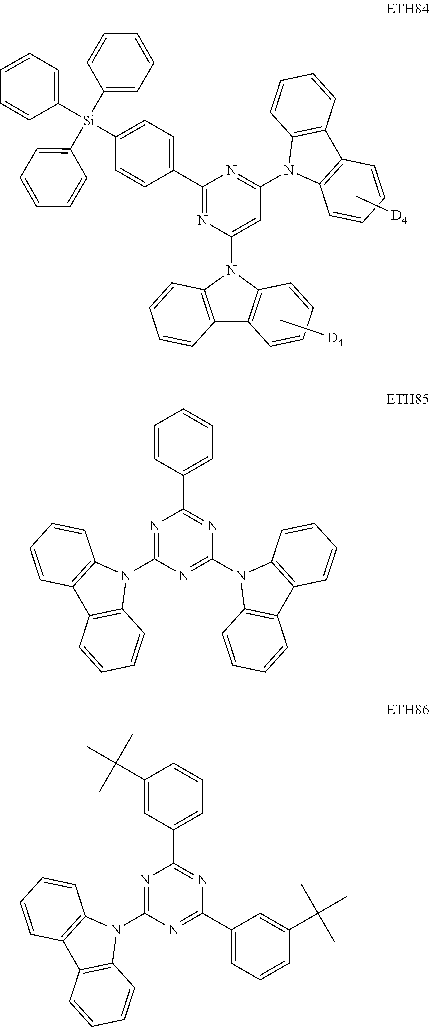

In an embodiment, the third compound may be represented by Formula M-b below. The third compound may be used as a phosphorescent dopant material or a sensitizer. For example, the emission layer EML may include the third compound as a sensitizer.

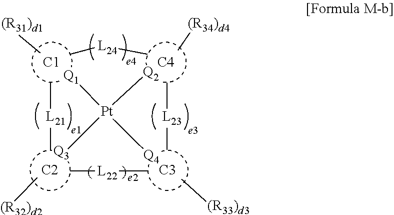

In Formula M-b, Q₁ to Q₄ may each independently be C or N. C1 to C4 may each independently be a substituted or unsubstituted hydrocarbon ring group having 5 to 30 ring-forming carbon atoms, or a substituted or unsubstituted heterocyclic group having 2 to 30 ring-forming carbon atoms.

In Formula M-b, e1 to e4 may each independently be 0 or 1. L₂₁ to L₂₄ may each independently be a direct linkage,

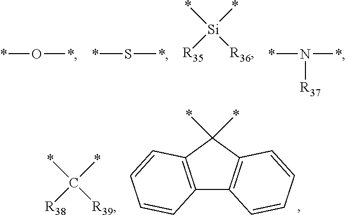

a substituted or unsubstituted divalent alkyl group having 1 to 20 carbon atoms, a substituted or unsubstituted arylene group having 6 to 30 ring-forming carbon atoms, or a substituted or unsubstituted heteroarylene group having 2 to 30 ring-forming carbon atoms.

In Formula M-b, d1 to d4 may each independently be an integer of 0 to 4. R₃₁ to R₃₉ may each independently be a hydrogen atom, a deuterium atom, a halogen atom, a cyano group, a substituted or unsubstituted amine group, a substituted or unsubstituted alkyl group having 1 to 20 carbon atoms, a substituted or unsubstituted aryl group having 6 to 30 ring-forming carbon atoms, a substituted or unsubstituted heteroaryl group having 2 to 30 ring-forming carbon atoms, or bonded to an adjacent group to form a ring.

In addition, the compound represented by Formula M-b may be used as a blue phosphorescent dopant or a green phosphorescent dopant. The compound represented by Formula M-b may be represented by any one selected from among compounds from Compound Group AD below. The light emitting element ED according to an embodiment may include any one selected from among compounds of Compound Group AD below. However, the compounds below are presented as an example, and the compound represented by Formula M-b is not limited to those represented by the compounds below.

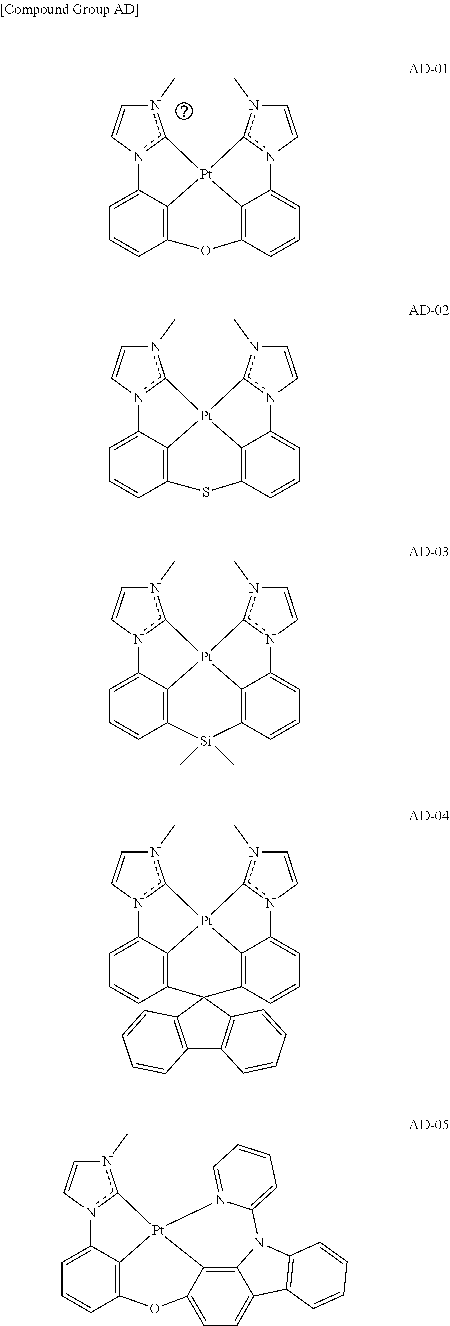

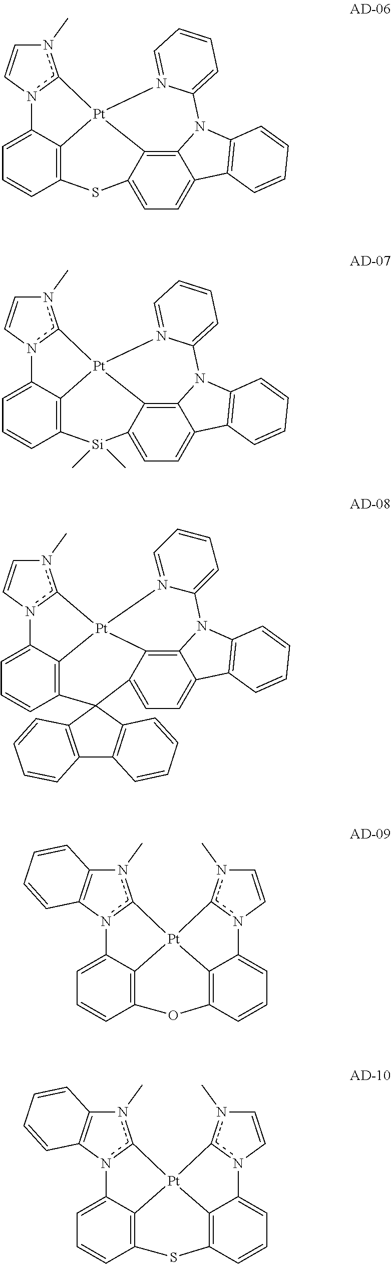

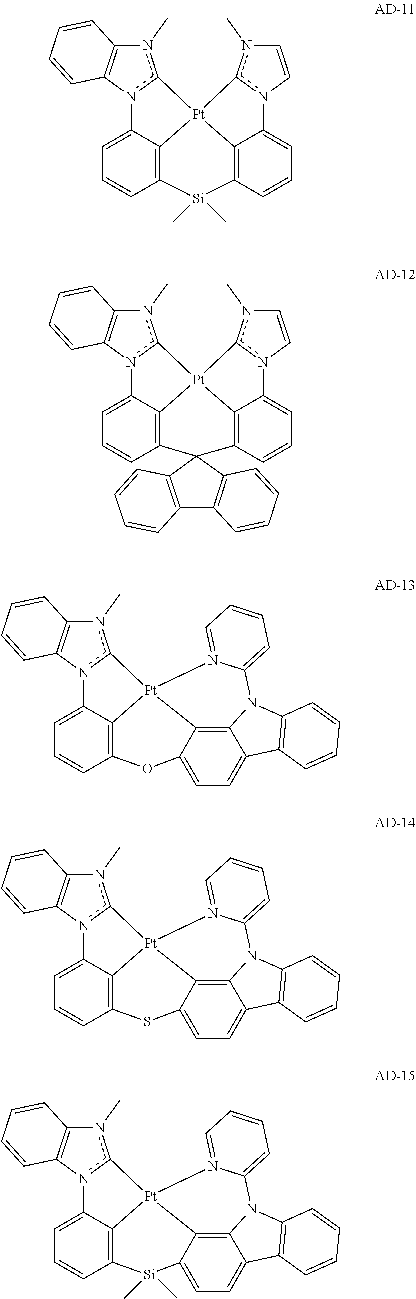

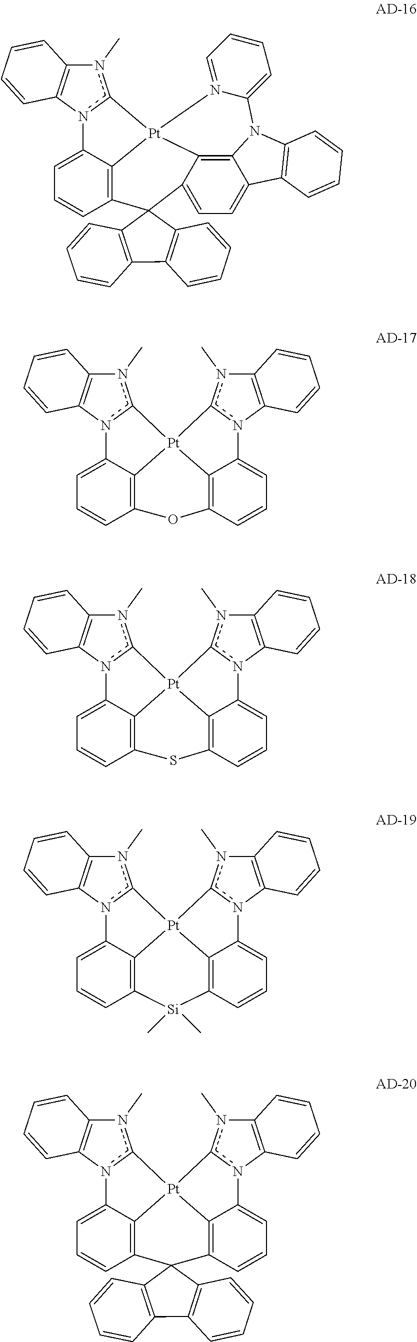

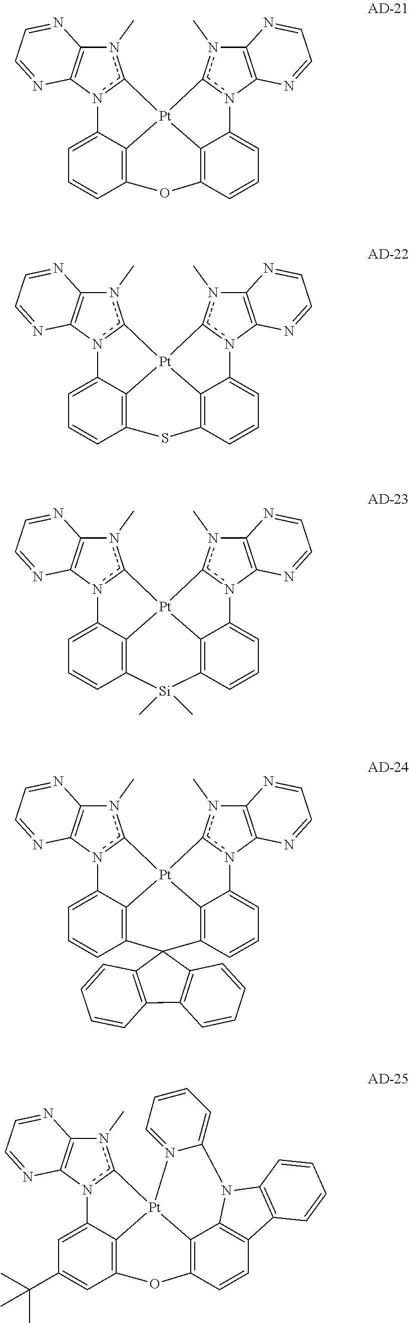

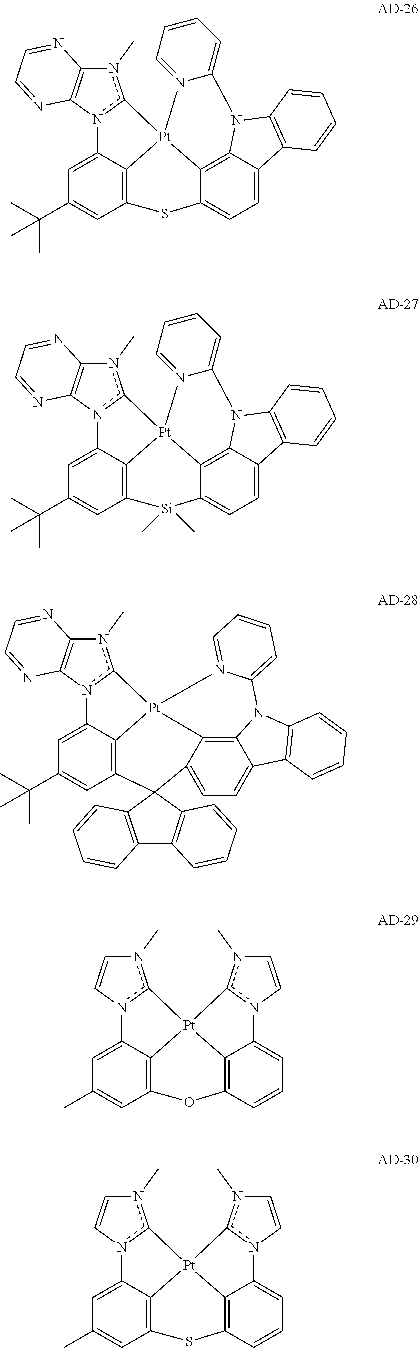

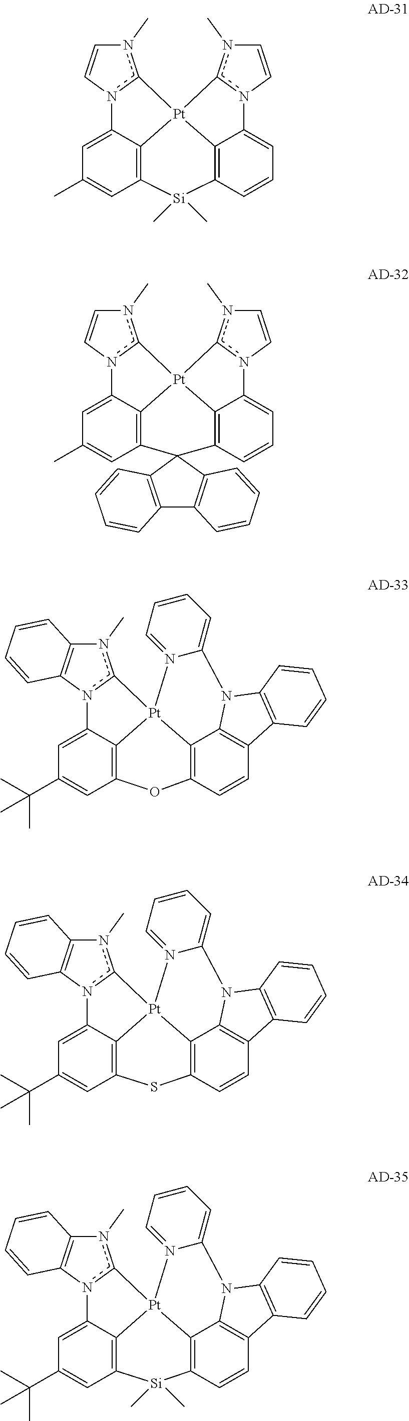

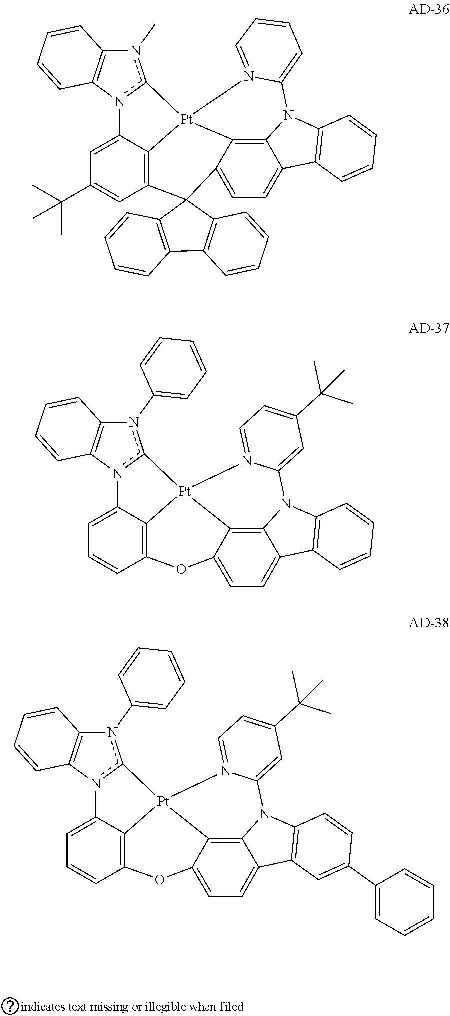

In an embodiment, the emission layer EML may include a compound represented by Formula E-2a or Formula E-2b below. The compound represented by Formula E-2a or Formula E-2b may be used as a phosphorescent host material.

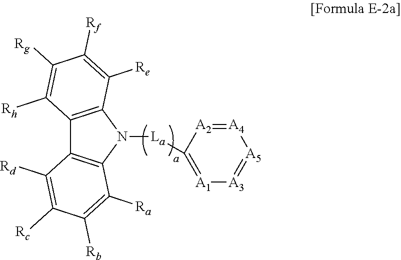

In Formula E-2a, a may be an integer of 0 to 10, and La may be a direct linkage, a substituted or unsubstituted arylene group having 6 to 30 ring-forming carbon atoms, or a substituted or unsubstituted heteroarylene group having 2 to 30 ring-forming carbon atoms. In some embodiments, when a is an integer of 2 or greater, a plurality of La's may each independently be a substituted or unsubstituted arylene group having 6 to 30 ring-forming carbon atoms, or a substituted or unsubstituted heteroarylene group having 2 to 30 ring-forming carbon atoms.

In addition, in Formula E-2a, A₁ to A₅ may each independently be N or CRI. Rₐ to Rᵢ may each independently be a hydrogen atom, a deuterium atom, a substituted or unsubstituted amine group, a substituted or unsubstituted thio group, a substituted or unsubstituted oxy group, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, a substituted or unsubstituted heteroaryl group having 2 to 30 ring-forming carbon atoms, or bonded to an adjacent group to form a ring. Rₐ to Rᵢ may be bonded to an adjacent group to form a hydrocarbon ring or a heterocycle containing N, O, S, and the like as a ring-forming atom.

In some embodiments, in Formula E-2a, two or three selected from A₁ to A₅ may be N, and the others may be CRI.

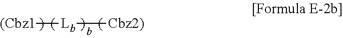

In Formula E-2b, Cbz1 and Cbz2 may each independently be an unsubstituted carbazole group or an aryl-substituted carbazole group having 6 to 30 ring-forming carbon atoms. L_{b} may be a direct linkage, a substituted or unsubstituted arylene group having 6 to 30 ring-forming carbon atoms, or a substituted or unsubstituted heteroarylene group having 2 to 30 ring-forming carbon atoms, b may be an integer of 0 to 10, and when b is an integer of 2 or greater, a plurality of L_{b}'s may each independently be a substituted or unsubstituted arylene group having 6 to 30 ring-forming carbon atoms, or a substituted or unsubstituted heteroarylene group having 2 to 30 ring-forming carbon atoms.

The compound represented by Formula E-2a or Formula E-2b may be represented by any one selected from among compounds from Compound Group E-2 below. However, the compounds listed in Compound Group E-2 below are presented as an example, and the compound represented by Formula E-2a or Formula E-2b is not limited to those listed in Compound Group E-2 below.

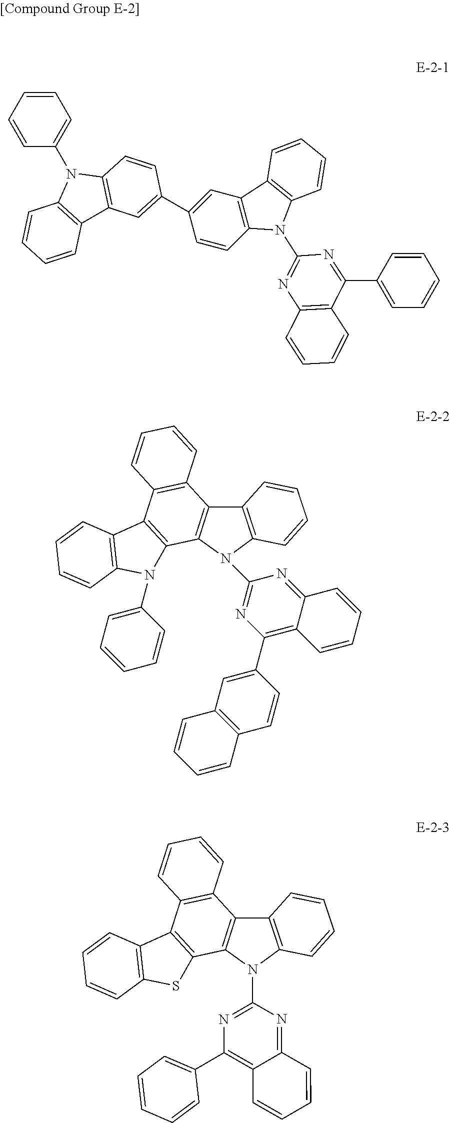 [SMILES: c1ccc(-c2nc(-n3c4ccccc4c4cc(-c5ccc6c(c5)c5ccccc5n6-c5ccccc5)ccc43)nc3ccccc23)cc1 ; c1ccc(-n2c3ccccc3c3c4ccccc4c4c5ccccc5n(-c5nc(-c6ccc7ccccc7c6)c6ccccc6n5)c4c32)cc1 ; c1ccc(-c2nc(-n3c4ccccc4c4c5ccccc5c5c6ccccc6sc5c43)nc3ccccc23)cc1]

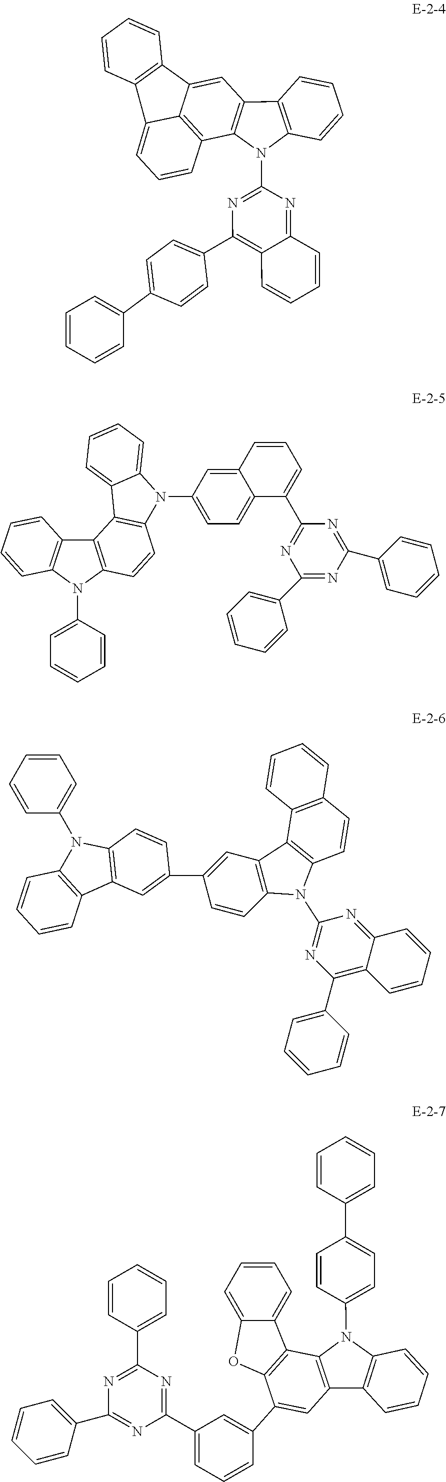 [SMILES: c1ccc(-c2ccc(-c3nc(-n4c5ccccc5c5cc6c7c(cccc7c54)-c4ccccc4-6)nc4ccccc34)cc2)cc1 ; c1ccc(-c2nc(-c3ccccc3)nc(-c3cccc4cc(-n5c6ccccc6c6c7c8ccccc8n(-c8ccccc8)c7ccc65)ccc34)n2)cc1 ; c1ccc(-c2nc(-n3c4ccc(-c5ccc6c(c5)c5ccccc5n6-c5ccccc5)cc4c4c5ccccc5ccc43)nc3ccccc23)cc1 ; c1ccc(-c2ccc(-n3c4ccccc4c4cc(-c5cccc(-c6nc(-c7ccccc7)nc(-c7ccccc7)n6)c5)c5oc6ccccc6c5c43)cc2)cc1]

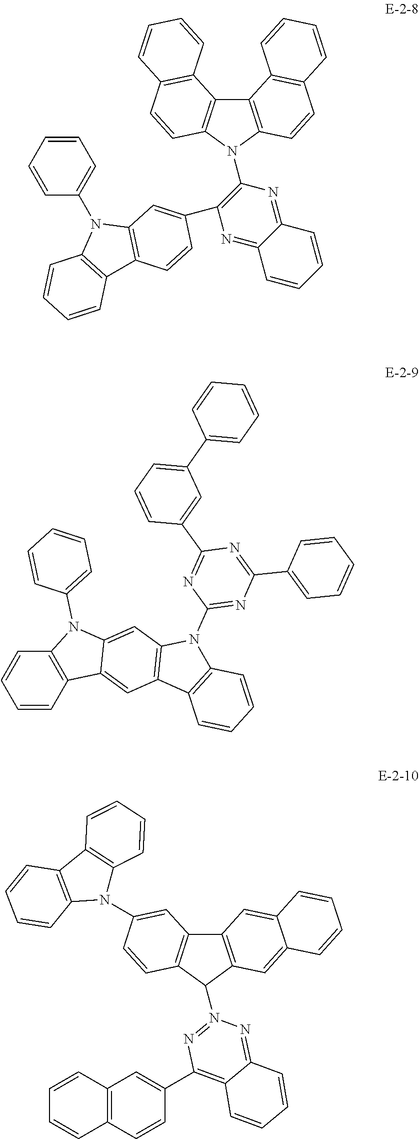

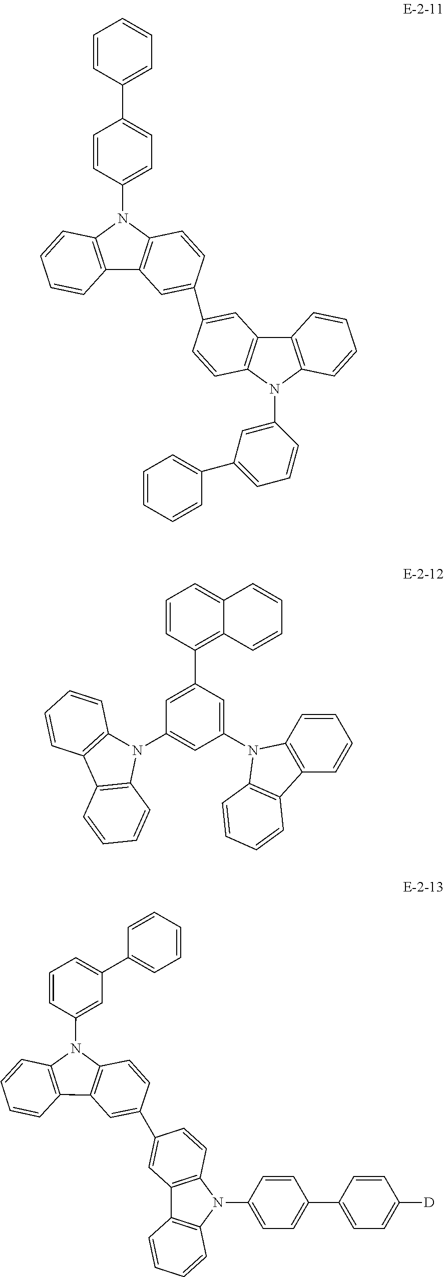 [SMILES: c1ccc(-c2ccc(-n3c4ccccc4c4cc(-c5ccc6c(c5)c5ccccc5n6-c5cccc(-c6ccccc6)c5)ccc43)cc2)cc1 ; c1ccc2c(-c3cc(-n4c5ccccc5c5ccccc54)cc(-n4c5ccccc5c5ccccc54)c3)cccc2c1 ; [2H]c1ccc(-c2ccc(-n3c4ccccc4c4cc(-c5ccc6c(c5)c5ccccc5n6-c5cccc(-c6ccccc6)c5)ccc43)cc2)cc1]

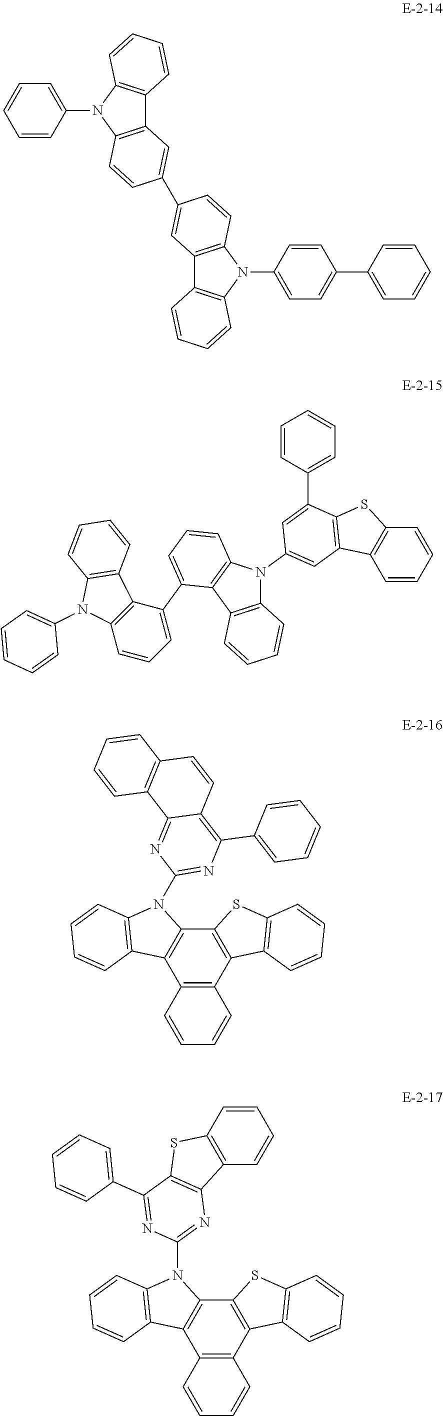 [SMILES: c1ccc(-c2ccc(-n3c4ccccc4c4cc(-c5ccc6c(c5)c5ccccc5n6-c5ccccc5)ccc43)cc2)cc1 ; c1ccc(-c2cc(-n3c4ccccc4c4c(-c5cccc6c5c5ccccc5n6-c5ccccc5)cccc43)cc3c2sc2ccccc23)cc1 ; c1ccc(-c2nc(-n3c4ccccc4c4c5ccccc5c5c6ccccc6sc5c43)nc3c2ccc2ccccc23)cc1 ; c1ccc(-c2nc(-n3c4ccccc4c4c5ccccc5c5c6ccccc6sc5c43)nc3c2sc2ccccc23)cc1]

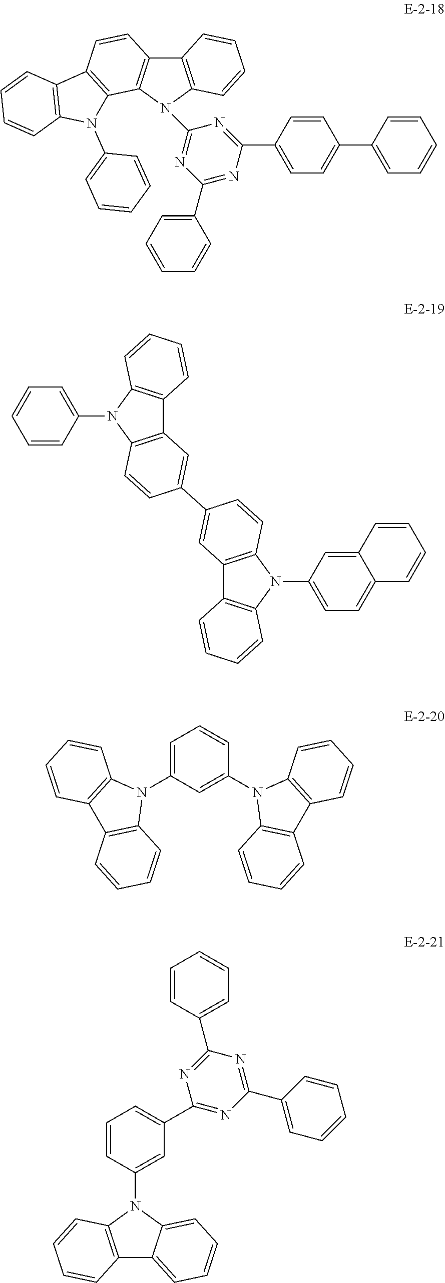 [SMILES: c1ccc(-c2ccc(-c3nc(-c4ccccc4)nc(-n4c5ccccc5c5ccc6c7ccccc7n(-c7ccccc7)c6c54)n3)cc2)cc1 ; c1ccc(-n2c3ccccc3c3cc(-c4ccc5c(c4)c4ccccc4n5-c4ccc5ccccc5c4)ccc32)cc1 ; c1cc(-n2c3ccccc3c3ccccc32)cc(-n2c3ccccc3c3ccccc32)c1 ; c1ccc(-c2nc(-c3ccccc3)nc(-c3cccc(-n4c5ccccc5c5ccccc54)c3)n2)cc1]

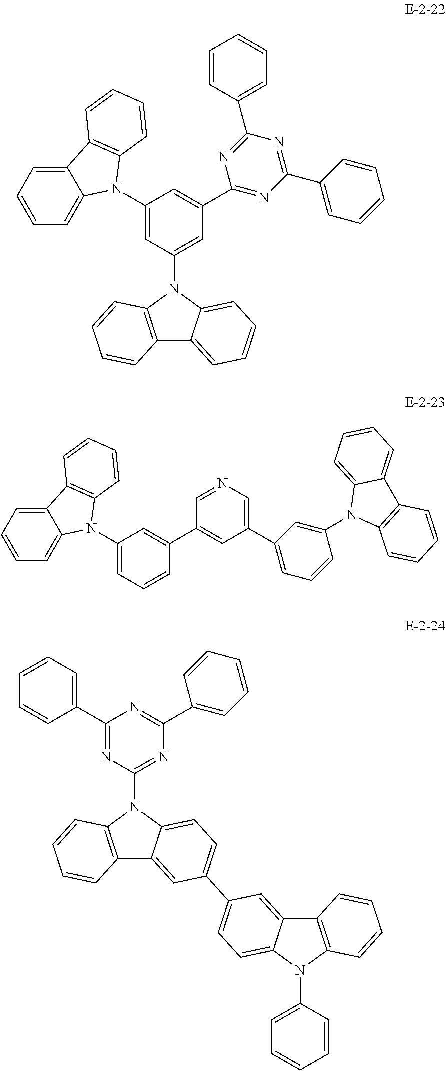 [SMILES: c1ccc(-c2nc(-c3ccccc3)nc(-c3cc(-n4c5ccccc5c5ccccc54)cc(-n4c5ccccc5c5ccccc54)c3)n2)cc1 ; c1cc(-c2cncc(-c3cccc(-n4c5ccccc5c5ccccc54)c3)c2)cc(-n2c3ccccc3c3ccccc32)c1 ; c1ccc(-c2nc(-c3ccccc3)nc(-n3c4ccccc4c4cc(-c5ccc6c(c5)c5ccccc5n6-c5ccccc5)ccc43)n2)cc1]

The emission layer EML may further include any suitable material generally used in the art as a host material. In some embodiments, the emission layer EML may include, as a host material, at least one selected from among bis(4-(9H-carbazol-9-yl)phenyl)diphenylsilane (BCPDS), (4-(1-(4-(diphenylamino)phenyl)cyclohexyl)phenyl)diphenyl-phosphine oxide (POPCPA), bis[2-(diphenylphosphino)phenyl]ether oxide (DPEPO), 4,4′-bis(N-carbazolyl)-1,1′-biphenyl (CBP), 1,3-bis(carbazol-9-yl)benzene (mCP), 2,8-bis(diphenylphosphoryl)dibenzofuran (PPF), 4,4′,4″-tris(carbazol-9-yl)-triphenylamine (TCTA), and 1,3,5-tris(1-phenyl-1H-benzo[d]imidazol-2-yl)benzene (TPBi). However, the embodiment of the present disclosure is not limited thereto, and for example, tris(8-hydroxyquinolino)aluminum (Alq3), 9,10-di(naphthalene-2-yl)anthracene (ADN), 3-tert-butyl-9,10-di(naphth-2-yl)anthracene (TBADN), distyrylarylene (DSA), 4,4′-bis(9-carbazolyl)-2,2′-dimethylbiphenyl (CDBP), 2-methyl-9,10-bis(naphthalen-2-yl)anthracene (MADN), hexaphenyl cyclotriphosphazene (CP1), 1,4-bis(triphenylsilyl)benzene (UGH2), hexaphenylcyclotrisiloxane (DPSiO3), octaphenylcyclotetrasiloxane (DPSiO4), and/or the like may be used as a host material.

The emission layer EML may include a compound represented by Formula M-a below. The compound represented by Formula M-a below may be used as a phosphorescent dopant material.

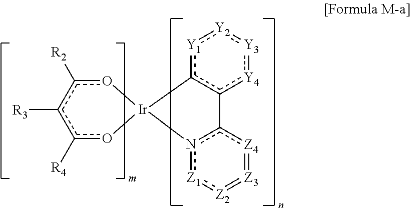

In Formula M-a above, Y₁ to Y₄, and Z₁ to Z₄ may each independently be CR₁ or N, and R₁ to R₄ may each independently be a hydrogen atom, a deuterium atom, a substituted or unsubstituted amine group, a substituted or unsubstituted thio group, a substituted or unsubstituted oxy group, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, a substituted or unsubstituted heteroaryl group having 2 to 30 ring-forming carbon atoms, or bonded to an adjacent group to form a ring. In Formula M-a, m is 0 or 1, and n is 2 or 3. In Formula M-a, when m is 0, n is 3, and when m is 1, n is 2.

The compound represented by Formula M-a may be represented by any one selected from among compounds M-a1 to M-a25 below. However, the compounds M-a1 to M-a25 below are presented as an example, and the compound represented by Formula M-a is not limited to those represented by the compounds M-a1 to M-a25 below.

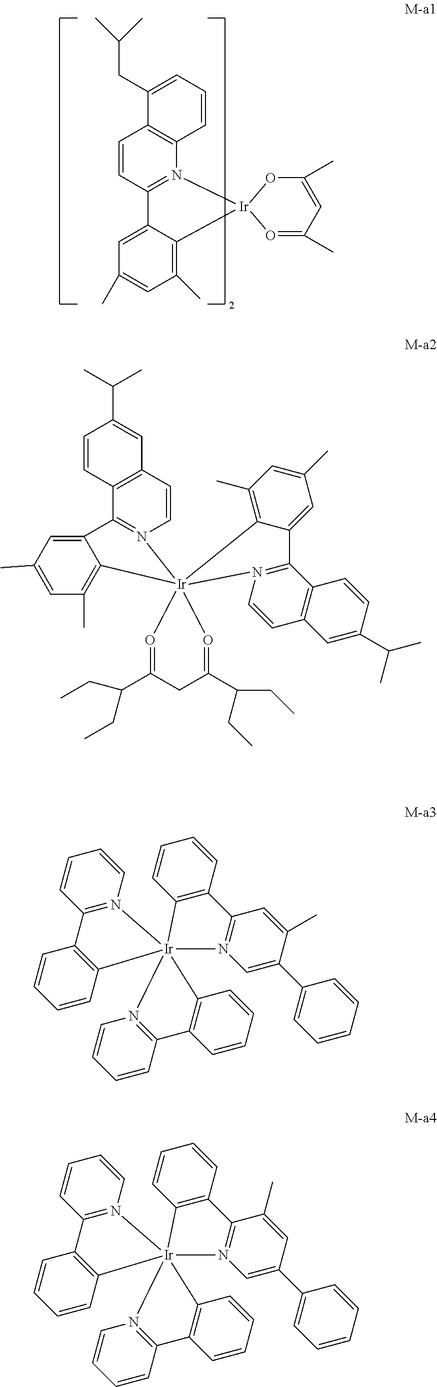

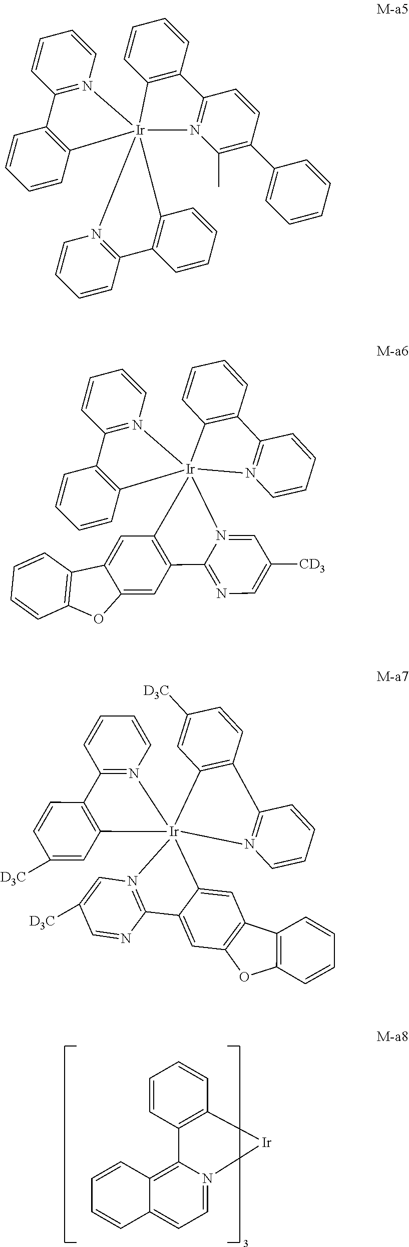

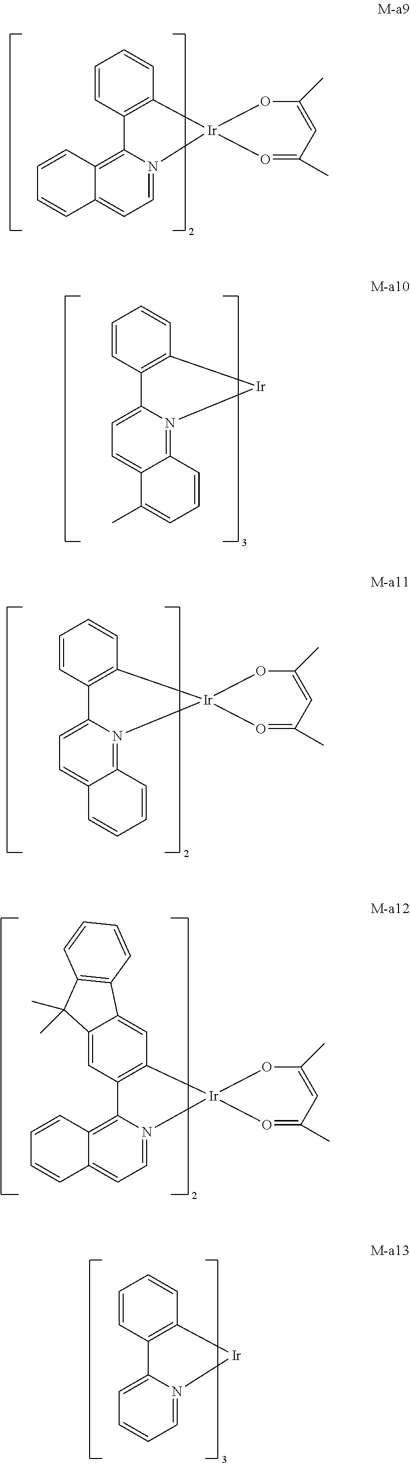

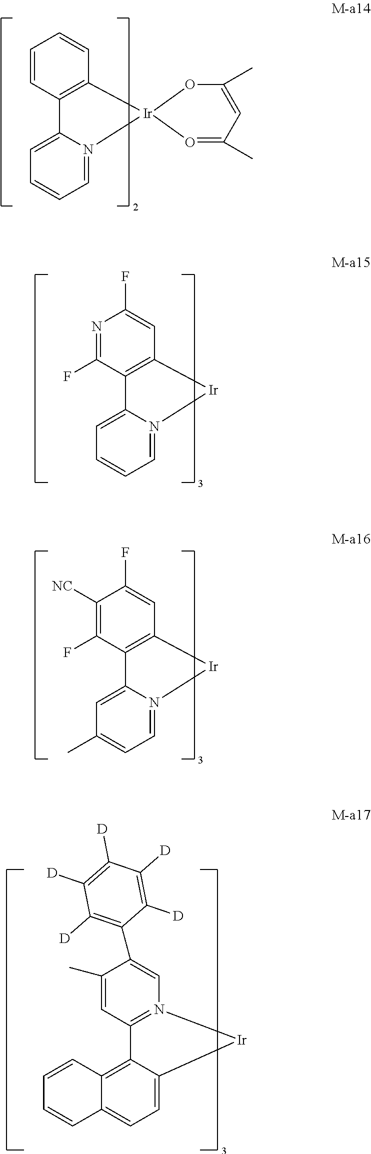

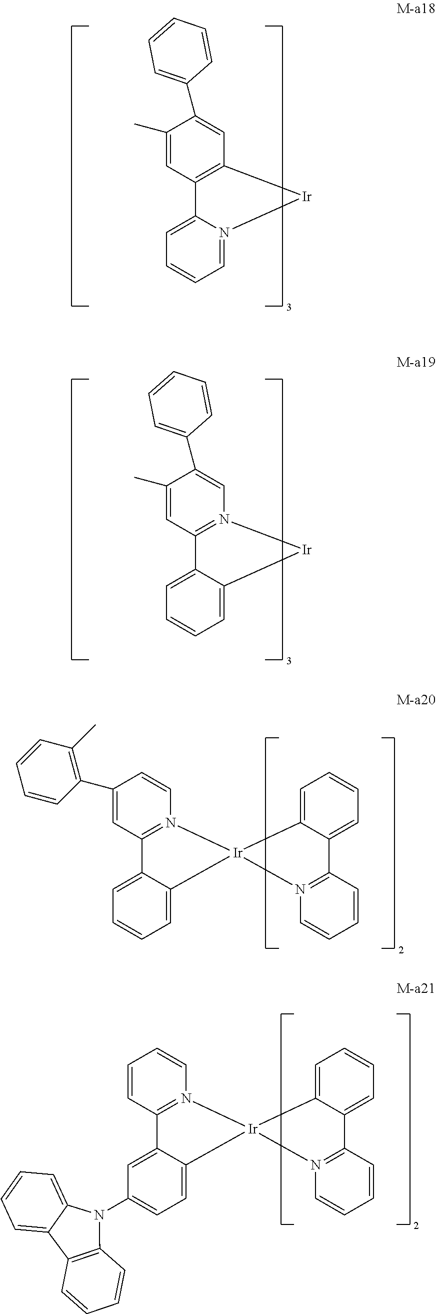

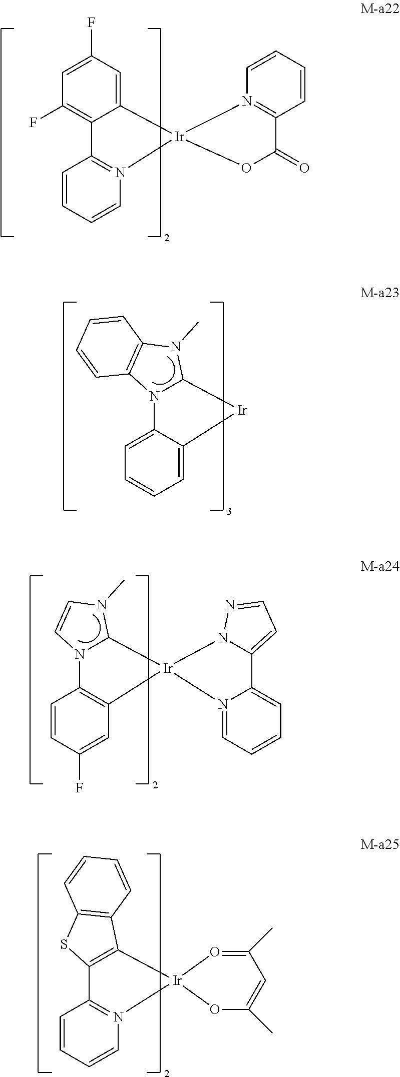

The compounds M-a1 and M-a2 may be used as a red dopant material, and the compounds M-a3 to M-a7 may be used as a green dopant material.

The emission layer EML may include a compound represented by any one selected from among Formulas F-a to F-c below. The compounds represented by Formulas F-a to F-c below may be used as a fluorescence dopant material.

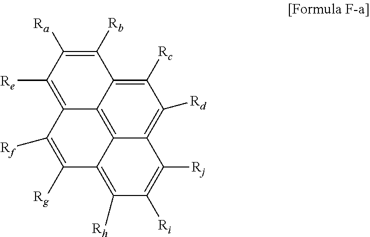

In Formula F-a above, two selected from Rₐ to Rⱼ may each independently be substituted with *—NAr₁Ar₂. The others among Rₐ to Rⱼ which are not substituted with *—NAr₁Ar₂ may each independently be a hydrogen atom, a deuterium atom, a halogen atom, a cyano group, a substituted or unsubstituted amine group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. In *—NAr₁Ar₂, Ar₁ and Ar₂ may each independently be a substituted or unsubstituted aryl group having 6 to 30 ring-forming carbon atoms, or a substituted or unsubstituted heteroaryl group having 2 to 30 ring-forming carbon atoms. For example, at least one selected from Ar₁ and Ar₂ may be a heteroaryl group containing O or S as a ring-forming atom.

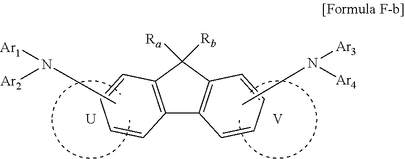

In Formula F-b above, Rₐ and R_{b} may each independently be a hydrogen atom, a deuterium atom, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, or bonded to an adjacent group to form a ring. Ar₁ to Ar₄ may each independently be a substituted or unsubstituted aryl group having 6 to 30 ring-forming carbon atoms, or a substituted or unsubstituted heteroaryl group having 2 to 30 ring-forming carbon atoms.

In Formula F-b, U and V may each independently be a substituted or unsubstituted hydrocarbon ring having 5 to 30 ring-forming carbon atoms, or a substituted or unsubstituted heterocycle having 2 to 30 ring-forming carbon atoms. In Formula F-b, the number of rings represented by U and V may each independently be 0 or 1.

For example, in Formula F-b, when the number of U or V is 1, one ring forms a fused ring in a portion indicated by U or V, and when the number of U or V is 0, it means that no ring indicated by U or V is present. In some embodiments, when the number of U is 0 and the number of V is 1, or when the number of U is 1 and the number of V is 0, a fused ring having a fluorene core of Formula F-b may be a cyclic compound having four rings. In addition, when both U and V are 0, the fused ring having a fluorene core of Formula F-b may be a cyclic compound having three rings. In addition, when both U and V are 1, the fused ring having a fluorene core of Formula F-b may be a cyclic compound having five rings.

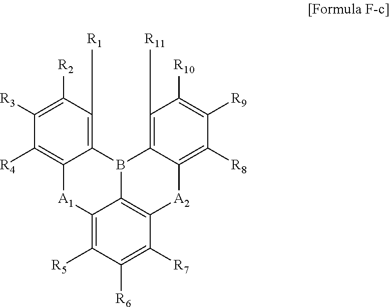

In Formula F-c, A₁ and A₂ may each independently be O, S, Se, or NRₘ, and Rₘ may be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. R₁ to R₁₁ may each independently be a hydrogen atom, a deuterium atom, a halogen atom, a cyano group, a substituted or unsubstituted amine group, a substituted or unsubstituted boron group, a substituted or unsubstituted oxy group, a substituted or unsubstituted thio group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, or bonded to an adjacent group to form a ring.

In Formula F-c, A₁ and A₂ may each independently be bonded to substituents of neighboring rings to form a fused ring. For example, when A₁ and A₂ are each independently NRₘ, A₁ may be bonded to R₄ or R₅ to form a ring. In addition, A₂ may be bonded to R₇ or R₈ to form a ring.

The emission layer EML may include any suitable dopant material generally used in the art. In some embodiments, the emission layer EML may include styryl derivatives (e.g., 1,4-bis[2-(3-N-ethylcarbazolyl)vinyl]benzene (BCzVB), 4-(di-p-tolylamino)-4′-[(di-p-tolylamino)styryl]stilbene (DPAVB), and/or N-(4-((E)-2-(6-((E)-4-(diphenylamino)styryl)naphthalen-2-yl)vinyl)phenyl)-N-phenylbenzenamine (N-BDAVBi)), perylene and/or derivatives thereof (e.g., 2,5,8,11-tetra-t-butylperylene (TBP)), pyrene and/or derivatives thereof (e.g., 1,1-dipyrene, 1,4-dipyrenylbenzene, 1,4-bis(N,N-diphenylamino)pyrene), and/or the like.

The emission layer EML may include any suitable phosphorescent dopant material generally used in the art. In some embodiments, as a phosphorescent dopant, a metal complex including iridium (Ir), platinum (Pt), osmium (Os), gold (Au), titanium (Ti), zirconium (Zr), hafnium (Hf), europium (Eu), and terbium (Tb), or thulium I may be used. In some embodiments, iridium(III) bis(4,6-difluorophenylpyridinato-N,C2′)picolinate (Firpic), bis(2,4-difluorophenylpyridinato)-tetrakis(1-pyrazolyl)borate iridium(III) (Fir6), platinum octaethyl porphyrin (PtOEP), and/or the like may be used as a phosphorescent dopant. However, the embodiment of the present disclosure is not limited thereto.

The emission layer EML may include a quantum dot material. The core of a quantum dot may be selected from a Group II-VI compound, a Group I-II-VI compound, a Group II-IV-VI compound, a Group I-II-IV-VI compound, a Group III-VI compound, a Group I-III-VI compound, a Group III-V element, a Group III-II-V compound, a Group II-IV-V compound, a Group IV-VI compound, a Group IV element, a Group IV compound, and a combination thereof.

The Group II-VI compound may be selected from the group consisting of a binary compound selected from the group consisting of CdSe, CdTe, CdS, ZnS, ZnSe, ZnTe, ZnO, HgS, HgSe, HgTe, MgSe, MgS, and a mixture thereof, a ternary compound selected from the group consisting of CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, MgZnS, and a mixture thereof, and a quaternary compound selected from the group consisting of HgZnTeS, CdZnSeS, CdZnSeTe, CdZnSTe, CdHgSeS, CdHgSeTe, CdHgSTe, HgZnSeS, HgZnSeTe, and a mixture thereof. In some embodiments, the Group II-VI compound may further include a Group I metal and/or a Group IV element. The Group I-II-VI compound may be selected from CuSnS and CuZnS, and the Group III-IV-VI compound may be selected from ZnSnS and the like. The Group II-II-IV-VI compound may be selected from quaternary compounds selected from the group consisting of Cu₂ZnSnS₂, Cu₂ZnSnS₄, Cu₂ZnSnSe₄, Ag₂ZnSnS₂, and a mixture thereof.

The Group III-VI compound may include a binary compound such as In₂S₃ and/or In₂Se₃, a ternary compound such as InGaS₃ and/or InGaSe₃, or any combination thereof.

The Group II-III-VI compound may be selected from a ternary compound selected from the group consisting of AgInS, AgInS₂, CuInS, CuInS₂, AgGaS₂, CuGaS₂ CuGaO₂, AgGaO₂, AgAlO₂, and a mixture thereof, and/or a quaternary compound such as AgInGaS₂ and/or CuInGaS₂.

The Group III-V compound may be selected from the group consisting of a binary compound selected from the group consisting of GaN, GaP, GaAs, GaSb, AlN, AlP, AlAs, AlSb, InN, InP, InAs, InSb, and a mixture thereof, a ternary compound selected from the group consisting of GaNP, GaNAs, GaNSb, GaPAs, GaPSb, AlNP, AlNAs, AlNSb, AlPAs, AlPSb, InGaP, InAlP, InNP, InNAs, InNSb, InPAs, InPSb, and a mixture thereof, and a quaternary compound selected from the group consisting of GaAlNP, GaAlNAs, GaAlNSb, GaAlPAs, GaAlPSb, GaInNP, GaInNAs, GaInNSb, GaInPAs, GaInPSb, InAlNP, InAlNAs, InAlNSb, InAlPAs, InAlPSb, and a mixture thereof. In some embodiments, the Group III-V compound may further include a Group II metal. For example, InZnP and/or the like may be selected as a Group III-II-V compound.

The Group III-IV-V compound may be a ternary compound selected from the group consisting of ZnSnP, ZnSnP₂, ZnSnAs₂, ZnGeP₂, ZnGeAs₂, CdSnP₂, and CdGeP₂ and a mixture thereof.

The Group IV-VI compound may be selected from the group consisting of a binary compound selected from the group consisting of SnS, SnSe, SnTe, PbS, PbSe, PbTe, and a mixture thereof, a ternary compound selected from the group consisting of SnSeS, SnSeTe, SnSTe, PbSeS, PbSeTe, PbSTe, SnPbS, SnPbSe, SnPbTe, and a mixture thereof, and a quaternary compound selected from the group consisting of SnPbSSe, SnPbSeTe, SnPbSTe, and a mixture thereof. The Group IV element may be selected from the group consisting of Si, Ge, and a mixture thereof. The Group IV compound may be a binary compound selected from the group consisting of SiC, SiGe, and a mixture thereof.

In this case, the binary compound, the ternary compound, and/or the quaternary compound may be present in particles having a uniform concentration distribution, or may be present in the same particles having a partially different concentration distribution. In addition, a core/shell structure in which one quantum dot surrounds another quantum dot may be present. The core/shell structure may have a concentration gradient in which the concentration of an element present in the shell becomes lower along a direction towards the core.

In some embodiments, a quantum dot may have the core/shell structure including a core having nano-crystals, and a shell surrounding the core, which are described above. The shell of the quantum dot may serve as a protection layer to prevent or reduce the chemical deformation of the core so as to keep semiconductor properties, and/or a charging layer to impart electrophoresis properties to the quantum dot. The shell may be a single layer or a plurality of layers. Examples of the shell of the quantum dot may be a metal and/or non-metal oxide, a semiconductor compound, or a combination thereof.

For example, the metal and/or non-metal oxide may be a binary compound such as SiO₂, Al₂O₃, TiO₂, ZnO, MnO, Mn₂O₃, Mn₃O₄, CuO, FeO, Fe₂O₃, Fe₃O₄, CoO, Co₃O₄, NiO, and/or a ternary compound such as MgAl₂O₄, CoFe₂O₄, NiFe₂O₄, and/or CoMn₂O₄, but the embodiment of the present disclosure is not limited thereto.

In addition, the semiconductor compound may be, for example, CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, etc., but the embodiment of the present disclosure is not limited thereto.

The quantum dot may have, in an emission wavelength spectrum, a full width of half maximum (FWHM) of about 45 nm or less, about 40 nm or less, or, for example, about 30 nm or less, and in this range, the color purity and/or the color reproducibility may be improved. In addition, light emitted through the quantum dot is emitted in all (or substantially all) directions, and thus a wide viewing angle may be improved.

In addition, the form of a quantum dot is not particularly limited as long as it is a form generally used in the art. For example, a quantum dot in the form of spherical, pyramidal, multi-arm, and/or cubic nanoparticles, nanotubes, nanowires, nanofibers, nanoplatelets, and/or the like may be used.

The quantum dot may control the colors of emitted light according to the particle size thereof, and thus the quantum dot may have various suitable light emitting colors such as blue, red, green, and/or the like.

In the light emitting element ED of an embodiment, the electron transport region ETR may be provided on the emission layer EML. The electron transport region ETR may include at least one selected from a hole blocking layer HBL, an electron transport layer ETL, and an electron injection layer EIL. In addition, the electron transport region ETR may include two electron transport layers ETL-1 and ETL-2.

However, this is presented as an example, and the embodiment of the present disclosure is not limited thereto. Hereinafter, descriptions of the electron transport layer ETL may also be applied to the first electron transport layer ETL-1 and the second electron transport layer ETL-2.

The electron transport region ETR may have a single layer formed of a single material, a single layer formed of a plurality of different materials, or a multilayer structure having a plurality of layers formed of a plurality of different materials.

For example, the electron transport region ETR may have a single layer structure of an electron injection layer EIL or an electron transport layer ETL, and may have a single layer structure formed of an electron injection material and an electron transport material. In addition, the electron transport region ETR may have a single layer structure formed of a plurality of different materials, or may have a structure in which an electron transport layer ETL/electron injection layer EIL, or a hole blocking layer HBL/electron transport layer ETL/electron injection layer EIL are stacked in order from the emission layer EML, but is not limited thereto. The electron transport region ETR may have a thickness of, for example, about 1000 Å to about 1500 Å.

The electron transport region ETR may be formed using various suitable methods such as a vacuum deposition method, a spin coating method, a cast method, a Langmuir-Blodgett (LB) method, an inkjet printing method, a laser printing method, and/or a laser induced thermal imaging (LITI) method.

The electron transport region ETR may include a compound represented by Formula ET-1 described above. The electron transport region ETR may include an anthracene-based compound. However, the embodiment of the present disclosure is not limited thereto, and the electron transport region ETR may include, for example, tris(8-hydroxyquinolinato)aluminum (Alq3), 1,3,5-tri[(3-pyridyl)-phen-3-yl]benzene, 2,4,6-tris(3′-(pyridin-3-yl)biphenyl-3-yl)-1,3,5-triazine, 2-(4-(N-phenylbenzoimidazolyl-1-ylphenyl)-9,10-dinaphthylanthracene, 1,3,5-tri(1-phenyl-1H-benzo[d]imidazol-2-yl)benzene (TPBi), 2,9-dimethyl-4,7-diphenyl-1,10-phenanthroline (BCP), 4,7-diphenyl-1,10-phenanthroline (Bphen), 3-(4-biphenylyl)-4-phenyl-5-tert-butylphenyl-1,2,4-triazole (TAZ), 4-(naphthalen-1-yl)-3,5-diphenyl-4H-1,2,4-triazole (NTAZ), 2-(4-biphenylyl)-5-(4-tert-butylphenyl)-1,3,4-oxadiazole (tBu-PBD), bis(2-methyl-8-quinolinolato-N1,O8)-(1,1′-biphenyl-4-olato)aluminum (BAlq), berylliumbis(benzoquinolin-10-olate (Bebq₂), 9,10-di(naphthalene-2-yl)anthracene (ADN), 1,3-bis[3,5-di(pyridin-3-yl)phenyl]benzene (BmPyPhB), or a mixture thereof.

The electron transport region ETR may include at least one selected from compounds ET1 to ET38 below.

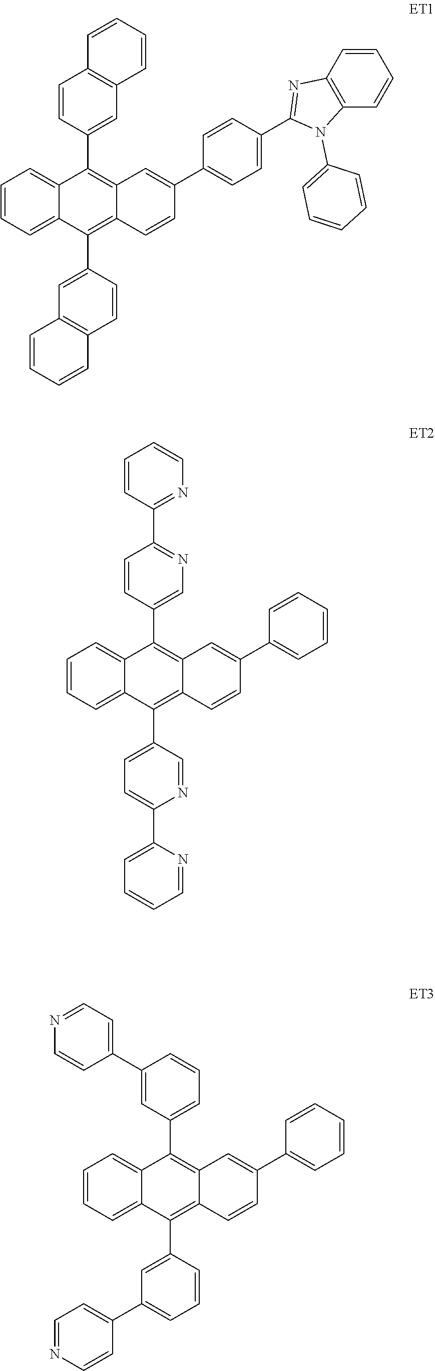 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

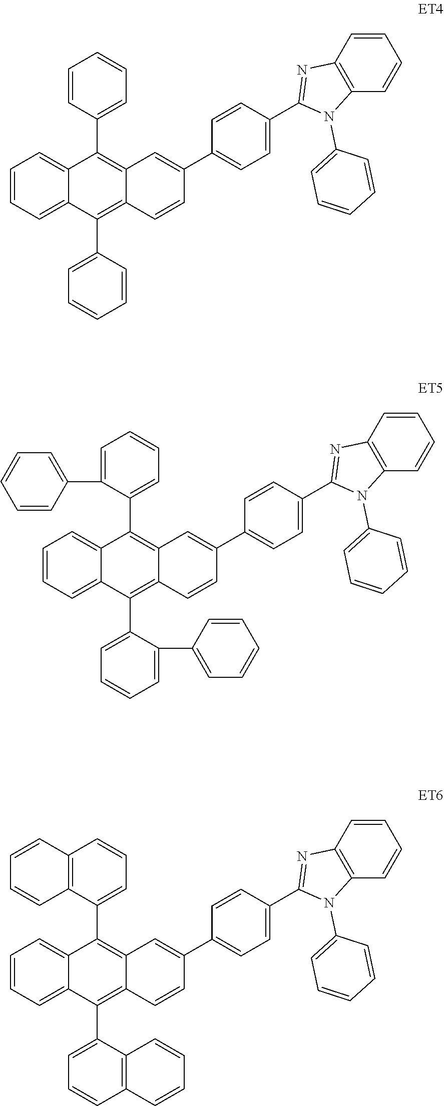 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1]

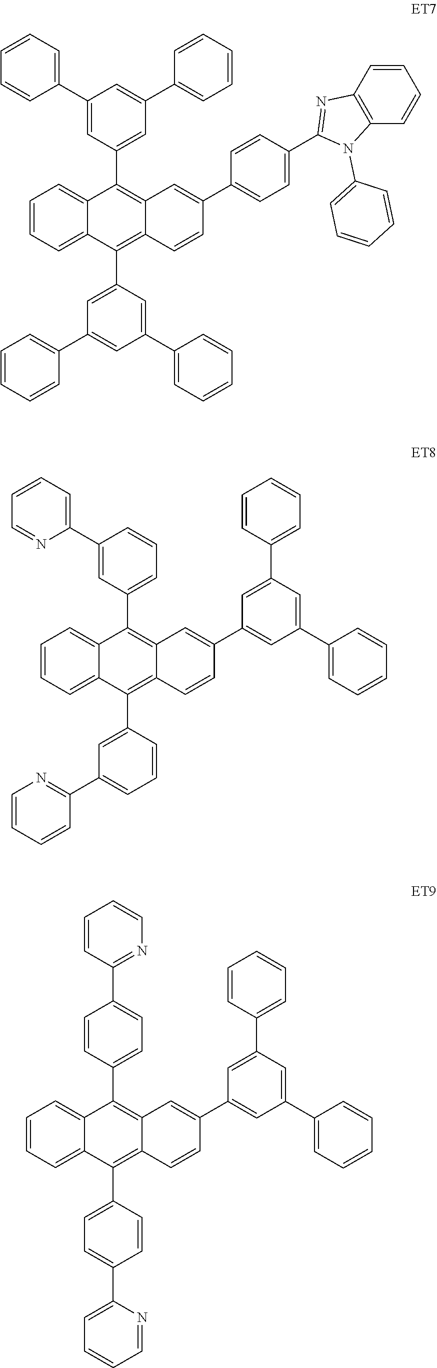 [SMILES: c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1 ; c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1]

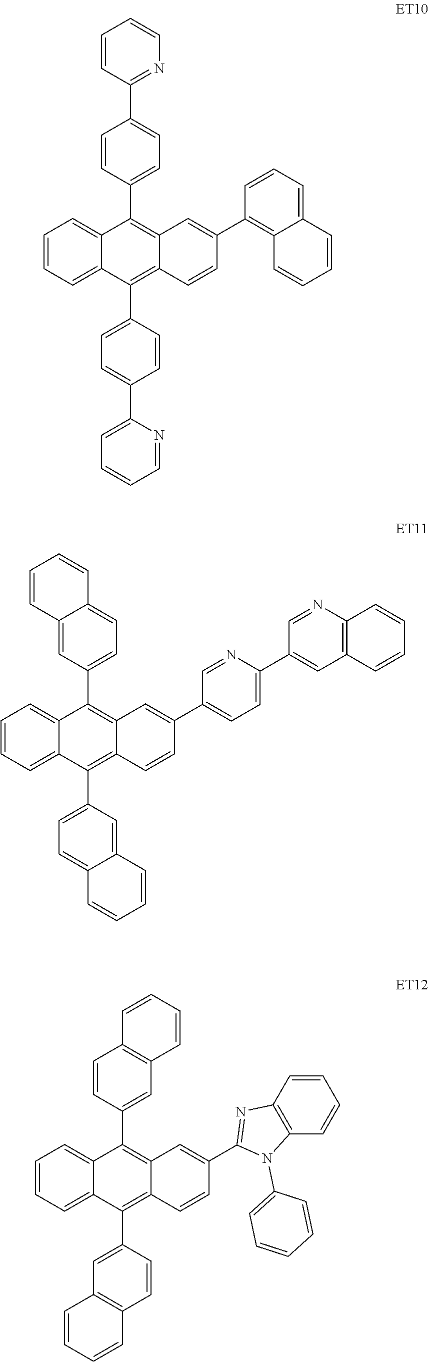 [SMILES: c1ccc(-c2ccc(-c3c4ccccc4c(-c4ccc(-c5ccccn5)cc4)c4cc(-c5cccc6ccccc56)ccc34)cc2)nc1 ; c1ccc2cc(-c3c4ccccc4c(-c4ccc5ccccc5c4)c4cc(-c5ccc(-c6cnc7ccccc7c6)nc5)ccc34)ccc2c1 ; c1ccc(-n2c(-c3ccc4c(-c5ccc6ccccc6c5)c5ccccc5c(-c5ccc6ccccc6c5)c4c3)nc3ccccc32)cc1]

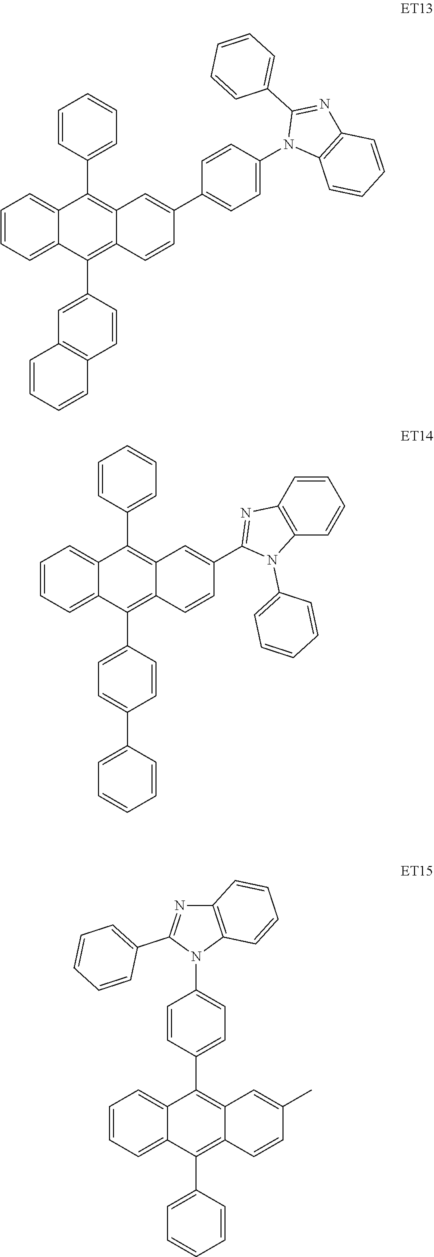 [SMILES: c1ccc(-c2c3ccccc3c(-c3ccc4ccccc4c3)c3ccc(-c4ccc(-n5c(-c6ccccc6)nc6ccccc65)cc4)cc23)cc1 ; c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1]

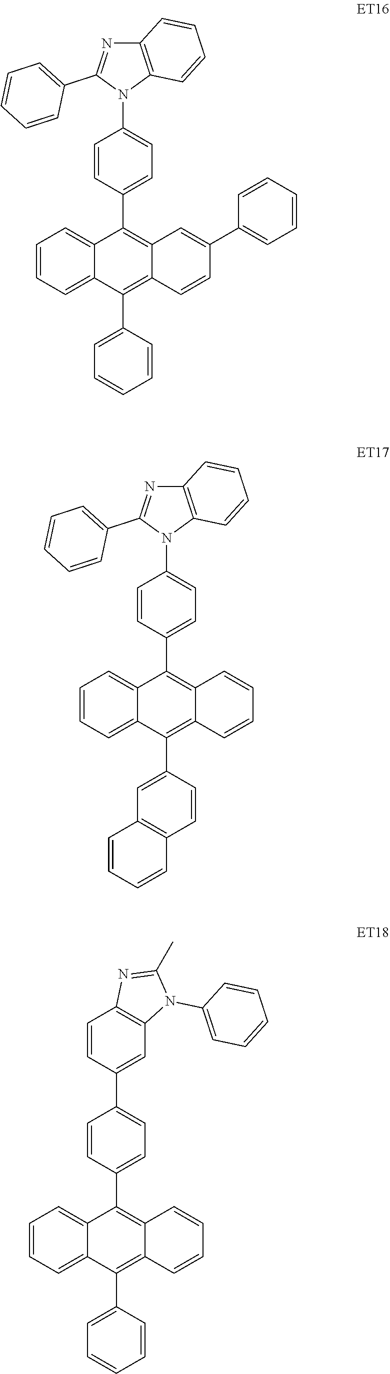 [SMILES: c1ccc(-c2ccc3c(-c4ccccc4)c4ccccc4c(-c4ccc(-n5c(-c6ccccc6)nc6ccccc65)cc4)c3c2)cc1 ; c1ccc(-c2nc3ccccc3n2-c2ccc(-c3c4ccccc4c(-c4ccc5ccccc5c4)c4ccccc34)cc2)cc1 ; Cc1nc2ccc(-c3ccc(-c4c5ccccc5c(-c5ccccc5)c5ccccc45)cc3)cc2n1-c1ccccc1]

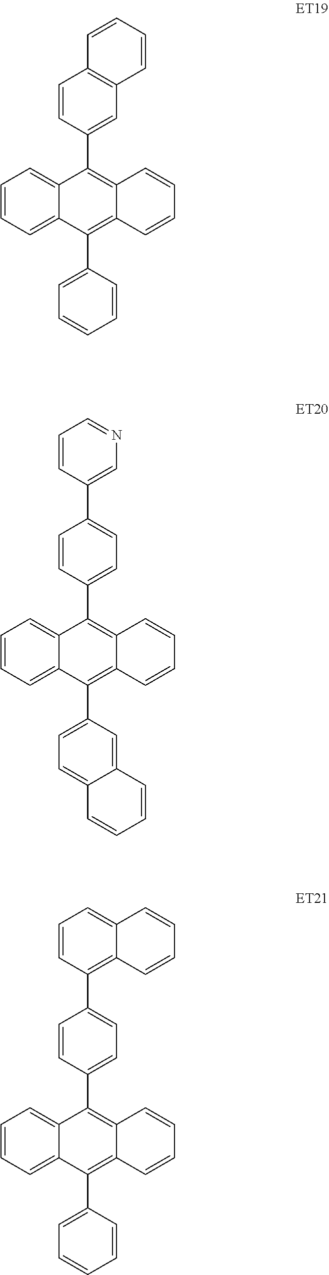

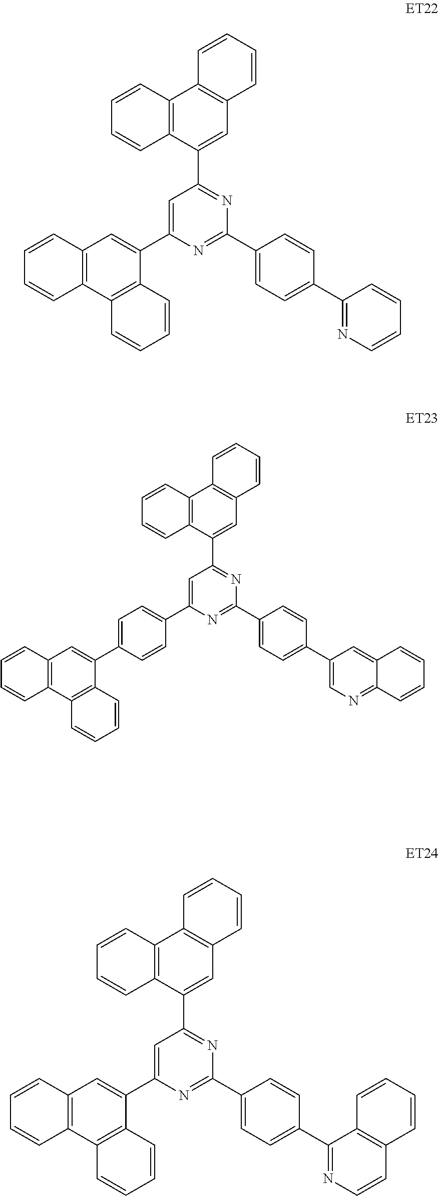 [SMILES: c1ccc(-c2ccc(-c3nc(-c4cc5ccccc5c5ccccc45)cc(-c4cc5ccccc5c5ccccc45)n3)cc2)nc1 ; c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1]

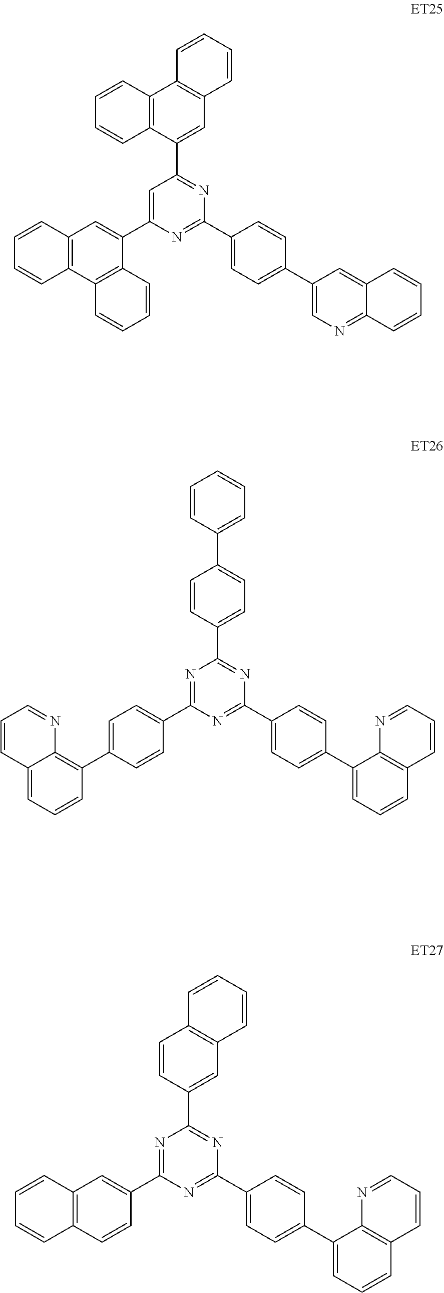 [SMILES: c1ccc2ncc(-c3ccc(-c4nc(-c5cc6ccccc6c6ccccc56)cc(-c5cc6ccccc6c6ccccc56)n4)cc3)cc2c1 ; c1ccc(-c2ccc(-c3nc(-c4ccc(-c5cccc6cccnc56)cc4)nc(-c4ccc(-c5cccc6cccnc56)cc4)n3)cc2)cc1 ; c1ccc2cc(-c3nc(-c4ccc(-c5cccc6cccnc56)cc4)nc(-c4ccc5ccccc5c4)n3)ccc2c1]

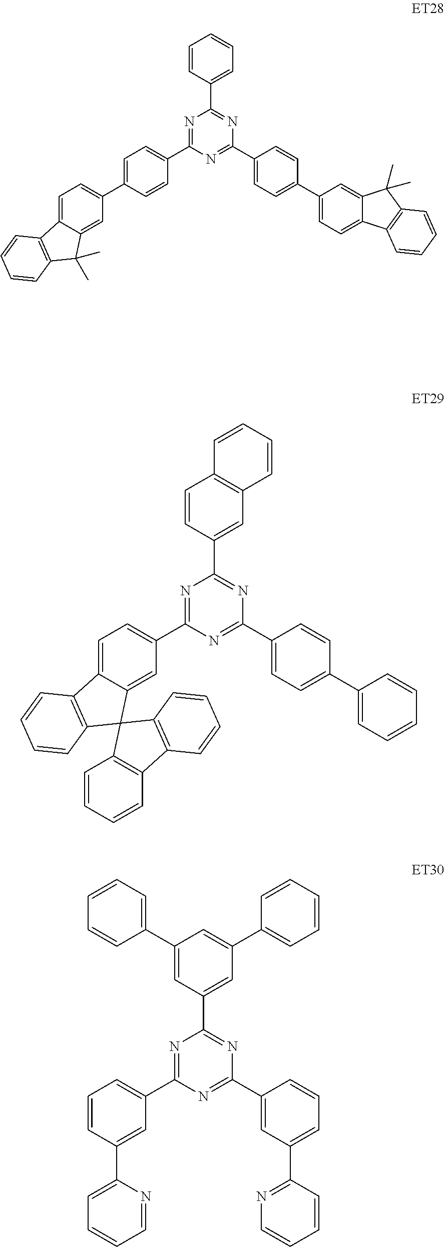 [SMILES: CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21 ; c1ccc(-c2ccc(-c3nc(-c4ccc5c(c4)C4(c6ccccc6-c6ccccc64)c4ccccc4-5)nc(-c4ccc5ccccc5c4)n3)cc2)cc1 ; c1ccc(-c2cc(-c3ccccc3)cc(-c3nc(-c4cccc(-c5ccccn5)c4)nc(-c4cccc(-c5ccccn5)c4)n3)c2)cc1]

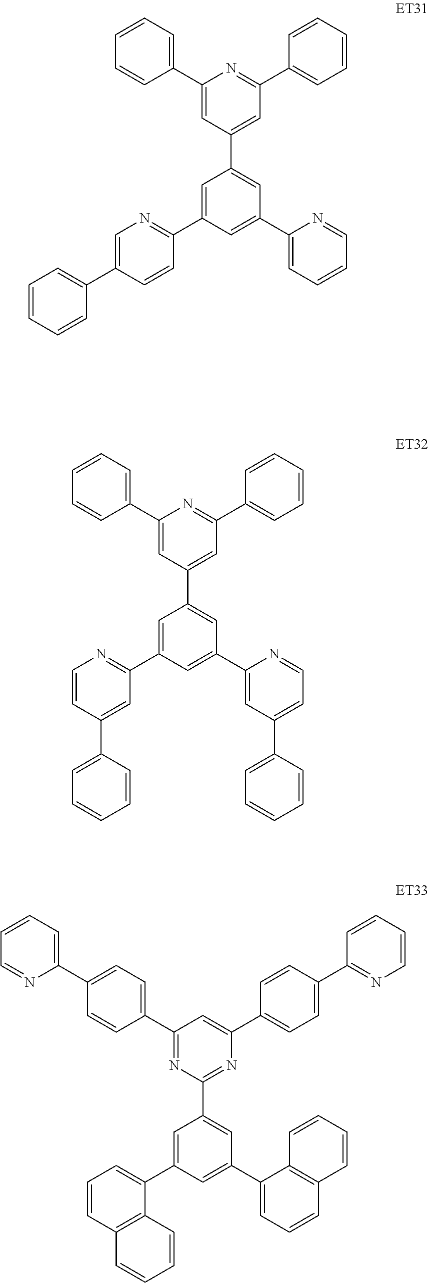 [SMILES: c1ccc(-c2ccc(-c3cc(-c4cc(-c5ccccc5)nc(-c5ccccc5)c4)cc(-c4ccccn4)c3)nc2)cc1 ; c1ccc(-c2ccnc(-c3cc(-c4cc(-c5ccccc5)nc(-c5ccccc5)c4)cc(-c4cc(-c5ccccc5)ccn4)c3)c2)cc1 ; c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1]

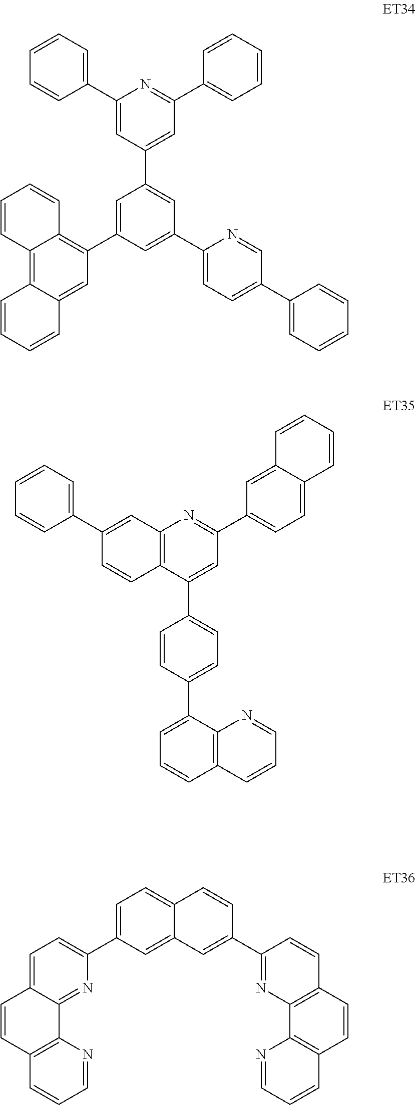 [SMILES: c1ccc(-c2ccc(-c3cc(-c4cc(-c5ccccc5)nc(-c5ccccc5)c4)cc(-c4cc5ccccc5c5ccccc45)c3)nc2)cc1 ; c1ccc(-c2ccc3c(-c4ccc(-c5cccc6cccnc56)cc4)cc(-c4ccc5ccccc5c4)nc3c2)cc1 ; c1cnc2c(c1)ccc1ccc(-c3ccc4ccc(-c5ccc6ccc7cccnc7c6n5)cc4c3)nc12]

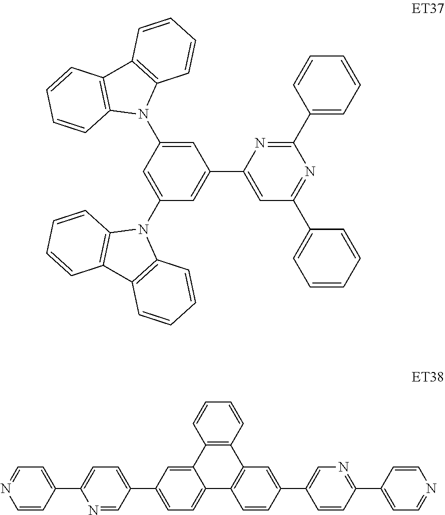 [SMILES: c1ccc(-c2cc(-c3cc(-n4c5ccccc5c5ccccc54)cc(-n4c5ccccc5c5ccccc54)c3)nc(-c3ccccc3)n2)cc1 ; c1ccc2c(c1)c1cc(-c3ccc(-c4ccncc4)nc3)ccc1c1ccc(-c3ccc(-c4ccncc4)nc3)cc21]

In addition, the electron transport region ETR may include halogenated metals such as LiF, NaCl, CsF, RbCl, RbI, CuI, and/or KI, lanthanide metals such as Yb, and/or co-deposition materials of a halogenated metal and a lanthanide metal. For example, the electron transport region ETR may include KI:Yb, RbI:Yb, LiF:Yb, and/or the like as a co-deposition material. In some embodiments, for the electron transport region ETR, a metal oxide such as Li₂O and/or BaO, and/or 8-hydroxyl-lithium quinolate (Liq), and/or the like may be used, but the embodiment of the present disclosure is not limited thereto. The electron transport region ETR may also be formed of a mixture material of an electron transport material and an insulating organo-metal salt (e.g., an electrically insulating organo-metal salt). The organo metal salt may be a material having an energy band gap of about 4 eV or greater. In some embodiments, the organo-metal salt may include, for example, metal acetates, metal benzoates, metal acetoacetates, metal acetylacetonates, and/or metal stearates.

The electron transport region ETR may further include, for example, at least one selected from 2,9-dimethyl-4,7-diphenyl-1,10-phenanthroline (BCP), diphenyl(4-(triphenylsilyl)phenyl)phosphine oxide (TSPO1), and 4,7-diphenyl-1,10-phenanthroline (Bphen) in addition to the materials described above, but the embodiment of the present disclosure is not limited thereto.

The electron transport region ETR may include the compounds of the electron transport region described above in at least one selected from among the electron injection layer EIL, the electron transport layer ETL, and the hole blocking layer HBL.

When the electron transport region ETR includes the electron transport layer ETL, the electron transport layer ETL may have a thickness of about 100 Å to about 1000 Å, for example, about 150 Å to about 500 Å. When the thickness of the electron transport layer ETL satisfies the above-described range, suitable or satisfactory electron transport properties may be obtained without a substantial increase in driving voltage. When the electron transport region ETR includes the electron injection layer EIL, the electron injection layer EIL may have a thickness of about 1 Å to about 100 Å, for example, about 3 Å to about 90 Å. When the thickness of the electron injection layer EIL satisfies the above-described ranges, suitable or satisfactory electron injection properties may be obtained without a substantial increase in driving voltage.

The second electrode EL2 may be provided on the electron transport region ETR. The second electrode EL2 may be a common electrode. The second electrode EL2 may be a cathode or an anode but the embodiment of the present disclosure is not limited thereto. For example, when the first electrode EL1 is an anode, the second electrode EL2 may be a cathode, and when the first electrode EL1 is a cathode, the second electrode EL2 may be an anode. The second electrode EL2 may include at least one selected from Ag, Mg, Cu, Al, Pt, Pd, Au, Ni, Nd, Ir, Cr, Li, Ca, LiF, Mo, Ti, W, In, Sn, and Zn, two or more compounds selected therefrom, two or more mixtures selected therefrom, and/or an oxide thereof.

The second electrode EL2 may be a transmissive electrode, a transflective electrode, or a reflective electrode. When the second electrode EL2 is a transmissive electrode, the second electrode EL2 may be formed of a transparent metal oxide, for example, indium tin oxide (ITO), indium zinc oxide (IZO), zinc oxide (ZnO), indium tin zinc oxide (ITZO), and/or the like.

When the second electrode EL2 is a transflective electrode or a reflective electrode, the second electrode EL2 may include Ag, Mg, Cu, Al, Pt, Pd, Au, Ni, Nd, Ir, Cr, Li, Ca, LiF/Ca, LiF/Al, Mo, Ti, Yb, W, a compound thereof, and/or a mixture thereof (e.g., AgMg, AgYb, and/or MgYb). In some embodiments, the second electrode EL2 may have a multilayer structure including a reflective film or a transflective film formed of the above-described materials, and a transparent conductive film formed of indium tin oxide (ITO), indium zinc oxide (IZO), zinc oxide (ZnO), indium tin zinc oxide (ITZO), and/or the like. For example, the second electrode EL2 may include the above-described metal materials, a combination of two or more metal materials selected from the above-described metal materials, and/or oxides of the above-described metal materials.

In some embodiments, the second electrode EL2 may be connected with an auxiliary electrode. When the second electrode EL2 is connected with the auxiliary electrode, the resistance of the second electrode EL2 may decrease.

In some embodiments, a capping layer CPL may be further on the second electrode EL2 of the light emitting element ED of an embodiment. The capping layer CPL may include a multilayer or a single layer.

In an embodiment, the capping layer CPL may be an organic layer and/or an inorganic layer. For example, when the capping layer CPL includes an inorganic material, the inorganic material may include an alkali metal compound such as LiF, an alkaline earth metal compound such as MgF₂, SiON, SiNₓ, SiO_{y}, and/or the like.

For example, when the capping layer CPL includes an organic material, the organic material may include a-NPD, NPB, TPD, m-MTDATA, Alq3 CuPc, N4,N4,N4′,N4′-tetra(biphenyl-4-yl) biphenyl-4,4′-diamine (TPD15), 4,4′,4″-tris(carbazol-9-yl)triphenylamine (TCTA), and/or the like or may include epoxy resins and/or acrylates such as methacrylates. However, the embodiment of the present disclosure is not limited thereto, and the capping layer CPL may include compounds P1 to P5 below.

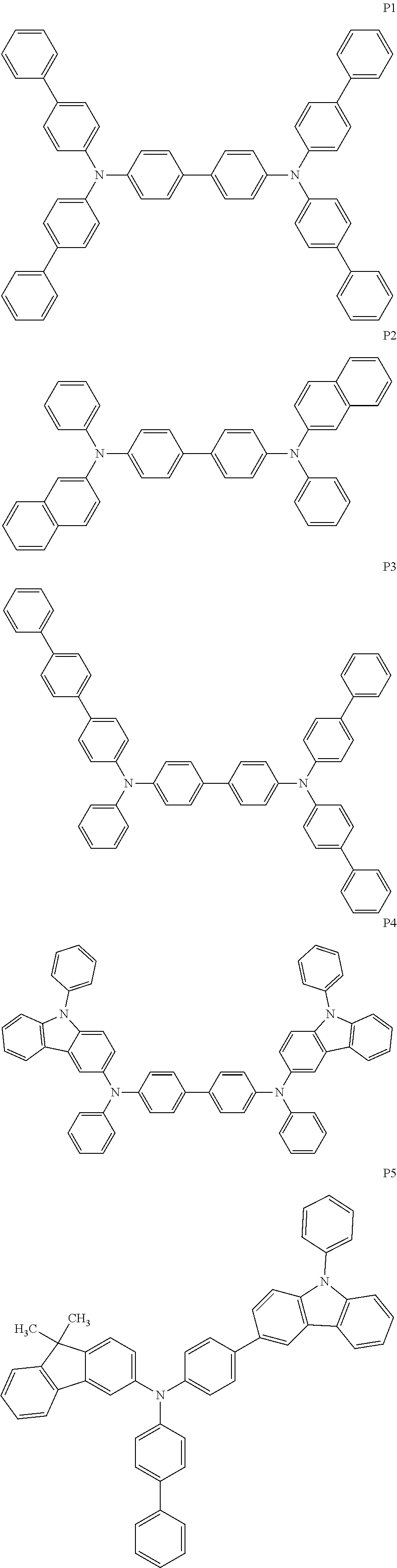

In some embodiments, the capping layer CPL may have a refractive index of about 1.6 or greater. For example, the capping layer CPL may have a refractive index of about 1.6 or greater in a wavelength range of about 550 nm to about 660 nm.

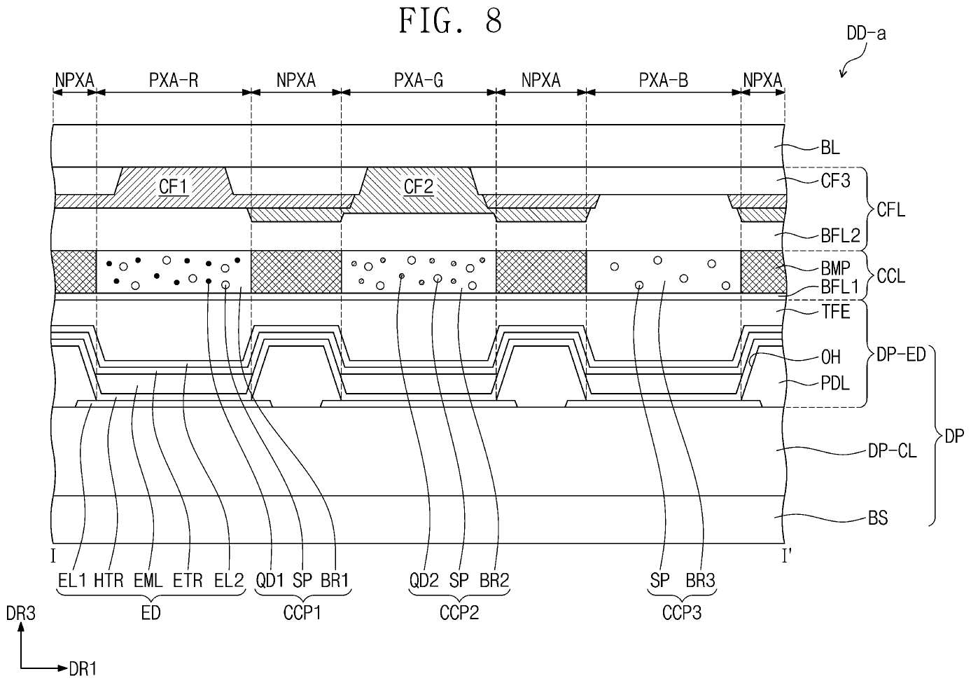

FIGS. 8-11 are each a cross-sectional view of a display device according to an embodiment. Hereinafter, in the description of the display device according to an embodiment with reference to FIGS. 8-11, content overlapping the one described above with reference to FIGS. 1-7 will not be described again, and the differences will be mainly described.

Referring to FIG. 8, a display device DD according to an embodiment may include a display panel DP having a display element layer DP-ED, a light control layer CCL on the display panel DP, and a color filter layer CFL. In an embodiment shown in FIG. 8, the display panel DP may include a base layer BS, a circuit layer DP-CL provided on the base layer BS, and a display element layer DP-ED, and the element layer DP-ED may include a light emitting element ED.

The light emitting element ED may include a first electrode EL1, a hole transport region HTR on the first electrode EL1, an emission layer EML on the hole transport region HTR, an electron transport region ETR on the emission layer EML, and a second electrode EL2 on the electron transport region ETR. The light emitting element ED may include an amine compound of an embodiment. A structure of the light emitting element ED shown in FIG. 8 may be the same (or substantially the same) as the structure of the light emitting element of FIGS. 3-7 described above.

Referring to FIG. 8, the emission layer EML may be in the openings OH defined in the pixel defining films PDL. For example, the emission layer EML separated by the pixel defining films PDL and provided corresponding to each of light emitting regions PXA-R, PXA-G, and PXA-B may emit light in the same wavelength ranges. In the display device DD-a of an embodiment, the emission layer EML may emit blue light. In some embodiments, the emission layer EML may be provided as a common layer throughout the light emitting regions PXA-R, PXA-G, and PXA-B.

The light control layer CCL may be on the display panel DP. The light control layer CCL may include a light converter. The light converter may be a quantum dot and/or a phosphor. The light converter may wavelength-convert the provided light and emit the wavelength-converted light. In some embodiments, the light control layer CCL may be a layer containing quantum dots and/or phosphors.

The light control layer CCL may include a plurality of light control units CCP1, CCP2, and CCP3. The light control units CCP1, CCP2, and CCP3 may be spaced apart from each other.

Referring to FIG. 8, a division pattern BMP may be between the light control units CCP1, CCP2, and CCP3 spaced apart from each other, but the embodiment of the present disclosure is not limited thereto. In FIG. 8, the division pattern BMP is shown to non-overlap the light control units CCP1, CCP2, and CCP3, but edges of the light control units CCP1, CCP2, and CCP3 may overlap at least a portion of the division pattern BMP.

The light control layer CCL may include a first light control unit CCP1 including a first quantum dot QD1 to convert a first color light provided from the light emitting element ED into a second color light, a second light control unit CCP2 including a second quantum dot QD2 to convert the first color light into a third color light, and a third light control unit CCP3 to convert the first color light.

In an embodiment, the first light control unit CCP1 may provide red light, which is the second color light, and the second light control unit CCP2 may provide green light, which is the third color light. The third light control unit CCP3 may transmit and provide blue light, which is the first color light provided from the light emitting element ED. For example, the first quantum dot QD1 may be a red quantum dot and the second quantum dot QD2 may be a green quantum dot. The same descriptions above may be applied to the quantum dots QD1 and QD2.

In addition, the light control layer CCL may further include scatterers SP (e.g., light scatterers SP). The first light control unit CCP1 may include the first quantum dot QD1 and the scatterers SP, the second light control unit CCP2 may include the second quantum dot QD2 and the scatterers SP, and the third light control unit CCP3 may not include a quantum dot but may include the scatterers SP.

The scatterers SP may be inorganic particles. For example, the scatterers SP may include at least one selected from among TiO₂, ZnO, Al₂O₃, SiO₂, and hollow silica. The scatterers SP may include any one selected from among TiO₂, ZnO, Al₂O₃, SiO₂, and hollow silica, or may be a mixture of two or more materials selected from TiO₂, ZnO, Al₂O₃, SiO₂, and hollow silica.

The first light control unit CCP1, the second light control unit CCP2, and the third light control unit CCP3 may each include base resins BR1, BR2, and BR3 for dispersing the quantum dots QD1 and QD2 and the scatterers SP. In an embodiment, the first light control unit CCP1 may include the first quantum dot QD1 and the scatterers SP dispersed in the first base resin BR1, the second light control unit CCP2 may include the second quantum dot QD2 and the scatterers SP dispersed in the second base resin BR2, and the third light control unit CCP3 may include the scatterers SP dispersed in the third base resin BR3.

The base resins BR1, BR2, and BR3 are a medium in which the quantum dots QD1 and QD2 and the scatterers SP are dispersed, and may be formed of various suitable resin compositions, which may be generally referred to as a binder. For example, the base resins BR1, BR2, and BR3 may be an acrylic-based resin, a urethane-based resin, a silicone-based resin, an epoxy-based resin, etc. The base resins BR1, BR2, and BR3 may be a transparent resin. In an embodiment, the first base resin BR1, the second base resin BR2, and the third base resin BR3 may each be the same as or different from each other.

The light control layer CCL may include a barrier layer BFL1. The barrier layer BFL1 may serve to prevent or reduce introduction of moisture and/or oxygen (which hereinafter may be referred to as “moisture/oxygen”). The barrier layer BFL1 may prevent or reduce exposure of the light control units CCP1, CCP2, and CCP3 to moisture/oxygen. In some embodiments, the barrier layer BFL1 may cover the light control units CCP1, CCP2, and CCP3. In addition, a barrier layer BFL2 may be provided between the light control units CCP1, CCP2, and CCP3 and the color filter layer CFL.

The barrier layers BFL1 and BFL2 may include at least one inorganic layer. In some embodiments, the barrier layers BFL1 and BFL2 may be formed of an inorganic material. For example, the barrier layers BFL1 and BFL2 may be formed including silicon nitride, aluminum nitride, zirconium nitride, titanium nitride, hafnium nitride, tantalum nitride, silicon oxide, aluminum oxide, titanium oxide, tin oxide, cerium oxide, silicon oxynitride, and/or a metal thin film in which light transmittance is secured, and/or the like. In some embodiments, the barrier layers BFL1 and BFL2 may further include an organic film. The barrier layers BFL1 and BFL2 may be formed of a single layer or a plurality of layers.

In the display device DD-a of an embodiment, the color filter layer CFL may be on the light control layer CCL. For example, the color filter layer CFL may be directly on the light control layer CCL. In this case, the barrier layer BFL2 may be omitted.

The color filter layer CFL may include filters CF1, CF2, and CF3. In some embodiments, the color filter layer CFL may include a first filter CF1 that transmits a second color light, a second filter CF2 that transmits a third color light, and a third filter CF3 that transmits a first color light. For example, the first filter CF1 may be a red filter, the second filter CF2 may be a green filter, and the third filter CF3 may be a blue filter. The filters CF1, CF2, and CF3 may each include a polymer photosensitive resin, a pigment and/or a dye. The first filter CF1 may include a red pigment and/or a red dye, the second filter CF2 may include a green pigment and/or a green dye, and the third filter CF3 may include a blue pigment and/or a blue dye. In some embodiments, the embodiment of the present disclosure is not limited thereto, and the third filter CF3 may not include a pigment or a dye. The third filter CF3 may include a polymer photosensitive resin, but not include a pigment or a dye. The third filter CF3 may be transparent. The third filter CF3 may be formed of a transparent photosensitive resin.

In addition, in an embodiment, the first filter CF1 and the second filter CF2 may be yellow filters. The first filter CF1 and the second filter CF2 may not be separated and may be provided as a single body. The first to third filters CF1, CF2, and CF3 may correspond to the red light emitting region PXA-R, the green light emitting region PXA-G, and the blue light emitting region PXA-B, respectively.

In some embodiments, the color filter layer CFL may further include a light blocking unit. The light blocking unit may be a black matrix. The light blocking unit may be formed including an organic light blocking material and/or an inorganic light blocking material, each including a black pigment and/or a black dye. The light blocking unit may prevent or reduce light leakage, and separate boundaries between the adjacent filters CF1, CF2, and CF3.

The base substrate BL may be on the color filter layer CFL. The base substrate BL may be a member providing a base surface on which the color filter layer CFL and the light control layer CCL are provided. The base substrate BL may be a glass substrate, a metal substrate, a plastic substrate, and/or the like. However, the embodiment of the present disclosure is not limited thereto, and the base substrate BL may be an inorganic layer, an organic layer, or a composite material layer. In some embodiments, unlike what is shown, the base substrate BL may be omitted in an embodiment.

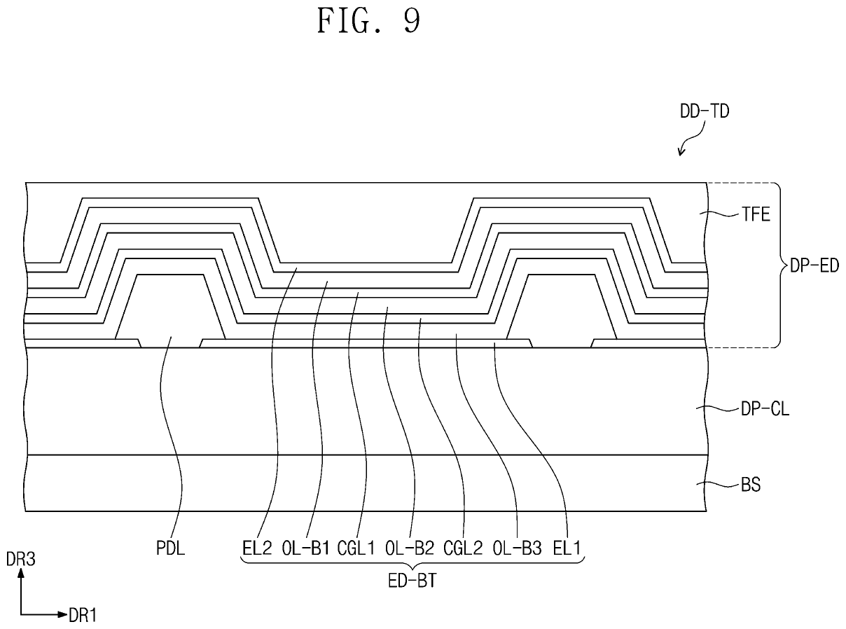

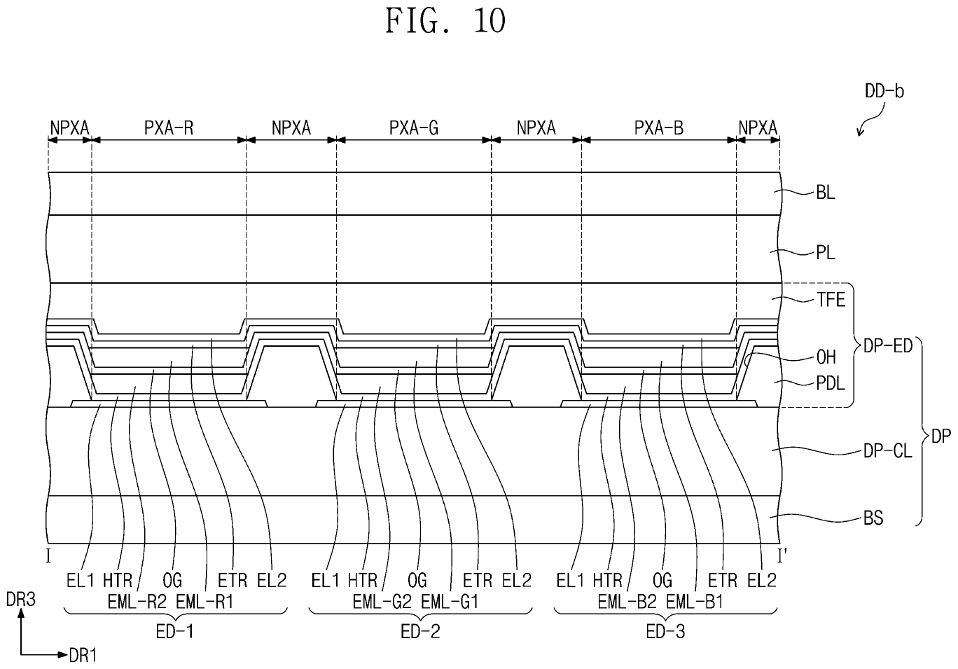

FIG. 9 is a cross-sectional view showing a portion of a display device according to an embodiment. FIG. 9 may be a cross-sectional view showing another embodiment of the display panel DP of FIG. 8. In a display device DD-TD of an embodiment, a light emitting element ED-BT may include a plurality of light emitting structures OL-B1, OL-B2, and OL-B3. At least one selected from the plurality of light emitting structures OL-B1, OL-B2, and OL-B3 may include the amine compound of an embodiment.

The light emitting element ED-BT may include the first electrode EL1 and the second electrode EL2 facing each other, and the plurality of light emitting structures OL-B1, OL-B2, and OL-B3 provided by being sequentially stacked in a thickness direction between the first electrode EL1 and the second electrode EL2. The light emitting structures OL-B1, OL-B2, and OL-B3 each may include the emission layer EML (FIG. 8), a hole transport region HTR and an electron transport region ETR having the emission layer EML (FIG. 8) therebetween. In some embodiments, the light emitting element ED-BT included in the display device DD-TD of an embodiment may be a light emitting element having a tandem structure including a plurality of emission layers.

In an embodiment shown in FIG. 9, light emitted from each of the light emitting structures OL-B1, OL-B2, and OL-B3 may all be blue light. However, the embodiment of the present disclosure is not limited thereto, and wavelength ranges of light emitted from each of the light emitting structures OL-B1, OL-B2, and OL-B3 may be different from each other. For example, the light emitting element ED-BT including the plurality of light emitting structures OL-B1, OL-B2, and OL-B3 emitting light in different wavelength ranges may emit white light.

Charge generation layers CGL1 and CGL2 may be between neighboring light emitting structures OL-B1, OL-B2, and OL-B3. The charge generation layers CGL1 and CGL2 may include a p-type charge generation layer and/or an n-type charge generation layer.

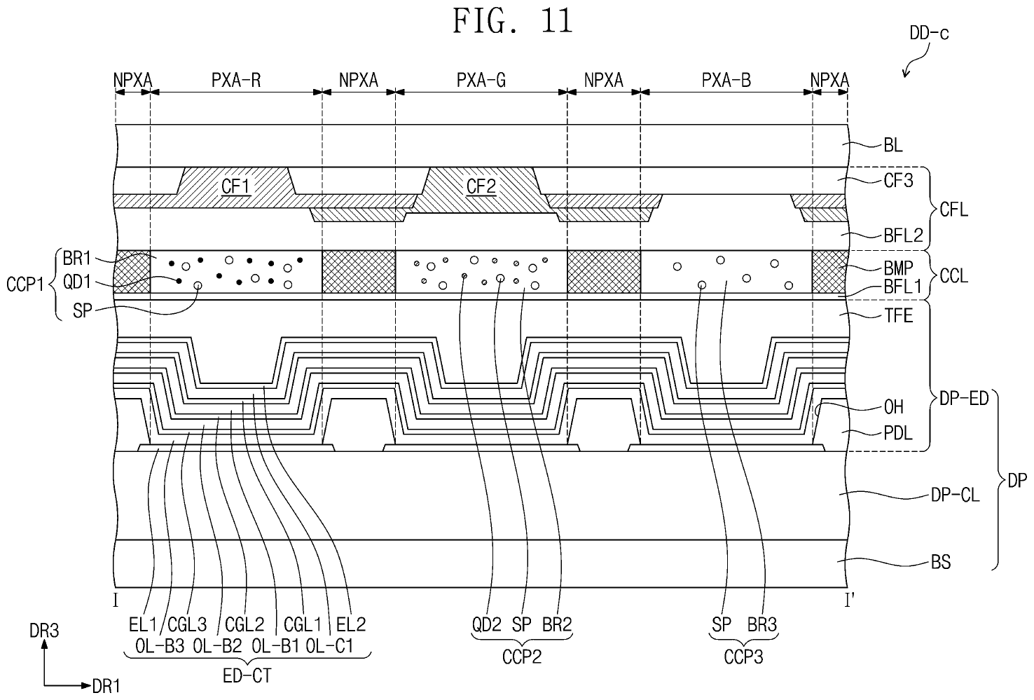

Referring to FIG. 10, a display device DD-b according to an embodiment may include light emitting elements ED-1, ED-2, and ED-3 in which two emission layers are stacked. Compared to the display device DD according to an embodiment shown in FIG. 2, the difference is that in an embodiment shown in FIG. 10, the first to third light emitting elements ED-1, ED-2, and ED-3 each include two emission layers stacked in a thickness direction. In each of the first to third light emitting elements ED-1, ED-2, and ED-3, the two emission layers may emit light in the same wavelength range.

The first light emitting element ED-1 may include a first red emission layer EML-R1 and a second red emission layer EML-R2. The second light emitting element ED-2 may include a first green emission layer EML-G1 and a second green emission layer EML-G2. In addition, the third light emitting element ED-3 may include a first blue emission layer EML-B1 and a second blue emission layer EML-B2. A light emitting auxiliary portion OG may be between the first red emission layer EML-R1 and the second red emission layer EML-R2, between the first green emission layer EML-G1 and the second green emission layer EML-G2, and between the first blue emission layer EML-B1 and the second blue emission layer EML-B2.

The light emitting auxiliary portion OG may include a single layer or a plurality of layers. The light emitting auxiliary portion OG may include a charge generation layer. In some embodiments, the light emitting auxiliary portion OG may include an electron transport region, a charge generation layer, and a hole transport region that are sequentially stacked. The light emitting auxiliary portion OG may be provided as a common layer throughout the first to third light emitting elements ED-1, ED-2, and ED-3. However, the embodiment of the present disclosure is not limited thereto, and the light emitting auxiliary portion OG may be provided to be patterned inside the openings OH defined in the pixel defining films PDL.

The first red emission layer EML-R1, the first green emission layer EML-G1, and the first blue emission layer EML-B1 may be between the light emitting auxiliary portion OG and the electron transport region ETR. The second red emission layer EML-R2, the second green emission layer EML-G2, and the second blue emission layer EML-B2 may be between the hole transport region HTR and the light emitting auxiliary portion OG.

In some embodiments, the light emitting element ED-1 may include the first electrode EL1, the hole transport region HTR, the second red emission layer EML-R2, the emission auxiliary portion OG, the first red emission layer EML-R1, the electron transport region ETR, and the second electrode EL2, which are sequentially stacked. The second light emitting element ED-2 may include the first electrode EL1, the hole transport region HTR, the second green emission layer EML-G2, the emission auxiliary portion OG, the first green emission layer EML-G1, the electron transport region ETR, and the second electrode EL2, which are sequentially stacked. The third light emitting element ED-3 may include the first electrode EL1, the hole transport region HTR, the second blue emission layer EML-B2, the emission auxiliary portion OG, the first blue emission layer EML-B1, the electron transport region ETR, and the second electrode EL2, which are sequentially stacked.

In some embodiments, an optical auxiliary layer PL may be on the display element layer DP-ED. The optical auxiliary layer PL may include a polarizing layer. The optical auxiliary layer PL may be on the display panel DP to control reflected light in the display panel DP due to external light. Unlike what is shown, the optical auxiliary layer PL may be omitted in the display device according to an embodiment.

Unlike FIGS. 9-10, the display device DD-c of FIG. 11 is illustrated to include four light emitting structures OL-B1, OL-B2, OL-B3, and OL-C1. The light emitting element ED-BT may include the first electrode EL1 and the second electrode EL2 facing each other, and the first to fourth light emitting structures L-B1, OL-B2, OL-B3, and OL-C1 sequentially stacked in a thickness direction between the first electrode EL1 and the second electrode EL2. At least one selected from the first to fourth light emitting structures OL-B1, OL-B2, OL-B3, and OL-C1 may include the amine compound of an embodiment.

Among the four light emitting structures, the first to third light emitting structures OL-B1, OL-B2, and OL-B3 may emit blue light, and the fourth light emitting structure OL-C1 may emit green light. However, the embodiment of the present disclosure is not limited thereto, and the first to fourth light emitting structures OL-B1, OL-B2, OL-B3, and OL-C1 may emit light having different wavelength ranges.

Between the third light emitting structure OL-B3, the second light emitting structure OL-B2, the first light emitting structure OL-B1, and the fourth light emitting structure OL-C1, charge generation layers CGL3, CGL2, and CGL1 may be provided. The charge generation layers CGL3, CGL2 and CGL1 between the neighboring light emitting structures OL-B3, OL-B2, OL-B1, and OL-C1 may include a p-type charge generation layer and/or an n-type charge generation layer.

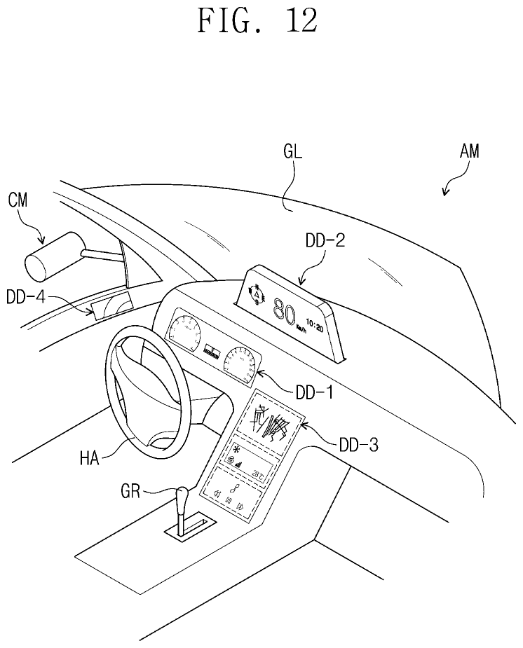

FIG. 12 is a view showing a vehicle AM in which first to fourth display devices DD-1, DD-2, DD-3, and DD-4 are provided. At least one selected from the first to fourth display devices DD-1, DD-2, DD-3, and DD-4 may have the same configuration as the display devices DD, DD-TD, DD-a, DD-b, and/or DD-c of embodiments described with reference to FIGS. 1, 2, and 8-11.

FIG. 12 shows a car as the vehicle AM, but this is presented as an example, and the first to fourth display devices DD-1, DD-2, DD-3, and DD-4 may be on other means of transportation, such as bicycles, motorcycles, trains, ships, and/or airplanes. In addition, at least one selected from the first to fourth display devices DD-1, DD-2, DD-3, and DD-4 having the same configuration as the display devices DD, DD-TD, DD-a, DD-b, and/or DD-c of embodiments may be adopted for personal computers, laptop computers, personal digital terminals, game consoles, portable electronic devices, televisions, monitors, outdoor billboards, and/or the like. In addition, these are merely presented as an embodiment, and thus the display device may be adopted for other electronic devices without departing from the spirit or scope of the present disclosure.

At least one selected from the first to fourth display devices DD-1, DD-2, DD-3, and DD-4 may include the light emitting element ED of an embodiment described with reference to FIGS. 3-7. The light emitting element ED according to an embodiment may include an amine compound of an embodiment. At least one selected from the first to fourth display devices DD-1, DD-2, DD-3, and DD-4 includes a light emitting element ED containing the amine compound of an embodiment, thereby increasing lifespan.

Referring to FIG. 12, the vehicle AM may include a wheel HA and a gear GR for operating the vehicle AM, and have a front window GL facing a driver.

The first display device DD-1 may be in a first region overlapping the wheel HA. For example, the first display device DD-1 may be a digital cluster that displays first information of the vehicle AM. The first information may include a first scale indicating driving speed of the vehicle AM, a second scale indicating engine revolutions (e.g., revolutions per minute (RPM)), and an image indicating fuel gauge, and/or the like. The first scale and the second scale may be displayed as digital images.

The second display device DD-2 may be in a second region facing a driver seat and overlapping the front window GL. The driver seat may be a seat in which (or in front of which) the wheel HA is provided. For example, the second display device DD-2 may be a heads up display HUD displaying second information of the vehicle AM. The second display device DD-2 may be optically transparent. The second information includes digital numbers indicating driving speed of the vehicle AM and may further include information such as current time. Unlike what is shown, the second information of the second display device DD-2 may be projected and displayed on the front window GL.

The third display device DD-3 may be in a third region adjacent to the gear GR. For example, the third display device DD-3 may be a center information display CID for a vehicle, which is between a driver seat and a front passenger seat and displays third information. The passenger seat may be a seat spaced apart from the driver seat with the gear GR therebetween. The third information may include information about road conditions (e.g., navigation information), music or radio play, dynamic video (and/or image) play, temperature inside the vehicle AM, and/or the like.

The fourth display device DD-4 may be in a fourth region spaced apart from the wheel HA and the gear GR and adjacent to a side of the vehicle AM. For example, the fourth display device DD-4 may be a digital side mirror that displays fourth information. The fourth display device DD-4 may display images of conditions outside the vehicle AM, which are taken by a camera module CM outside the vehicle AM. The fourth information may include images of conditions outside the vehicle AM.

The first to fourth information described above are presented as an example, and the first to fourth display devices DD-1, DD-2, DD-3, and DD-4 may further display information about inside and/or outside a vehicle. The first to fourth information may include different information. However, the embodiment of the present disclosure is not limited thereto, and some of the first to fourth information may include the same information.

Hereinafter, with reference to Examples and Comparative Examples, an amine compound according to an embodiment of the present disclosure and a light emitting element of an embodiment will be described in more detail. In addition, Examples below are shown only for the understanding of the present disclosure, and the scope of the present disclosure is not limited thereto.

### Examples

### 1. Synthesis of Amine Compounds of Examples

A process of synthesizing amine compounds according to an embodiment of the present disclosure will be described in more detail by presenting a process of synthesizing Compounds 1, 13, 17, 22, 26, and 37 as an example. In addition, a process of synthesizing amine compounds, which will be described hereinafter, is provided as an example, and thus a process of synthesizing compounds according to an embodiment of the present disclosure is not limited to the Examples below.

### (1) Synthesis of Amine Compound 1

Amine compound 1 according to an embodiment may be synthesized by, for example, processes of Reaction Formula 1 below.

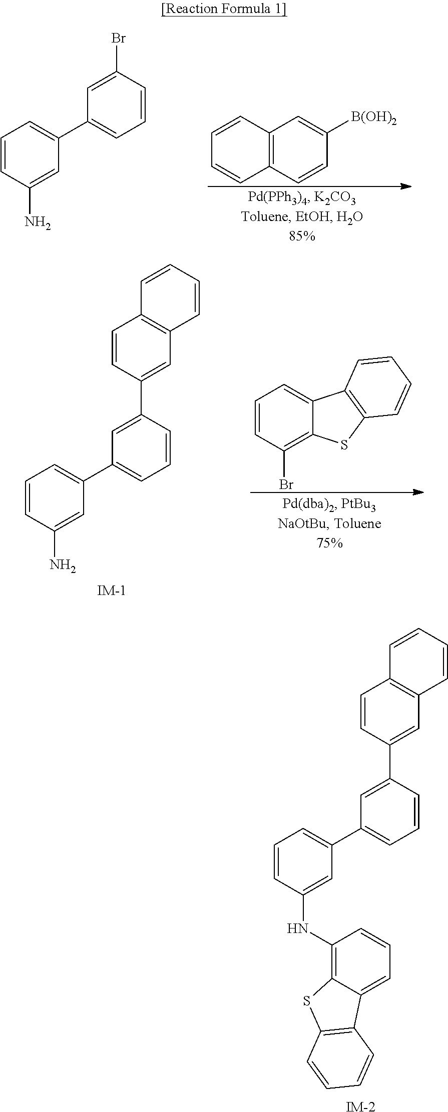

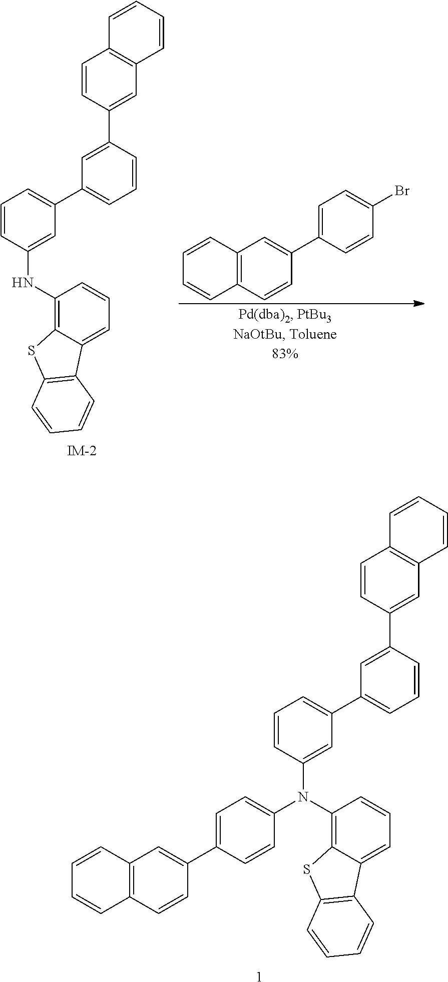

### Synthesis of Compound IM-1

In an Ar atmosphere, 3′-bromo-[1,1′-biphenyl]-3-amine (25.00 g, 100.8 mmol), naphthalen-2-ylboronic acid (19.06 g, 1.1 equiv, 110.8 mmol), Pd(PPh₃)₄ (11.64 g, 0.10 equiv, 10.08 mmol), K₂CO₃ (27.85 g, 2.0 equiv, 201.5 mmol), toluene (400 mL), EtOH (200 mL), and H₂O (100 mL) were sequentially added to a three-neck flask (2000 mL), and heated and stirred under reflux. The resultant solution was cooled to room temperature, and then water and toluene were added to a reaction solvent, and an organic layer was separated and collected. The organic layer was washed with brine, and dried over MgSO₄. MgSO₄ was filtered and the organic layer was concentrated to obtain a crude product. The crude product was purified to obtain Compound IM-1 (25.25 g, yield: 85%). A molecular ion peak was observed at m/z (mass number) 269 as measured by fast atom bombardment mass spectrometry (FAB MS), thereby identifying Compound IM-1.

### Synthesis of Compound IM-2

In an Ar atmosphere, Compound IM-1 (10.00 g, 33.85 mmol), Pd(dba)₂ (0.97 g, 0.05 equiv, 1.7 mmol), NaOtBu (3.25 g, 1.0 equiv, 33.9 mmol), toluene (340 mL), 4-bromodibenzo[b,d]thiophene (8.91 g, 1.0 equiv, 33.9 mmol), and PtBu₃ (1.37 g, 0.2 equiv, 6.77 mmol) were sequentially added to a three-neck flask (1000 mL), and heated and stirred under reflux. The resultant solution was cooled to room temperature, and then water was added to a reaction solvent, and an organic layer was separated and collected. Toluene was added to the water layer to further extract an organic layer. The extracted organic layer was washed with brine and dried over MgSO₄. MgSO₄ was filtered and the organic layer was concentrated to obtain a crude product. The crude product was purified to obtain Compound IM-2 (12.15 g, yield: 75%). A molecular ion peak was observed at m/z (mass number) 477 as measured by FAB MS, thereby identifying Compound IM-2.

### Synthesis of Compound 1

In an Ar atmosphere, Compound IM-2 (12.15 g, 25.44 mmol), Pd(dba)₂ (0.73 g, 0.05 equiv, 1.3 mmol), NaOtBu (2.44 g, 1.0 equiv, 25.4 mmol), toluene (250 mL), 2-(4-bromophenyl)naphthalene (7.20 g, 1.0 equiv, 25.4 mmol), and PtBu₃ (1.03 g, 0.2 equiv, 5.09 mmol) were sequentially added to a three-neck flask (1000 mL), and heated and stirred under reflux. The resultant solution was cooled to room temperature, and then water was added to a reaction solvent, and an organic layer was separated and collected. Toluene was added to the water layer to further extract an organic layer. The extracted organic layer was washed with brine and dried over MgSO₄. MgSO₄ was filtered and the organic layer was concentrated to obtain a crude product. The crude product was purified to obtain Compound 1 (14.41 g, yield: 83%). A molecular ion peak was observed at m/z (mass number) 679 as measured by FAB MS, thereby identifying Compound 1.

### (2) Synthesis of Amine Compound 13

Amine compound 13 according to an embodiment may be synthesized by, for example, processes of Reaction Formula 2 below.

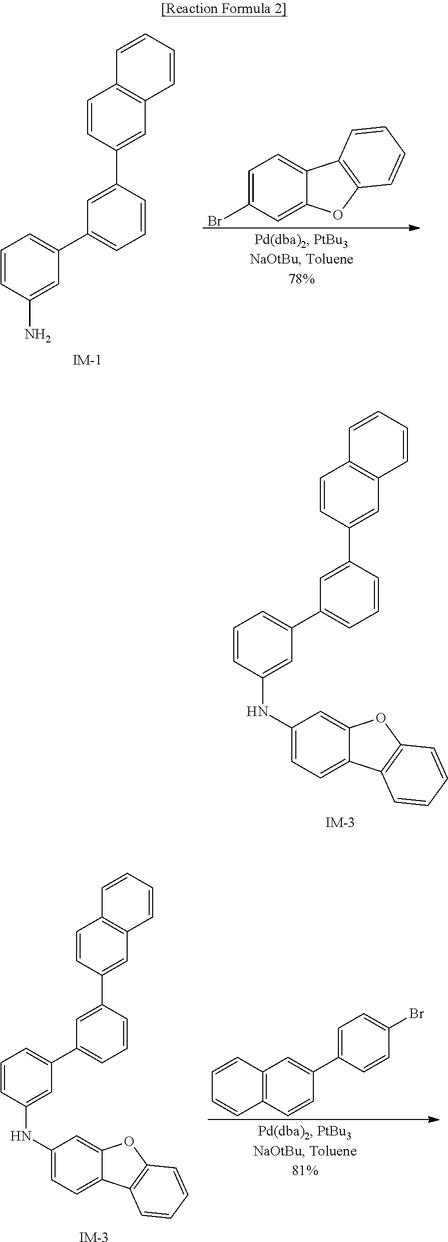

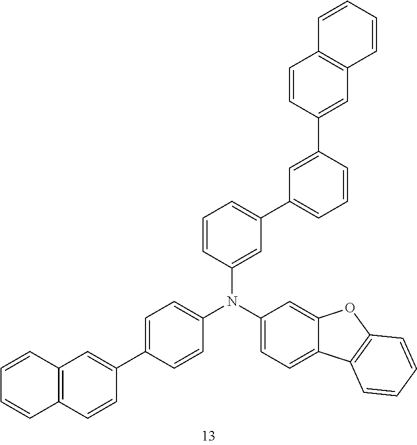 [SMILES: c1cc(-c2cccc(N(c3ccc(-c4ccc5ccccc5c4)cc3)c3ccc4c(c3)oc3ccccc34)c2)cc(-c2ccc3ccccc3c2)c1]

### Synthesis of Compound IM-3

In an Ar atmosphere, Compound IM-1 (12.00 g, 40.62 mmol), Pd(dba)₂ (1.17 g, 0.05 equiv, 2.03 mmol), NaOtBu (3.90 g, 1.0 equiv, 40.6 mmol), toluene (400 mL), 3-bromodibenzo[b,d]furan (10.04 g, 1.0 equiv, 40.62 mmol), and PtBu₃ (1.64 g, 0.2 equiv, 8.12 mmol) were sequentially added to a three-neck flask (1000 mL), and heated and stirred under reflux. The resultant solution was cooled to room temperature, and then water was added to a reaction solvent, and an organic layer was separated and collected. Toluene was added to the water layer to further extract an organic layer. The extracted organic layer was washed with brine and dried over MgSO₄. MgSO₄ was filtered and the organic layer was concentrated to obtain a crude product. The crude product was purified to obtain Compound IM-3 (14.63 g, yield: 78%). A molecular ion peak was observed at m/z (mass number) 461 as measured by FAB MS, thereby identifying Compound IM-3.

### Synthesis of Compound 13

In an Ar atmosphere, Compound IM-3 (14.63 g, 31.70 mmol), Pd(dba)₂ (0.91 g, 0.05 equiv, 1.6 mmol), NaOtBu (3.05 g, 1.0 equiv, 31.7 mmol), toluene (320 mL), 2-(4-bromophenyl)naphthalene (8.98 g, 1.0 equiv, 31.7 mmol), and PtBu₃ (1.28 g, 0.2 equiv, 6.34 mmol) were sequentially added to a three-neck flask (1000 mL), and heated and stirred under reflux. The resultant solution was cooled to room temperature, and then water was added to a reaction solvent, and an organic layer was separated and collected. Toluene was added to the water layer to further extract an organic layer. The extracted organic layer was washed with brine and dried over MgSO₄. MgSO₄ was filtered and the organic layer was concentrated to obtain a crude product. The crude product was purified to obtain Compound 13 (17.14 g, yield: 81%).

A molecular ion peak was observed at m/z (mass number) 663 as measured by FAB MS, thereby identifying Compound 13.

### (3) Synthesis of Amine Compound 17

Amine compound 17 according to an embodiment may be synthesized by, for example, processes of Reaction Formula 3 below.

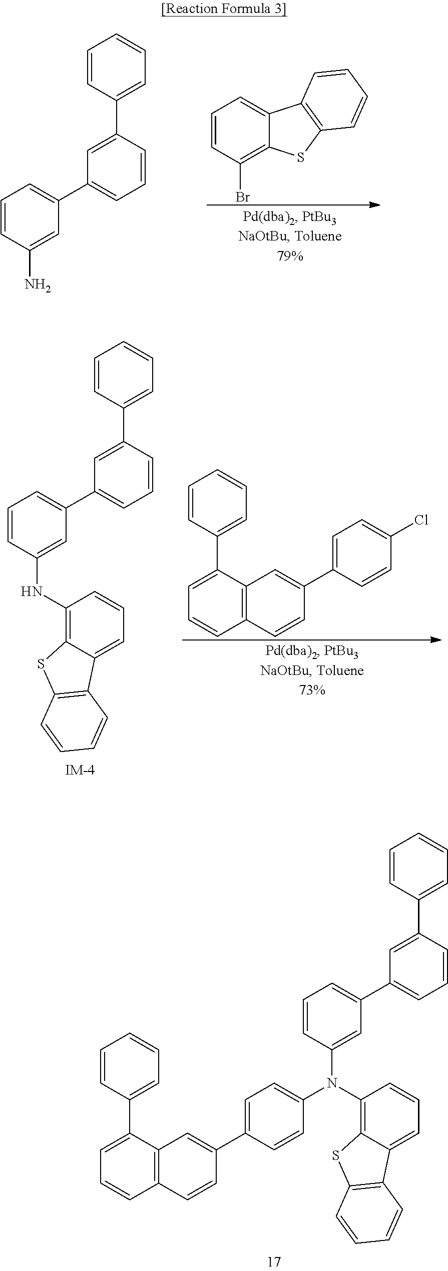

### Synthesis of Compound IM-4

In an Ar atmosphere, [1,1′,3′,1″-terphenyl]-3-amine (13.00 g, 52.99 mmol), Pd(dba)₂ (1.52 g, 0.05 equiv, 2.65 mmol), NaOtBu (5.09 g, 1.0 equiv, 53.0 mmol), toluene (530 mL), 4-bromodibenzo[b,d]thiophene (13.94 g, 1.0 equiv, 52.99 mmol), and PtBu₃ (2.14 g, 0.2 equiv, 10.6 mmol) were sequentially added to a three-neck flask (2000 mL), and heated and stirred under reflux. The resultant solution was cooled to room temperature, and then water was added to a reaction solvent, and an organic layer was separated and collected. Toluene was added to the water layer to further extract an organic layer. The extracted organic layer was washed with brine and dried over MgSO₄. MgSO₄ was filtered and the organic layer was concentrated to obtain a crude product. The crude product was purified to obtain Compound IM-4 (17.83 g, yield: 79%). A molecular ion peak was observed at m/z (mass number) 427 as measured by FAB MS, thereby identifying Compound IM-4.

### Synthesis of Compound 17

In an Ar atmosphere, Compound IM-4 (8.70 g, 20.4 mmol), Pd(dba)₂ (0.58 g, 0.05 equiv, 1.0 mmol), NaOtBu (2.35 g, 1.2 equiv, 24.4 mmol), toluene (200 mL), 7-(4-chlorophenyl)-1-phenylnaphthalene (6.41 g, 1.0 equiv, 20.4 mmol), and PtBu₃ (0.82 g, 0.2 equiv, 4.07 mmol) were sequentially added to a three-neck flask (500 mL), and heated and stirred under reflux. The resultant solution was cooled to room temperature, and then water was added to a reaction solvent, and an organic layer was separated and collected. Toluene was added to the water layer to further extract an organic layer. The extracted organic layer was washed with brine and dried over MgSO₄. MgSO₄ was filtered and the organic layer was concentrated to obtain a crude product. The crude product was purified to obtain Compound 17 (10.45 g, yield: 73%). A molecular ion peak was observed at m/z (mass number) 705 as measured by FAB MS, thereby identifying Compound 17.

### (4) Synthesis of Amine Compound 22

Amine compound 22 according to an embodiment may be synthesized by, for example, processes of Reaction Formula 4 below.

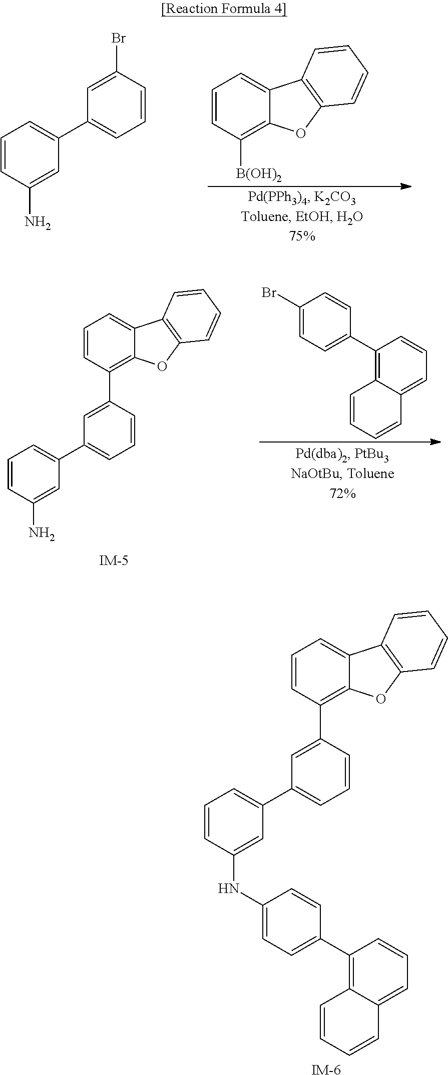

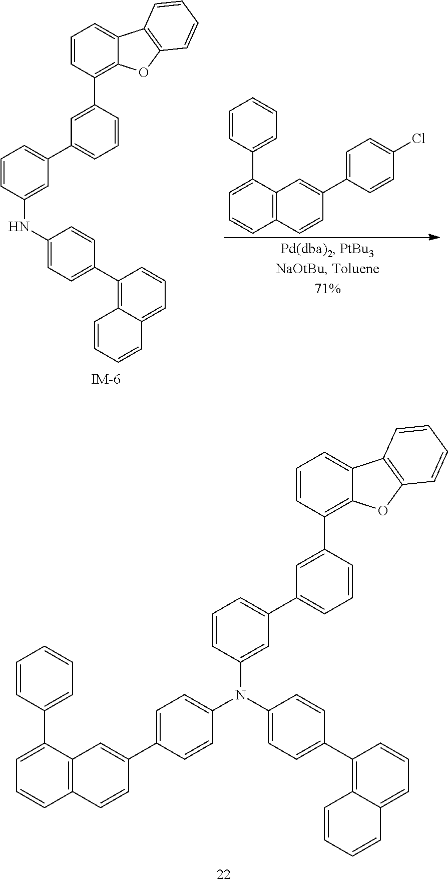

### Synthesis of Compound IM-5

In an Ar atmosphere, 3′-bromo-[1,1′-biphenyl]-3-amine (15.00 g, 60.45 mmol), dibenzo[b,d]furan-4-ylboronic acid (15.38 g, 1.2 equiv, 72.55 mmol), Pd(PPh₃)₄ (6.99 g, 0.10 equiv, 6.05 mmol), K₂CO₃ (16.71 g, 2.0 equiv, 120.9 mmol), toluene (240 mL), EtOH (120 mL), and H₂O (60 mL) were sequentially added to a three-neck flask (1000 mL), and heated and stirred under reflux. The resultant solution was cooled to room temperature, and then water and toluene were added to a reaction solvent, and an organic layer was separated and collected. The organic layer was washed with brine, and dried over MgSO₄. MgSO₄ was filtered and the organic layer was concentrated to obtain a crude product. The crude product was purified to obtain Compound IM-5 (15.23 g, yield: 75%). A molecular ion peak was observed at m/z (mass number) 335 as measured by FAB MS, thereby identifying Compound IM-5.

### Synthesis of Compound IM-6

In an Ar atmosphere, Compound IM-5 (9.00 g, 26.8 mmol), Pd(dba)₂ (0.77 g, 0.05 equiv, 1.3 mmol), NaOtBu (2.58 g, 1.0 equiv, 26.8 mmol), toluene (270 mL), 1-(4-bromophenyl)naphthalene (7.60 g, 1.0 equiv, 26.8 mmol), and PtBu₃ (1.09 g, 0.2 equiv, 5.37 mmol) were sequentially added to a three-neck flask (1000 mL), and heated and stirred under reflux. The resultant solution was cooled to room temperature, and then water was added to a reaction solvent, and an organic layer was separated and collected. Toluene was added to the water layer to further extract an organic layer. The extracted organic layer was washed with brine and dried over MgSO₄. MgSO₄ was filtered and the organic layer was concentrated to obtain a crude product. The crude product was purified to obtain Compound IM-6 (10.35 g, yield: 72%). A molecular ion peak was observed at m/z (mass number) 537 as measured by FAB MS, thereby identifying Compound IM-4.

### Synthesis of Compound 22

In an Ar atmosphere, Compound IM-6 (10.35 g, 19.25 mmol), Pd(dba)₂ (0.55 g, 0.05 equiv, 0.96 mmol), NaOtBu (2.22 g, 1.2 equiv, 23.1 mmol), toluene (190 mL), 7-(4-chlorophenyl)-1-phenylnaphthalene (6.06 g, 1.0 equiv, 19.3 mmol), and PtBu₃ (0.78 g, 0.2 equiv, 3.85 mmol) were sequentially added to a three-neck flask (500 mL), and heated and stirred under reflux. The resultant solution was cooled to room temperature, and then water was added to a reaction solvent, and an organic layer was separated and collected. Toluene was added to the water layer to further extract an organic layer. The extracted organic layer was washed with brine and dried over MgSO₄. MgSO₄ was filtered and the organic layer was concentrated to obtain a crude product. The crude product was purified to obtain Compound 22 (11.14 g, yield: 71%).

A molecular ion peak was observed at m/z (mass number) 815 as measured by FAB MS, thereby identifying Compound 22.

### (5) Synthesis of Amine Compound 26

Amine compound 26 according to an embodiment may be synthesized by, for example, processes of Reaction Formula 5 below.

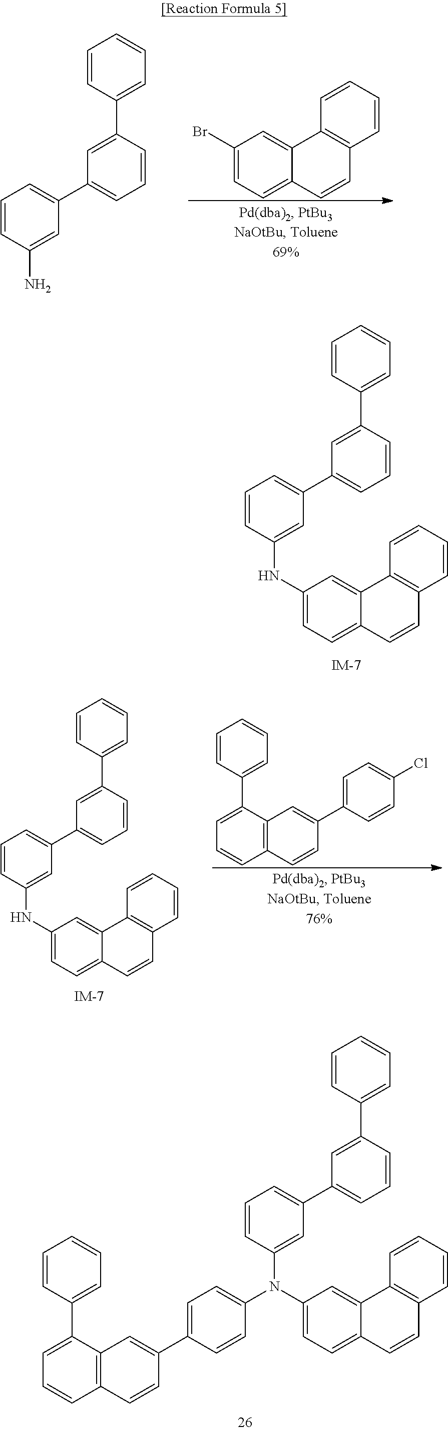

### Synthesis of Compound IM-7

In an Ar atmosphere, [1,1′,3′,1″-terphenyl]-3-amine (14.31 g, 58.33 mmol), Pd(dba)₂ (1.68 g, 0.05 equiv, 2.92 mmol), NaOtBu (5.61 g, 1.0 equiv, 58.3 mmol), toluene (580 mL), 3-bromophenanthrene (15.00 g, 1.0 equiv, 58.33 mmol), and PtBu₃ (2.36 g, 0.2 equiv, 11.7 mmol) were sequentially added to a three-neck flask (2000 mL), and heated and stirred under reflux. The resultant solution was cooled to room temperature, and then water was added to a reaction solvent, and an organic layer was separated and collected. Toluene was added to the water layer to further extract an organic layer. The extracted organic layer was washed with brine and dried over MgSO₄. MgSO₄ was filtered and the organic layer was concentrated to obtain a crude product. The crude product was purified to obtain Compound IM-7 (16.99 g, yield: 69%). A molecular ion peak was observed at m/z (mass number) 421 as measured by FAB MS, thereby identifying Compound IM-7.

### Synthesis of Compound 26

In an Ar atmosphere, Compound IM-7 (16.99 g, 40.30 mmol), Pd(dba)₂ (1.16 g, 0.05 equiv, 2.02 mmol), NaOtBu (4.65 g, 1.2 equiv, 48.4 mmol), toluene (400 mL), 7-(4-chlorophenyl)-1-phenylnaphthalene (12.69 g, 1.0 equiv, 40.30 mmol), and PtBu₃ (1.63 g, 0.2 equiv, 8.06 mmol) were sequentially added to a three-neck flask (1000 mL), and heated and stirred under reflux. The resultant solution was cooled to room temperature, and then water was added to a reaction solvent, and an organic layer was separated and collected. Toluene was added to the water layer to further extract an organic layer. The extracted organic layer was washed with brine and dried over MgSO₄. MgSO₄ was filtered and the organic layer was concentrated to obtain a crude product. The crude product was purified to obtain Compound 26 (21.42 g, yield: 76%).

A molecular ion peak was observed at m/z (mass number) 699 as measured by FAB MS, thereby identifying Compound 26.

### (6) Synthesis of Amine Compound 37

Amine compound 37 according to an embodiment may be synthesized by, for example, processes of Reaction Formula 6 below.

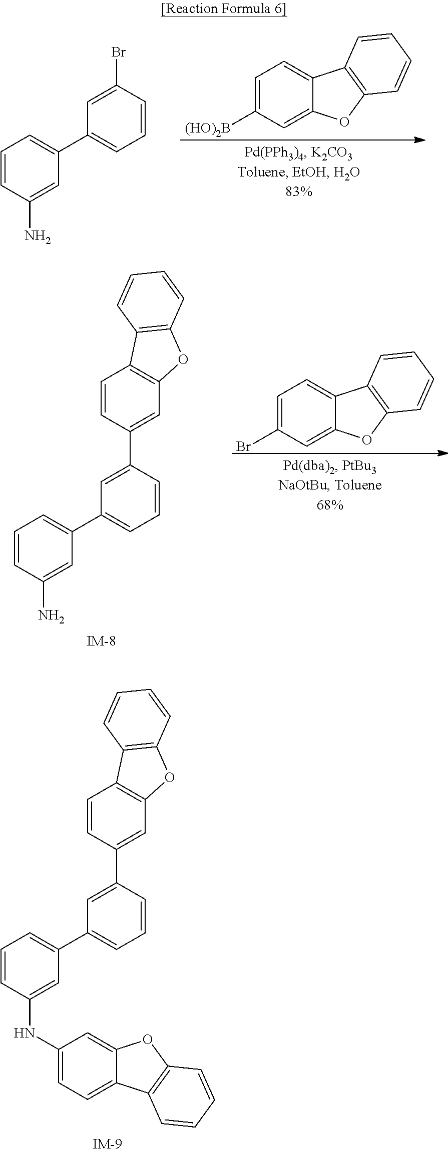

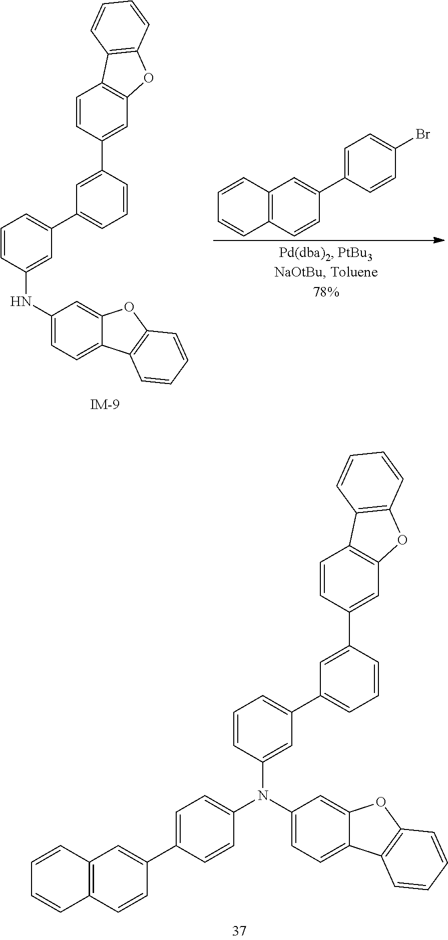

### Synthesis of Compound IM-8

In an Ar atmosphere, 3′-bromo-[1,1′-biphenyl]-3-amine (20.00 g, 80.61 mmol), dibenzo[b,d]furan-3-ylboronic acid (18.80 g, 1.1 equiv, 88.67 mmol), Pd(PPh₃)₄ (9.31 g, 0.10 equiv, 8.06 mmol), K₂CO₃ (22.28 g, 2.0 equiv, 161.2 mmol), toluene (320 mL), EtOH (160 mL), and H₂O (80 mL) were sequentially added to a three-neck flask (2000 mL), and heated and stirred under reflux. The resultant solution was cooled to room temperature, and then water and toluene were added to a reaction solvent, and an organic layer was separated and collected. The organic layer was washed with brine, and dried over MgSO₄. MgSO₄ was filtered and the organic layer was concentrated to obtain a crude product. The crude product was purified to obtain Compound IM-8 (22.44 g, yield: 83%). A molecular ion peak was observed at m/z (mass number) 335 as measured by FAB MS, thereby identifying Compound IM-8.

### Synthesis of Compound IM-9

In an Ar atmosphere, Compound IM-8 (12.00 g, 35.78 mmol), Pd(dba)₂ (1.03 g, 0.05 equiv, 1.79 mmol), NaOtBu (3.44 g, 1.0 equiv, 35.8 mmol), toluene (360 mL), 3-bromodibenzo[b,d]furan (8.84 g, 1.0 equiv, 35.8 mmol), and PtBu₃ (1.45 g, 0.2 equiv, 7.16 mmol) were sequentially added to a three-neck flask (1000 mL), and heated and stirred under reflux. The resultant solution was cooled to room temperature, and then water was added to a reaction solvent, and an organic layer was separated and collected. Toluene was added to the water layer to further extract an organic layer. The extracted organic layer was washed with brine and dried over MgSO₄. MgSO₄ was filtered and the organic layer was concentrated to obtain a crude product. The crude product was purified to obtain Compound IM-9 (12.16 g, yield: 68%). A molecular ion peak was observed at m/z (mass number) 501 as measured by FAB MS, thereby identifying Compound IM-9.

### Synthesis of Compound 37

In an Ar atmosphere, Compound IM-9 (12.16 g, 24.24 mmol), Pd(dba)₂ (0.70 g, 0.05 equiv, 1.2 mmol), NaOtBu (2.33 g, 1.0 equiv, 24.2 mmol), toluene (240 mL), 2-(4-bromophenyl)naphthalene (6.86 g, 1.0 equiv, 24.2 mmol), and PtBu₃ (0.98 g, 0.2 equiv, 4.9 mmol) were sequentially added to a three-neck flask (1000 mL), and heated and stirred under reflux. The resultant solution was cooled to room temperature, and then water was added to a reaction solvent, and an organic layer was separated and collected. Toluene was added to the water layer to further extract an organic layer. The extracted organic layer was washed with brine and dried over MgSO₄. MgSO₄ was filtered and the organic layer was concentrated to obtain a crude product. The crude product was purified to obtain Compound 37 (13.33 g, yield: 78%). A molecular ion peak was observed at m/z (mass number) 703 as measured by FAB MS, thereby identifying Compound 37.

### 2. Preparation and Evaluation of Light Emitting Elements

### (1) Preparation of Light Emitting Elements

Light emitting elements including amine compounds according to an embodiment or Comparative Example compounds in a hole transport layer were prepared through a method below. Light emitting elements of Examples 1 to 6 were prepared respectively using amine compounds according to an embodiment, Compound 1, 13, 17, 22, 26, and 37 as a hole transport material of a second hole transport layer. Light emitting elements of Comparative Examples 1 to 15 were prepared using Comparative Example Compounds C1 to C15 as a hole transport material of a second hole transport layer.

A glass substrate on which an ITO having a thickness of 1500 Å was patterned was subjected to ultrasonic cleaning using isopropyl alcohol and pure water each for 5 minutes. The glass substrate was irradiated with UV for 30 minutes, and ozone-treated. Thereafter, Compound H-1-1 and Compound HI1 were deposited at a weight ratio of 98:2 to have a thickness of 100 Å, thereby forming a hole injection layer.

Compound H-1-1 was deposited to have a thickness of 1200 Å on the hole injection layer to form a first hole transport layer. Then, in Examples 1 to 6 and Comparative Examples 1 to 15, Example Compounds or Comparative Example Compounds were deposited to have a thickness of 100 Å to form a second hole transport layer.

Thereafter, a host and a dopant were co-deposited at a weight ratio of 98:2 to form an emission layer having a thickness of 300 Å. Compound BH was provided as a host and Compound BD was provided as a dopant.

On the emission layer, Compound ET37 was deposited to have a thickness of 100 Å to form a first electron transport layer, and Compound ET38 was deposited to have a thickness of 200 Å to form a second electron transport layer. Thereafter, LiF was deposited to have a thickness of 10 Å to form an electron injection layer. Then, a second electrode was formed to have a thickness of 1200 Å using Ag and Mg at a weight ratio of 10:90.

### (Materials Used for Preparation of Light Emitting Elements)

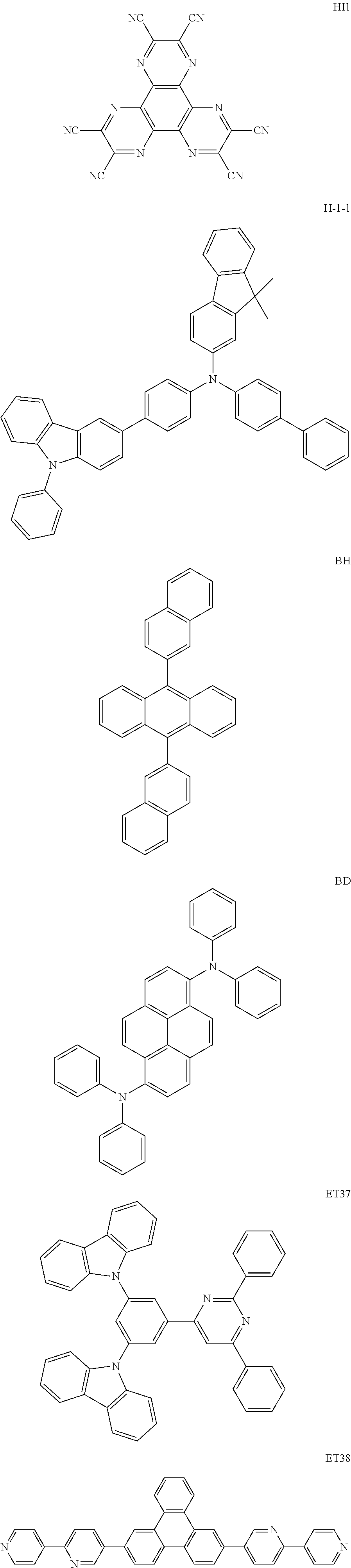

### Example Compounds

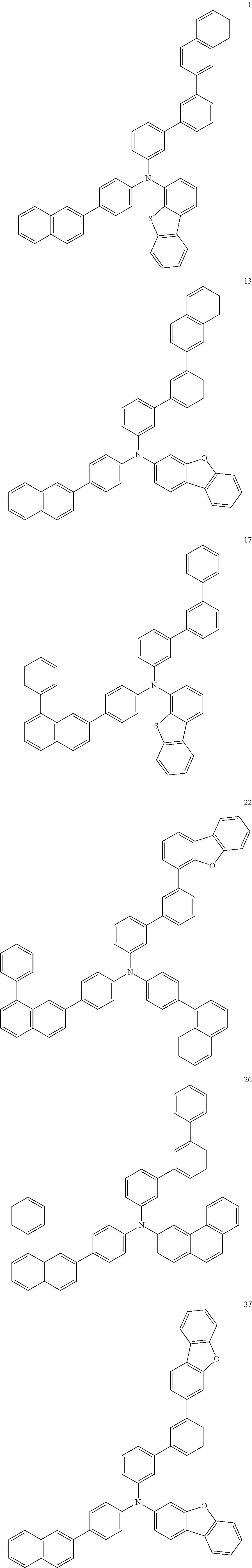

### Comparative Example Compounds

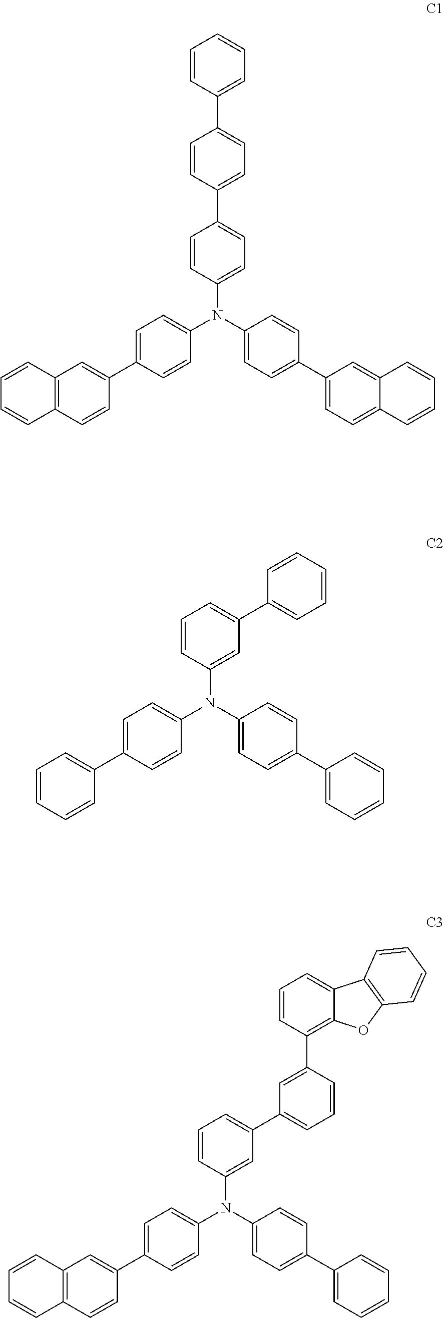 [SMILES: c1ccc(-c2ccc(-c3ccc(N(c4ccc(-c5ccc6ccccc6c5)cc4)c4ccc(-c5ccc6ccccc6c5)cc4)cc3)cc2)cc1 ; c1ccc(-c2ccc(N(c3ccc(-c4ccccc4)cc3)c3cccc(-c4ccccc4)c3)cc2)cc1 ; c1ccc(-c2ccc(N(c3ccc(-c4ccc5ccccc5c4)cc3)c3cccc(-c4cccc(-c5cccc6c5oc5ccccc56)c4)c3)cc2)cc1]

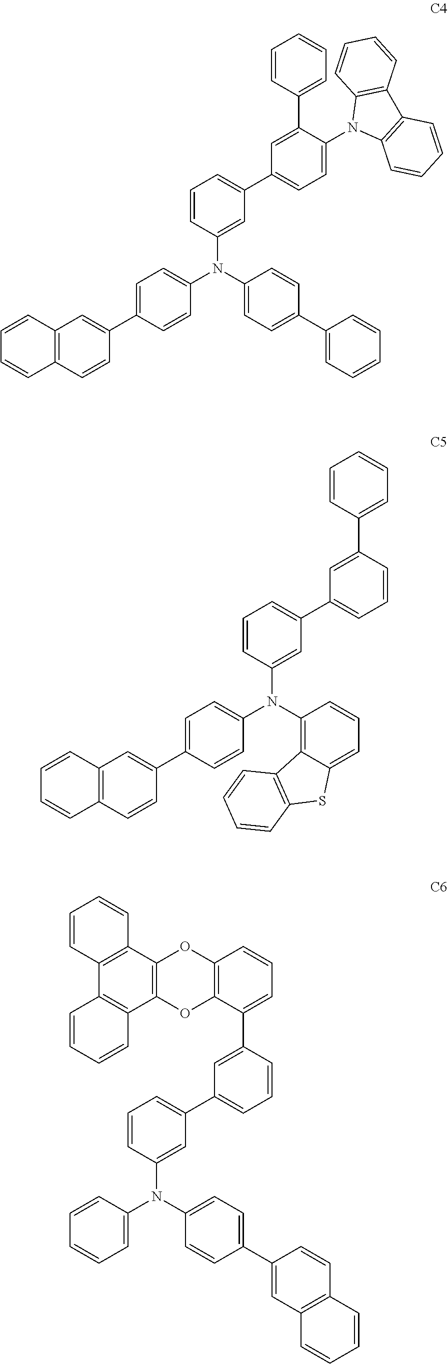 [SMILES: c1ccc(-c2ccc(N(c3ccc(-c4ccc5ccccc5c4)cc3)c3cccc(-c4ccc(-n5c6ccccc6c6ccccc65)c(-c5ccccc5)c4)c3)cc2)cc1 ; c1ccc(-c2cccc(-c3cccc(N(c4ccc(-c5ccc6ccccc6c5)cc4)c4cccc5sc6ccccc6c45)c3)c2)cc1 ; c1ccc(N(c2ccc(-c3ccc4ccccc4c3)cc2)c2cccc(-c3cccc(-c4cccc5c4Oc4c(c6ccccc6c6ccccc46)O5)c3)c2)cc1]

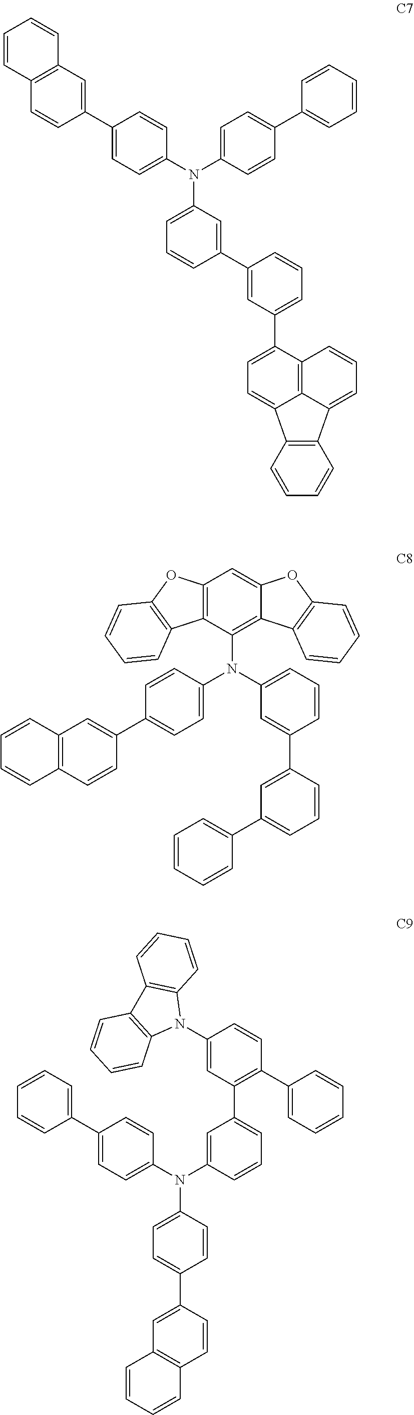 [SMILES: c1ccc(-c2ccc(N(c3ccc(-c4ccc5ccccc5c4)cc3)c3cccc(-c4cccc(-c5ccc6c7c(cccc57)-c5ccccc5-6)c4)c3)cc2)cc1 ; c1ccc(-c2cccc(-c3cccc(N(c4ccc(-c5ccc6ccccc6c5)cc4)c4c5c(cc6oc7ccccc7c46)oc4ccccc45)c3)c2)cc1 ; c1ccc(-c2ccc(N(c3ccc(-c4ccc5ccccc5c4)cc3)c3cccc(-c4cc(-n5c6ccccc6c6ccccc65)ccc4-c4ccccc4)c3)cc2)cc1]

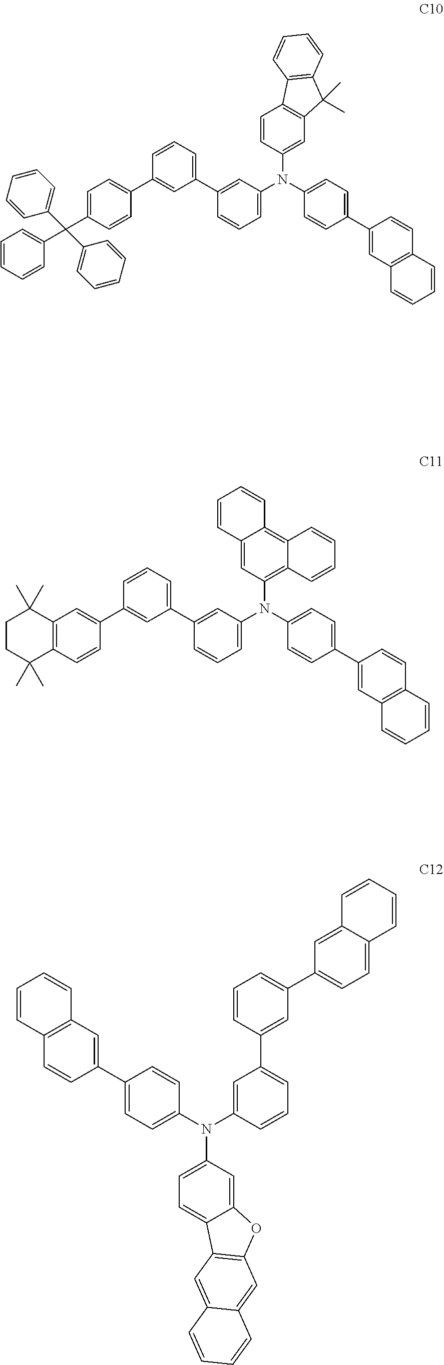 [SMILES: CC1(C)c2ccccc2-c2ccc(N(c3ccc(-c4ccc5ccccc5c4)cc3)c3cccc(-c4cccc(-c5ccc(C(c6ccccc6)(c6ccccc6)c6ccccc6)cc5)c4)c3)cc21 ; CC1(C)CCC(C)(C)c2cc(-c3cccc(-c4cccc(N(c5ccc(-c6ccc7ccccc7c6)cc5)c5cc6ccccc6c6ccccc56)c4)c3)ccc21 ; c1cc(-c2cccc(N(c3ccc(-c4ccc5ccccc5c4)cc3)c3ccc4c(c3)oc3cc5ccccc5cc34)c2)cc(-c2ccc3ccccc3c2)c1]

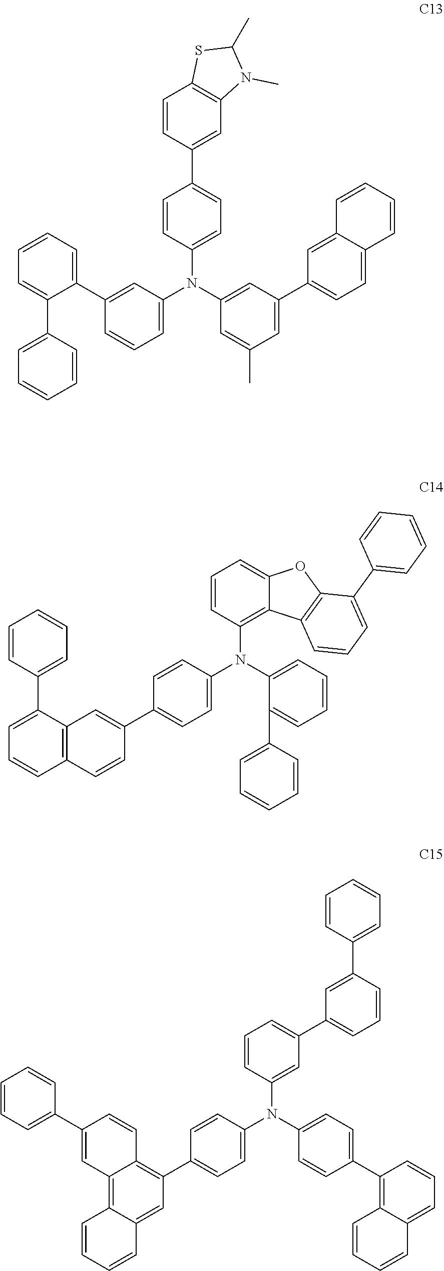 [SMILES: Cc1cc(-c2ccc3ccccc3c2)cc(N(c2ccc(-c3ccc4c(c3)N(C)C(C)S4)cc2)c2cccc(-c3ccccc3-c3ccccc3)c2)c1 ; c1ccc(-c2ccccc2N(c2ccc(-c3ccc4cccc(-c5ccccc5)c4c3)cc2)c2cccc3oc4c(-c5ccccc5)cccc4c23)cc1 ; c1ccc(-c2cccc(-c3cccc(N(c4ccc(-c5cccc6ccccc56)cc4)c4ccc(-c5cc6ccccc6c6cc(-c7ccccc7)ccc56)cc4)c3)c2)cc1]

### (2) Evaluation of Light Emitting Elements

Table 1 shows results of evaluation of light emitting elements for Examples 1 to 6, and Comparative Examples 1 to 15. In Table 1, the driving voltage and half-life (LT₅₀) of the prepared light emitting elements compared and shown. In Table 1, lifespan indicates the relative ratio of the luminance half-life at 10 mA/cm², which is a relative ratio with the lifespan of Comparative Example 1 as 100%. The driving voltage indicates a difference between Comparative Example 1 and Comparative Examples 2 to 15 and Examples 1 to 6 when the voltage of the light emitting element of Comparative Example 1 is set to 0 V upon turned on at 10 mA/cm².

TABLE 1

| Example of | | | | |
|---|---|---|---|---|
| element | Second hole transport | | | |
| preparation | layer | Voltage | | Lifespan |
| Example 1 | Compound 1 | −0.10 | V | 126% |
| Example 2 | Compound 13 | −0.09 | V | 121% |
| Example 3 | Compound 17 | −0.12 | V | 119% |
| Example 4 | Compound 26 | −0.08 | V | 131% |
| Example 5 | Compound 22 | −0.05 | V | 107% |
| Example 6 | Compound 37 | −0.06 | V | 109% |
| Comparative | Comparative Example | 0.00 | V | 100% |
| Example 1 | Compound C1 | | | |
| Comparative | Comparative Example | −0.02 | V | 85% |
| Example 2 | Compound C2 | | | |
| Comparative | Comparative Example | +0.01 | V | 90% |
| Example 3 | Compound C3 | | | |
| Comparative | Comparative Example | +0.05 | V | 87% |
| Example 4 | Compound C4 | | | |
| Comparative | Comparative Example | −0.02 | V | 80% |
| Example 5 | Compound C5 | | | |
| Comparative | Comparative Example | +0.01 | V | 84% |
| Example 6 | Compound C6 | | | |
| Comparative | Comparative Example | +0.07 | V | 100% |
| Example 7 | Compound C7 | | | |
| Comparative | Comparative Example | +0.03 | V | 83% |
| Example 8 | Compound C8 | | | |
| Comparative | Comparative Example | +0.03 | V | 91% |
| Example 9 | Compound C9 | | | |
| Comparative | Comparative Example | +0.01 | V | 79% |
| Example 10 | Compound C10 | | | |
| Comparative | Comparative Example | +0.02 | V | 75% |
| Example 11 | Compound C11 | | | |
| Comparative | Comparative Example | +0.07 | V | 92% |
| Example 12 | Compound C12 | | | |
| Comparative | Comparative Example | +0.04 | V | 71% |
| Example 13 | Compound C13 | | | |
| Comparative | Comparative Example | +0.06 | V | 66% |
| Example 14 | Compound C14 | | | |
| Comparative | Comparative Example | +0.04 | V | 99% |
| Example 15 | Compound C15 | | | |

Referring to Table 1, it can be seen that, compared to the light emitting elements of Comparative Examples 1, 3, and 4, the light emitting elements of Examples 1 to 6 and Comparative Examples 2 and 5 have reduced driving voltage. In addition, it can be seen that the light emitting elements of Examples 1 to 6 have a greater reduction in driving voltage than the light emitting elements of Comparative Examples 2 and 5. It can be seen that, compared to the light emitting element of Comparative Example 1 to 15, the light emitting elements of Examples 1 to 6 have a relatively long lifespan. The light emitting elements of Examples 1 to 6 include Compounds 1, 13, 17, 22, 26, and 37, and Compounds 1, 13, 17, 22, 26, and 37 are amine compounds of an embodiment.

As described above, the amine compound of an embodiment introduces a naphthyl group into a second ring group to maintain the characteristics of the amine group and also improve charge tolerance, thereby increasing the lifespan of a light emitting element. In addition, the amine compound of an embodiment includes a third ring group that is an aryl group, a dibenzofuran group, or a dibenzothiophene group, and a first ring group that is a meta-meta biphenyl group, and may thus prevent or reduce stacking of materials. Accordingly, the amine compound of an embodiment may easily move charges through hopping to reduce the driving voltage of a light emitting element. Accordingly, a light emitting element including the amine compound of an embodiment may have reduced driving voltage and increased lifespan.

The light emitting element of Comparative Example 1 includes Comparative Example Compound C1, and Comparative Example Compound C1 does not include a first ring group that is a meta-meta biphenyl group. Accordingly, the light emitting element of Comparative Example 1 exhibits relatively high driving voltage.

The light emitting element of Comparative Example 2 includes Comparative Example Compound C2, and Comparative Example Compound C2 does not include a naphthyl group, and thus fails to maintain the characteristics of an amine group. Accordingly, the light emitting element of Comparative Example Compound C2 is deteriorated and the light emitting element of Comparative Example 2 exhibits relatively short lifespan.

The light emitting element of Comparative Example 3 includes Comparative Example Compound C3, and Comparative Example Compound C3 corresponds to a case in which a1 to a4 are all 0 and L¹-Ar² is an unsubstituted biphenyl group in Formula 1. As described above, the case in which a1 to a4 are all 0, and L¹-Ar² is an unsubstituted biphenyl group in Formula 1 is excluded from the amine compound of an embodiment. The light emitting element of Comparative Example 12 includes Comparative Example Compound C12, and Comparative Example Compound C12 includes benzonaphthofuran bonded to an amine group.

Comparative Example Compound C3 includes an unsubstituted biphenyl group at the position of L¹-Ar² of Formula 1, and thus has high planarity and causes stacking of molecules. Comparative Example Compound C12 includes benzonaphthofuran, has high planarity, and causes stacking of molecules. Accordingly, the light emitting elements of Comparative Examples 3 and 12 including Comparative Example Compounds C3 and C12 have increased driving voltages as charge transfer is not easy.

In Compound 22, a substituent adjacent to an amine group is substituted, and accordingly, the stacking of molecules may be prevented or reduced. Accordingly, the movement of charge through hopping is easy, and thus the light emitting element of Example 5 including Compound 22 may have reduced driving voltage and increased lifespan.

The light emitting element of Comparative Example 4 includes Comparative Example Compound C4, and the light emitting element of Comparative Example 9 includes Comparative Example Compound C9. Comparative Example Compounds C4 and C9 include carbazole groups. A carbazole group is a heteroaryl group including nitrogen atoms as hetero atoms, and Comparative Example Compounds C4 and C9 including a nitrogen-containing polycyclic ring group such as a carbazole group exhibit a significant change in charge balance of molecules. Accordingly, the light emitting elements of Comparative Examples 4 and 9 including Comparative Examples Compounds C4 and C9 exhibit relatively shorter lifespan than the light emitting element of Example 5.

The light emitting element of Comparative Example 5 includes Comparative Example Compound C5, and Comparative Example Compound C5 includes a 1-dibenzothiophene group. Sulfur atoms, ring-forming atoms of the 1-dibenzothiophene group, have a large atomic radius to be in the form that unshared electron pairs protrude from the outside of the molecule to get easily affected by electric charges. Accordingly, Comparative Example Compound C5 has reduced stability of materials, and thus the light emitting element of Comparative Example 5 exhibits shorter lifespan than the light emitting elements of Examples 2 and 3.

The light emitting element of Comparative Example 6 includes Comparative Example Compound C6, and the light emitting element of Comparative Example 8 includes Comparative Example Compound C8. Comparative Examples Compounds C6 and C8 correspond to cases in which Ar¹ is a heteroaryl group including two heteroatoms in Formula 1. As described above, in Formula 1, Ar¹ may be an aryl group or a first heteroaryl group including one hetero atom. Comparative Example Compounds C6 and C8 include a heteroaryl group containing two heteroatoms, and thus exhibit a significant change in charge balance of molecules. Accordingly, the light emitting elements of Comparative Examples 6 and 8 including Comparative Example Compounds C6 and C8 exhibit relatively shorter lifespan than the light emitting elements of] Examples 1 to 6.

The light emitting element of Comparative Example 7 includes Comparative Example Compound C7, and Comparative Example Compound C7 includes a fluoranthene group. Comparative Example Compound C7 corresponds to a case in which Ar¹ is an aryl group having more than 15 ring-forming carbon atoms in Formula 1. Comparative Example Compound C7 including a fluoranthene group tends to have high molecular planarity, and the molecules are stacked to disturb the movement of electric charges. Accordingly, compared to the light emitting element of Example 4, the light emitting element of Comparative Example 7 has increased driving voltage.

The light emitting element of Comparative Example 10 includes Comparative Example Compound C10, and Comparative Example compound 10 includes a dimethylfluorene group. The light emitting element of Comparative Example 11 includes Comparative Example Compound C11, and Comparative Example Compound C11 includes two or more sp3 carbons. The light emitting element of Comparative Example 13 includes Comparative Example Compound C13, and Comparative Example Compound C13 includes a benzothiazole group. When Comparative Example Compounds C10, C11, and C13 are included in a second hole transport layer between a first hole transport layer and an emission layer, Comparative Example Compounds C10, C11, and C13 are strongly affected by electrons and holes, and accordingly, each substituent is ring-opened to cause a significant reduction in stability of materials. More specifically, when Comparative Example Compounds C10, C11, and C13 are strongly affected by electrons and holes, bonds between carbon atoms forming a ring are broken in aromatic/non-aromatic ring groups directly or indirectly bonded to an amine group to cause reduction in stability of materials. Therefore, unlike the light emitting elements of Comparative Examples 10, 11 and 13, the light emitting elements of Examples 1 and 2 formed of Compounds 1 and 13 which do not include a dimethylfluorene group, two or more sp3 carbons, and a benzothiazole group may exhibit excellent lifespan.

The light emitting element of Comparative Example 14 includes Comparative Example Compound C14, and Comparative Example Compound C14 includes an ortho-biphenyl group (o-biphenyl group). In Comparative Example Compound C14 including an ortho-biphenyl group, distortion takes place around nitrogen atoms of an amine group, resulting in reduced stability of materials. Accordingly, compared to the light emitting element of Example 5, the light emitting element of Comparative Example 14 exhibits short lifespan.

The light emitting element of Comparative Example 15 includes Comparative Example Compound C15. Comparative Example Compound C15 includes a phenyl group substituted with a naphthyl group, but the binding position of the phenyl group to the naphthyl group is different from that of the amine compound of an embodiment. Accordingly, the light emitting element of Comparative Example 15 exhibits relatively high driving voltage and short lifespan.

A light emitting element of an embodiment may include an amine compound of an embodiment in at least one functional layer between a first electrode and a second electrode. The amine compound of an embodiment may include first to third ring groups bonded to an amine group. The first ring group may include a meta-meta biphenyl group, the second ring group may include a phenyl group substituted with a naphthyl group, and the third ring group may include an aryl group, a dibenzofuran group, or a dibenzothiophene group. Accordingly, a light emitting element including the amine compound of an embodiment may have reduced driving voltage and increased lifespan.

A light emitting element of an embodiment includes an amine compound of an embodiment, and may thus exhibit reduced driving voltage and long lifespan.

An amine compound of an embodiment may contribute to a reduction in driving voltage and an increase in lifespan of a light emitting element.

Although the subject matter of the present disclosure has been described with reference to example embodiments of the present disclosure, it will be understood that the subject matter of the present disclosure should not be limited to these example embodiments but various changes and modifications may be made by those skilled in the art without departing from the spirit and scope of the present disclosure.

Accordingly, the technical scope of the present disclosure is not intended to be limited to the contents set forth in the detailed description of the specification, but is intended to be defined by the appended claims, and equivalents thereof.

What is claimed is:

1. A light emitting element comprising:
a first electrode;
a second electrode on the first electrode; and
at least one functional layer between the first electrode and the second electrode, and comprising an amine compound represented by Formula 1 below: 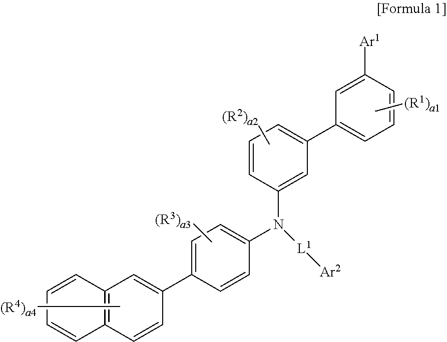
wherein in Formula 1 above,
Ar¹ is a substituted or unsubstituted aryl group having 6 to 15 ring-forming carbon atoms, or a substituted or unsubstituted first heteroaryl group having 5 to 30 ring-forming carbon atoms,
the first heteroaryl group comprises only one hetero atom, and the one hetero atom is an oxygen atom or a sulfur atom,
Ar² is a substituted or unsubstituted aryl group having 6 to 30 ring-forming carbon atoms, a substituted or unsubstituted dibenzofuran group, or a substituted or unsubstituted dibenzothiophene group,
a1 to a3 are each independently an integer of 0 to 4,
a4 is an integer of 0 to 7,
R¹, R³, and R⁴ are each independently a hydrogen atom, a deuterium atom, a substituted or unsubstituted alkyl group having 1 to 10 carbon atoms, or a substituted or unsubstituted aryl group having 6 to 30 ring-forming carbon atoms,
R² is a hydrogen atom, a deuterium atom, or a substituted or unsubstituted alkyl group having 1 to 10 carbon atoms,
L¹ is a direct linkage or a substituted or unsubstituted arylene group having 6 to 30 ring-forming carbon atoms,
Ar¹ does not include two or more carbon atoms containing sp3 hybridized orbitals as a ring-forming atom,
when a1 to a4 are all 0, Ar² is not a 1-dibenzothiophene group,
when a1 to a4 are all 0, L¹-Ar² is not an unsubstituted biphenyl group, and
the amine compound does not include a fluorenyl group in which an alkyl group is substituted on the 9th carbon atom of the fluorenyl group.

2. The light emitting element of claim 1, wherein in Formula 1 above, Ar¹ and Ar² are each independently a substituted or unsubstituted phenyl group, a substituted or unsubstituted naphthyl group, a substituted or unsubstituted phenanthryl group, a substituted or unsubstituted dibenzofuran group, or a substituted or unsubstituted dibenzothiophene group.

3. The light emitting element of claim 1, wherein in Formula 1 above, Ar¹ is represented by any one selected from among AR1-1 to AR1-12 below: 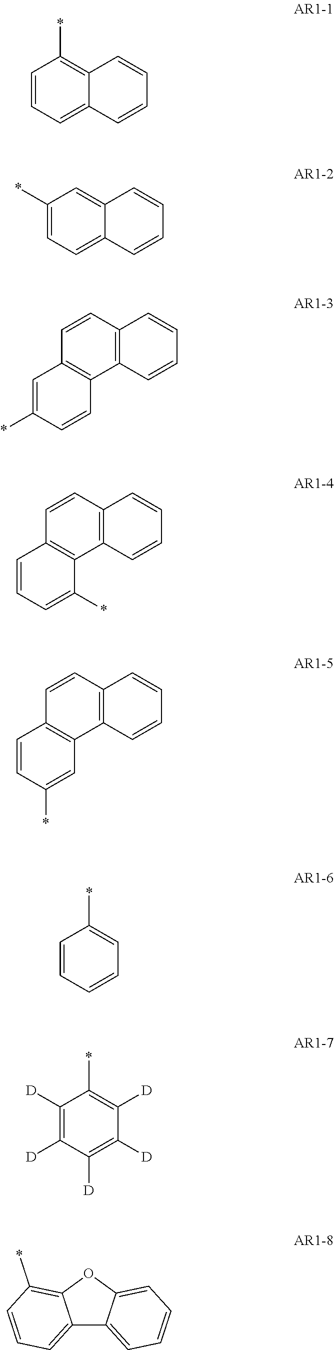 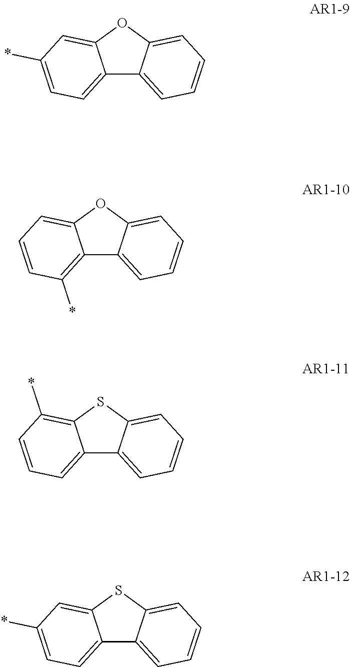 [SMILES: *c1ccc2c(c1)oc1ccccc12 ; *c1cccc2oc3ccccc3c12 ; *c1cccc2c1sc1ccccc12 ; *c1ccc2c(c1)sc1ccccc12]
wherein in AR1-7 above, D is a deuterium atom.

4. The light emitting element of claim 1, wherein in Formula 1 above, Ar² is represented by any one selected from among AR2-1 to AR2-24 below: 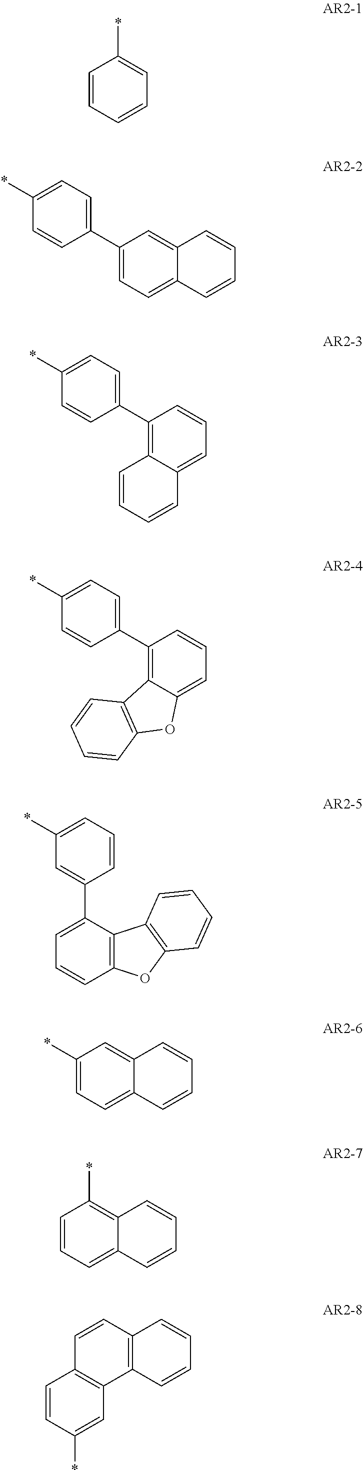 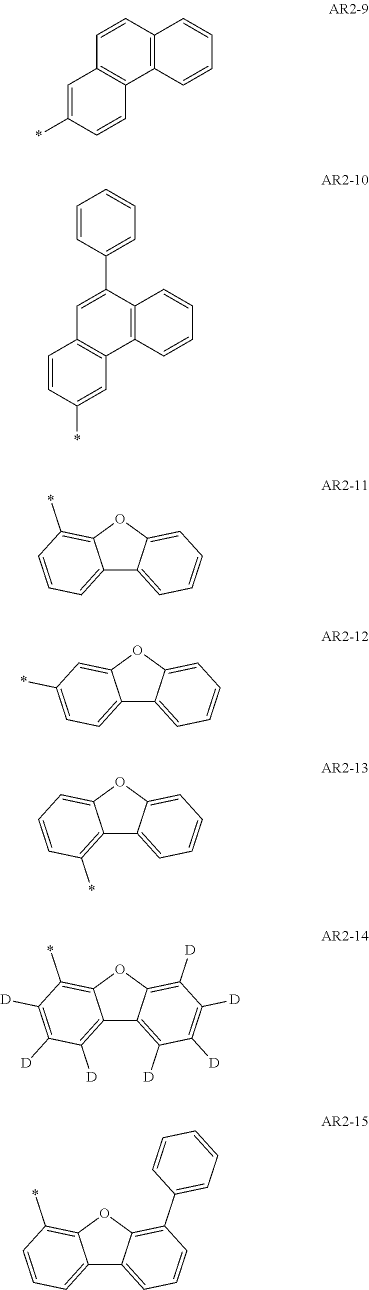 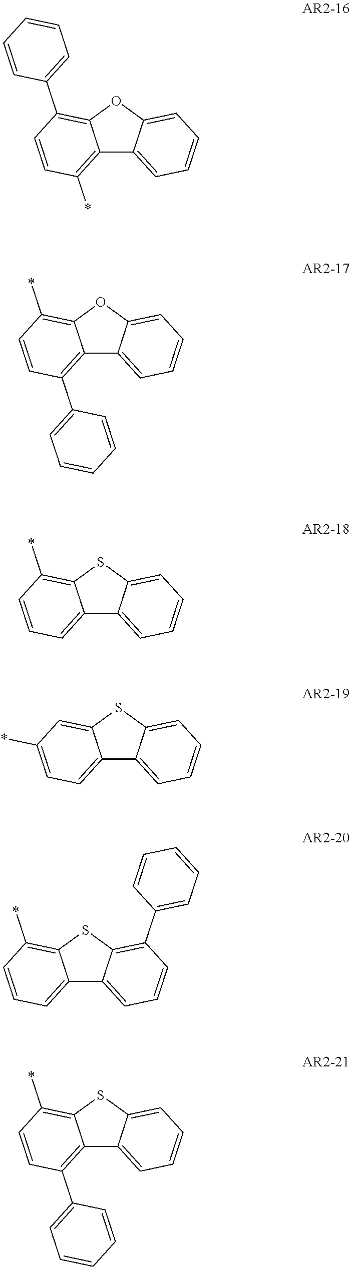 [SMILES: *c1ccc(-c2ccccc2)c2oc3ccccc3c12 ; *c1ccc(-c2ccccc2)c2c1oc1ccccc12 ; *c1cccc2c1sc1ccccc12 ; *c1ccc2c(c1)sc1ccccc12 ; *c1cccc2c1sc1c(-c3ccccc3)cccc12 ; *c1ccc(-c2ccccc2)c2c1sc1ccccc12] 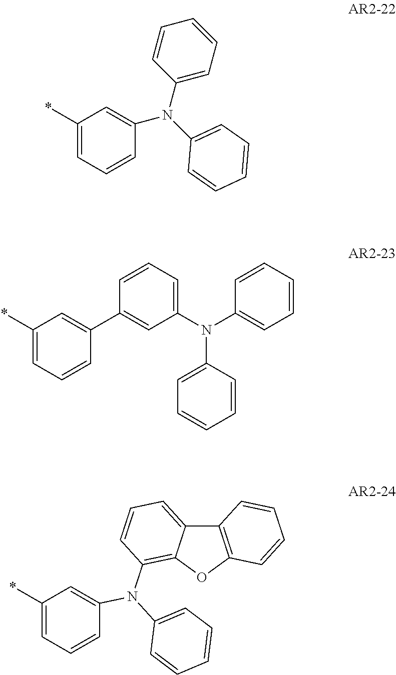 [SMILES: *c1cccc(N(c2ccccc2)c2ccccc2)c1 ; *c1cccc(-c2cccc(N(c3ccccc3)c3ccccc3)c2)c1 ; *c1cccc(N(c2ccccc2)c2cccc3c2oc2ccccc23)c1]
wherein in AR2-14 above, D is a deuterium atom.

5. The light emitting element of claim 1, wherein in Formula 1 above, L¹ is a direct linkage, a substituted or unsubstituted phenylene group, or a substituted or unsubstituted divalent phenanthryl group.

6. The light emitting element of claim 1, wherein the amine compound is represented by any one selected from among Formulas 1-1 to 1-4 below: 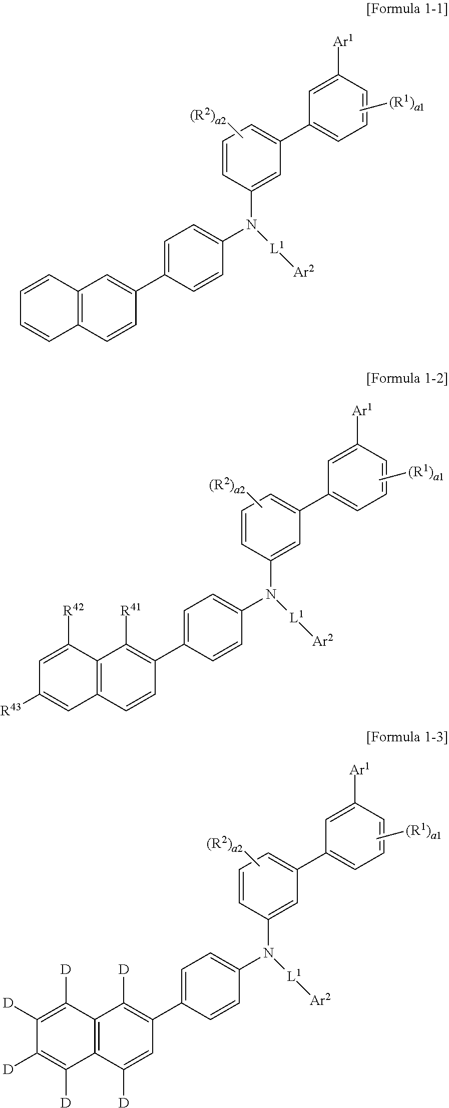 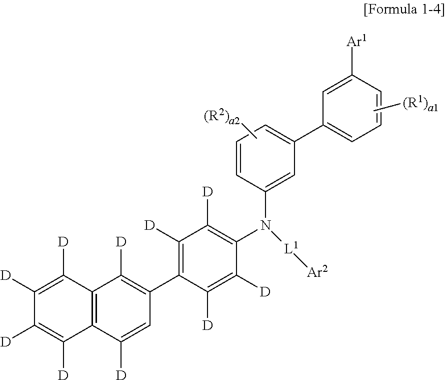
wherein in Formula 1-2 above,
at least one selected from R⁴¹ to R⁴³ is an unsubstituted phenyl group, and the others are hydrogen atoms,
wherein in Formulas 1-3 and 1-4 above,
D is a deuterium atom, and
wherein in Formulas 1-1 to 1-4 above,
Ar¹, Ar², a1, a2, R¹, R², and L¹ are the same as defined with respect to Formula 1 above.

7. The light emitting element of claim 6, wherein the amine compound is represented by any one selected from among Formulas 1-2 Å to 1-2D below: 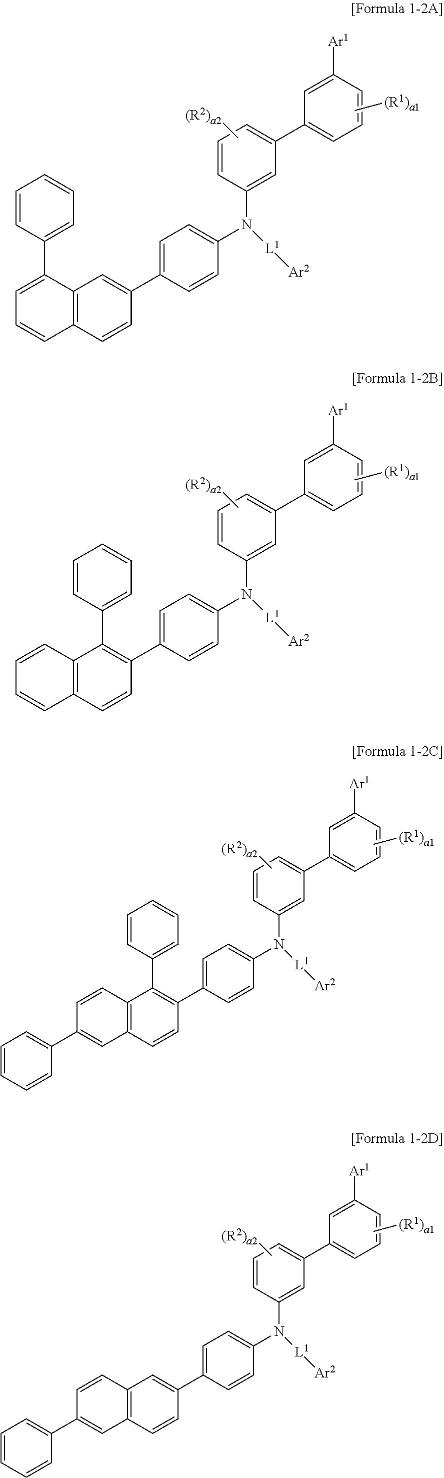
wherein in Formulas 1-2 Å to 1-2D above,
Ar¹, Ar², a1, a2, R¹, R², and L¹ are the same as defined with respect to Formula 1-2 above.

8. The light emitting element of claim 1, wherein the amine compound is a monoamine compound or a diamine compound.

9. The light emitting element of claim 1, wherein the at least one functional layer comprises an emission layer, a hole transport region between the first electrode and the emission layer, and an electron transport region between the emission layer and the second electrode, and
the hole transport region comprises the amine compound.

10. The light emitting element of claim 9, wherein the hole transport region comprises a hole injection layer on the first electrode, a hole transport layer on the hole injection layer, and an electron blocking layer on the hole transport layer, and
at least one selected from the hole injection layer, the hole transport layer, and the electron blocking layer comprises the amine compound.

11. The light emitting element of claim 9, wherein the hole transport region comprises a first hole transport layer and a second hole transport layer, which are sequentially stacked between the first electrode and the emission layer,
the first hole transport layer and the second hole transport layer comprise different hole transport materials, and
the second hole transport layer comprises the amine compound.

12. The light emitting element of claim 1, wherein the amine compound is represented by any one selected from among compounds of Compound Group 1 below: 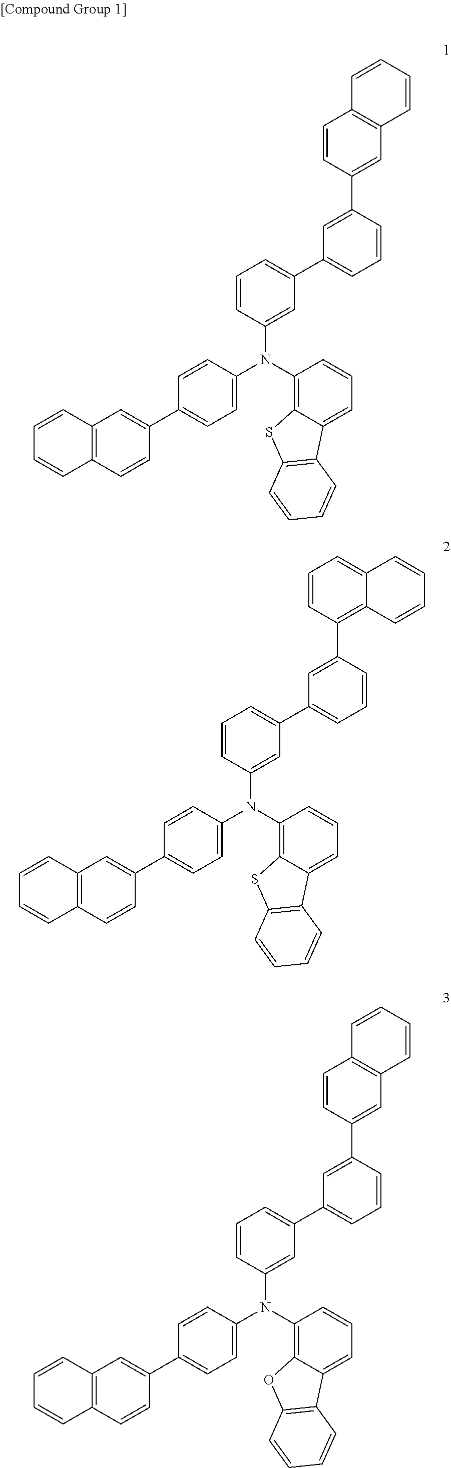 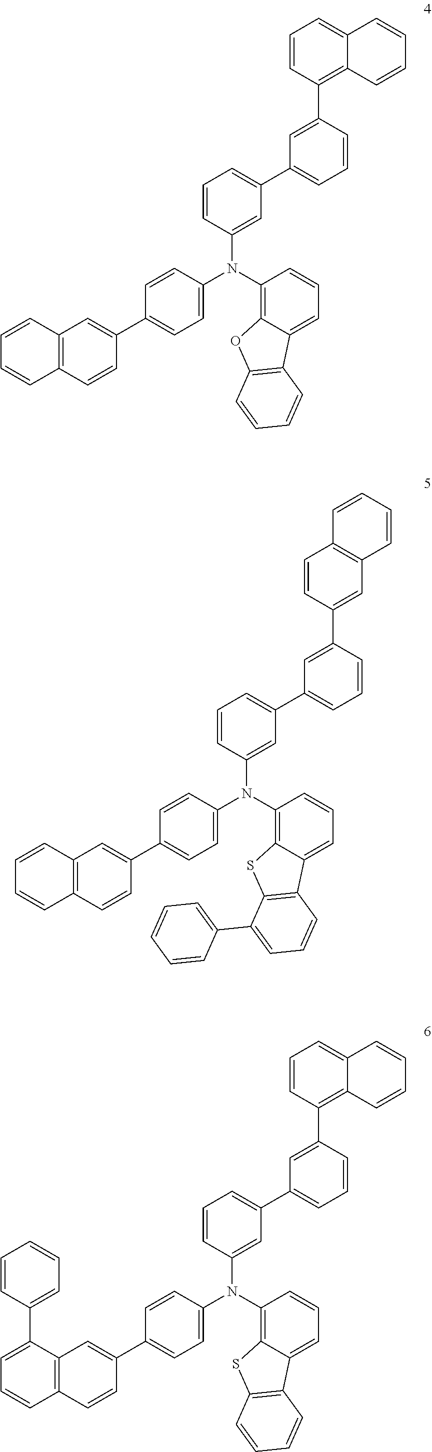 [SMILES: c1cc(-c2cccc(N(c3ccc(-c4ccc5ccccc5c4)cc3)c3cccc4c3oc3ccccc34)c2)cc(-c2cccc3ccccc23)c1 ; c1ccc(-c2cccc3c2sc2c(N(c4ccc(-c5ccc6ccccc6c5)cc4)c4cccc(-c5cccc(-c6ccc7ccccc7c6)c5)c4)cccc23)cc1 ; c1ccc(-c2cccc3ccc(-c4ccc(N(c5cccc(-c6cccc(-c7cccc8ccccc78)c6)c5)c5cccc6c5sc5ccccc56)cc4)cc23)cc1] 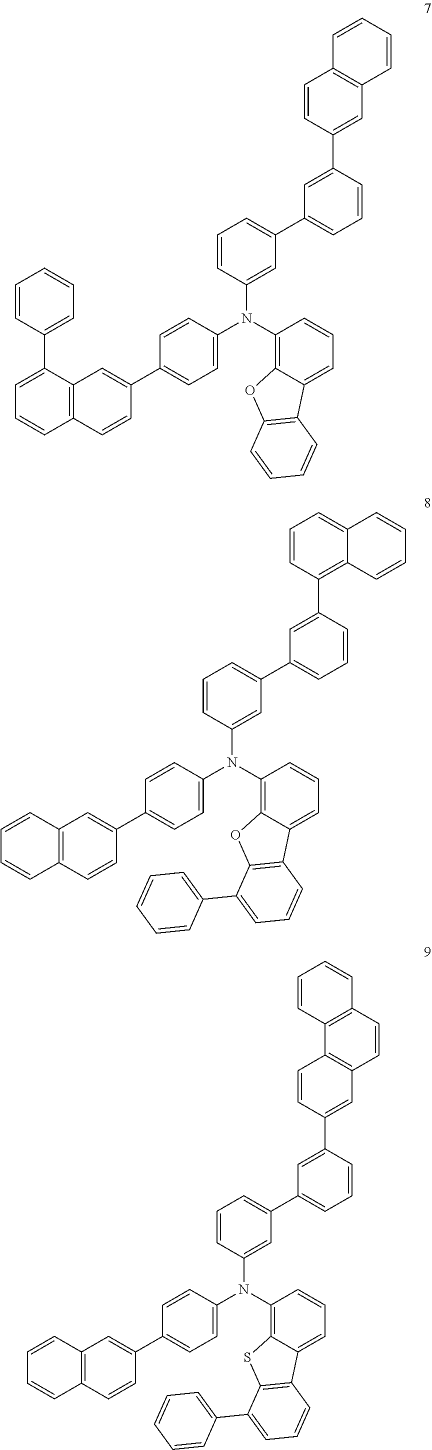 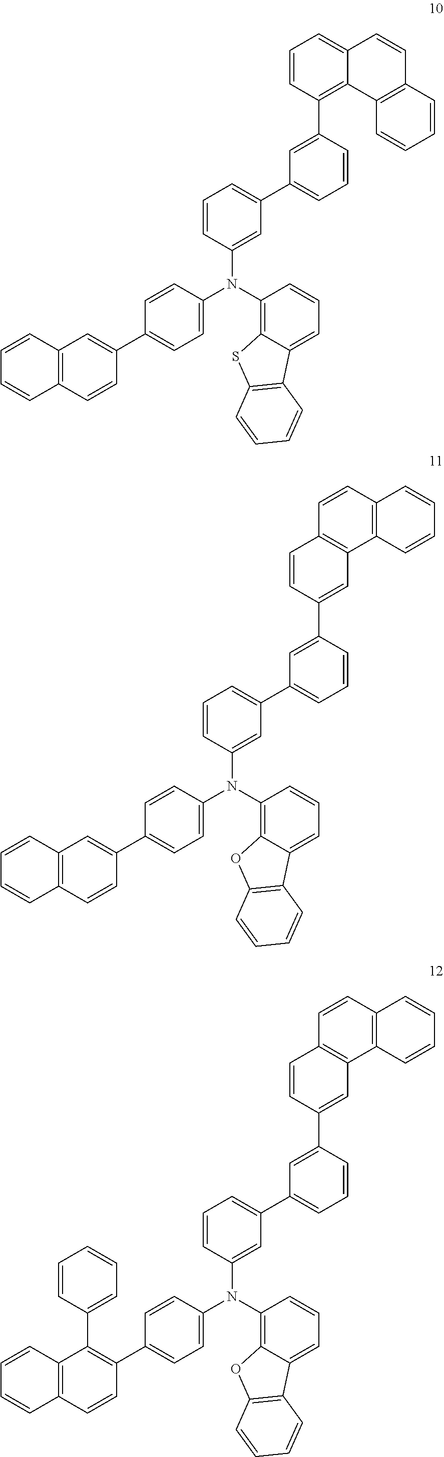 [SMILES: c1cc(-c2cccc(N(c3ccc(-c4ccc5ccccc5c4)cc3)c3cccc4c3sc3ccccc34)c2)cc(-c2cccc3ccc4ccccc4c23)c1 ; c1cc(-c2cccc(N(c3ccc(-c4ccc5ccccc5c4)cc3)c3cccc4c3oc3ccccc34)c2)cc(-c2ccc3ccc4ccccc4c3c2)c1 ; c1ccc(-c2c(-c3ccc(N(c4cccc(-c5cccc(-c6ccc7ccc8ccccc8c7c6)c5)c4)c4cccc5c4oc4ccccc45)cc3)ccc3ccccc23)cc1] 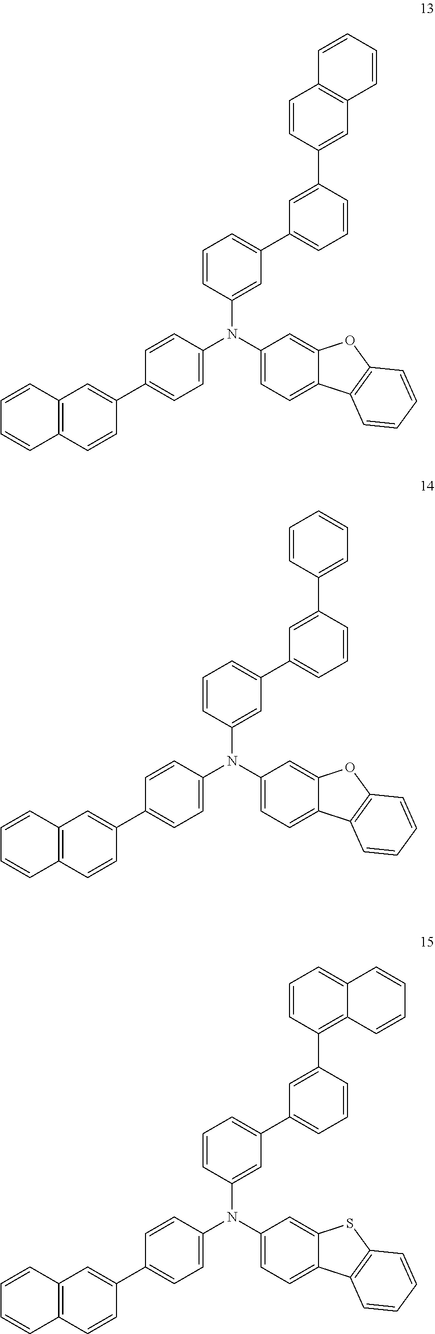 [SMILES: c1cc(-c2cccc(N(c3ccc(-c4ccc5ccccc5c4)cc3)c3ccc4c(c3)oc3ccccc34)c2)cc(-c2ccc3ccccc3c2)c1 ; c1ccc(-c2cccc(-c3cccc(N(c4ccc(-c5ccc6ccccc6c5)cc4)c4ccc5c(c4)oc4ccccc45)c3)c2)cc1 ; c1cc(-c2cccc(N(c3ccc(-c4ccc5ccccc5c4)cc3)c3ccc4c(c3)sc3ccccc34)c2)cc(-c2cccc3ccccc23)c1] 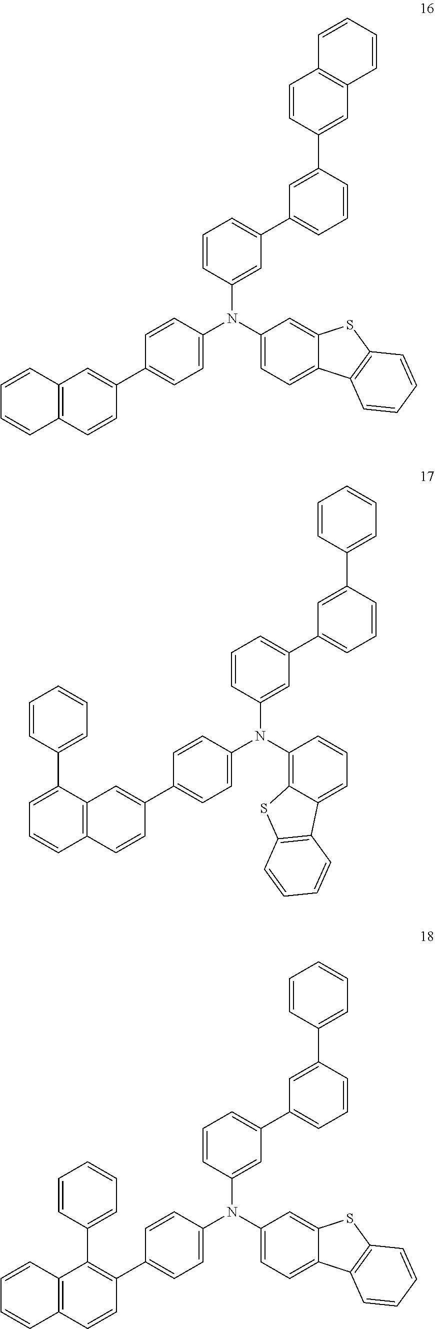 [SMILES: c1cc(-c2cccc(N(c3ccc(-c4ccc5ccccc5c4)cc3)c3ccc4c(c3)sc3ccccc34)c2)cc(-c2ccc3ccccc3c2)c1 ; c1ccc(-c2cccc(-c3cccc(N(c4ccc(-c5ccc6cccc(-c7ccccc7)c6c5)cc4)c4cccc5c4sc4ccccc45)c3)c2)cc1 ; c1ccc(-c2cccc(-c3cccc(N(c4ccc(-c5ccc6ccccc6c5-c5ccccc5)cc4)c4ccc5c(c4)sc4ccccc45)c3)c2)cc1] 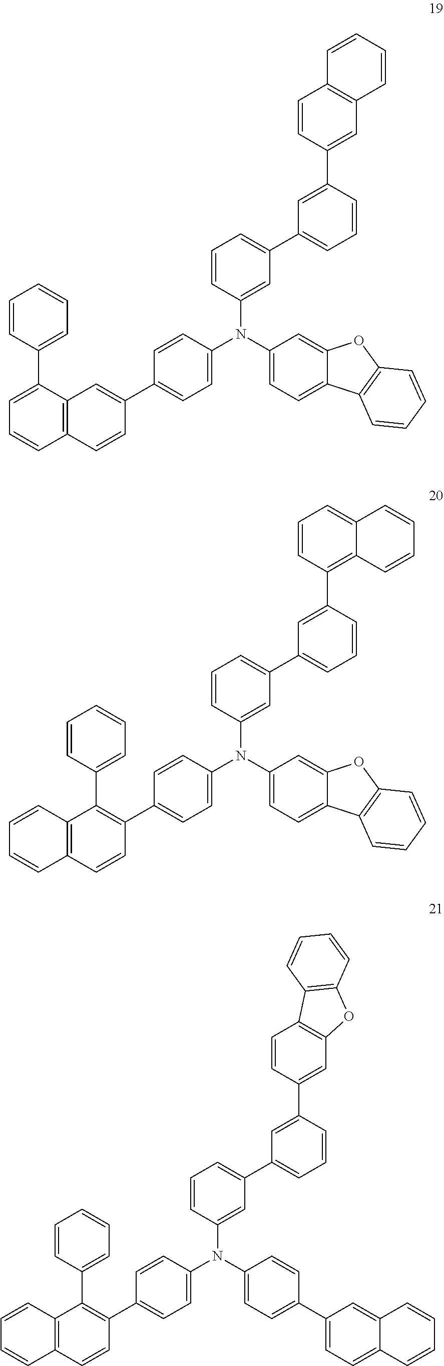 [SMILES: c1ccc(-c2cccc3ccc(-c4ccc(N(c5cccc(-c6cccc(-c7ccc8ccccc8c7)c6)c5)c5ccc6c(c5)oc5ccccc56)cc4)cc23)cc1 ; c1ccc(-c2c(-c3ccc(N(c4cccc(-c5cccc(-c6cccc7ccccc67)c5)c4)c4ccc5c(c4)oc4ccccc45)cc3)ccc3ccccc23)cc1 ; c1ccc(-c2c(-c3ccc(N(c4ccc(-c5ccc6ccccc6c5)cc4)c4cccc(-c5cccc(-c6ccc7c(c6)oc6ccccc67)c5)c4)cc3)ccc3ccccc23)cc1] 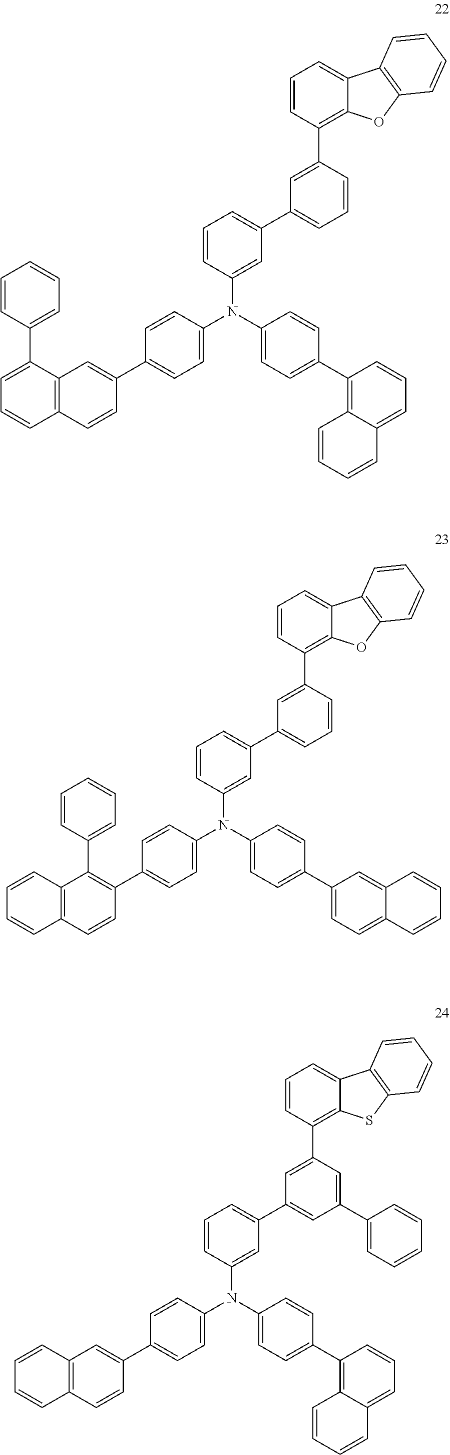 [SMILES: c1ccc(-c2cccc3ccc(-c4ccc(N(c5ccc(-c6cccc7ccccc67)cc5)c5cccc(-c6cccc(-c7cccc8c7oc7ccccc78)c6)c5)cc4)cc23)cc1 ; c1ccc(-c2c(-c3ccc(N(c4ccc(-c5ccc6ccccc6c5)cc4)c4cccc(-c5cccc(-c6cccc7c6oc6ccccc67)c5)c4)cc3)ccc3ccccc23)cc1 ; c1ccc(-c2cc(-c3cccc(N(c4ccc(-c5ccc6ccccc6c5)cc4)c4ccc(-c5cccc6ccccc56)cc4)c3)cc(-c3cccc4c3sc3ccccc34)c2)cc1] 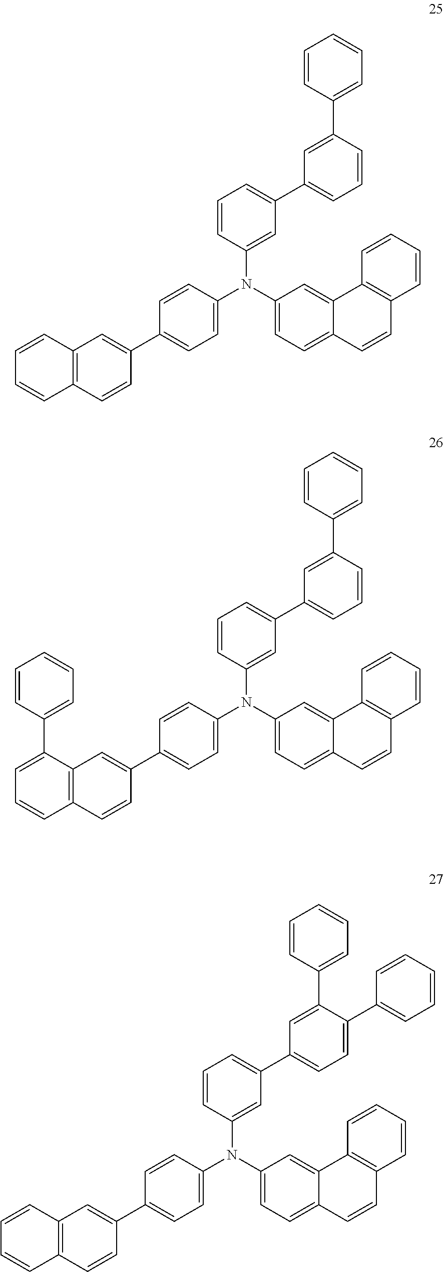 [SMILES: c1ccc(-c2cccc(-c3cccc(N(c4ccc(-c5ccc6ccccc6c5)cc4)c4ccc5ccc6ccccc6c5c4)c3)c2)cc1 ; c1ccc(-c2cccc(-c3cccc(N(c4ccc(-c5ccc6cccc(-c7ccccc7)c6c5)cc4)c4ccc5ccc6ccccc6c5c4)c3)c2)cc1 ; c1ccc(-c2ccc(-c3cccc(N(c4ccc(-c5ccc6ccccc6c5)cc4)c4ccc5ccc6ccccc6c5c4)c3)cc2-c2ccccc2)cc1] 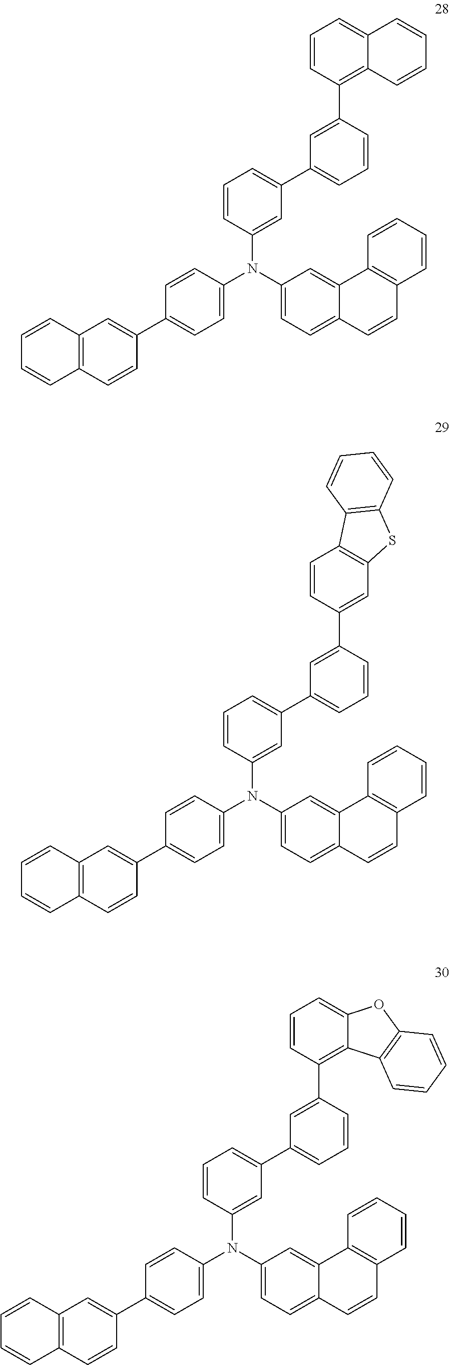 [SMILES: c1cc(-c2cccc(N(c3ccc(-c4ccc5ccccc5c4)cc3)c3ccc4ccc5ccccc5c4c3)c2)cc(-c2cccc3ccccc23)c1 ; c1cc(-c2cccc(N(c3ccc(-c4ccc5ccccc5c4)cc3)c3ccc4ccc5ccccc5c4c3)c2)cc(-c2ccc3c(c2)sc2ccccc23)c1 ; c1cc(-c2cccc(N(c3ccc(-c4ccc5ccccc5c4)cc3)c3ccc4ccc5ccccc5c4c3)c2)cc(-c2cccc3oc4ccccc4c23)c1] 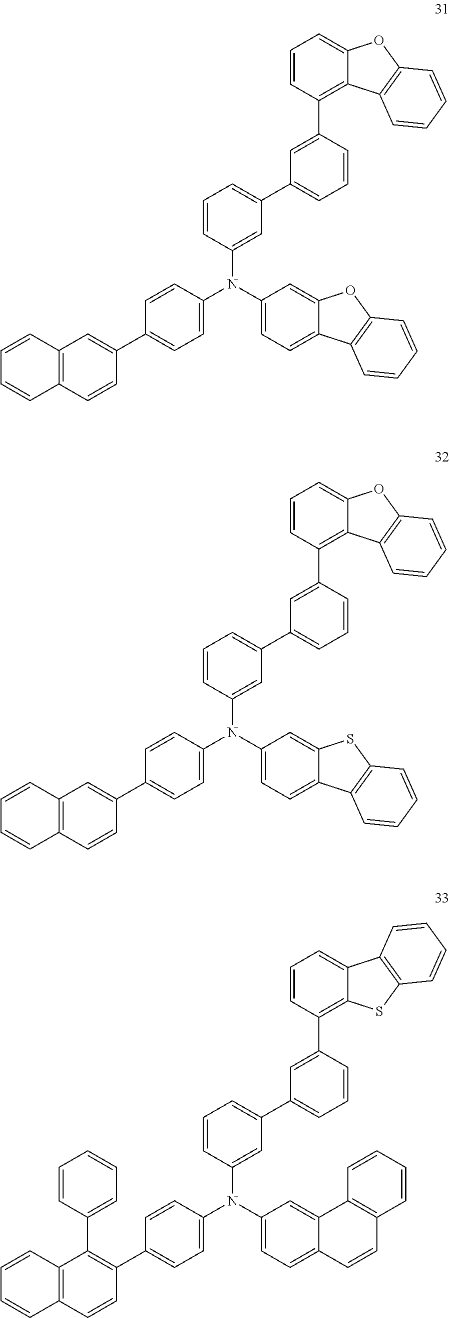 [SMILES: c1cc(-c2cccc(N(c3ccc(-c4ccc5ccccc5c4)cc3)c3ccc4c(c3)oc3ccccc34)c2)cc(-c2cccc3oc4ccccc4c23)c1 ; c1cc(-c2cccc(N(c3ccc(-c4ccc5ccccc5c4)cc3)c3ccc4c(c3)sc3ccccc34)c2)cc(-c2cccc3oc4ccccc4c23)c1 ; c1ccc(-c2c(-c3ccc(N(c4cccc(-c5cccc(-c6cccc7c6sc6ccccc67)c5)c4)c4ccc5ccc6ccccc6c5c4)cc3)ccc3ccccc23)cc1] 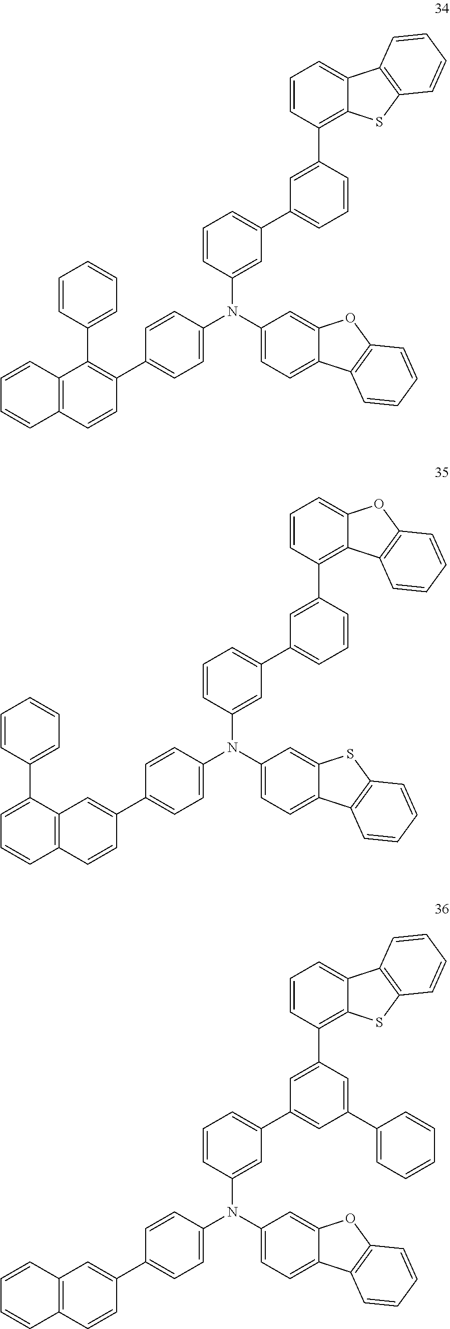 [SMILES: c1ccc(-c2c(-c3ccc(N(c4cccc(-c5cccc(-c6cccc7c6sc6ccccc67)c5)c4)c4ccc5c(c4)oc4ccccc45)cc3)ccc3ccccc23)cc1 ; c1ccc(-c2cccc3ccc(-c4ccc(N(c5cccc(-c6cccc(-c7cccc8oc9ccccc9c78)c6)c5)c5ccc6c(c5)sc5ccccc56)cc4)cc23)cc1 ; c1ccc(-c2cc(-c3cccc(N(c4ccc(-c5ccc6ccccc6c5)cc4)c4ccc5c(c4)oc4ccccc45)c3)cc(-c3cccc4c3sc3ccccc34)c2)cc1] 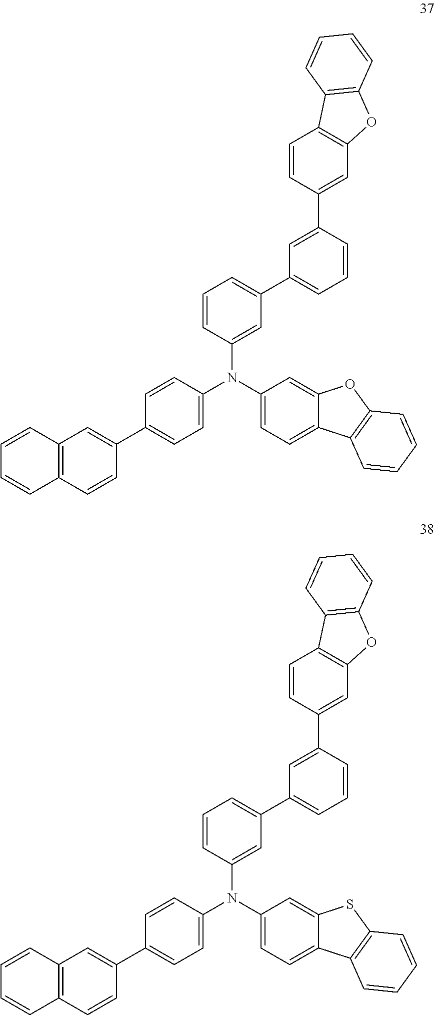 [SMILES: c1cc(-c2cccc(N(c3ccc(-c4ccc5ccccc5c4)cc3)c3ccc4c(c3)oc3ccccc34)c2)cc(-c2ccc3c(c2)oc2ccccc23)c1 ; c1cc(-c2cccc(N(c3ccc(-c4ccc5ccccc5c4)cc3)c3ccc4c(c3)sc3ccccc34)c2)cc(-c2ccc3c(c2)oc2ccccc23)c1] 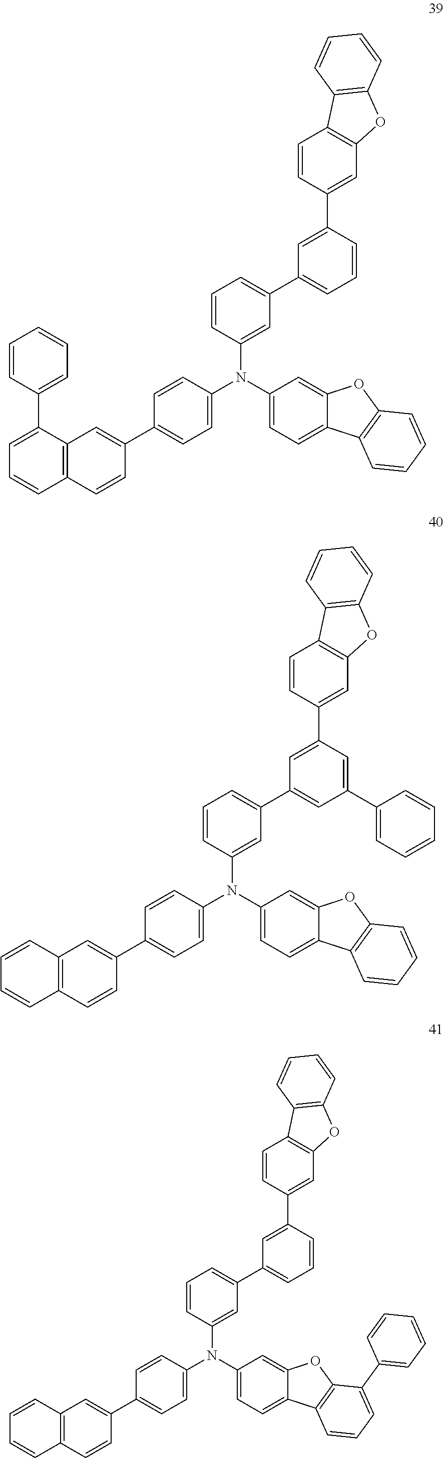 [SMILES: c1ccc(-c2cccc3ccc(-c4ccc(N(c5cccc(-c6cccc(-c7ccc8c(c7)oc7ccccc78)c6)c5)c5ccc6c(c5)oc5ccccc56)cc4)cc23)cc1 ; c1ccc(-c2cc(-c3cccc(N(c4ccc(-c5ccc6ccccc6c5)cc4)c4ccc5c(c4)oc4ccccc45)c3)cc(-c3ccc4c(c3)oc3ccccc34)c2)cc1 ; c1ccc(-c2cccc3c2oc2cc(N(c4ccc(-c5ccc6ccccc6c5)cc4)c4cccc(-c5cccc(-c6ccc7c(c6)oc6ccccc67)c5)c4)ccc23)cc1] 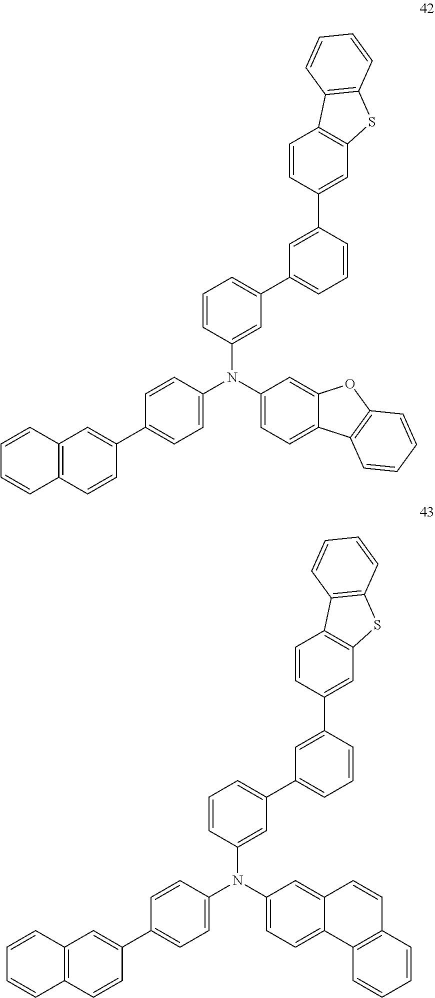 [SMILES: c1cc(-c2cccc(N(c3ccc(-c4ccc5ccccc5c4)cc3)c3ccc4c(c3)oc3ccccc34)c2)cc(-c2ccc3c(c2)sc2ccccc23)c1 ; c1cc(-c2cccc(N(c3ccc(-c4ccc5ccccc5c4)cc3)c3ccc4c(ccc5ccccc54)c3)c2)cc(-c2ccc3c(c2)sc2ccccc23)c1] 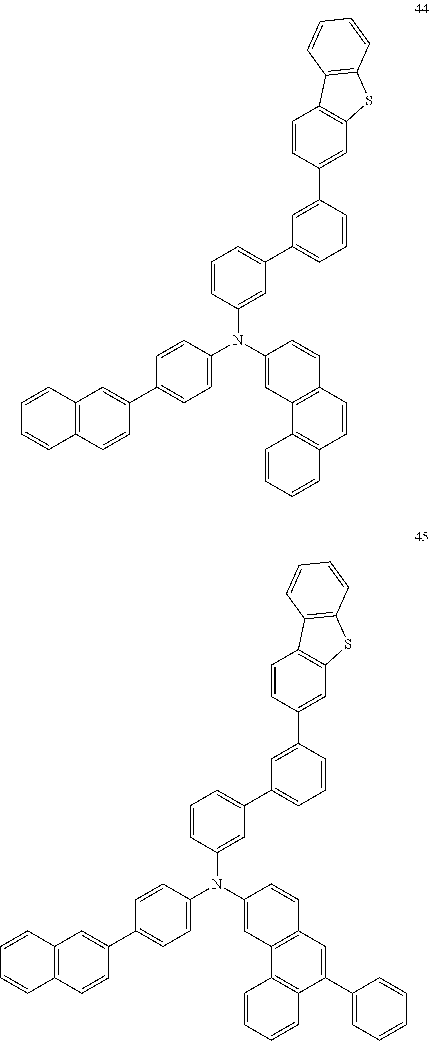 [SMILES: c1cc(-c2cccc(N(c3ccc(-c4ccc5ccccc5c4)cc3)c3ccc4ccc5ccccc5c4c3)c2)cc(-c2ccc3c(c2)sc2ccccc23)c1 ; c1ccc(-c2cc3ccc(N(c4ccc(-c5ccc6ccccc6c5)cc4)c4cccc(-c5cccc(-c6ccc7c(c6)sc6ccccc67)c5)c4)cc3c3ccccc23)cc1] 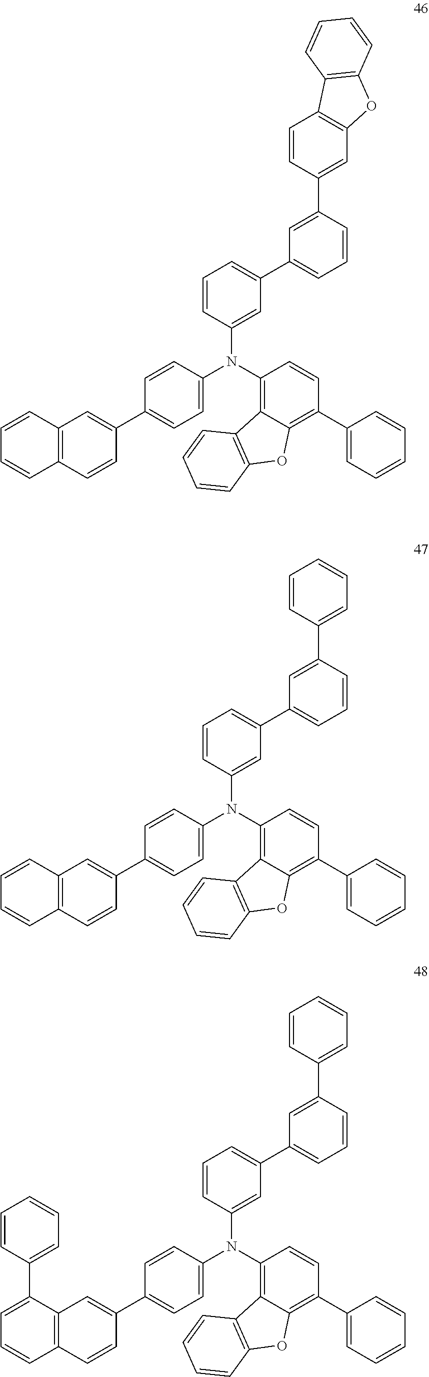 [SMILES: c1ccc(-c2ccc(N(c3ccc(-c4ccc5ccccc5c4)cc3)c3cccc(-c4cccc(-c5ccc6c(c5)oc5ccccc56)c4)c3)c3c2oc2ccccc23)cc1 ; c1ccc(-c2cccc(-c3cccc(N(c4ccc(-c5ccc6ccccc6c5)cc4)c4ccc(-c5ccccc5)c5oc6ccccc6c45)c3)c2)cc1 ; c1ccc(-c2cccc(-c3cccc(N(c4ccc(-c5ccc6cccc(-c7ccccc7)c6c5)cc4)c4ccc(-c5ccccc5)c5oc6ccccc6c45)c3)c2)cc1] 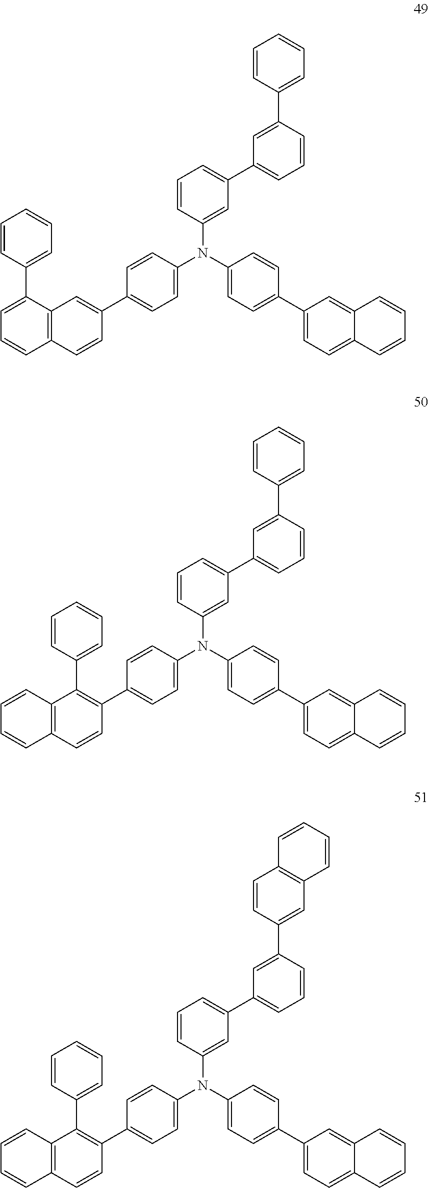 [SMILES: c1ccc(-c2cccc(-c3cccc(N(c4ccc(-c5ccc6ccccc6c5)cc4)c4ccc(-c5ccc6cccc(-c7ccccc7)c6c5)cc4)c3)c2)cc1 ; c1ccc(-c2cccc(-c3cccc(N(c4ccc(-c5ccc6ccccc6c5)cc4)c4ccc(-c5ccc6ccccc6c5-c5ccccc5)cc4)c3)c2)cc1 ; c1ccc(-c2c(-c3ccc(N(c4ccc(-c5ccc6ccccc6c5)cc4)c4cccc(-c5cccc(-c6ccc7ccccc7c6)c5)c4)cc3)ccc3ccccc23)cc1] 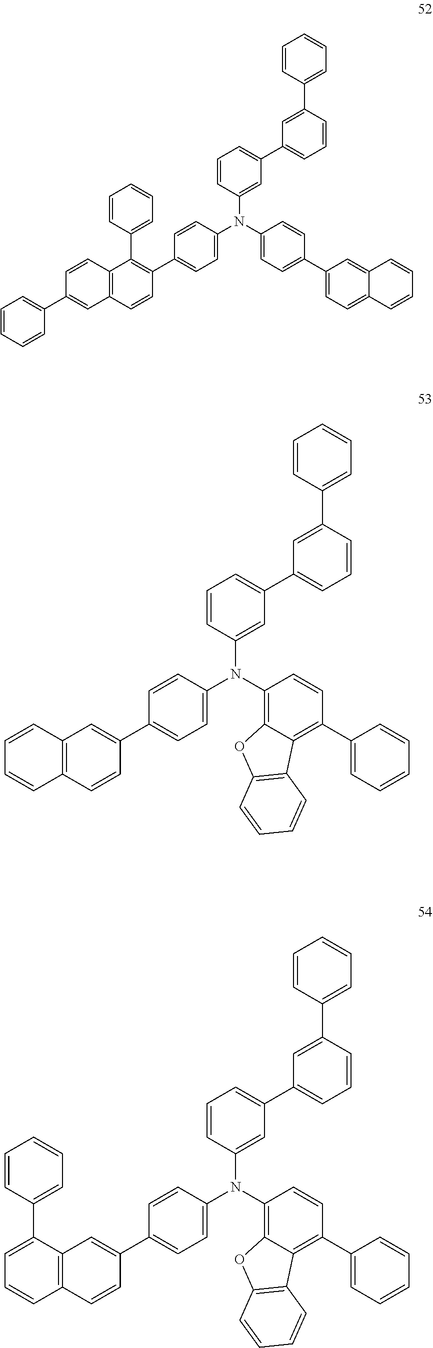 [SMILES: c1ccc(-c2cccc(-c3cccc(N(c4ccc(-c5ccc6ccccc6c5)cc4)c4ccc(-c5ccc6cc(-c7ccccc7)ccc6c5-c5ccccc5)cc4)c3)c2)cc1 ; c1ccc(-c2cccc(-c3cccc(N(c4ccc(-c5ccc6ccccc6c5)cc4)c4ccc(-c5ccccc5)c5c4oc4ccccc45)c3)c2)cc1 ; c1ccc(-c2cccc(-c3cccc(N(c4ccc(-c5ccc6cccc(-c7ccccc7)c6c5)cc4)c4ccc(-c5ccccc5)c5c4oc4ccccc45)c3)c2)cc1] 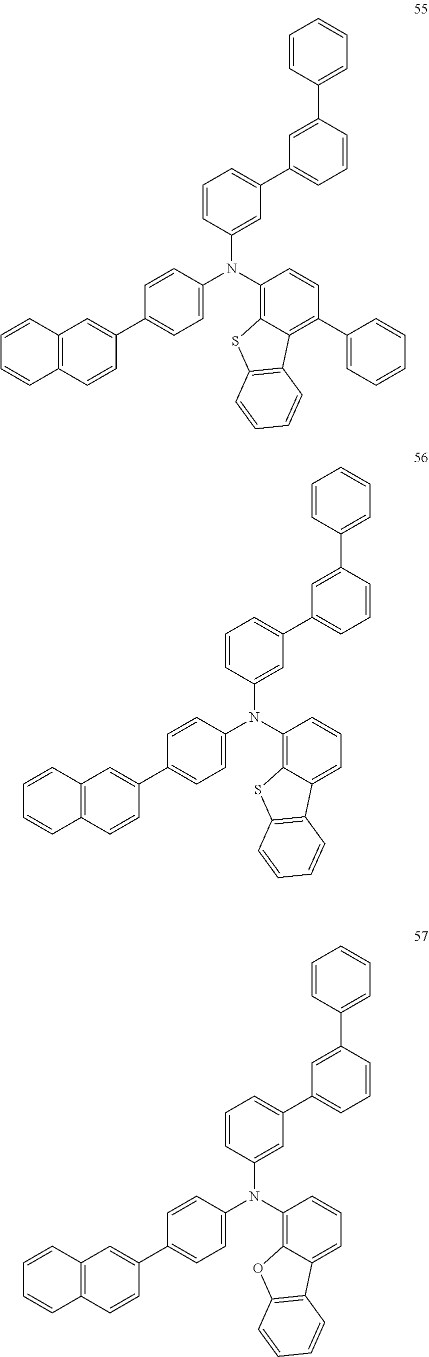 [SMILES: c1ccc(-c2cccc(-c3cccc(N(c4ccc(-c5ccc6ccccc6c5)cc4)c4ccc(-c5ccccc5)c5c4sc4ccccc45)c3)c2)cc1 ; c1ccc(-c2cccc(-c3cccc(N(c4ccc(-c5ccc6ccccc6c5)cc4)c4cccc5c4sc4ccccc45)c3)c2)cc1 ; c1ccc(-c2cccc(-c3cccc(N(c4ccc(-c5ccc6ccccc6c5)cc4)c4cccc5c4oc4ccccc45)c3)c2)cc1] 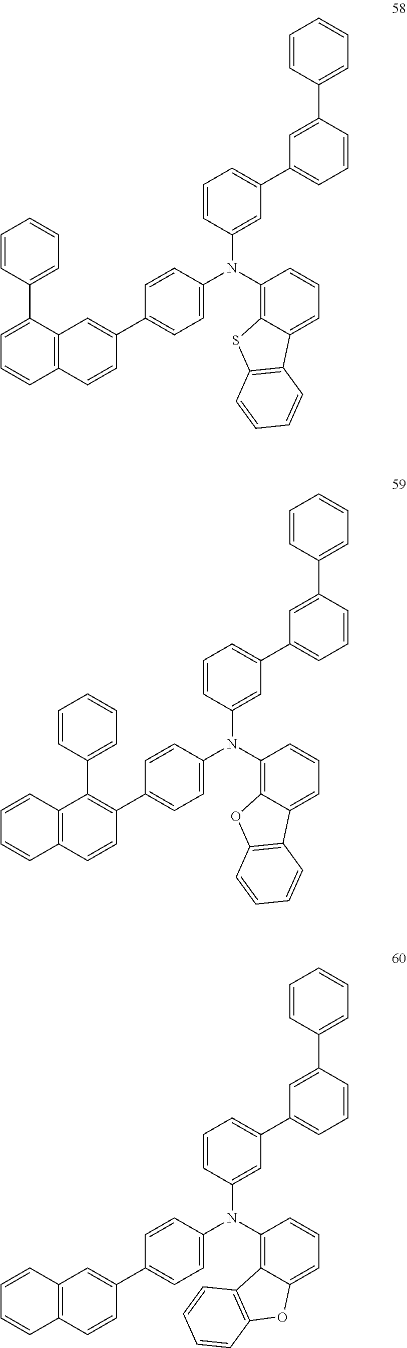 [SMILES: c1ccc(-c2cccc(-c3cccc(N(c4ccc(-c5ccc6cccc(-c7ccccc7)c6c5)cc4)c4cccc5c4sc4ccccc45)c3)c2)cc1 ; c1ccc(-c2cccc(-c3cccc(N(c4ccc(-c5ccc6ccccc6c5-c5ccccc5)cc4)c4cccc5c4oc4ccccc45)c3)c2)cc1 ; c1ccc(-c2cccc(-c3cccc(N(c4ccc(-c5ccc6ccccc6c5)cc4)c4cccc5oc6ccccc6c45)c3)c2)cc1] 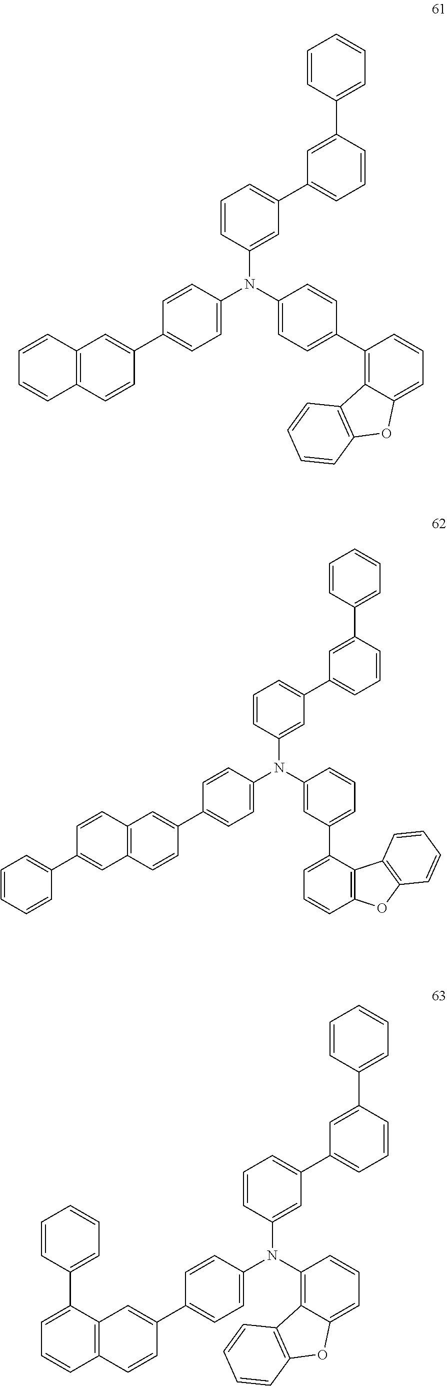 [SMILES: c1ccc(-c2cccc(-c3cccc(N(c4ccc(-c5ccc6ccccc6c5)cc4)c4ccc(-c5cccc6oc7ccccc7c56)cc4)c3)c2)cc1 ; c1ccc(-c2cccc(-c3cccc(N(c4ccc(-c5ccc6cc(-c7ccccc7)ccc6c5)cc4)c4cccc(-c5cccc6oc7ccccc7c56)c4)c3)c2)cc1 ; c1ccc(-c2cccc(-c3cccc(N(c4ccc(-c5ccc6cccc(-c7ccccc7)c6c5)cc4)c4cccc5oc6ccccc6c45)c3)c2)cc1] 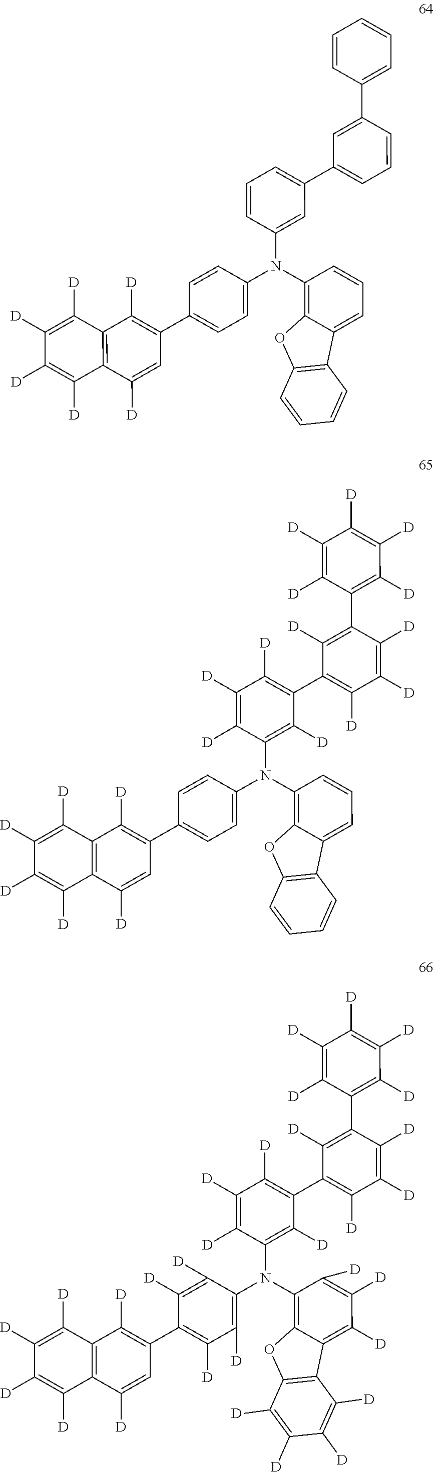 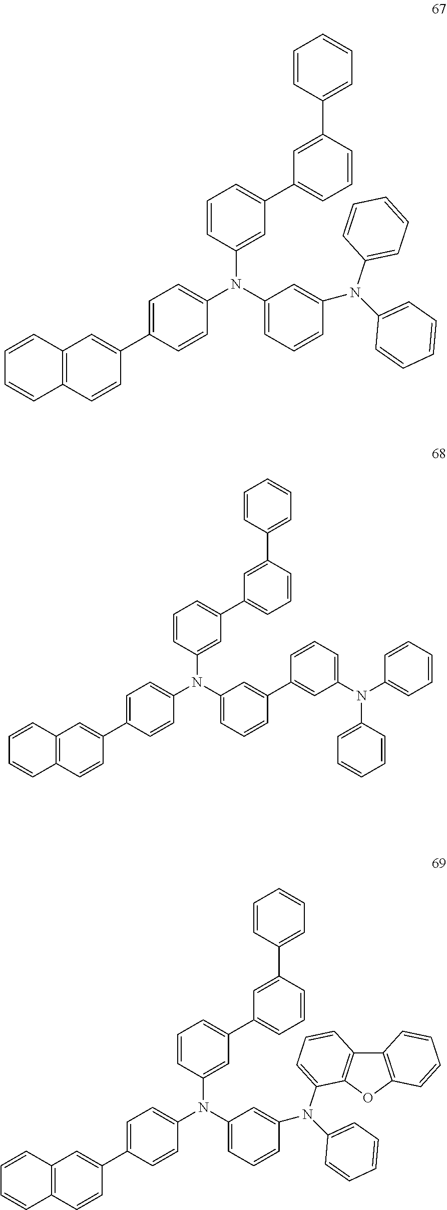 [SMILES: c1ccc(-c2cccc(-c3cccc(N(c4ccc(-c5ccc6ccccc6c5)cc4)c4cccc(N(c5ccccc5)c5ccccc5)c4)c3)c2)cc1 ; c1ccc(-c2cccc(-c3cccc(N(c4ccc(-c5ccc6ccccc6c5)cc4)c4cccc(-c5cccc(N(c6ccccc6)c6ccccc6)c5)c4)c3)c2)cc1 ; c1ccc(-c2cccc(-c3cccc(N(c4ccc(-c5ccc6ccccc6c5)cc4)c4cccc(N(c5ccccc5)c5cccc6c5oc5ccccc56)c4)c3)c2)cc1]
wherein in Compound Group 1 above, D is a deuterium atom.

13. An amine compound represented by Formula 1 below: 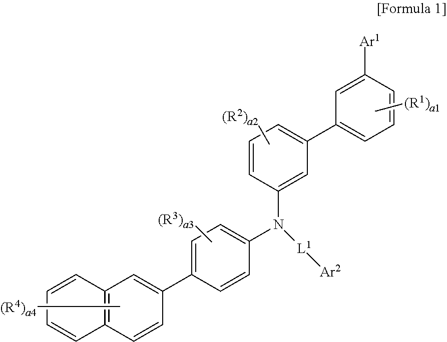
wherein in Formula 1 above,
Ar¹ is a substituted or unsubstituted aryl group having 6 to 15 ring-forming carbon atoms, or a substituted or unsubstituted first heteroaryl group having 5 to 30 ring-forming carbon atoms,
the first heteroaryl group includes only one hetero atom, and the one hetero atom is an oxygen atom or a sulfur atom,
Ar² is a substituted or unsubstituted aryl group having 6 to 30 ring-forming carbon atoms, a substituted or unsubstituted dibenzofuran group, or a substituted or unsubstituted dibenzothiophene group,
a1 to a3 are each independently an integer of 0 to 4,
a4 is an integer of 0 to 7, and
R¹, R³, and R⁴ are each independently a hydrogen atom, a deuterium atom, a substituted or unsubstituted alkyl group having 1 to 10 carbon atoms, or a substituted or unsubstituted aryl group having 6 to 30 ring-forming carbon atoms,
R² is a hydrogen atom, a deuterium atom, or a substituted or unsubstituted alkyl group having 1 to 10 carbon atoms,
L¹ is a direct linkage or a substituted or unsubstituted arylene group having 6 to 30 ring-forming carbon atoms,
Ar¹ does not include two or more carbon atoms containing sp3 hybridized orbitals as a ring-forming atom,
when a1 to a4 are all 0, Ar² is not a 1-dibenzothiophene group,
when a1 to a4 are all 0, L¹-Ar² is not an unsubstituted biphenyl group, and
the amine compound does not include a fluorenyl group in which an alkyl group is substituted on the 9th carbon atom of the fluorenyl group.

14. The amine compound of claim 13, wherein in Formula 1 above, Ar¹ and Ar² are each independently a substituted or unsubstituted phenyl group, a substituted or unsubstituted naphthyl group, a substituted or unsubstituted phenanthryl group, a substituted or unsubstituted dibenzofuran group, or a substituted or unsubstituted dibenzothiophene group.

15. The amine compound of claim 13, wherein in Formula 1 above, Ar¹ is represented by any one selected from among AR1-1 to AR1-12 below: 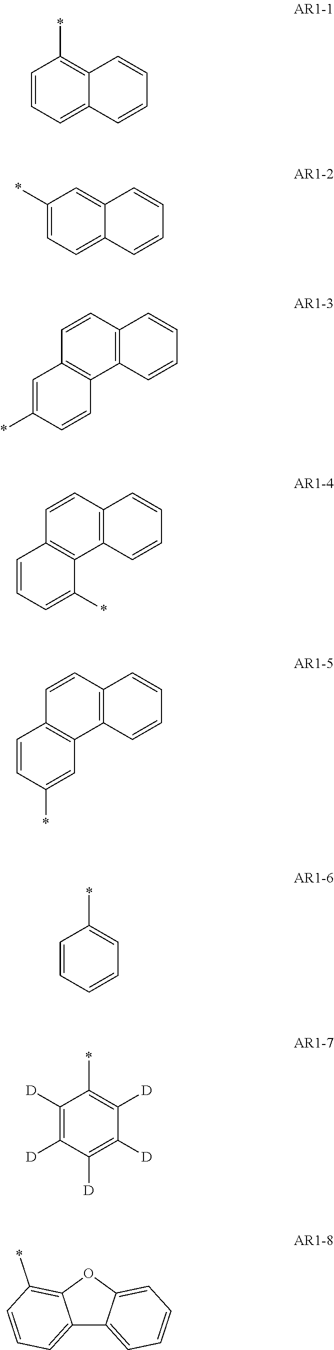 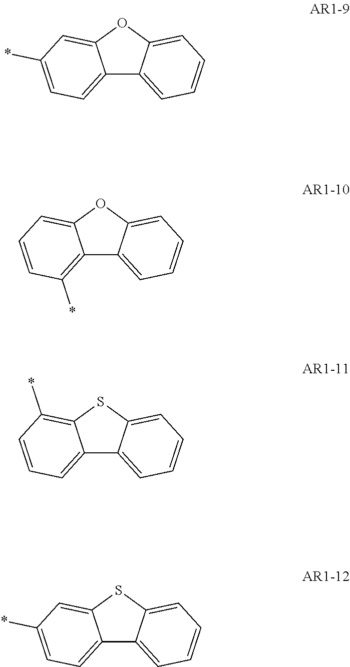 [SMILES: *c1ccc2c(c1)oc1ccccc12 ; *c1cccc2oc3ccccc3c12 ; *c1cccc2c1sc1ccccc12 ; *c1ccc2c(c1)sc1ccccc12]
wherein in AR1-7 above, D is a deuterium atom.

16. The amine compound of claim 13, wherein in Formula 1 above, Ar² is represented by any one selected from among AR2-1 to AR2-24 below: 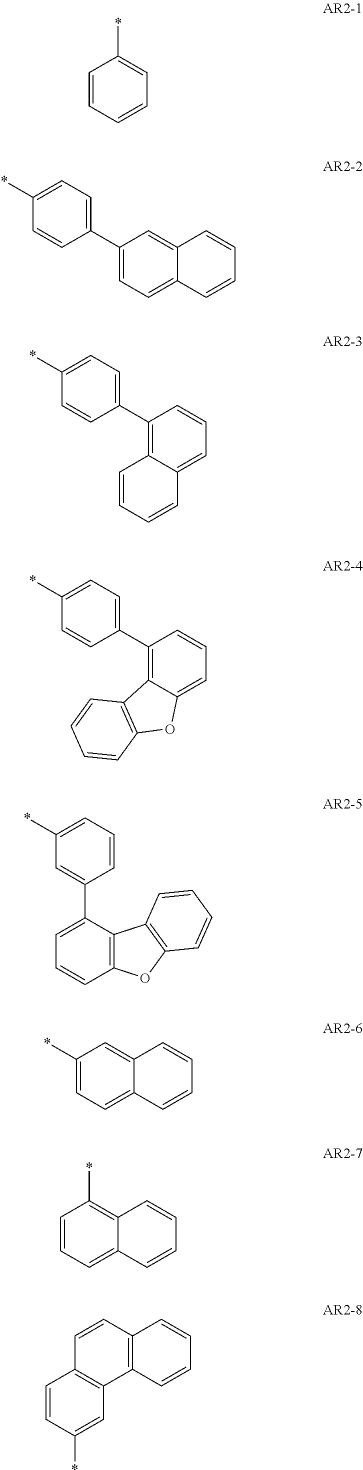 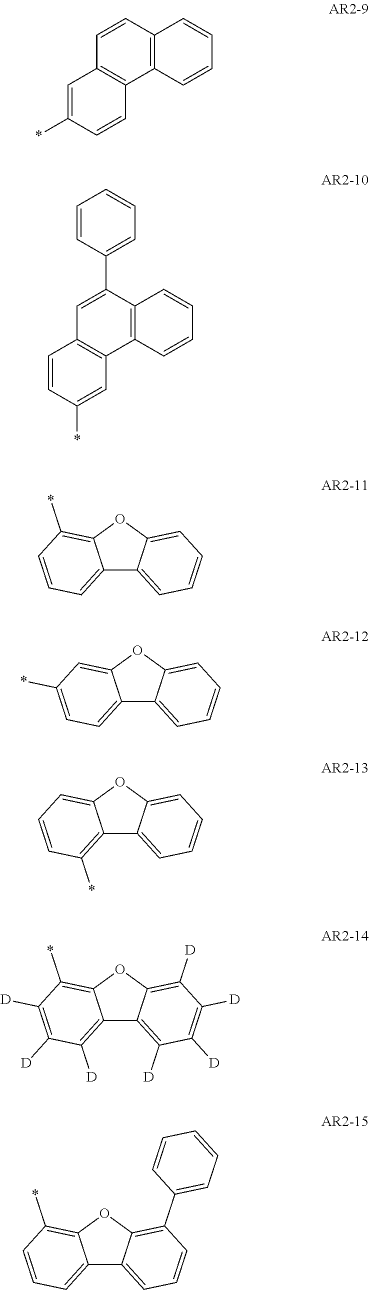 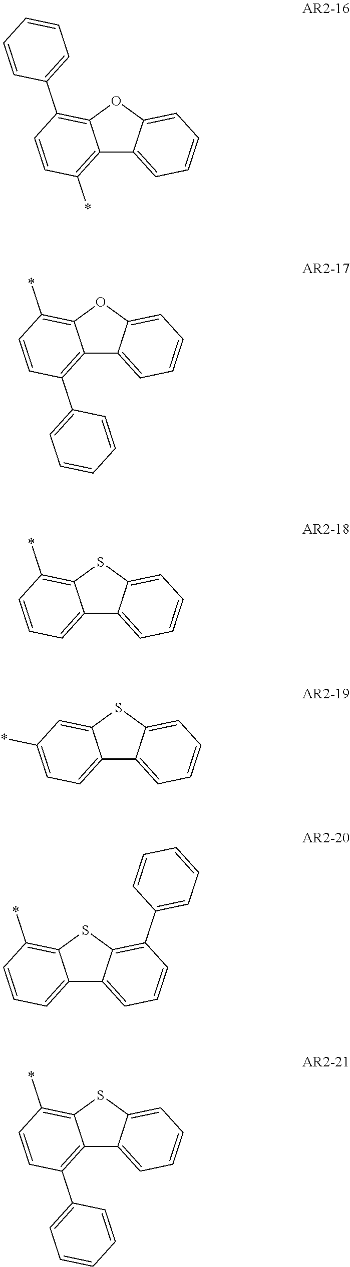 [SMILES: *c1ccc(-c2ccccc2)c2oc3ccccc3c12 ; *c1ccc(-c2ccccc2)c2c1oc1ccccc12 ; *c1cccc2c1sc1ccccc12 ; *c1ccc2c(c1)sc1ccccc12 ; *c1cccc2c1sc1c(-c3ccccc3)cccc12 ; *c1ccc(-c2ccccc2)c2c1sc1ccccc12] 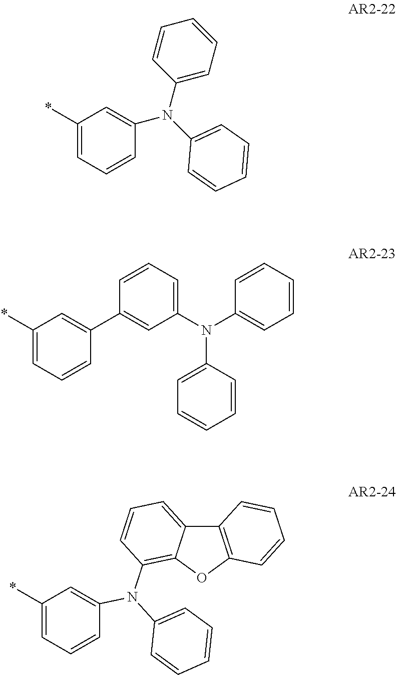 [SMILES: *c1cccc(N(c2ccccc2)c2ccccc2)c1 ; *c1cccc(-c2cccc(N(c3ccccc3)c3ccccc3)c2)c1 ; *c1cccc(N(c2ccccc2)c2cccc3c2oc2ccccc23)c1]
wherein in AR2-14 above, D is a deuterium atom.

17. The amine compound of claim 13, wherein in Formula 1 above, L¹ is a direct linkage, a substituted or unsubstituted phenylene group, or a substituted or unsubstituted divalent phenanthryl group.

18. The amine compound of claim 13, wherein the amine compound is represented by any one selected from among Formulas 1-1 to 1-4 below: 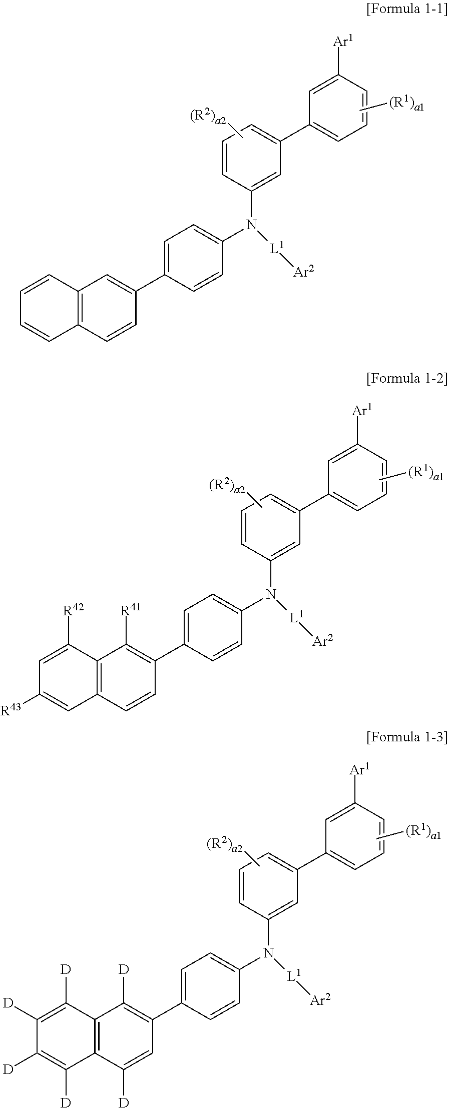 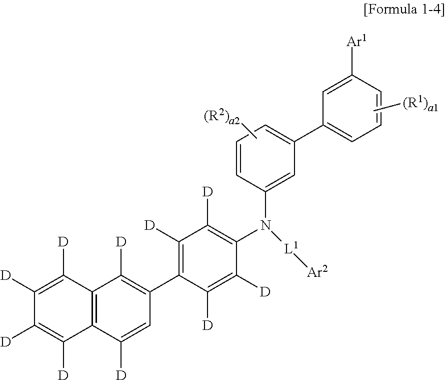
wherein in Formula 1-2 above,
at least one selected from R⁴¹ to R⁴³ is an unsubstituted phenyl group, and the others are hydrogen atoms,
wherein in Formulas 1-3 and 1-4 above,
D is a deuterium atom, and
wherein in Formulas 1-1 to 1-4 above,
Ar¹, Ar², a1, a2, R¹, R², and L¹ are the same as defined with respect to Formula 1 above.

19. The amine compound of claim 18, wherein the amine compound is represented by any one selected from among Formulas 1-2 Å to 1-2D below: 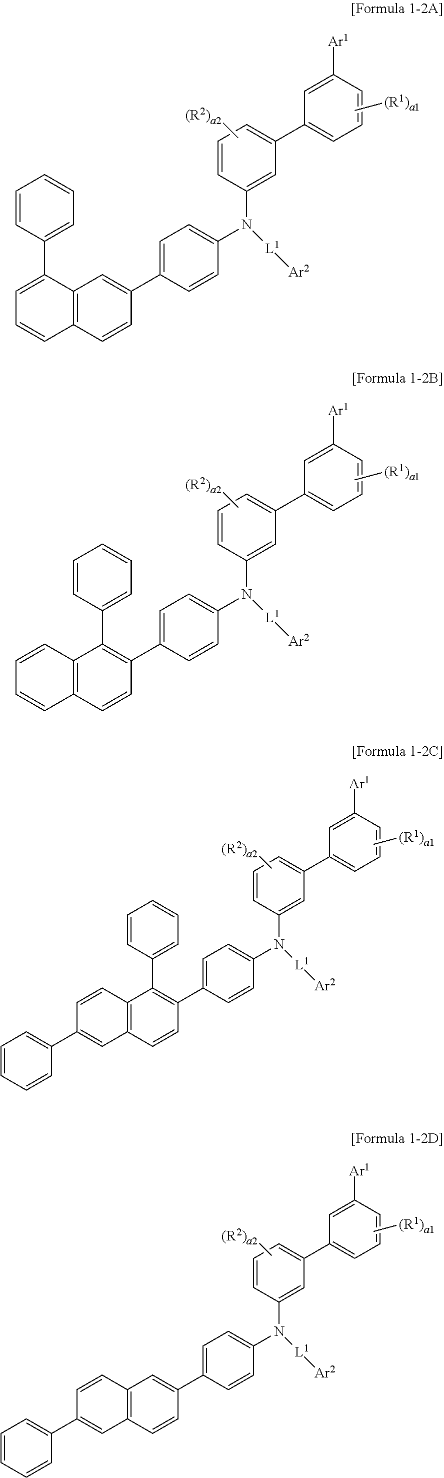
wherein in Formulas 1-2 Å to 1-2D above,
Ar1, Ar², a1, a2, R¹, R², and L¹ are the same as defined with respect to Formula 1-2 above.

20. The amine compound of claim 13, wherein Formula 1 above is represented by any one selected from among compounds of Compound Group 1 below: 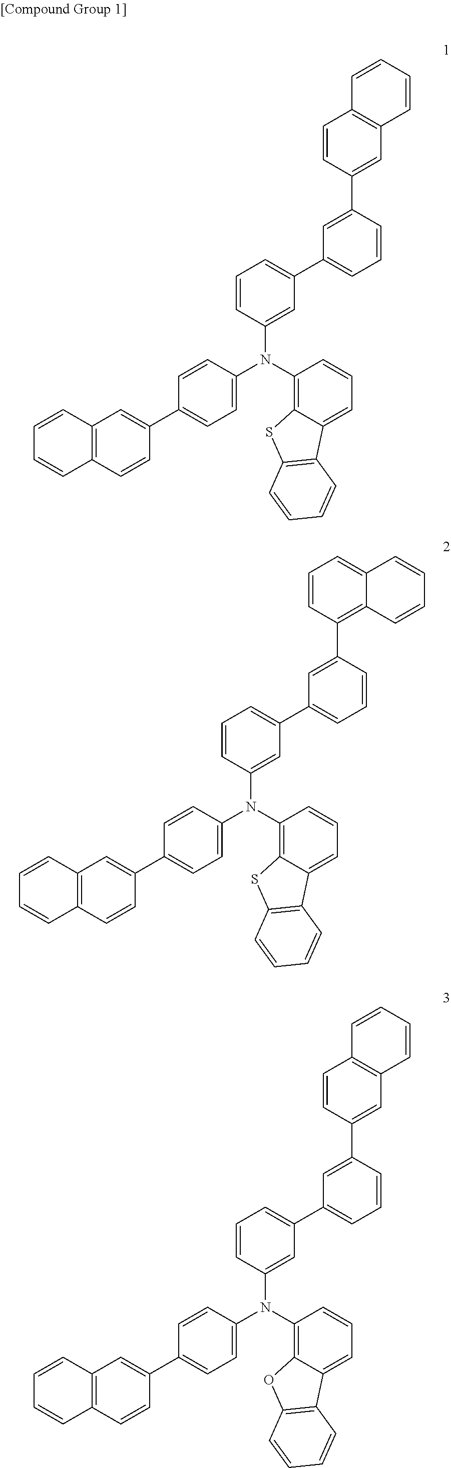 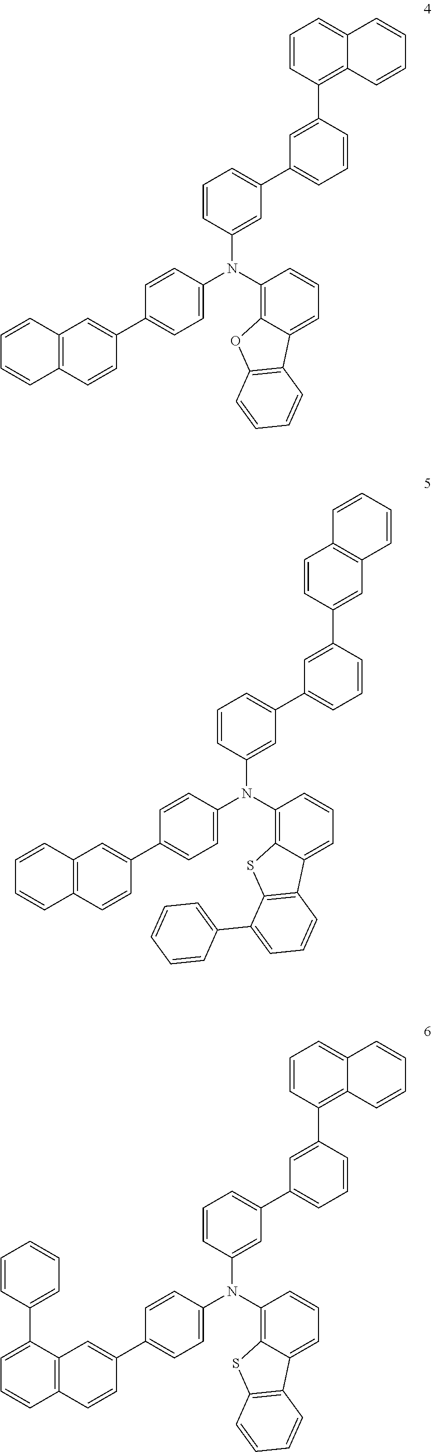 [SMILES: c1cc(-c2cccc(N(c3ccc(-c4ccc5ccccc5c4)cc3)c3cccc4c3oc3ccccc34)c2)cc(-c2cccc3ccccc23)c1 ; c1ccc(-c2cccc3c2sc2c(N(c4ccc(-c5ccc6ccccc6c5)cc4)c4cccc(-c5cccc(-c6ccc7ccccc7c6)c5)c4)cccc23)cc1 ; c1ccc(-c2cccc3ccc(-c4ccc(N(c5cccc(-c6cccc(-c7cccc8ccccc78)c6)c5)c5cccc6c5sc5ccccc56)cc4)cc23)cc1] 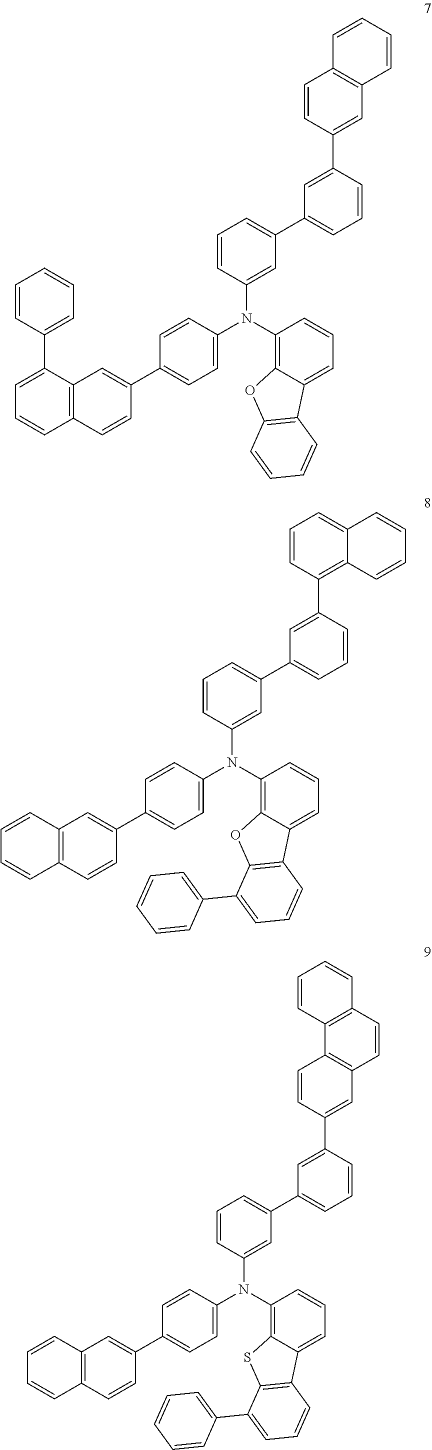 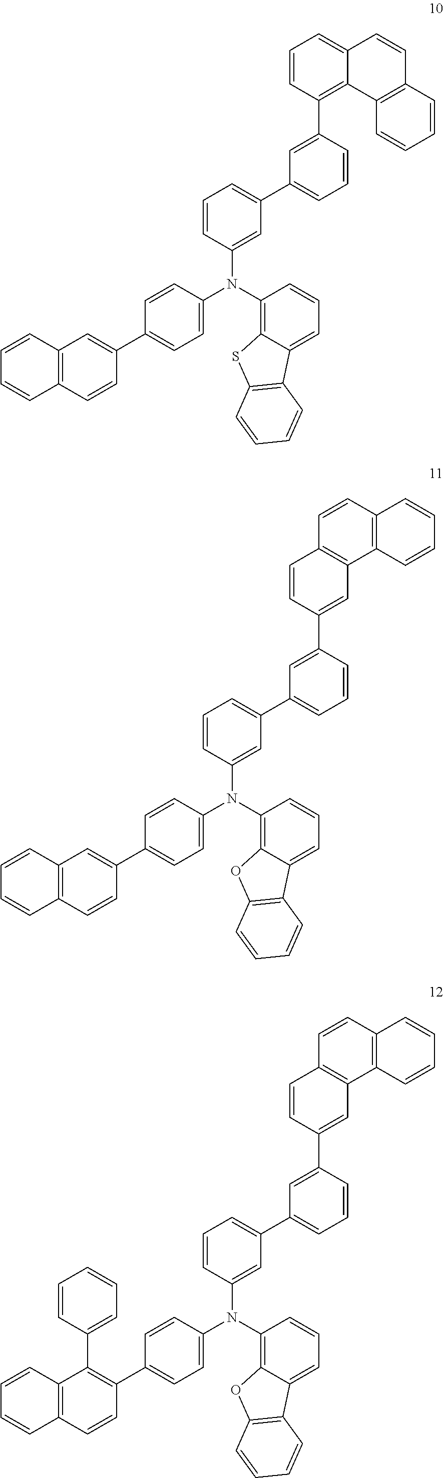 [SMILES: c1cc(-c2cccc(N(c3ccc(-c4ccc5ccccc5c4)cc3)c3cccc4c3sc3ccccc34)c2)cc(-c2cccc3ccc4ccccc4c23)c1 ; c1cc(-c2cccc(N(c3ccc(-c4ccc5ccccc5c4)cc3)c3cccc4c3oc3ccccc34)c2)cc(-c2ccc3ccc4ccccc4c3c2)c1 ; c1ccc(-c2c(-c3ccc(N(c4cccc(-c5cccc(-c6ccc7ccc8ccccc8c7c6)c5)c4)c4cccc5c4oc4ccccc45)cc3)ccc3ccccc23)cc1] 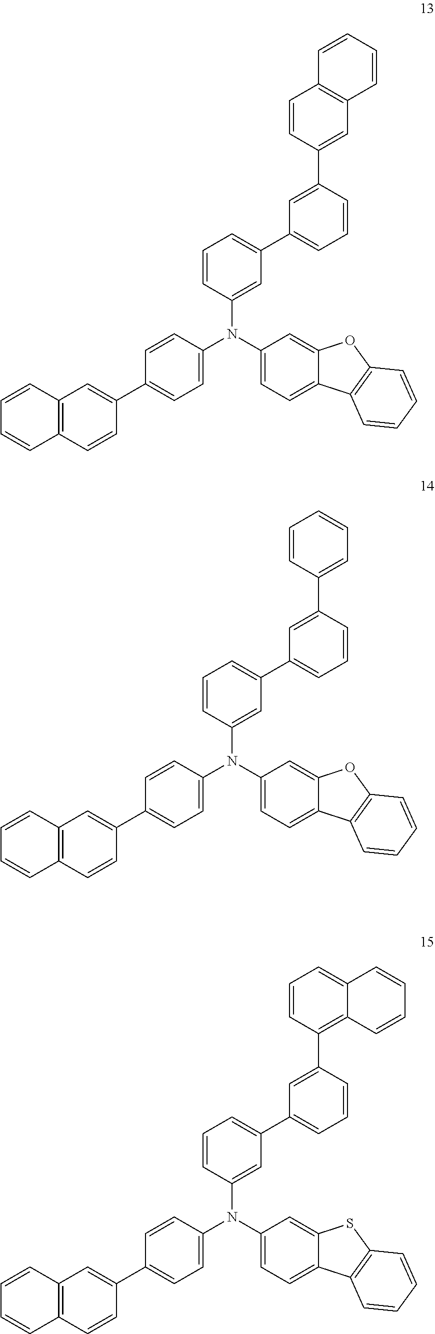 [SMILES: c1cc(-c2cccc(N(c3ccc(-c4ccc5ccccc5c4)cc3)c3ccc4c(c3)oc3ccccc34)c2)cc(-c2ccc3ccccc3c2)c1 ; c1ccc(-c2cccc(-c3cccc(N(c4ccc(-c5ccc6ccccc6c5)cc4)c4ccc5c(c4)oc4ccccc45)c3)c2)cc1 ; c1cc(-c2cccc(N(c3ccc(-c4ccc5ccccc5c4)cc3)c3ccc4c(c3)sc3ccccc34)c2)cc(-c2cccc3ccccc23)c1] 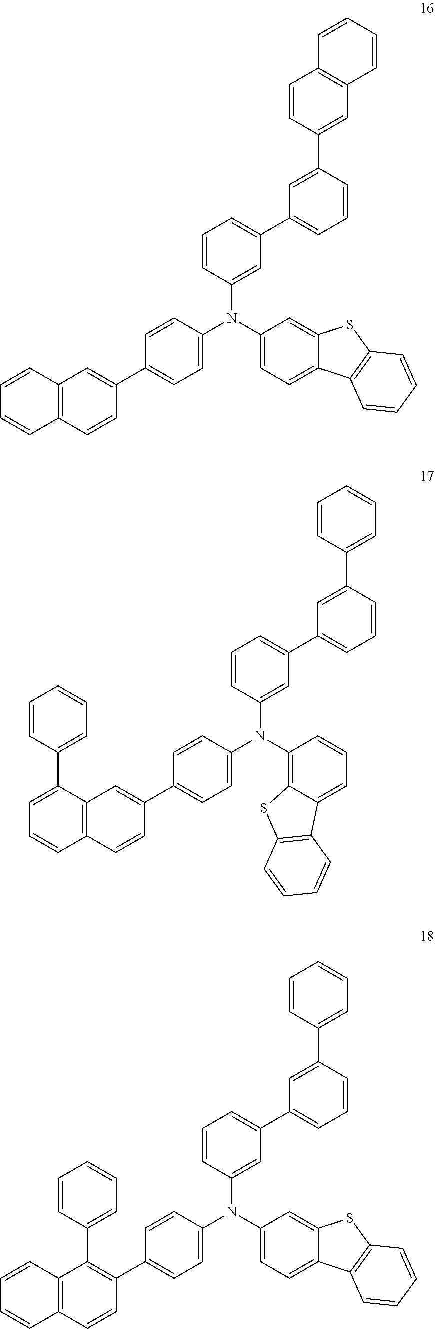 [SMILES: c1cc(-c2cccc(N(c3ccc(-c4ccc5ccccc5c4)cc3)c3ccc4c(c3)sc3ccccc34)c2)cc(-c2ccc3ccccc3c2)c1 ; c1ccc(-c2cccc(-c3cccc(N(c4ccc(-c5ccc6cccc(-c7ccccc7)c6c5)cc4)c4cccc5c4sc4ccccc45)c3)c2)cc1 ; c1ccc(-c2cccc(-c3cccc(N(c4ccc(-c5ccc6ccccc6c5-c5ccccc5)cc4)c4ccc5c(c4)sc4ccccc45)c3)c2)cc1] 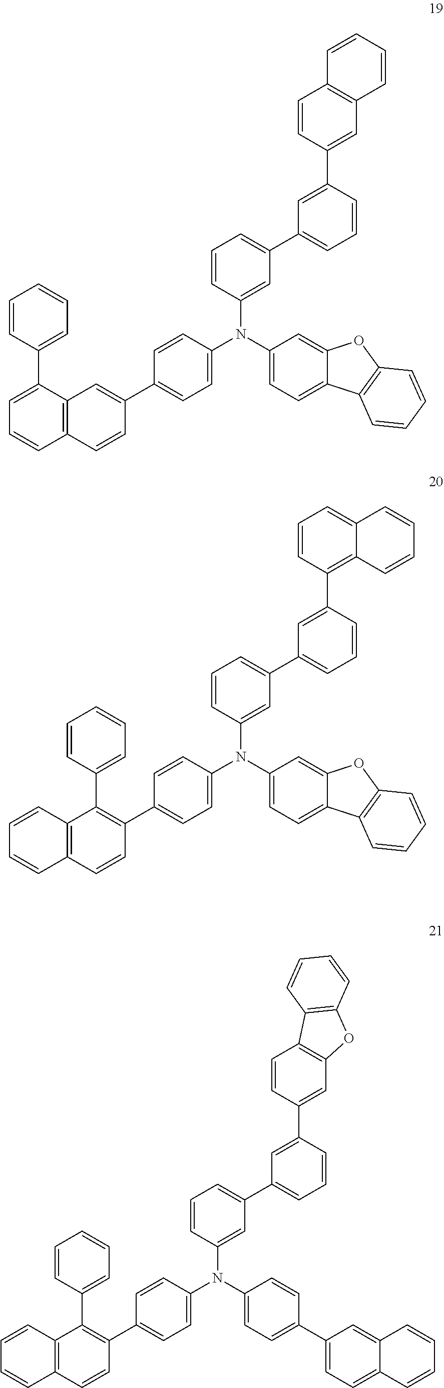 [SMILES: c1ccc(-c2cccc3ccc(-c4ccc(N(c5cccc(-c6cccc(-c7ccc8ccccc8c7)c6)c5)c5ccc6c(c5)oc5ccccc56)cc4)cc23)cc1 ; c1ccc(-c2c(-c3ccc(N(c4cccc(-c5cccc(-c6cccc7ccccc67)c5)c4)c4ccc5c(c4)oc4ccccc45)cc3)ccc3ccccc23)cc1 ; c1ccc(-c2c(-c3ccc(N(c4ccc(-c5ccc6ccccc6c5)cc4)c4cccc(-c5cccc(-c6ccc7c(c6)oc6ccccc67)c5)c4)cc3)ccc3ccccc23)cc1] 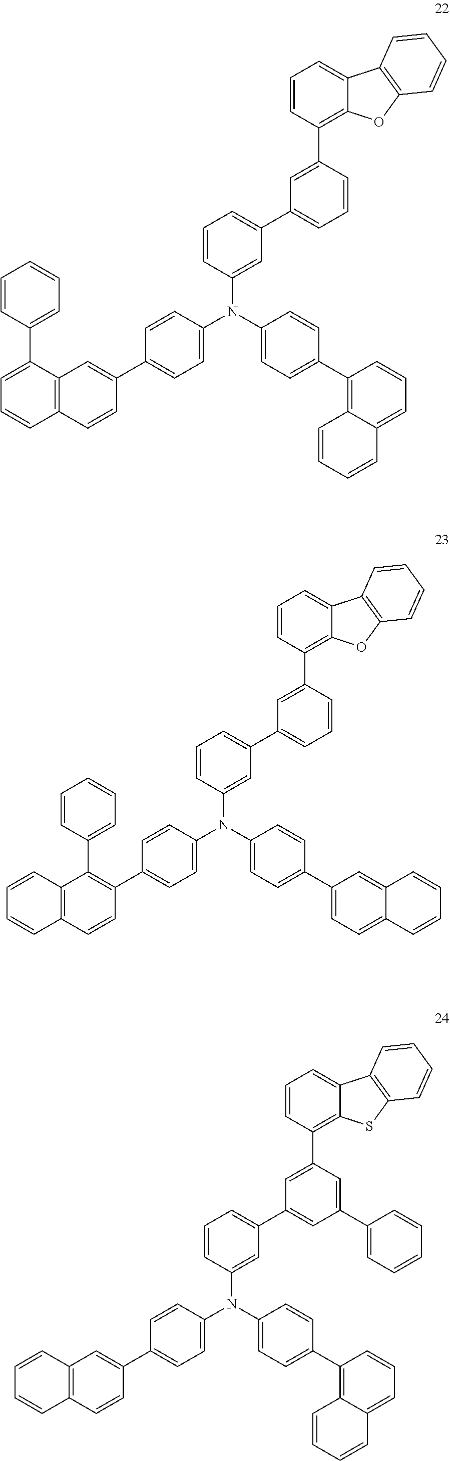 [SMILES: c1ccc(-c2cccc3ccc(-c4ccc(N(c5ccc(-c6cccc7ccccc67)cc5)c5cccc(-c6cccc(-c7cccc8c7oc7ccccc78)c6)c5)cc4)cc23)cc1 ; c1ccc(-c2c(-c3ccc(N(c4ccc(-c5ccc6ccccc6c5)cc4)c4cccc(-c5cccc(-c6cccc7c6oc6ccccc67)c5)c4)cc3)ccc3ccccc23)cc1 ; c1ccc(-c2cc(-c3cccc(N(c4ccc(-c5ccc6ccccc6c5)cc4)c4ccc(-c5cccc6ccccc56)cc4)c3)cc(-c3cccc4c3sc3ccccc34)c2)cc1] 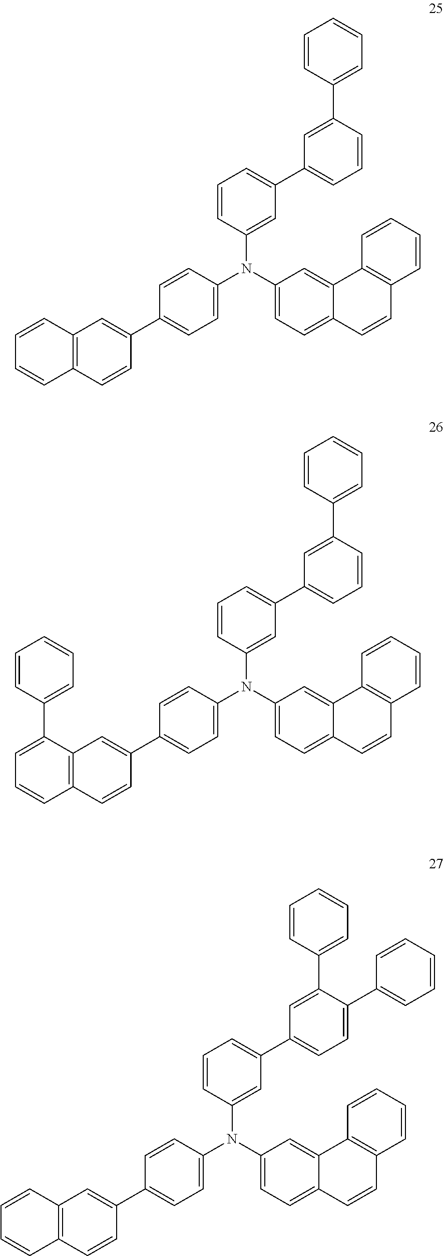 [SMILES: c1ccc(-c2cccc(-c3cccc(N(c4ccc(-c5ccc6ccccc6c5)cc4)c4ccc5ccc6ccccc6c5c4)c3)c2)cc1 ; c1ccc(-c2cccc(-c3cccc(N(c4ccc(-c5ccc6cccc(-c7ccccc7)c6c5)cc4)c4ccc5ccc6ccccc6c5c4)c3)c2)cc1 ; c1ccc(-c2ccc(-c3cccc(N(c4ccc(-c5ccc6ccccc6c5)cc4)c4ccc5ccc6ccccc6c5c4)c3)cc2-c2ccccc2)cc1] 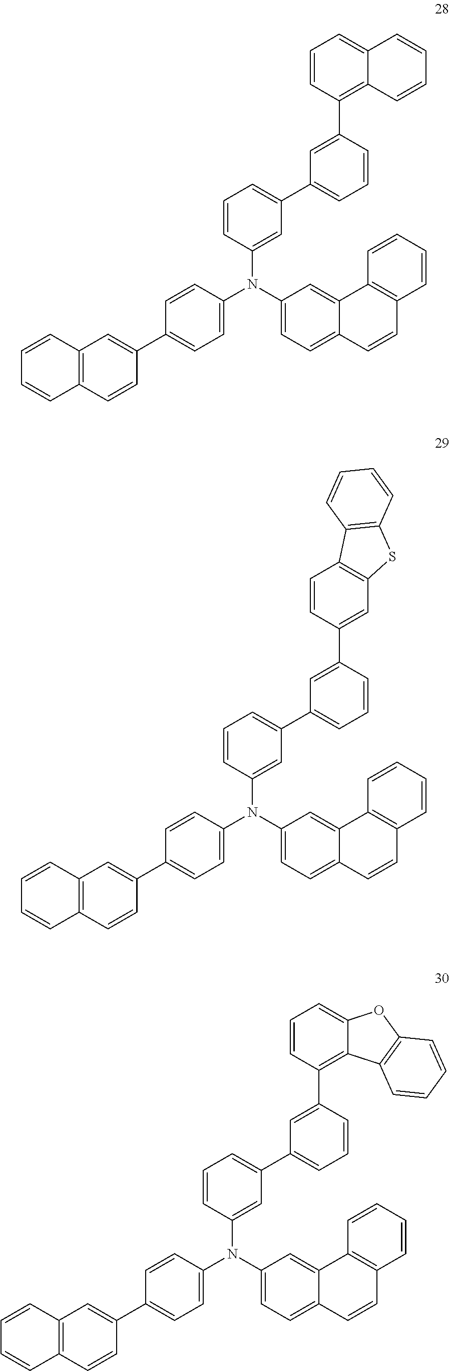 [SMILES: c1cc(-c2cccc(N(c3ccc(-c4ccc5ccccc5c4)cc3)c3ccc4ccc5ccccc5c4c3)c2)cc(-c2cccc3ccccc23)c1 ; c1cc(-c2cccc(N(c3ccc(-c4ccc5ccccc5c4)cc3)c3ccc4ccc5ccccc5c4c3)c2)cc(-c2ccc3c(c2)sc2ccccc23)c1 ; c1cc(-c2cccc(N(c3ccc(-c4ccc5ccccc5c4)cc3)c3ccc4ccc5ccccc5c4c3)c2)cc(-c2cccc3oc4ccccc4c23)c1] 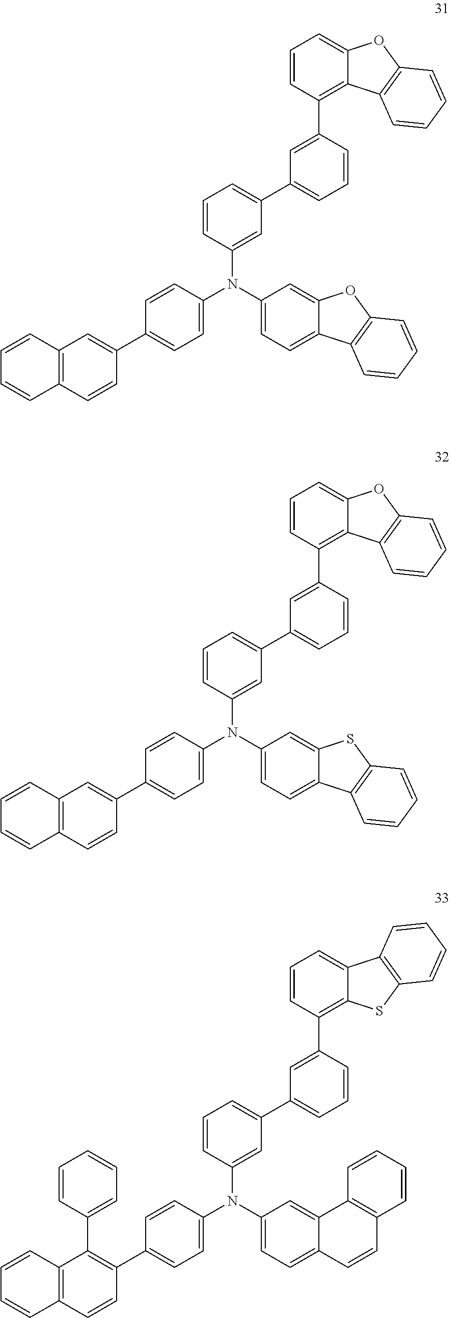 [SMILES: c1cc(-c2cccc(N(c3ccc(-c4ccc5ccccc5c4)cc3)c3ccc4c(c3)oc3ccccc34)c2)cc(-c2cccc3oc4ccccc4c23)c1 ; c1cc(-c2cccc(N(c3ccc(-c4ccc5ccccc5c4)cc3)c3ccc4c(c3)sc3ccccc34)c2)cc(-c2cccc3oc4ccccc4c23)c1 ; c1ccc(-c2c(-c3ccc(N(c4cccc(-c5cccc(-c6cccc7c6sc6ccccc67)c5)c4)c4ccc5ccc6ccccc6c5c4)cc3)ccc3ccccc23)cc1] 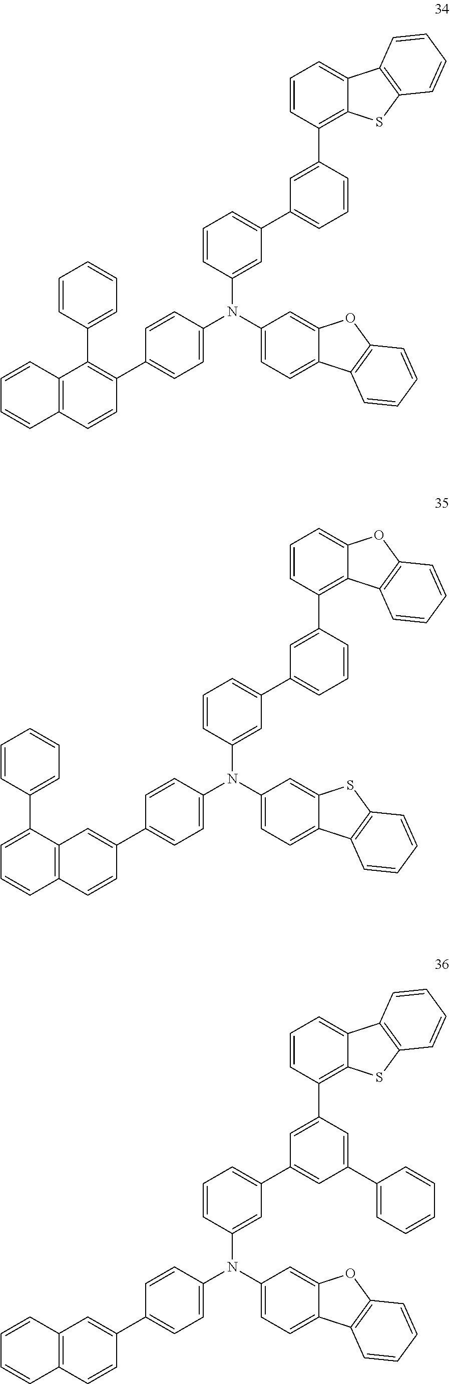 [SMILES: c1ccc(-c2c(-c3ccc(N(c4cccc(-c5cccc(-c6cccc7c6sc6ccccc67)c5)c4)c4ccc5c(c4)oc4ccccc45)cc3)ccc3ccccc23)cc1 ; c1ccc(-c2cccc3ccc(-c4ccc(N(c5cccc(-c6cccc(-c7cccc8oc9ccccc9c78)c6)c5)c5ccc6c(c5)sc5ccccc56)cc4)cc23)cc1 ; c1ccc(-c2cc(-c3cccc(N(c4ccc(-c5ccc6ccccc6c5)cc4)c4ccc5c(c4)oc4ccccc45)c3)cc(-c3cccc4c3sc3ccccc34)c2)cc1] 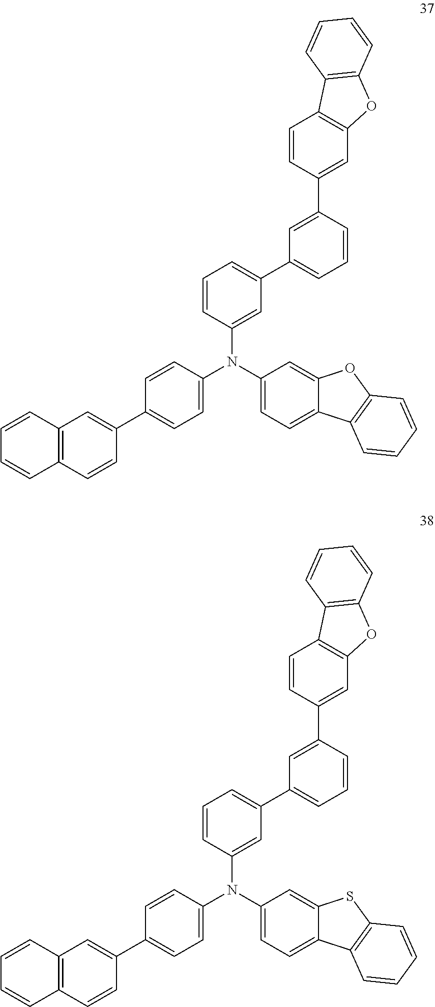 [SMILES: c1cc(-c2cccc(N(c3ccc(-c4ccc5ccccc5c4)cc3)c3ccc4c(c3)oc3ccccc34)c2)cc(-c2ccc3c(c2)oc2ccccc23)c1 ; c1cc(-c2cccc(N(c3ccc(-c4ccc5ccccc5c4)cc3)c3ccc4c(c3)sc3ccccc34)c2)cc(-c2ccc3c(c2)oc2ccccc23)c1] 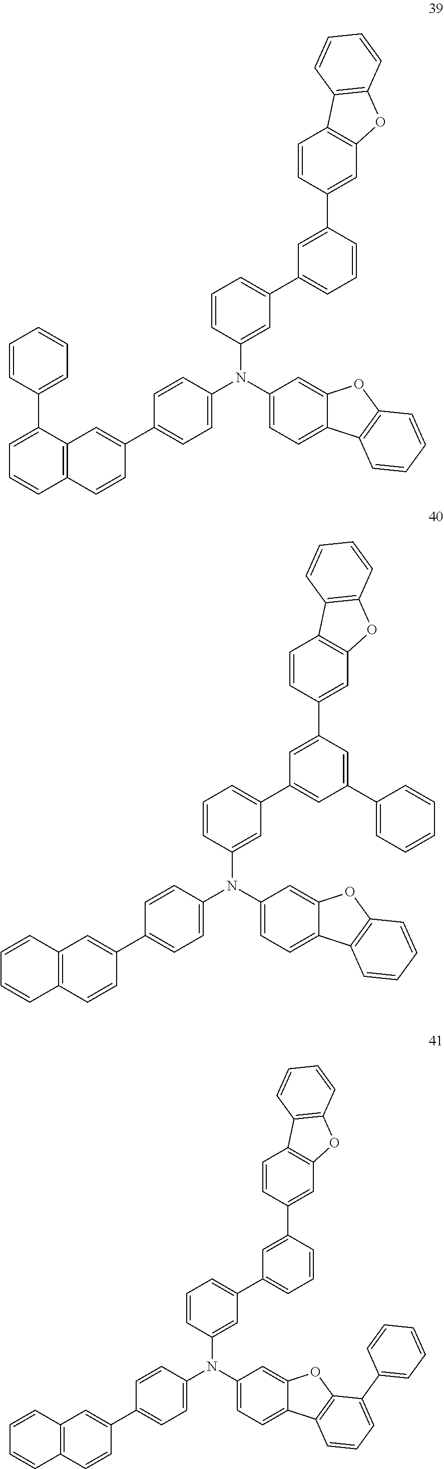 [SMILES: c1ccc(-c2cccc3ccc(-c4ccc(N(c5cccc(-c6cccc(-c7ccc8c(c7)oc7ccccc78)c6)c5)c5ccc6c(c5)oc5ccccc56)cc4)cc23)cc1 ; c1ccc(-c2cc(-c3cccc(N(c4ccc(-c5ccc6ccccc6c5)cc4)c4ccc5c(c4)oc4ccccc45)c3)cc(-c3ccc4c(c3)oc3ccccc34)c2)cc1 ; c1ccc(-c2cccc3c2oc2cc(N(c4ccc(-c5ccc6ccccc6c5)cc4)c4cccc(-c5cccc(-c6ccc7c(c6)oc6ccccc67)c5)c4)ccc23)cc1] 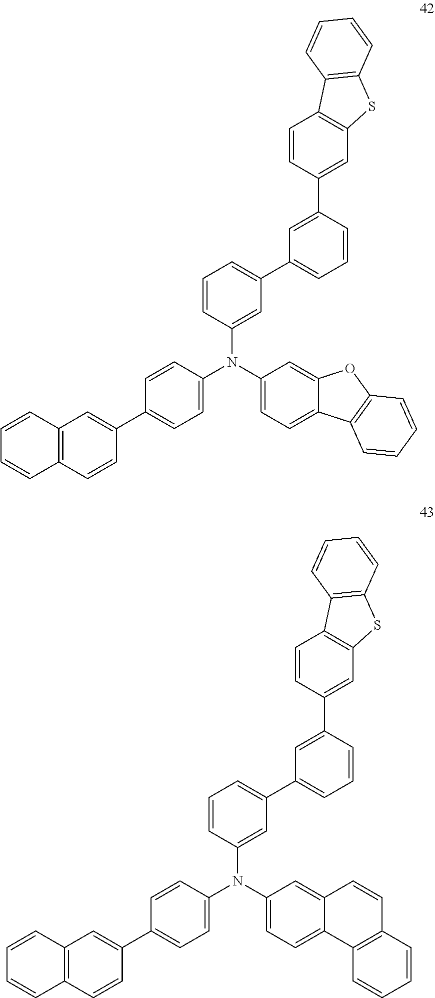 [SMILES: c1cc(-c2cccc(N(c3ccc(-c4ccc5ccccc5c4)cc3)c3ccc4c(c3)oc3ccccc34)c2)cc(-c2ccc3c(c2)sc2ccccc23)c1 ; c1cc(-c2cccc(N(c3ccc(-c4ccc5ccccc5c4)cc3)c3ccc4c(ccc5ccccc54)c3)c2)cc(-c2ccc3c(c2)sc2ccccc23)c1] 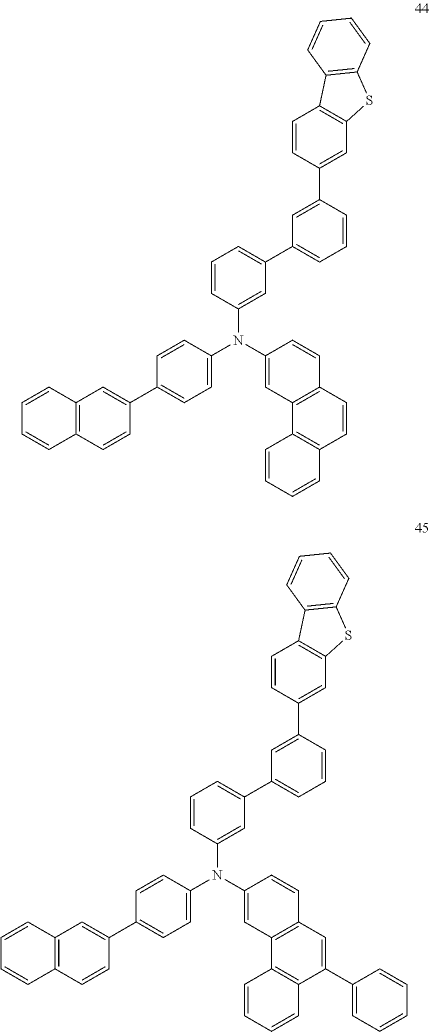 [SMILES: c1cc(-c2cccc(N(c3ccc(-c4ccc5ccccc5c4)cc3)c3ccc4ccc5ccccc5c4c3)c2)cc(-c2ccc3c(c2)sc2ccccc23)c1 ; c1ccc(-c2cc3ccc(N(c4ccc(-c5ccc6ccccc6c5)cc4)c4cccc(-c5cccc(-c6ccc7c(c6)sc6ccccc67)c5)c4)cc3c3ccccc23)cc1] 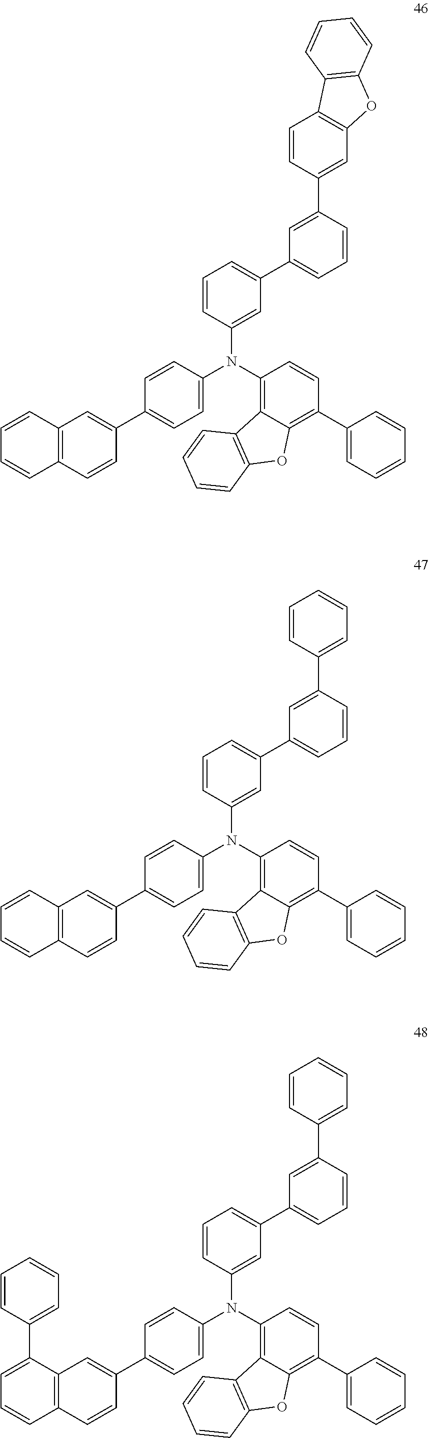 [SMILES: c1ccc(-c2ccc(N(c3ccc(-c4ccc5ccccc5c4)cc3)c3cccc(-c4cccc(-c5ccc6c(c5)oc5ccccc56)c4)c3)c3c2oc2ccccc23)cc1 ; c1ccc(-c2cccc(-c3cccc(N(c4ccc(-c5ccc6ccccc6c5)cc4)c4ccc(-c5ccccc5)c5oc6ccccc6c45)c3)c2)cc1 ; c1ccc(-c2cccc(-c3cccc(N(c4ccc(-c5ccc6cccc(-c7ccccc7)c6c5)cc4)c4ccc(-c5ccccc5)c5oc6ccccc6c45)c3)c2)cc1] 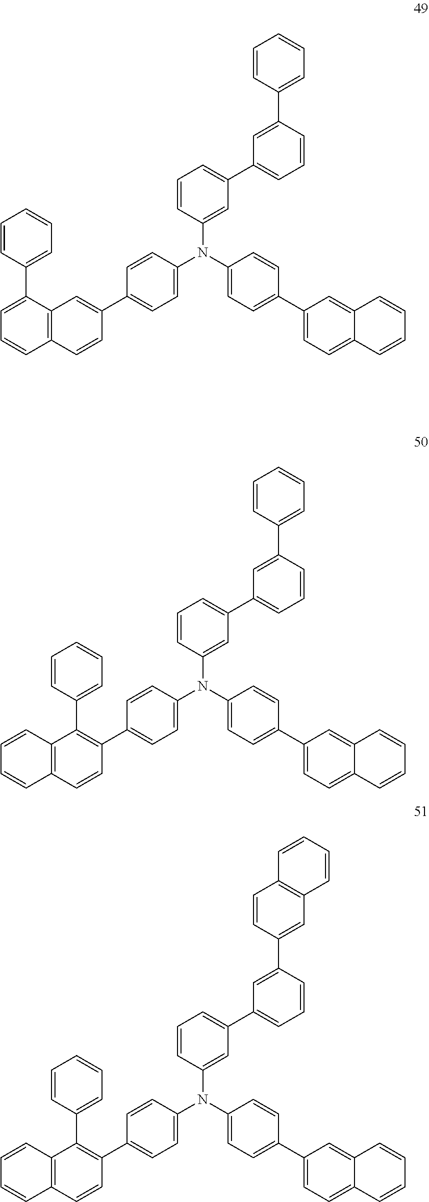 [SMILES: c1ccc(-c2cccc(-c3cccc(N(c4ccc(-c5ccc6ccccc6c5)cc4)c4ccc(-c5ccc6cccc(-c7ccccc7)c6c5)cc4)c3)c2)cc1 ; c1ccc(-c2cccc(-c3cccc(N(c4ccc(-c5ccc6ccccc6c5)cc4)c4ccc(-c5ccc6ccccc6c5-c5ccccc5)cc4)c3)c2)cc1 ; c1ccc(-c2c(-c3ccc(N(c4ccc(-c5ccc6ccccc6c5)cc4)c4cccc(-c5cccc(-c6ccc7ccccc7c6)c5)c4)cc3)ccc3ccccc23)cc1] 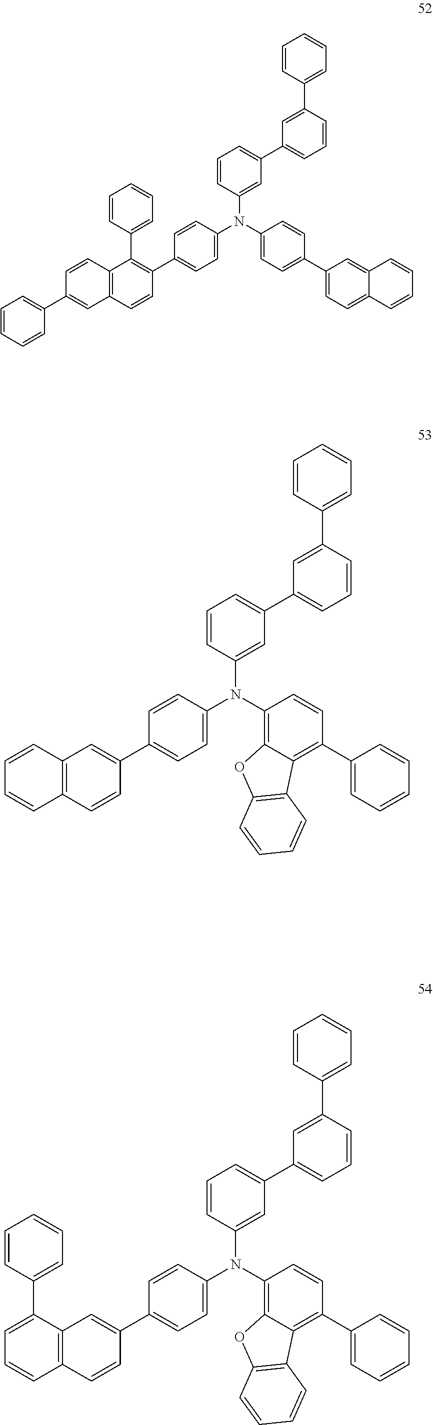 [SMILES: c1ccc(-c2cccc(-c3cccc(N(c4ccc(-c5ccc6ccccc6c5)cc4)c4ccc(-c5ccc6cc(-c7ccccc7)ccc6c5-c5ccccc5)cc4)c3)c2)cc1 ; c1ccc(-c2cccc(-c3cccc(N(c4ccc(-c5ccc6ccccc6c5)cc4)c4ccc(-c5ccccc5)c5c4oc4ccccc45)c3)c2)cc1 ; c1ccc(-c2cccc(-c3cccc(N(c4ccc(-c5ccc6cccc(-c7ccccc7)c6c5)cc4)c4ccc(-c5ccccc5)c5c4oc4ccccc45)c3)c2)cc1] 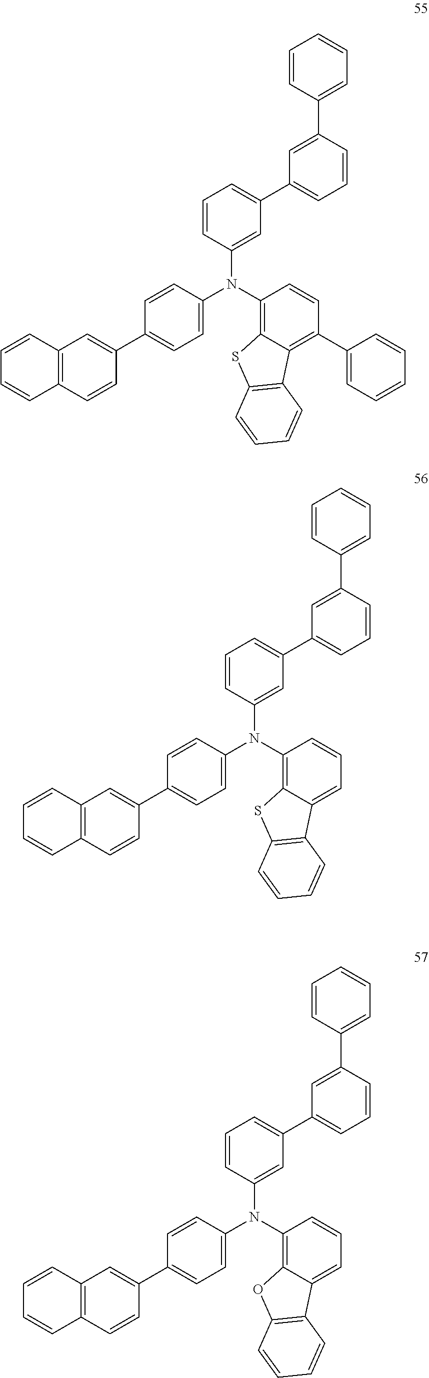 [SMILES: c1ccc(-c2cccc(-c3cccc(N(c4ccc(-c5ccc6ccccc6c5)cc4)c4ccc(-c5ccccc5)c5c4sc4ccccc45)c3)c2)cc1 ; c1ccc(-c2cccc(-c3cccc(N(c4ccc(-c5ccc6ccccc6c5)cc4)c4cccc5c4sc4ccccc45)c3)c2)cc1 ; c1ccc(-c2cccc(-c3cccc(N(c4ccc(-c5ccc6ccccc6c5)cc4)c4cccc5c4oc4ccccc45)c3)c2)cc1] 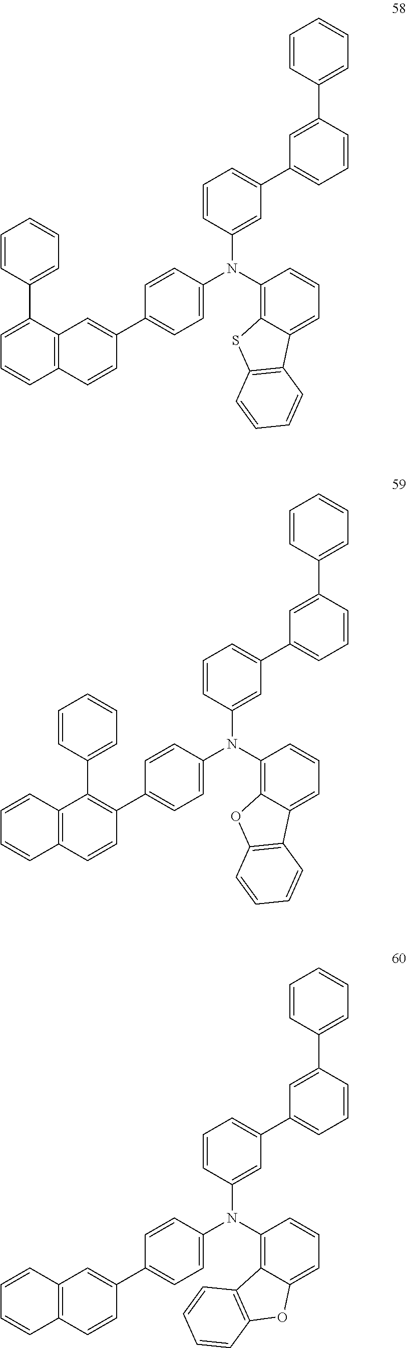 [SMILES: c1ccc(-c2cccc(-c3cccc(N(c4ccc(-c5ccc6cccc(-c7ccccc7)c6c5)cc4)c4cccc5c4sc4ccccc45)c3)c2)cc1 ; c1ccc(-c2cccc(-c3cccc(N(c4ccc(-c5ccc6ccccc6c5-c5ccccc5)cc4)c4cccc5c4oc4ccccc45)c3)c2)cc1 ; c1ccc(-c2cccc(-c3cccc(N(c4ccc(-c5ccc6ccccc6c5)cc4)c4cccc5oc6ccccc6c45)c3)c2)cc1] 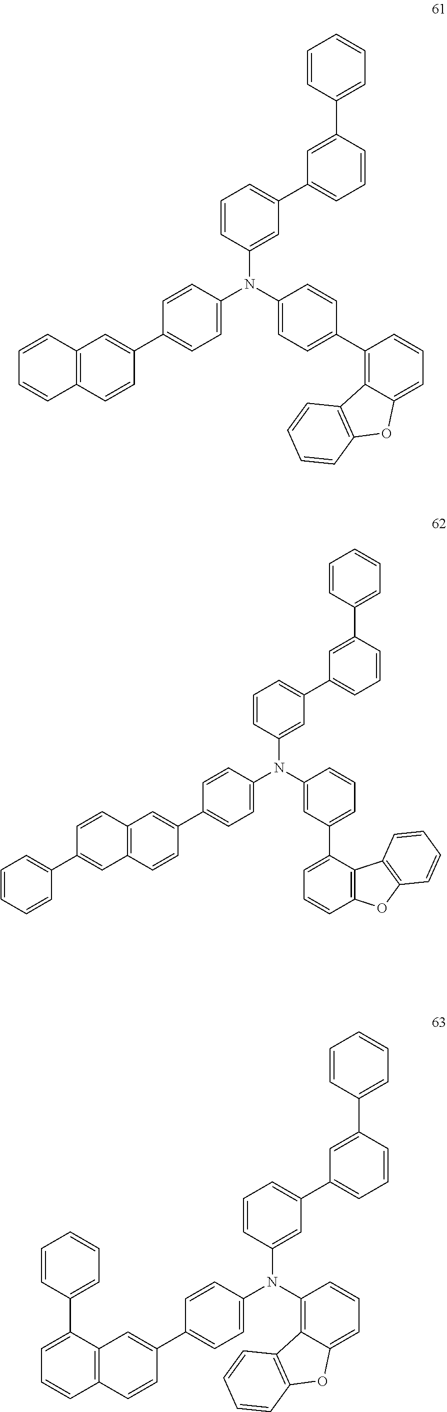 [SMILES: c1ccc(-c2cccc(-c3cccc(N(c4ccc(-c5ccc6ccccc6c5)cc4)c4ccc(-c5cccc6oc7ccccc7c56)cc4)c3)c2)cc1 ; c1ccc(-c2cccc(-c3cccc(N(c4ccc(-c5ccc6cc(-c7ccccc7)ccc6c5)cc4)c4cccc(-c5cccc6oc7ccccc7c56)c4)c3)c2)cc1 ; c1ccc(-c2cccc(-c3cccc(N(c4ccc(-c5ccc6cccc(-c7ccccc7)c6c5)cc4)c4cccc5oc6ccccc6c45)c3)c2)cc1] 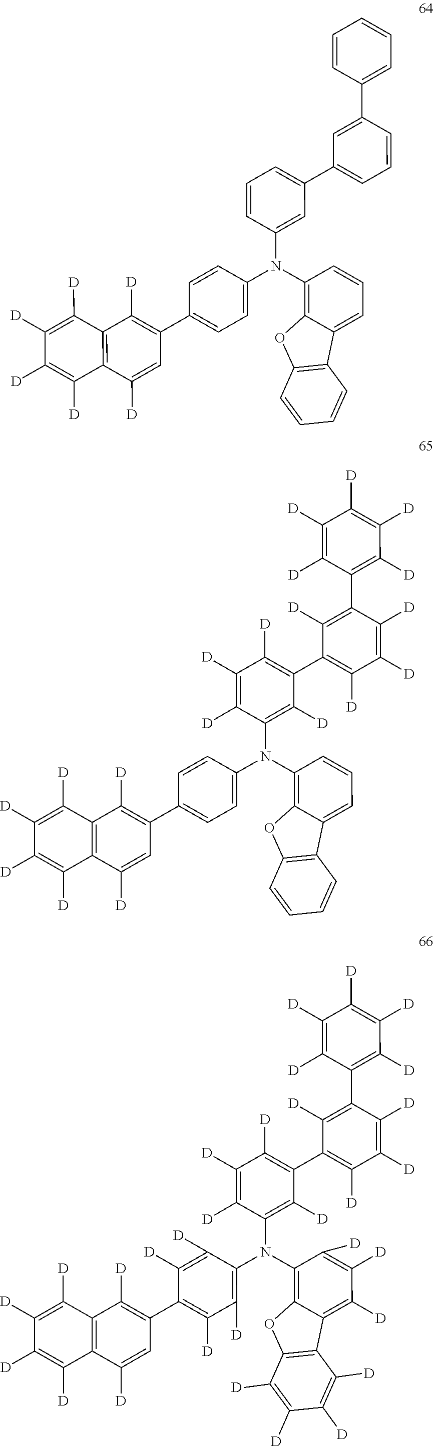 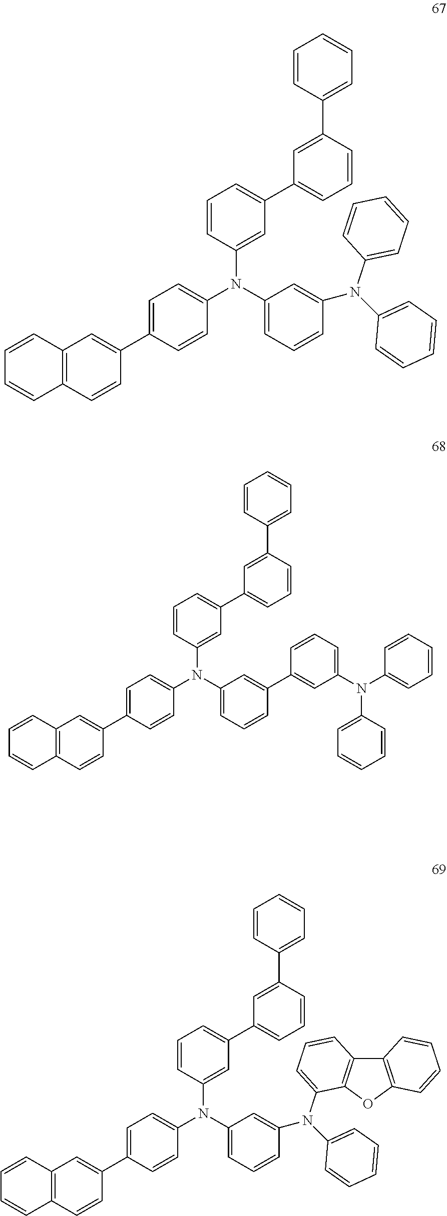 [SMILES: c1ccc(-c2cccc(-c3cccc(N(c4ccc(-c5ccc6ccccc6c5)cc4)c4cccc(N(c5ccccc5)c5ccccc5)c4)c3)c2)cc1 ; c1ccc(-c2cccc(-c3cccc(N(c4ccc(-c5ccc6ccccc6c5)cc4)c4cccc(-c5cccc(N(c6ccccc6)c6ccccc6)c5)c4)c3)c2)cc1 ; c1ccc(-c2cccc(-c3cccc(N(c4ccc(-c5ccc6ccccc6c5)cc4)c4cccc(N(c5ccccc5)c5cccc6c5oc5ccccc56)c4)c3)c2)cc1]
wherein in Compound Group 1 above, D is a deuterium atom.
